(19) United States
(12) Patent Application Publication
(10) Pub. No.: US 2014/0140931 A1
(43) Pub. Date: May 22, 2014

(54) ANTIOXIDANT, NEUROPROTECTIVE AND ANTINEOPLASTIC NANOPARTICLES COMPRISING A THERAPEUTIC AGENT ON AN AMPHIPHILIC SPACER OR AN AMPHIPHILIC POLYMER

(71) Applicants: John S. Yu, Los Angeles, CA (US); Bong Seop Lee, Torrance, CA (US)

(72) Inventors: John S. Yu, Los Angeles, CA (US); Bong Seop Lee, Torrance, CA (US)

(73) Assignee: CEDARS-SINAI MEDICAL CENTER, Los Angeles, CA (US)

(21) Appl. No.: 14/232,871

(22) PCT Filed: Jul. 27, 2012
(86) PCT No.: PCT/US12/48703

§ 371 Date: Jan. 14, 2014

Related U.S. Application Data
(60) Provisional application No. 61/512,664, filed on Jul. 28, 2011.

(51) Int. Cl. A61K 47/48; A61K 49/00

(52) U.S. Cl. CPC A61K 47/48869; A61K 49/0093
USPC 424/9.6; 514/283; 514/440; 514/510; 514/532; 514/539; 514/414; 424/489

(57) ABSTRACT

This invention relates to antioxidant, neuroprotective and antineoplastic nanoparticles comprising a therapeutic agent on an amphiphilic spacer or an amphiphilic polymer. Methods of synthesizing the antioxidant derivatives of camptothecin and anti-oxidant derivatives of camptothecin analogs, NSAIDs and statins, spontaneous emulsification or nanoprecipitation thereof to produce antioxidant, neuroprotective and anti-neoplastic nanoparticles comprising a therapeutic agent on an amphiphilic spacer or an amphiphilic polymer and their use in treating cancerous diseases are also provided. A further aspect of this invention is the use of these neuroprotective and anti-neoplastic nanoparticles for the preparation of delivery devices of other pharmaceuticals and/or drugs.

### FIELD OF INVENTION

This invention relates to antioxidant and antineoplastic nanoparticles comprising a therapeutic agent on an amphiphilic spacer or an amphiphilic polymer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Antineoplastic Effect of Camptothecin

Camptothecin is a plant alkaloid first isolated from the wood and barks of Camptotheca acuminate (Nyssaceae) and exhibits its antineoplastic effect by the inhibition of DNA relaxation by DNA topoisomerase I. However, camptothecin is essentially insoluble in water, and therefore, numerous derivatives have been developed to increase the water solubility (Thomas et al., Camptothecin: Current perspectives. BIOORG. MED. CHEM., 12, 2004, 1585-1604: Pizzolato et al., The Camptothecin. THE LANCET, 361, 2003, 2235-2242).

Camptothecin consists of a pentacyclic structure having a lactone in the E-ring, which is essential for antitumor effects of the molecule. It has been demonstrated that the main transformation and elimination pathways of the drug comprise lactone hydrolysis and urinary excretion. In fact, the lactone form is 50% hydrolyzed to an open ring 30 minutes after administration. The sodium salt showed a lower activity than camptothecin, because at pH 7.4 the inactive form (open ring) predominates on the lactone active form (closed ring).

### Non-Steroidal Anti-Inflammatory Drugs

Non-steroidal anti-inflammatory drugs (NSAIDs) are widely used in the treatment of pain, fever, and inflammation. The major mechanism by which NSAIDs exert their anti-inflammatory activity is the inhibition of cyclooxygenase-derived prostaglandin synthesis, which is also responsible for adverse side effects, such as irritation and ulceration of the gastrointestinal (GI) mucosa (Whittle, 2003). There are two types of COX enzymes, namely COX-1 and COX-2. COX-1 is expressed constitutively in many tissues, whereas COX-2 is expressed only at the site of inflammation (S. Kargan et al. GASTROENTEROL., 111: 445-454, 1996). The prostaglandins whose production is mediated by COX-1 are responsible for the maintenance of gastric mucosal integrity. Thus, the GI side effects are generally believed to result from the combined effect of the irritation caused by the free carboxylic groups in NSAIDs and blockage of prostaglandin biosynthesis in the GI tract (Dannhardt and Kiefer, 2001). In addition to the side effect which is attributed to their inhibitory effect on the activity of cyclooxygenase, the acidic moiety of these NSAIDs also contributes to the gastrointestinal side effect observed in response to these drugs (Tammara et al., 1993).

Epidemiologic studies have documented that a subset of NSAIDs decrease the risk for Alzheimer's disease (AD). The efficacy of NSAIDs in AD might be attributable to either anti-inflammatory or anti-amyloidogenic activities. It has been reported that ibuprofen, indomethacin and sulindac sulphide decrease the highly amyloidogenic Aβ42 peptide independently of COX activity (NATURE, 414:212-216 (2001)).

NSAIDs have also been shown to inhibit angiogenesis through direct effects on endothelial cells.

Although inflammatory oxidant hypochlorous acid (HOCl) generated by the myeloperoxidase (MPO)-H₂O₂/Cl⁻ system comprises an important mechanism of host defense against infection, the overproduction and extracellularly generated HOCl is cytotoxic and is believed to be implicated in the pathogenesis of numerous diseases including neurodegenerative disorders, atherosclerosis, chronic inflammatory conditions, and cancer (Malle et al., BR J PHARMACOL 2007: 1-17).

Hypochlorous acid is a powerful oxidizing agent that can react with many biological molecules. In the presence of physiological concentration of chloride ions, H₂O₂ is efficiently halogenated by the heme enzyme MPO to yield hypochlorous acid, by far the most abundant oxidant generated by activated phagocyte cells (Krasowska et al., BRAIN RES. 997:176-184 (2004)). Hypochlorous acid can chlorinate cytosolic proteins and nuclear DNA bases and induce lipid peroxidation in phospholipid and lipoprotein (Spickett C M., PHARMACOL THERAPEUTICS 115:400-409 (2007)). Importantly, the damages caused by HOCl to the intracellular glutathione and protein thiols are irreversible and can be replaced only by resynthesis (Dalle-Donne et al., FREE RADIC BIOL MED 32(9):927-937 (2002)). Furthermore, HOCl can be converted into damaging hydroxyl radicals (Candeias et al., FEBS LETT 333(1,2):151-153 (1993)). Most NSAIDs are able to scavenge hypochlorous acid in the aqueous environment and some NSAIDs inhibit the MPO by direct interaction with the enzyme (Neve et al., EUROPEAN J PHARMACOL 417:37-43 (2001)).

### Anticancer Effects of NSAIDs

A number of epidemiologic studies, clinical trials, and animal studies have shown that NSAIDs may be effective in the prevention and treatment of certain cancers. (Keller et al., Chemoprevention strategies using NSAIDs and COX-2 inhibitors. CANCER BIOL THER (2003) 2:S140-9; Gupta et al., Colorectal cancer prevention and treatment by inhibition of cyclooxygenase-2. NAT REV CANCER (2001) 1:11-21; Umar et al., Development of COX inhibitors in cancer prevention and therapy. AM J CLIN ONCOL (2003) 26:S48-57; Harris et al., Aspirin, ibuprofen, and other non-steroidal anti-inflammatory drugs in cancer prevention: a critical review of nonselective COX-2 blockade [review]. ONCOL REP 2005; 13: 559-83). It has also been suggested that the long term use of certain NSAIDs reduces the risk of colorectal, breast, and ovarian cancer. Taketo et al., Cyclooxygenase-2 inhibitors in tumorigenesis. J NATL CANCER INST (1998) 90:1529-36; Sandler et al. A randomized trial of aspirin to prevent colorectal adenomas. N ENGL J MED (2003) 348:891-9; Saji et al. Novel sensitizing agents: potential contribution of COX-2 inhibitor for endocrine therapy of breast cancer. BREAST CANCER (2004) 11:129-33.

The molecular mechanisms by which NSAIDs exhibit antineoplastic effects are poorly understood and a matter of intensive investigation. The chemopreventive and antitumorigenic effects of NSAIDs are partially attributed to the induction of apoptosis followed by inhibition of COX-2. Lin et al., The role of cyclooxygenase-2 inhibition for the prevention and treatment of prostate carcinoma. CLIN PROSTATE CANCER (2003) 2:119-26; Mann et al., Cyclooxygenase-2 and gastrointestinal cancer. CANCER J (2004) 10:145-52; Basler et al., Nonsteroidal anti-inflammatory drugs and cyclooxygenase-2 selective inhibitors for prostate cancer chemoprevention. J UROL 2004; 171: S59-62; discussion S62-53; Sabichi et al., COX-2 inhibitors and other nonsteroidal anti-inflammatory drugs in genitourinary cancer. SEMIN ONCOL 2004; 31:36-44.

Various studies have also suggested that a COX-2-independent mechanism may also be involved because apoptosis induction by NSAIDs does not always correlate with their ability to inhibit COX-2. Chuang et al., COX-2 inhibition is neither necessary nor sufficient for celecoxib to suppress tumor cell proliferation and focus formation in vitro. MOL CANCER (2008) 7:38; Marx et al., J. Cancer research; Anti-inflammatories inhibit cancer growth—but how? SCIENCE 2001; 291:581-2; Elder et al., Induction of apoptotic cell death in human colorectal carcinoma cell lines by a cyclooxygenase-2 (COX-2)-selective nonsteroidal anti-inflammatory drug: independence from COX-2 protein expression. CARCINOGENESIS (2001) 22:17-25; Jiang et al., Subtraction hybridization identifies a novel melanoma differentiation associated gene, mda-7, modulated during human melanoma differentiation, growth and progression. ONCOGENE (1995) 11:2477-86.

### Antioxidant Effect of α-Lipoic Acid

Molecules containing a dithiolane moiety are widely investigated due to their antioxidant properties. α-Lipoic acid (thioctic acid, 1,2-dithiolane-3-pentanoic acid), which has dithiolane ring in its molecule, is a widely distributed natural substance which was originally discovered as a growth factor. Physiologically, it acts as a coenzyme of the oxidative decarboxylation of α-keto carboxylic acid (e.g., pyruvates) and as an antioxidant, and it is able to regenerate vitamin C, vitamin E, glutathione and coenzyme Q10. In pathological conditions, lipoic acid is applied in the treatment of diabetic polyneuropathy, liver cirrhosis and metal intoxications.

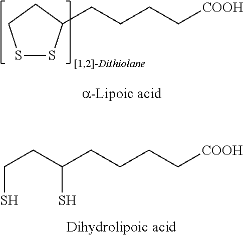

Lipoic acid and dihydrolipoic acid are capable of trapping a number of radicals both in a lipid and in an aqueous environment. Lipoic acid and dihydrolipoic acid act as antioxidants not only by direct radical trapping and/or metal chelation but also by recycling other antioxidants (e.g., vitamin C, vitamin E) and by reducing glutathione, which in turn recycles vitamin E. The two thiol groups present in [1,2]-dithiolane ring system confer it a unique antioxidant potential. The disulfides with a cyclic five-member ring such as lipoic acid have been found to be more effective in reductive and/or nucleophilic attack than open-chain derivatives such as cystine or glutathione.

The antioxidant potential of a compound may be evaluated based on the properties such as (1) specificity of free radical scavenging, (2) interaction with other antioxidants, (3) metal-chelating activity, (4) effects on gene expression, (5) absorption and bioavailability, (6) location (in aqueous or membrane domains, or both), and (7) ability to repair oxidative damage (Packer et al., FREE RADICAL BIOLOGY & MEDICINE. 19(2):227-250, 1995). According to the above criteria, the [1,2]-dithiolane containing lipoic acid/dihydrolipoic acid redox system has been regarded as a universal antioxidant.

There have been many attempts to develop lipoic acid derivatives or complexes having antioxidant activity. U.S. Pat. Nos. 6,090,842; 6,013,663; 6,117,899; 6,127,394; 6,150,358; 6,204,288, 6,235,772; 6,288,106; 6,353,011; 6,369,098; 6,387,945; 6,605,637; 6,887,891; 6,900,338; and 6,936,715 are some examples.

In many other U.S. patents, the natural and synthetic lipoic acid derivatives and their metabolites are disclosed for use in preventing skin aging and in the treatment of free radical mediated diseases, including inflammatory, proliferative, neurodegenerative, metabolic and infectious diseases.

### Inhibitory Activity on NO-Synthase and Trapping the Reactive Oxygen Species (ROS)

Various conditions or disease conditions have demonstrated a potential role of nitric oxide (NO) and the ROS's and the metabolism of glutathione in their physiopathology. Conditions or disease conditions where nitrogen monoxide and the metabolism of glutathione as well as the redox status of thiol groups are involved include but are not limited to: cardiovascular and cerebrovascular disorders (e.g., atherosclerosis, migraine, arterial hypertension, septic shock, ischemic or hemorrhagic cardiac or cerebral infarctions, ischemias and thromboses); disorders of the central or peripheral nervous system (e.g., neurodegenerative nervous system); neurodegenerative diseases including cerebral infarctions, sub-arachnoid hemorrhaging, ageing, senile dementias (e.g., Alzheimer's disease), Huntington's chorea, Parkinson's disease, prion disease (e.g., Creutzfeld Jacob disease), amyotrophic lateral sclerosis, pain, cerebral and spinal cord traumas; proliferative and inflammatory diseases (e.g., atherosclerosis), amyloidoses, and inflammations of the gastro-intestinal system; organ transplantation; diabetes and its complications (e.g., retinopathies, nephropathies and polyneuropathies, multiple sclerosis, myopathies); cancer; autosomal genetic diseases (e.g., Unverricht-Lundborg disease); neurological diseases associated with intoxications (e.g., cadmium poisoning, inhalation of n-hexane, pesticides, herbicides), associated with treatments (e.g., radiotherapy) or disorders of genetic origin (e.g., Wilson's disease); and impotence linked to diabetes.

These conditions and disease conditions are characterized by an excessive production or a dysfunction of nitrogen monoxide and/or the metabolism of glutathione and of the redox status of the thiol groups (Duncan and Heales, Nitric Oxide and Neurological Disorders, MOLECULAR ASPECTS OF MEDICINE. 26:67-96, 2005; Kerwin et al., Nitric Oxide: A New Paradigm For Second Messengers, J. MED. CHEM. 38:4343-4362, 1995; Packer et al., FREE RADICAL BIOLOGY & MEDICINE. 19:227-250, 1995). U.S. Pat. Nos. 6,605,637, 6,887,891, and 6,936,715 disclose that lipoic acid derivatives inhibit the activity of NO-synthase enzymes producing nitrogen monoxide NO and regenerate endogenous antioxidants which trap the ROS and which intervene in a more general fashion in the redox status of thiol groups. U.S. Pat. Nos. 5,693,664, 5,948,810, and 6,884,420 disclose the use of racemic α-lipoic acid or their metabolites, salts, amides or esters for the synthesis of drugs for the treatment of diabetes mellitus of types I and II. U.S. Pat. No. 5,925,668 discloses a method of treating free radical mediated diseases, and/or reducing the symptoms associated with such diseases whereby the compounds with antioxidant activity contain 1,2-dithiolane, reduced or oxidized forms. U.S. Pat. No. 6,251,935 discloses methods for the prevention or treatment of migraine comprising the administration of an active ingredient selected from the group consisting of racemic alpha-lipoic acid, enantiomers and pharmaceutically acceptable salts, amides, esters or thioesters thereof. U.S. Pat. Nos. 6,472,432 and 6,586,472 disclose the treatment of a chronic inflammatory disorder rosacea by application of a composition containing lipoic acid and/or lipoic acid derivatives. There is also strong evidence that the neuroprotective effects of lipoic acid and dihydrolipoic acid are mediated by antioxidant and free radical scavenging mechanisms (Packer et al., FREE RADICAL BIOLOGY & MEDICINE. 22:359-378, 1997).

### Neuroprotective and Neurorestorative Effects of Statins

Statins are cholesterol biosynthesis inhibitors used for lowering cholesterol level. Statins also show neuroprotective and neurorestorative benefits in animal models of traumatic brain injury (TBI) and stroke (Chen et al., Ann Neurol 53(6), 743-751, 2003; Jessberger et al., Learn Mem 16(2), 147-154, 2009; Chen et al., Life Sci 81(4), 288-298, 2007; Chen et al., J Cereb Blood Flow Metab 25(2), 281-290, 2005; Lu et al., J Neurotrauma, 21(1), 21-32, 2004; Lu et al., J Neurosurg, 101 (5):813-821, 2004. Wu et al., J Neurosurg, 109(4):691-698, 2008). Traumatic brain injury caused by stroke and trauma is a major health problem worldwide. Ischemia also plays an important role in pathogenesis of TBI. Statins enhance functional recovery after TBI, significantly reduce the neurological functional deficits, and increase neuronal survival (Chen et al., Ann Neurol, 53(6), 743-751, 2003; Lu et al., J Neurotrauma, 24(7): 1132-1146, 2007; Wang et al., Exp Neurol, 206(1), 59-69, 2007).

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a nanosphere comprising: tocopherol and a therapeutic agent or an imaging agent conjugated to a hydrophilic spacer, a hydrophobic spacer, an amphiphilic spacer, or an amphiphilic polymer.

In various embodiments, the nanosphere further comprises an antioxidant α-lipoic acid-containing hydrophobic compound having Formula A-Ia as described herein, wherein X may be selected from the group consisting of a substituted, unsubstituted, branched or unbranched chain of carbon atoms, and may optionally contain a heteroatom; Y may be selected from the group consisting of a branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic group; and n may be an integer of at least one. In various embodiments, the dithiolane moiety in Formula Ia may be an α-lipoic acid and is represented by Formula A-IIa as described herein.

In various embodiments, the nanosphere further comprises a hydrophobic nonsteroidal anti-inflammatory drug (NSAID) derivative having Formula B-I as described herein, wherein the A may be selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; and n may be an integer of at least two.

In various embodiments, the nanosphere further comprises a hydrophobic antioxidant and anti-inflammatory derivative of an nonsteroidal anti-inflammatory drug (NSAID) having Formula B-II as described herein, wherein X may be selected from the group consisting of a substituted, unsubstituted, branched or unbranched chain of carbon atoms and may optionally contain a heteroatom; A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; n may be an integer of at least one; and m may be an integer of at least one. In various embodiments, the hydrophobic antioxidant and anti-inflammatory derivative of an NSAID is Formula B-III as described herein, wherein ALA represents α-lipoic acid.

In various embodiments the nanosphere further comprises an antioxidant derivative of camptothecin and/or an antioxidant derivative of a camptothecin analog.

In various embodiments, the antioxidant derivative of camptothecin and/or an antioxidant derivative of a camptothecin analog may be Formula C-II, as described herein, wherein A and B may be independently selected from the group consisting of —OC(O)—, —OC(O)O—, and —OC(O)N(R)—, wherein R may be a hydrogen atom, or a substituted, unsubstituted, branched or unbranched chain of carbon atoms; X and Y may be linkers, each independently comprising a substituted, unsubstituted, branched or unbranched chain of carbon atoms and may optionally contain a heteroatom; and R₁, R₂, R₃, R₄, and R₅ may each be independently selected from the group consisting of hydrogen, alkyl, aryl, cycloaliphatic, and aralkyl and may each optionally contain a hetero atom.

In various embodiments, the antioxidant derivative of camptothecin and/or an antioxidant derivative of a camptothecin analog may be Formula C-IV, as described herein, wherein L₁ may be a moiety formed by esterification of two free esterifiable hydroxyl groups on a diol; and R₁, R₂, R₃, R₄, and R₅ may each be independently selected from the group consisting of hydrogen, alkyl, aryl, cycloaliphatic, and aralkyl group, and may optionally contain a hetero atom.

In various embodiments, the antioxidant derivative of camptothecin and/or an antioxidant derivative of a camptothecin analog may be selected from the group consisting of: Formula C-V, Formula C-VI, Formula C-VII, Formula C-VIII, Formula C-IX, Formula C-X, and Formula C-XLVI, as described herein.

In various embodiments, the antioxidant derivative of camptothecin and/or an antioxidant derivative of a camptothecin analog may be Formula C-III, as described herein, wherein A may be selected from the group consisting of —OC(O)—, —OC(O)O—, and —OC(O)N(R)—, wherein R may be a hydrogen atom, or a substituted, unsubstituted, branched or unbranched chain of carbon atoms; P may be selected from the group consisting of —OC(O)—, and —N(R)C(O)—, wherein R may be a hydrogen atom, or a substituted, unsubstituted, branched or unbranched chain of carbon atoms; X may be a linker comprising a substituted, unsubstituted, branched or unbranched chain of carbon atoms and may optionally contain a heteroatom; and R₁, R₂, R₃, R₄, and R₅ may each be independently selected from the group consisting of hydrogen, alkyl, aryl, cycloaliphatic, and aralkyl, and may each optionally contain a hetero atom.

In various embodiments, the antioxidant derivative of camptothecin and/or an antioxidant derivative of a camptothecin analog may be Formula C-XI, as described herein, wherein L₂ may be a moiety formed by using a diamine as the linker in the process of producing the compound; and R₁, R₂, R₃, R₄, and R₅ may each be independently selected from the group consisting of hydrogen, alkyl, aryl, cycloaliphatic, and aralkyl group, and may optionally contain a hetero atom.

In various embodiments, the antioxidant derivative of camptothecin and/or an antioxidant derivative of a camptothecin analog may be selected from the group consisting of: Formula C-XII, Formula C-XIII, Formula C-XIV, Formula C-XV, Formula C-XVI, Formula C-XVII, and Formula C-XLVII, as described herein.

In various embodiments, the antioxidant derivative of camptothecin and/or an antioxidant derivative of a camptothecin analog may be Formula C-XVIII, as described herein, wherein L₃ may be a moiety formed by using an aminoalcohol as the linker in the process of producing the compound; and R₁, R₂, R₃, R₄, and R₅ may each be independently selected from the group consisting of hydrogen, alkyl, aryl, cycloaliphatic, and aralkyl, and may each contain a hetero atom.

In various embodiments, the antioxidant derivative of camptothecin and/or an antioxidant derivative of a camptothecin analog may be selected from the group consisting of: Formula C-XIX, Formula C-XX, Formula C-XXI, Formula C-XXII, Formula C-XXIII, Formula C-XXIV, and Formula C-XLVIII, as described herein.

In various embodiments the antioxidant derivative of camptothecin and/or an antioxidant derivative of a camptothecin analog may be a compound produced by conjugation of an α-lipoic acid and camptothecin or a camptothecin analog modified by reacting with succinic anhydride or glutaric anhydride, wherein the camptothecin analog is represented by Formula C-I, as described herein, wherein R₁, R₂, R₃, R₄, and R₅ may each be independently selected from the group consisting of hydrogen, alkyl, aryl, cycloaliphatic, and aralkyl, and may optionally contain a hetero atom.

In various embodiments, the antioxidant derivative of camptothecin and/or an antioxidant derivative of a camptothecin analog may be selected from the group consisting of: Formula C-XXV, Formula C-XXVI, Formula C-XXVII, Formula C-XXVIII, Formula C-XXIX, Formula C-XXX, Formula C-XXXI, Formula C-XXXII, Formula C-XXXIII, Formula C-XXXIV, Formula C-XXXV, Formula C-XXVI, Formula C-XXXVII, Formula C-XXXVIII, Formula C-XXXIX, Formula C-XL, Formula C-XLI, Formula C-XLII, Formula C-XLIII, Formula C-XLIV, and Formula C-XLV, as described herein.

In various embodiments, the antioxidant derivative of camptothecin and/or an antioxidant derivative of a camptothecin analog may be selected from the group consisting of: Compound C-23, Compound C-1, Compound C-2, Compound C-10, Compound C-3, Compound C-4, Compound C-5, Compound C-11, Compound C-6, Compound C-7, Compound C-8, Compound C-12, Compound C-9, Compound C-13, Compound C-14, Compound C-15, Compound C-16, Compound C-17, Compound C-18, Compound C-19, Compound C-20, Compound C-21, and Compound C-22, as described herein.

Various embodiments of the present invention provide for a method of treating cancer in a subject in need thereof, comprising: providing a nanosphere as described herein; and administering a therapeutically effective amount of the nanosphere to the subject to treat the cancer. In various embodiments, the cancer may be brain cancer.

In various embodiments, the therapeutic agent may be selected from the group consisting of: a chemotherapeutic agent, statin, nonsteroidal anti-inflammatory drug (NSAID), erythropoietin, peptide, antisense nucleic acid, DNA, RNA, protein, and combinations thereof. In various embodiments, the therapeutic agent may be selected from the group consisting of paclitaxel, doxorubicin, temozolomide, 5-fluorouracil, camptothecin, and combinations thereof.

Various embodiments of the present invention provide for a method of diagnosing cancer in a subject in need thereof comprising: providing a nanosphere as described herein; administering an effective amount of the nanosphere to the subject; and imaging the subject to diagnose the cancer. In various embodiments, the imaging agent may be selected from the group consisting of: fluorescent dye, antibody against a protein overexpressed in cancer, and combinations thereof.

In various embodiments, the nanosphere may further comprise a statin lactone derivative having Formula D-I, D-II, D-III, D-IV, D-V or D-VI, as described herein.

In various embodiments, the statin lactone derivative may be selected from the group consisting of: Compound D-47, Compound D-48, Compound D-49, Compound D-50, Compound D-51, Compound D-52, Compound D-53, Compound D-54, Compound D-55, Compound D-56, Compound D-57, Compound D-58, Compound D-59, Compound D-60, Compound D-61, Compound D-62, Compound D-63, Compound D-64, Compound D-65, Compound D-66, Compound D-67, Compound D-68, Compound D-69, Compound D-70, Compound D-13, Compound D-14, Compound D-15, Compound D-16, Compound D-17, Compound D-18, Compound D-19, Compound D-20, Compound D-21, Compound D-22, Compound D-23, Compound D-24, Compound D-25, Compound D-26, Compound D-27, Compound D-28, Compound D-29, Compound D-30, Compound D-31, and Compound D-32, as described herein.

Various embodiments Various embodiments of the present invention provide for a method of lowering cholesterol levels, lowering the likelihood of cardiovascular disease, or treating cardiovascular disease in a subject in need thereof, comprising: providing a nanosphere as described herein; and administering a therapeutically effective amount of the nanosphere to the subject to lower the cholesterol levels, lower the likelihood of cardiovascular disease, or treat cardiovascular disease.

Various embodiments provide for a method of diagnosing cancer in a subject in need thereof comprising: providing a nanosphere as described herein; administering an effective amount of the nanosphere to the subject; and imaging the subject to diagnose the cancer. In various embodiments, the imaging agent may be selected from the group consisting of: fluorescent dye, antibody against a protein overexpressed in cancer, and combinations thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a schematic representation of the synthesis steps for antioxidant and antineoplastic nanoparticle comprising an amphiphilic spacer in accordance with various embodiments of the present invention.

FIG. 2 depicts another schematic representation of the synthesis steps for antioxidant and antineoplastic nanoparticle comprising an amphiphilic spacer in accordance with various embodiments of the present invention.

FIG. 3 depicts a schematic representation of an antioxidant and antineoplastic nanoparticle comprising an amphiphilic spacer in accordance with various embodiments of the present invention.

FIG. 4 depicts a schematic representation of an antioxidant and antineoplastic nanoparticle comprising a therapeutic agent conjugated to the amphiphilic spacer in accordance with various embodiments of the present invention. (a) The core nanoparticle was prepared from tocopherol with CPT-TEG-ALA; (b) The core nanoparticle was prepared from tocopherol without CPT-TEG-ALA; and (c) The core nanoparticle was prepared from tocopherol without CPT-TEG-ALA. The spacer contains primary amine (—NH2) instead of sulfhydryl (—SH).

FIG. 5 depicts a schematic representation of an antioxidant and antineoplastic nanoparticle comprising amphiphilic polymer in accordance with various embodiments of the present invention.

FIG. 6 depicts a schematic representation of an antioxidant and antineoplastic nanoparticle comprising therapeutic agent on amphiphilic polymer in accordance with various embodiments of the present invention. (a) the core nanoparticle was prepared from tocopherol with CPT-TEG-ALA; (b) the core nanoparticle was prepared from tocopherol without CPT-TEG-ALA.

FIG. 7 depicts a schematic representation of an antioxidant tocopherol nanoparticle comprising therapeutic or imaging agent on spacers in accordance with various embodiments of the present invention.

FIG. 8 depicts a schematic representation of an antioxidant and neuroprotective statin/tocopherol nanoparticle comprising therapeutic or imaging agent on spacers in accordance with various embodiments of the present invention.

FIG. 9 depicts a schematic representation of an antioxidant and neuroprotective NSAID/statin/tocopherol nanoparticle comprising therapeutic or imaging agent on spacers in accordance with various embodiments of the present invention.

FIG. 10 depicts preparation of camptothecin nanoprodrug in accordance with various embodiments of the present invention. a, Free camptothecin (CPT) incorporated with α-lipoic acid (ALA) and tetra(ethylene glycol) (TEG) into prodrug CPT-TEG-ALA. b, Prodrug and α-tocopherol undergo spontaneous emulsification into CPT-TEG-ALA/Toco nanoprodrug. c, Cy5.5 incorporated by conjugation to thiol moiety of 1-octadecanethiol.

FIG. 11 depicts characterization of camptothecin nanoprodrug in accordance with various embodiments of the present invention. a, Visualization of CPT-TEG-ALA/Toco nanoprodrug (I) and Toco nanosuspension (II) obtained from nanoparticle tracking analysis (NTA). b, Visualization of in vitro uptake of CPT-TEG-ALA/Toco nanoprodrug into U87 MG glioma cells by fluorescent detection of Cy5.5 functionalized nanoprodrug as determined by laser confocal microscopy. Scale bars, 100 μm for low and 20 μm for high magnification. c, Chromatogram of degraded camptothecin (P1), oxidized CPT-TEG-ALA prodrug (P2), and intact CPT-TEG-ALA prodrug (P3). Chromatogram I was taken from fresh prepared and partially oxidized nanoprodrug. Chromatogram II was taken from cell lysate prepared as described in herein.

FIG. 12 depicts tumor-specific localization of Cy5.5-fluorescent nanoprodrug CPT-TEG-ALA/Toco in accordance with various embodiments of the present invention. a, Representative fluorescent images of mouse with subcutaneous U87 MG glioma xenograft and harvested organs 72 h after intravenous injection of the fluorescent nanoprodrug. b, Comparison of the accumulation of Cy5.5-fluorescent CPT-TEG-ALA/Toco nanoprodrug (I) and free Cy5.5 (II) in the subcutaneous U87 MG glioma xenograft 96 h after intravenous injection of the fluorescent nanoprodrug. c, Tumor histology. U87 MG subcutaneous xenograft tumor sections (10 μm) were stained with H&E, fluorescent imaged for Cy5.5-flourescent CPT-TEG-ALA/Toco nanoprodrug, or immunostained with CD31. Black and white arrows, tumor vasculature. Scale bar, 20 μm. d, Fluorescent of brain and organs harvested 3 h and 5 h after intravenous injection of the Cy5.5-fluorescent CPT-TEG-ALA/Toco nanoprodug. e, Fluorescent image of brain and organs harvested 48 h after intravenous injection of the fluorescent CPT-TEG-ALA/Toco nanoprodug (L), images of both side of the brain (M) and longitudinal brain section in OCT block (R). f, Histology, fluorescent imaging, and immunostaining of brain sections containing U87 MG intracranial xenograft. Mice were sacrificed 48 h after injection of fluorescent CPT-TEG-ALA/Toco nanoprodrug, brain was separated immediately, frozen on dry ice and cryosectioned. HE staining exhibits well-defined barriers between healthy and tumor areas. Representative Cy5.5 fluorescent pictures show CPT-TEG-ALA/Toco nanoprodrugs localized specifically within U87 MG intracranial tumor. The strongest accumulation was observed near the tumor vasculature and in the tumor margins. Representative CD31 immunostaining shows a strong, abnormal blood vessel formation in the tumor area. Representative picture of Ki67 immunostaining shows proliferating tumor cells in the tumor area.

FIG. 13 depicts anti-tumor efficacy of CPT-TEG-ALA/Toco nanoprodrugin accordance with various embodiments of the present invention. a, Volume of subcutaneous U87 MG human tumor xenograft in mice after treatment with CPT-TEG-ALA/Toco nanoprodrug, irinotecan, α-tocopherol nanosuspension, and saline. Statistical significance was estimated by Student's t-test for last three measurements of CPT-TEG-ALA/Toco nanoprodrug and saline control. Points, means from six animals per group; bars, SD. b, Kaplan-Meier survival plot demonstrating survival benefit of CPT-TEG-ALA/Toco nanoprodrug for animals with intracranial U87 MG tumor xenograft. The figure shows percent survival of mice after treatment with CPT-TEG-ALA/Toco nanoprodrug, irinotecan, α-tocopherol nanosuspension, or saline. Statistical significance was estimated by log-rank method of CPT-TEG-ALA/Toco nanoprodrug compared with saline control.

FIG. 14 depicts proposed mechanisms of drug effect in accordance with various embodiments of the present invention. a, Schematic representation of nanoprodug activation in the oxidative environment of the brain tumor. α-Lipoic acid moiety of camptothecin prodrug scavenges ROS in the oxidative tumor microenvironment, accelerating the erosion of the nanoprodrug surface. This facilitates the hydrolytic or enzymatic degradation of the prodrug. Red arrow shows the site of hydrolysis. b, Nanoprodrug accumulation via EPR effect in the brain tumor tissue. Cy5.5 fluorescent image shows CPT-TEG-ALA/Toco nanoprodrugs localized specifically around tumor blood vessels within U87 MG intracranial tumor. Fluorescent signals from healthy brain tissues are negligible. CD31 immunostaining shows a strong, abnormal vasculature in the tumor area and micro-vessels in normal brain tissue. Nanoprodrugs are shown as black dots.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The abbreviation “CPT” as used herein refers to camptothecin {(S)-4-ethyl-4-hydroxy-1H-pyran-[3′,4′:6, 7]indolizino[1,2-b]quinoline-3,14(4H, 12H)-dione}, which is shown below. The compound is commercially available from numerous sources; e.g., from Sigma Chemical Co. (St. Louis, Mo.).

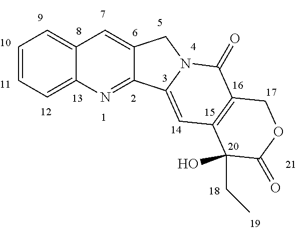 [SMILES: CC[C@@]1(O)C(=O)OCc2c1cc1n(c2=O)Cc2cc3ccccc3nc2-1]

“Camptothecin analogs” as used herein refer to compounds of Formula C-I:

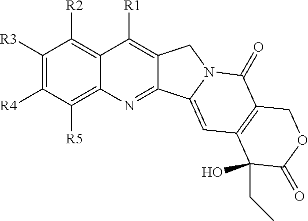

wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

“Antioxidant derivative of camptothecin” and “antioxidant camptothecin derivative,” as used herein refer to a derivative of camptothecin that contains an antioxidant [1,2]-dithiolane ring.

“Antioxidant derivative of a camptothecin analog” and “antioxidant camptothecin analog derivative” as used herein refer to a derivative of a camptothecin analog that contains an antioxidant [1,2]-dithiolane ring.

“Camptothecin nanosphere” and “camptothecin nanosphere prodrug” as used herein refer to a nanosphere comprising an antioxidant derivative of camptothecin or an antioxidant derivative of a camptothecin analog. The nanosphere may further comprise a multiple α-lipoic acid-containing hydrophobic compound, α-tocopherol, a nonsteroidal anti-inflammatory drug (NSAID) derivative, or combinations thereof.

“Cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer; including, but not limited to, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary carcinomas, neuroblastomas, and craniopharyngiomas.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Nanosphere” as used herein refers to a particle with a size, in at least one dimension, between about 10 nm to about 1000 nm; and may also include a nanoemulsion.

“Nanoprodrug” is used interchangeably with “nanosphere” throughout the application.

“Non-steroidal” as used herein distinguishes the anti-inflammatory drugs from steroids, which have a similar anti-inflammatory action.

“NSAID derivative” as used herein refers to a compound in which at least one NSAID molecule is coupled to a polyol; for example, through esterification.

“Polyol” as used herein refers to a compound that contains at least two free esterifiable hydroxyl groups.

“Therapeutic agent” as used herein refers to any substance used internally or externally as a medicine for the treatment, cure, prevention, slowing down, or lessening of a disease or disorder, even if the treatment, cure, prevention, slowing down, or lessening of the disease or disorder is ultimately unsuccessful.

“Therapeutically effective amount” as used herein refers to an amount which is capable of achieving beneficial results in a patient with a condition or a disease condition in which treatment is sought. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or alleviate the disease or disease condition even if the treatment is ultimately unsuccessful.

Chemotherapy for intracranial gliomas is hampered by limited delivery of therapeutic agents through the blood brain barrier (BBB). An optimal chemotherapeutic would selectively cross the blood tumor barrier, accumulate in the tumor and be activated from an innocuous prodrug from within the tumor. Here the inventors show a nanometer-sized assembly of anticancer prodrug (nanoprodrug) in which camptothecin (CPT) are chemically bonded to form a prodrug that is activated and released in the presence of oxidative stress. This oxidative stimuli-responsive nanoprodrug passes through the blood-brain barrier and accumulates specifically in glioblastoma multiforme (GBM) but not in healthy tissues and organs. Intracellular analysis demonstrated oxidized prodrugs and camptothecin release. The nanoprodrug was effective at inhibiting subcutaneous and intracranial tumors and led to significantly prolonged survivals in immunodeficient animals bearing human GBM.

Glioblastoma is the most common and aggressive type of malignant primary brain tumor in adults. Despite advances in neurosurgical intervention, radiation therapy, and chemotherapy, the median survival for glioblastoma remains less than 15 months after diagnosis^{1,2}. Tumors recur usually within 6 months of chemoradiation initiation. The treatment of intracranial glioma is limited by the inability to deliver chemotherapeutics at efficacious levels to the site of tumor³. The blood brain barrier (BBB) is a tightly regulated interface between the circulating blood and brain tissues formed by brain microvascular endothelial cells. The BBB maintains the homeostasis of the highly sensitive central nervous system (CNS) and protects the brain from neurotoxic substances prevalent in the peripheral circulatory system⁴. The BBB prevents free diffusion of most foreign molecules including therapeutic agents except for those that are small, uncharged, and lipid-soluble⁵. This remains the major obstacle for drug delivery into the brain. However, integrity of the BBB is severely compromised by many diseases in the brain, including brain tumors, neurodegenerative diseases, and traumatic brain injury (TBI)⁶⁻⁸. Vigorous tumor growth leads to induction of unregulated angiogenesis, resulting in defective vasculature with large pores and high permeability. This allows certain macromolecules and nanoparticles to penetrate through the BBB into the tumor and, due to the lack of lymphatic drainage, accumulate to therapeutic levels⁹. This phenomenon is termed the enhanced permeability and retention (EPR) effect, and it grants the opportunity for passive tumor specific targeting with macromolecular drugs and nanocarriers¹⁰.

Research in the field of cancer therapy using nanostructured materials has been receiving significant attention from the pharmaceutical industry due to their potential for precise targeting, improved tolerability, and drug efficacy¹¹. Another advantage of nanostructured materials is that water-insoluble therapeutics can be transported more efficiently in the aqueous physiological environment when integrated into stable nanostructures¹². The major problem encountered with camptothecin is its extremely low solubility in an aqueous environment. Its carboxylate form is more water-soluble, but the loss of the lactone form resulted in the loss of its anticancer efficacy¹³. The inventors had characterized the nanoprodrug prepared from CPT prodrug (CPT-TEG-ALA) and α-tocopherol (Toco) with regard to structure, ROS scavenging capability, enzymatic activation, release kinetics, and in vitro anticancer efficacy against U87 MG glioma cells¹⁴. Described herein, the inventors demonstrate cellular uptake, tumor specific targeting, and anti-tumor efficacy of the CPT nanoprodug in experimental mouse models bearing human subcutaneous and intracranial gliomas.

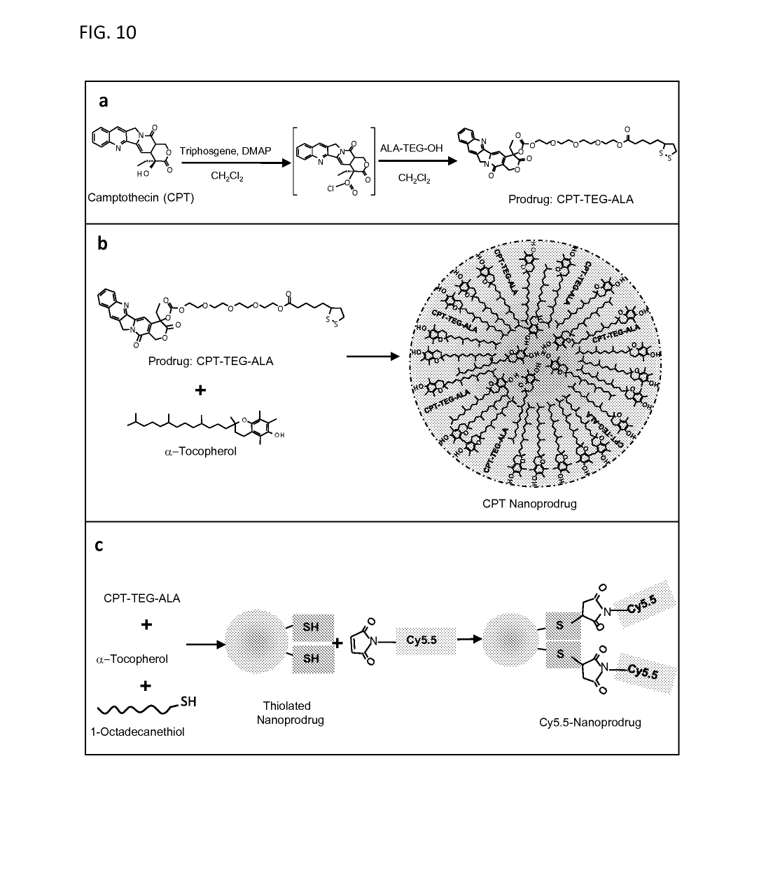

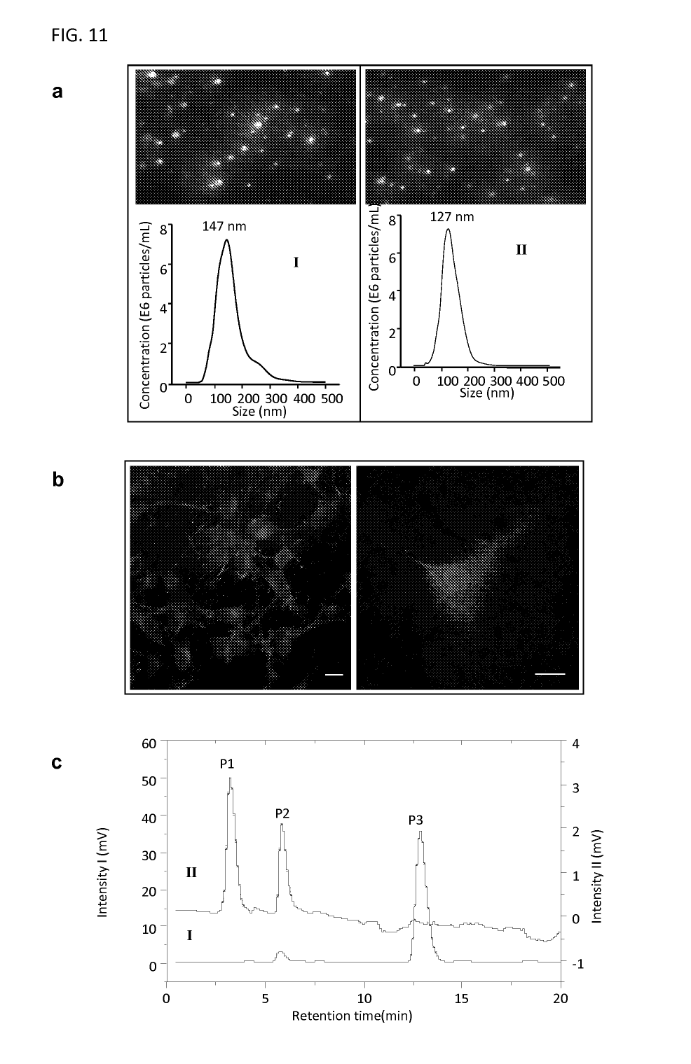

Camptothecin prodrug was synthesized by introducing biodegradable carbonate and ester bonds (FIG. 10). The biodegradable bonds ensure that the prodrug molecules break down hydrolytically or enzymatically by esterase. Spontaneous emulsification of the produg and α-tocopherol into nanoprodrug abates problems associated with free delivery of the highly hydrophobic prodrug. The hydrophobic interaction between prodrug molecules stabilizes the nanoprodrug in an aqueous environment, which maintains the integrity of the nanostructures. Moreover, transformation into nanoprodrug generates abundant reactive surface area where the prodrugs are activated upon contact with biological molecules, which increases the rate of prodrug activation and thus improves therapeutic efficacy¹⁵. FIG. 11a shows that the average size of the nanoprodrug CPT-TEG-ALA/Toco calculated by nanoparticle tracking analysis (NTA)¹⁶ is slightly larger than the nanosuspension prepared from α-tocopherol. This nanoprodug strategy is a versatile method of developing therapeutic nanoparticles by converting drugs into biodegradable prodrugs and transforming them into nanoprodrugs.

To visualize the cellular uptake of the nanoprodrug, the inventors prepared Cy5.5 labeled nanoprodrug. As shown in FIG. 11b, U87 MG glioma cells displayed effective cellular uptake within 5 hours of incubation. The inventors previously demonstrated that the α-lipoic acid moiety efficiently scavenged ROS, leading to accelerated destabilization of the nanoprodrug and increased prodrug activation′. This suggests that the nanoprodrug is activated more preferably in an oxidative environment, including highly inflammatory tumor tissues. ROS have been reported to be directly involved in the link between chronic inflammation and cancer. Inflammation is widely recognized to be a critical component of tumor progression, survival and migration by virtue of recruiting and stimulating inflammatory cells generating abundant ROS^{18,19}. There is considerable evidence that ROS play a major role in tumor initiation and progression in both animal models and humans^{20,21}. It has been reported that reducing inflammation in the tumor microenvironment inhibits tumor progression in a mouse model²². To demonstrate degradation of CPT-TEG-ALA prodrug upon cellular uptake, cells were incubated in the presence of nanoprodrugs and the cell lysate was analyzed. Only the oxidized form of CPT-TEG-ALA (P2) and camptothecin (P1) were detected in the cell lysate (FIG. 11e), suggesting that prodrug oxidation preceded prodrug activation.

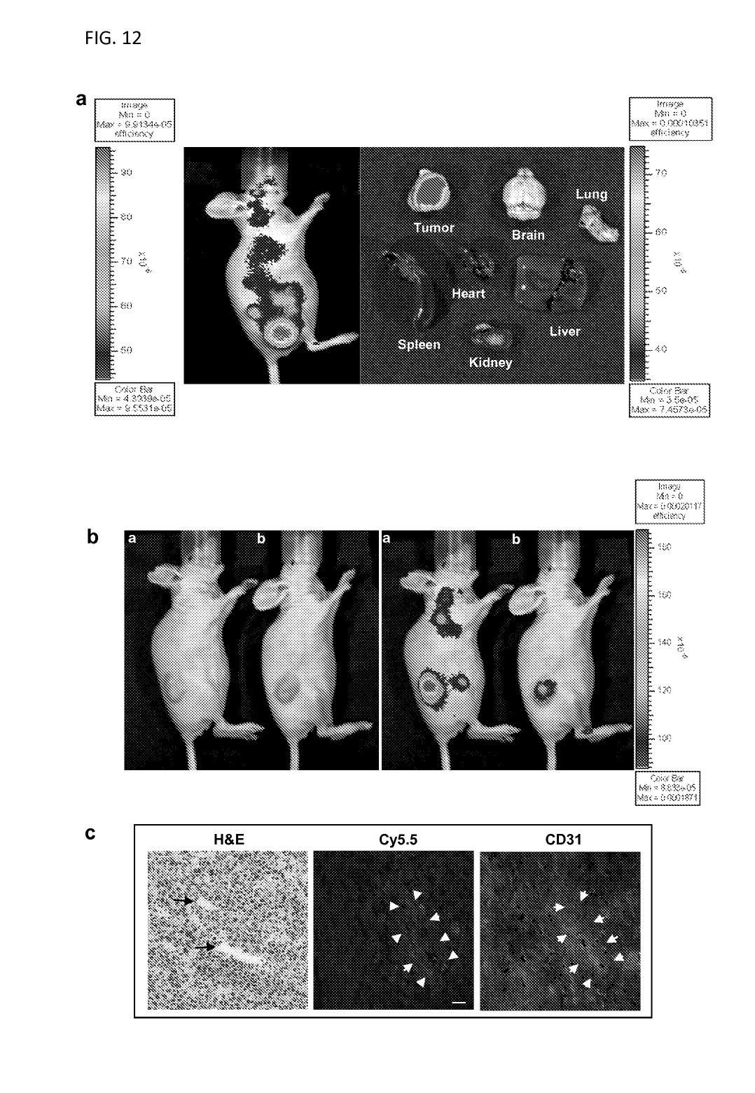

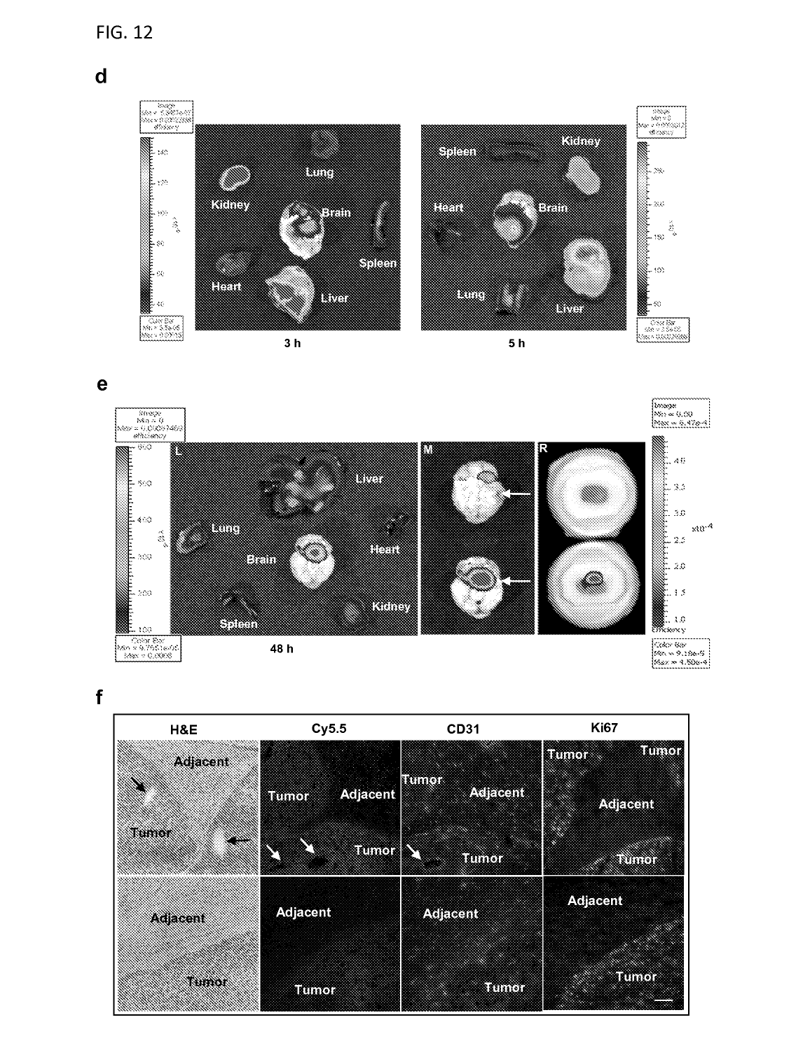

To elucidate the targeting ability of CPT-TEG-ALA/Toco nanoprodug on a human GBM tumor in vivo, nu/nu mice received subcutaneous implants of U87 MG tumor xenografts. The accumulation of the nanoprodrug occurred in tumor tissue, but not in the brain, liver, lung, heart, and spleen (FIG. 12a). FIG. 12b shows the accumulation of the nanoprodugs compared with free Cy5.5 dye, a trait attributed to the EPR effect. FIG. 12c shows abnormal tumor vasculature immunostained with CD31 and brighter Cy5.5 fluorescence around the vessels, suggesting an increased extravasation of the nanoprodrug through the highly permeable wall of the tumor blood vessels. The targeted accumulation was further shown in an intracranial xenograft of U87 MG glioma. The accumulation in the brain tumor occurred within 3-5 h after drug injection (FIG. 12d). The strong fluorescent signals from the kidney and liver dissipated 48 h after drug injection (FIG. 12e), while the signal from brain tumor intensified, suggesting a selective accumulation of the nanoprodug in the brain tumor. Noteworthy, nanoprodrugs were confined solely to the tumor, but not in the normal brain tissue. This feature highlights the ability of the CPT-TEG-ALA/Toco nanoprodrug to egress through the BBB in the tumor region, but not in the healthy brain tissue surrounding the tumor. The pattern of the fluorescent distribution in the dissected brain tumor tissue (FIG. 12f) shows its ideal targeting traits; strong accumulation confined to the tumor bed, no detection in adjacent healthy tissue, and maximum localization in the highly active, tortuous tumor boundary. Ki67 positive cells were localized in the periphery of the tumor, confirming the known tendency of tumors to proliferate outward into healthy tissue. These areas were also associated with areas of strong CD31 positive cells, suggesting that enhanced nanoprodrug accumulation preferably occurred in the rapidly proliferating tumor area with abnormal tumor vasculature.

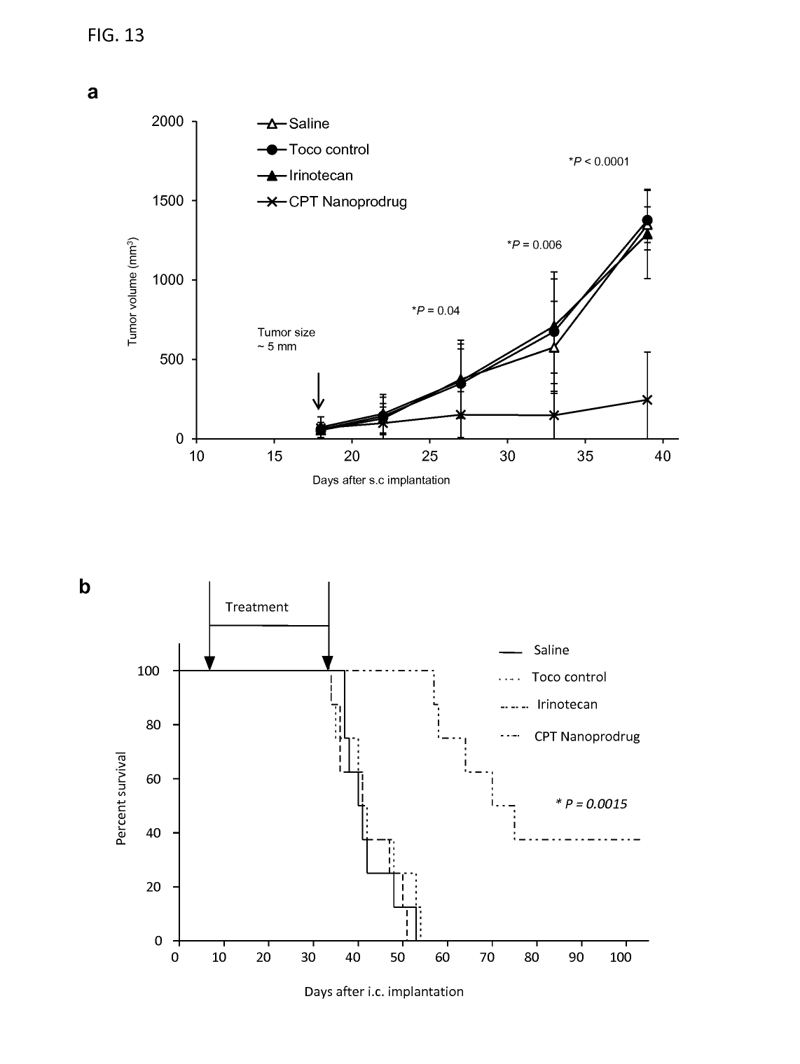

The inventors investigated the efficacy of the nanoprodrug on subcutaneous tumor growth of invasive U87 MG cells (FIG. 13a). Statistical analysis showed a significant reduction of tumor volume in treatment group compared with saline and α-tocopherol control groups, whereas there was no significant reduction when using a molar equivalent of the clinically used CPT analog, irinotecan. The nanoprodrug inhibited tumor growth to about 250 mm³ after 21 days of treatment, which is more than 80% reduction compared with the control treatments (1350 mm³). It was of interest whether the nanoprodrug would be effective in a more clinically relevant orthotopic model of intracranially implanted U87 MG cells. FIG. 13b displays the result of a survival study of the mice with intracranial GBM xenograft. The median survival time was 72.5, 41.0, 40.5, and 41.5 days for CPT-TEG-ALA/Toco nanoprodrug, irinotecan, saline, and α-tocopherol nanosuspension, respectively. The nanoprodrug was significantly more effective than the molar equivalent of irinotecan. There were long-term survivors only in the nanoprodrug group (log-rank, p=0.0015).

The highly specific accumulation of the nanoprodrug in tumor tissues followed by efficient intracellular uptake may be of importance for the treatment of cancers developing resistance to anticancer therapeutics, denoted multidrug resistance (MDR). MDR mediated by P-glycoprotein (Pgp) is the best characterized mechanism of MDR in brain tumors. Pgp has been found to be expressed in the cell membrane of brain tumor cells and in the endothelial cells of newly formed brain tumor blood vessels^{23,24}. This integral membrane transporter protein reduces intracellular drug levels by inhibiting drug uptake and promoting drug efflux²⁵. The use of nanoparticles that enter the cells by endocytosis has been suggested to overcome Pgp-mediated MDR. The exact mechanism by which nanoparticles circumvent the Pgp-mediated MDR is not yet clear. It has been suggested that Pgp recognizes hydrophobic drugs when they are present in the plasma membrane, but not when they are already in the cytoplasm²⁶. Therefore, nanoparticles that enter the cells by endocytosis without releasing drugs may overcome the Pgp-mediated MDR. The superior anti-cancer efficacy of camptothecin nanoprodrug compared with irinotecan may be attributed to the increased level of therapeutic drug in the cells, which is accomplished by the combination of the passive accumulation of the nanoprodrug in the tumor tissue (EPR effect) and efficient cellular uptake by endocytosis. This combined effect may contribute to overcoming Pgp-mediated MDR for the nanoprodrug, allowing drug accumulation in the cytoplasm, whereas both irinotecan and its active metabolite SN38 are substrates of P^{gp27′28.}

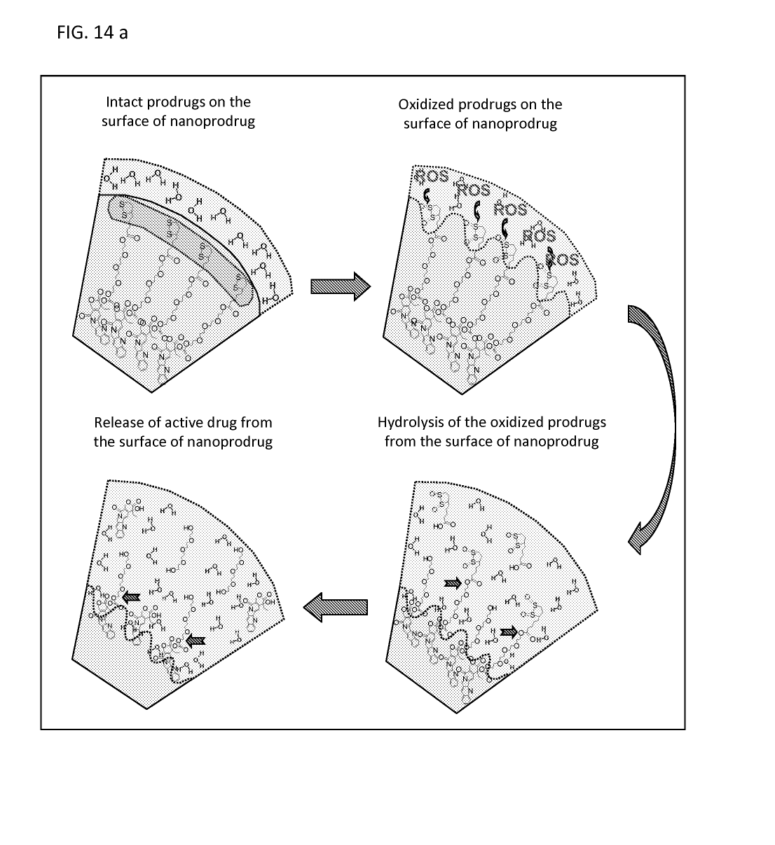

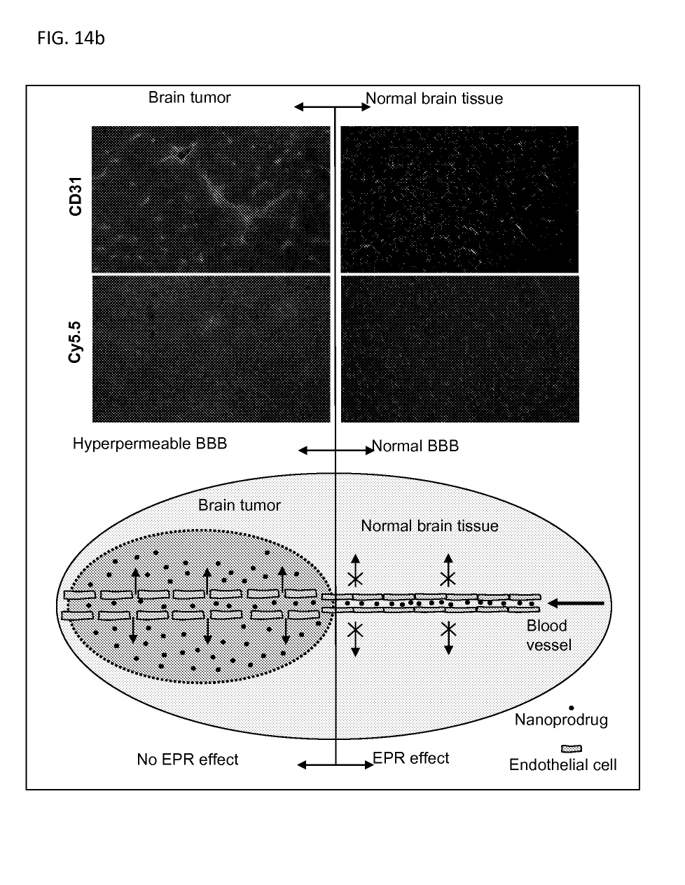

The oxidation of the α-lipoic acid-containing prodrugs resulted in the destabilization of the nanoprodrugs^{14,17}. This destabilization has been attributed to the increased hydrophilicity of the oxidized prodrugs on the surface of the nanoprodrug; the oxidized, hydrophilic prodrugs extrude into the aqueous environment, allowing enzymatic degradation of the prodrugs (FIG. 14a). In this way, the more prodrugs are degraded in an accelerated fashion by esterases as oxidation occurs on the surface of the nanoprodrug in the tumor microenvironment. This unique interaction between the oxidative destabilization and enzymatic prodrug activation characterizes the oxidative stimuli-responsive nanoprodug.

Angiogenesis occurs to meet the tumor's accelerated metabolic need, resulting in defective vasculature with large pores and high permeability. The EPR effect has been clearly documented for most human solid tumors, including both primary and metastatic in nature⁹. Considering the dysfunctional brain tumor vasculature, the inventors believe, but not wishing to be bound by any particular theory, that nanoparticle accumulation in a glioma model can be, like most solid tumor models, attributed to the EPR effect. Similarly, CPT-TEG-ALA/Toco nanoprodrug may be capable of passive targeting of brain tumor tissue via the EPR effect (FIG. 14b). Due to the nature of glioblastoma multiforme to infiltrate into brain parenchyma as it proliferates, the tumoral regions which are most actively dividing, invading, and inducing angiogenesis are at the margins, whereas necrosis is found in the center of the gliomas²⁹. This progression of vasculature is confirmed by the high CD31+ fluorescence at the margin between tumor mass and healthy cells (FIG. 12f). The nanoprodrug fluorescence is increased in areas of neo-angiogenesis where the tumor is actively expanding resulting in optimal efficacy. Cancer stem cells have been shown to reside in the perivascular niche and this delivery pattern may specifically target this virulent subset of tumor cells³⁰.

In summary, the inventors demonstrated that the increased permeability of blood vessels in the glioma xenograft allows particulate therapeutic nanoprodrug of camptothecin to pass through the blood vessel and selectively accumulate in both subcutaneous and intracranial glioma models. The inventors have demonstrated the increased tumor specific delivery and efficacy of this nanoprodrug in comparison to the molar equivalent of presently used clinical form of CPT, irinotecan. This platform of ROS-sensitive release of chemotherapeutics may enable a higher safety profile. The inventors have engineered other chemotherapeutics as well as other therapeutic agents into this nanoprodrug platform. This platform is adaptable to many agents for site-specific release in oxidative environments associated with inflammation.

Various embodiments of the present invention provide for various nanospheres comprising a therapeutic agent or diagnostic agent on an amphiphilic spacer. Other embodiments of the present invention provide for nanospheres comprising a therapeutic agent or a diagnostic agent on an amphiphilic polymer.

### Nanospheres

In various embodiments, the nanospheres are antioxidant nanospheres.

In certain embodiments, the nanopheres are formed with tocopherol. Thus, in certain embodiments, the nanospheres comprise tocopherol.

### α-Lipoic Acid-Containing Nanospheres

In certain embodiments, the nanospheres are formed with antioxidant α-lipoic acid-containing hydrophobic compounds. Thus, in certain embodiments, the nanospheres comprise antioxidant α-lipoic acid-containing hydrophobic compounds. These compounds are disclosed in U.S. Provisional Application Ser. No. 61/018,749, filed Jan. 3, 2008, and International Application Publication No. WO 2009/086547, filed Dec. 30, 2008, which are incorporated by reference in their entirety as though fully set forth. Examples of these antioxidant α-lipoic acid-containing hydrophobic compounds include, but are not limited to the following:

Antioxidant α-lipoic acid-containing hydrophobic compounds represented by Formula A-Ia

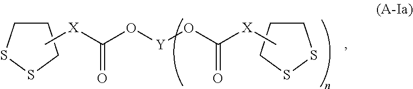

wherein X may be selected from the group consisting of a substituted, unsubstituted, branched or unbranched chain of carbon atoms, and may optionally contain a heteroatom; Y may be selected from the group consisting of a branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic group; and n may be an integer of at least one. In particular embodiments, n may be an integer from 1 to 4; and X may be an unsubstituted, unbranched chain of 1 to 6 carbon atoms.

In one embodiment, the dithiolane moiety in Formula Ia may be an α-lipoic acid and is represented by Formula A-IIa:

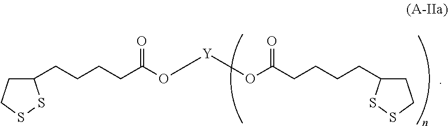

In various embodiments, Y may be a moiety formed by esterification of the hydroxyl groups of a polyol. In various embodiments, the polyol may be selected from the group consisting of

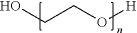

wherein n is an integer between 1 and 4 and

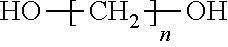

wherein n is an integer between 3 and 16.

One example of a particularly useful multiple α-lipoic acid-containing hydrophobic compound is represented as follows:

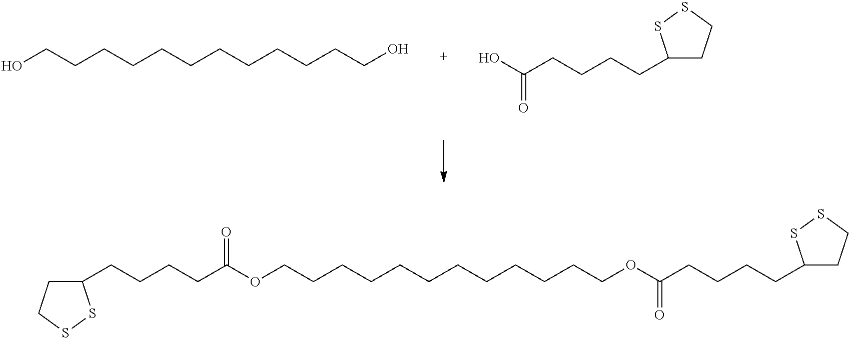 [SMILES: OCCCCCCCCCCCCO ; O=C(O)CCCCC1CCSS1 ; O=C(CCCCC1CCSS1)OCCCCCCCCCCCCOC(=O)CCCCC1CCSS1]

### NSAID Nanospheres

In certain embodiments, the nanospheres are formed with hydrophobic NSAID derivatives. Thus, in certain embodiments, the nanospheres comprise hydrophobic NSAID derivatives. In certain embodiments, the nanospheres are formed with hydrophobic antioxidant and anti-inflammatory derivatives of an NSAID. Thus, in certain embodiments, the nanospheres comprise hydrophobic antioxidant and anti-inflammatory derivatives of an NSAID.

International Application Publication No. WO2009/148698 provides examples of hydrophobic NSAID derivatives and hydrophobic antioxidant and anti-inflammatory derivatives of an NSAID, and is incorporated herein by reference as though fully set forth in its entirety.

### NSAID Derivatives and Nanospheres

Various embodiments of the present invention use NSAID nanospheres comprising a hydrophobic derivative of an NSAID (“NSAID derivative”). In one embodiment, the NSAID nanospheres of the present invention are capable of releasing the NSAID derivatives during a prolonged period of time, and thus reduce adverse gastrointestinal side effects caused by NSAIDs.

The NSAID nanospheres comprise derivatives of NSAIDs (“NSAID derivative”). Hydrophobic NSAID derivatives of the present invention may be represented by Formula B-I:

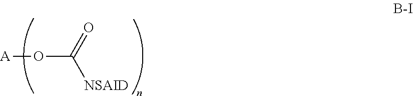

wherein the A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; and n is an integer of at least two, and in particular embodiments n may be an integer from 2-4. In various embodiments, A is a moiety that is formed by esterification of at least two free esterifiable hydroxyl groups on a polyol.

In various embodiments, polyols that are useful in the present invention include commercially available diols as follows:

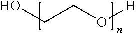

wherein n is an integer between 1 and 6.

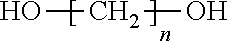

wherein n is an integer between 3 and 16.

In other embodiments, the polyols may be selected from the commercial available polyols as shown below:

TABLE 1

| Com- | |
|---|---|
| pound | Structure |
| 1 | 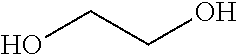 |
| 2 | 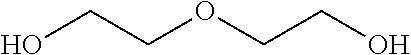 |
| 3 | 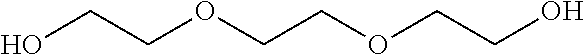 |
| 4 | 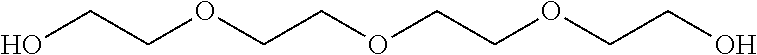 [SMILES: OCCOCCOCCOCCO] |
| 5 | 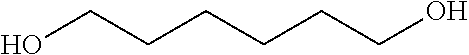 |
| 6 | 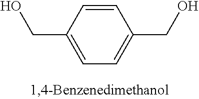 |
| 7 | 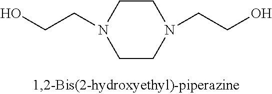 [SMILES: OCCN1CCN(CCO)CC1] |
| 8 | 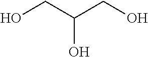 |
| 9 | 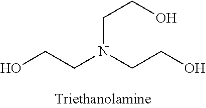 |
| 10 | 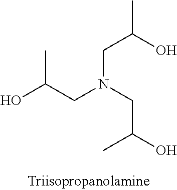 |
| 11 | 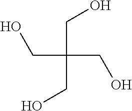 |
| 12 | 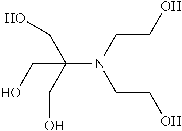 [SMILES: OCCN(CCO)C(CO)(CO)CO] |
| 13 | 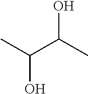 |
| 14 | 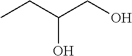 |
| 15 | 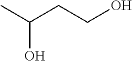 |
| 16 | 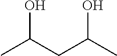 |
| 17 | 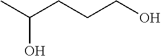 |
| 18 | 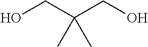 |
| 19 | 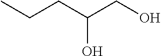 |
| 20 | 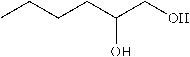 |
| 21 | 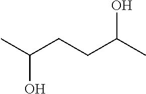 |
| 22 | 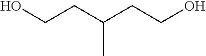 |
| 23 | 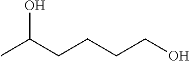 |
| 24 | 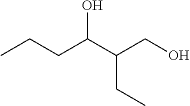 |
| 25 | 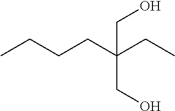 |
| 26 | 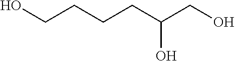 |
| 27 | 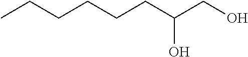 |
| 28 | 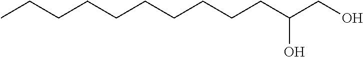 |
| 29 | 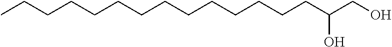 [SMILES: CCCCCCCCCCCCCCC(O)CO] |
| 30 | 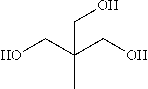 |
| 31 | 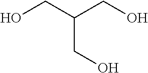 |
| 32 | 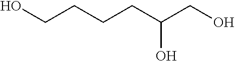 |
| 33 | 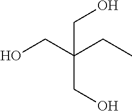 |
| 34 | 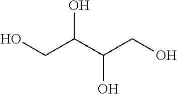 |
| 35 | 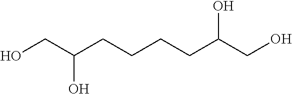 [SMILES: OCC(O)CCCCC(O)CO] |
| 36 | 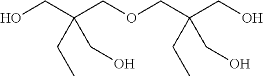 [SMILES: CCC(CO)(CO)COCC(CC)(CO)CO] |
| 37 | 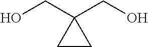 |
| 38 | 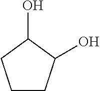 |
| 39 | 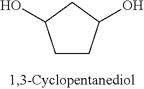 |
| 40 | 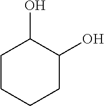 |
| 41 | 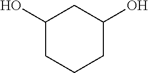 |
| 42 | 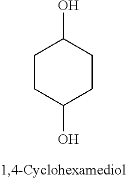 |
| 43 | 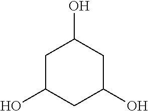 |
| 44 | 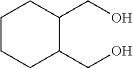 |
| 45 | 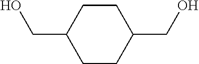 |
| 46 | 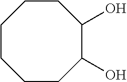 |
| 47 | 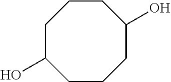 |
| 48 | 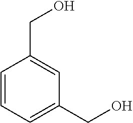 |
| 49 | 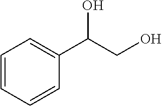 |
| 50 | 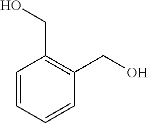 |
| 51 | 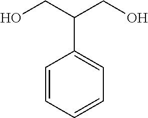 [SMILES: OCC(CO)c1ccccc1] |
| 52 | 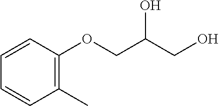 |
| 53 | 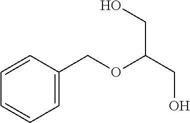 [SMILES: OCC(CO)OCc1ccccc1] |
| 54 | 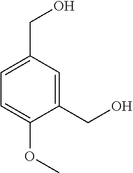 |
| 55 | 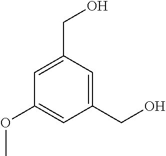 |
| 56 | 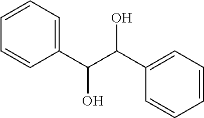 [SMILES: OC(c1ccccc1)C(O)c1ccccc1] |
| 57 | 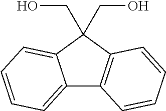 [SMILES: OCC1(CO)c2ccccc2-c2ccccc21] |
| 58 | 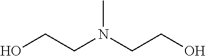 |
| 59 | 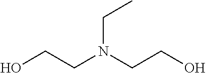 |
| 60 | 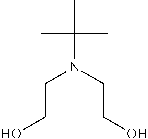 |
| 61 | 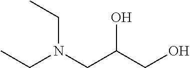 |
| 62 | 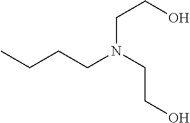 |
| 63 | 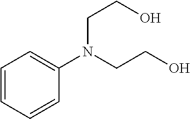 [SMILES: OCCN(CCO)c1ccccc1] |
| 64 | 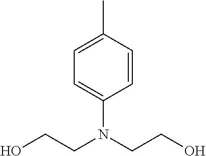 |
| 65 | 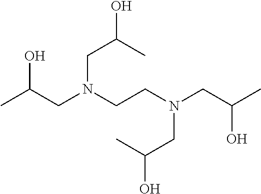 [SMILES: CC(O)CN(CCN(CC(C)O)CC(C)O)CC(C)O] |

The NSAID may be a non-steroidal anti-inflammatory drug containing a carboxylic acid. NSAIDs are well known in the art and one of skill in the art will be able to readily choose an NSAID without undue experimentation. The carboxylic group of the NSAIDs is temporarily masked via hydrolysable bond, and may therefore act as a prodrug and reduce the side effect and also has advantage in the controlled and sustained release of the drugs.

Examples of NSAIDs include but are not limited to aspirin, ibuprofen, flurbiprofen, ketoprofen, fenoprofen, fenbufen, naproxen, indomethacin, diclofenac, ketorolac, tolmetin, flufenamic acid, mefenamic acid, tolfenamic acid, meclofenamic acid, niflumic acid, sulindac, and sulindac sulfide.

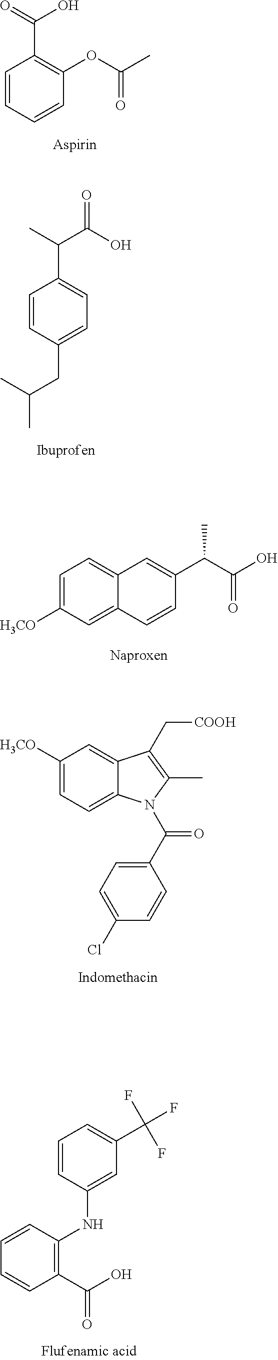 [SMILES: CC(=O)Oc1ccccc1C(=O)O ; CC(C)Cc1ccc(C(C)C(=O)O)cc1 ; COc1ccc2cc([C@H](C)C(=O)O)ccc2c1 ; COc1ccc2c(c1)c(CC(=O)O)c(C)n2C(=O)c1ccc(Cl)cc1 ; O=C(O)c1ccccc1Nc1cccc(C(F)(F)F)c1]

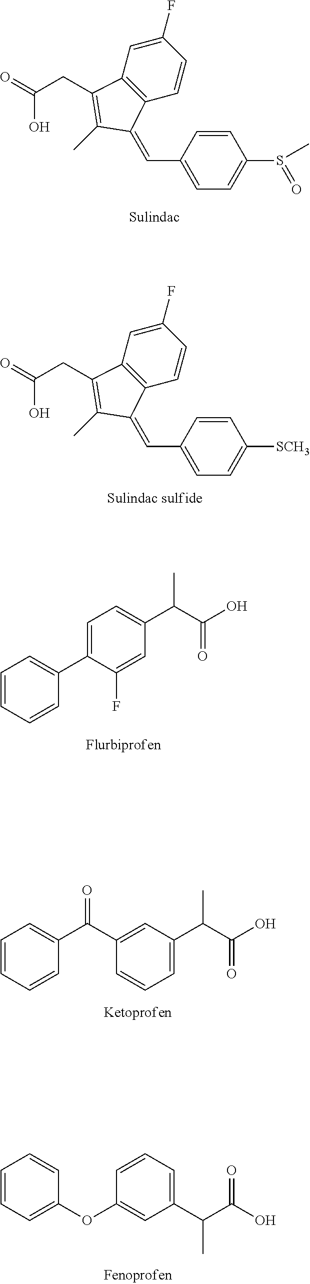 [SMILES: CC1=C(CC(=O)O)c2cc(F)ccc2/C1=C\c1ccc(S(C)=O)cc1 ; CSc1ccc(/C=C2/C(C)=C(CC(=O)O)c3cc(F)ccc32)cc1 ; CC(C(=O)O)c1ccc(-c2ccccc2)c(F)c1 ; CC(C(=O)O)c1cccc(C(=O)c2ccccc2)c1 ; CC(C(=O)O)c1cccc(Oc2ccccc2)c1]

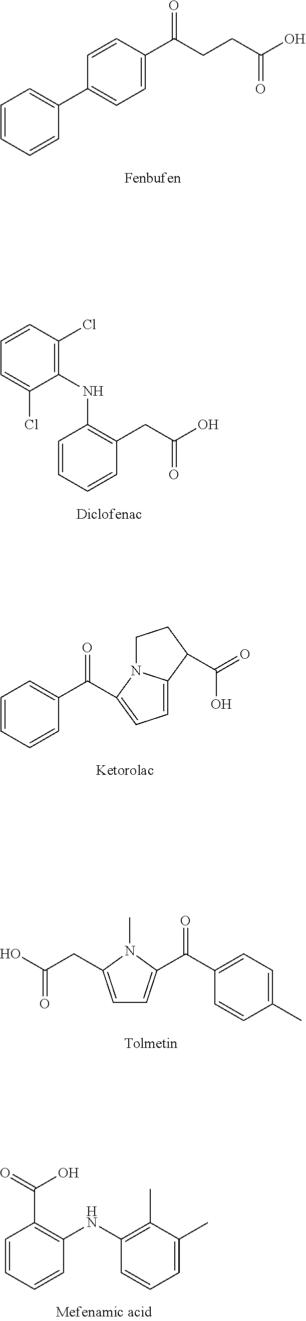 [SMILES: O=C(O)CCC(=O)c1ccc(-c2ccccc2)cc1 ; O=C(O)Cc1ccccc1Nc1c(Cl)cccc1Cl ; O=C(c1ccccc1)c1ccc2n1CCC2C(=O)O ; Cc1ccc(C(=O)c2ccc(CC(=O)O)n2C)cc1 ; Cc1cccc(Nc2ccccc2C(=O)O)c1C]

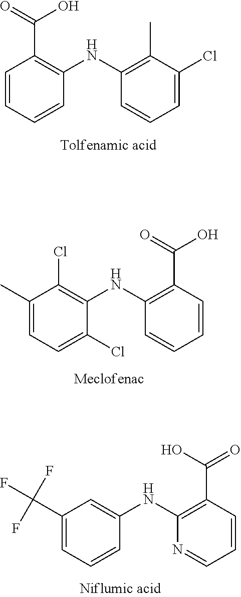 [SMILES: Cc1c(Cl)cccc1Nc1ccccc1C(=O)O ; Cc1ccc(Cl)c(Nc2ccccc2C(=O)O)c1Cl ; O=C(O)c1cccnc1Nc1cccc(C(F)(F)F)c1]

As such, examples of particularly useful hydrophobic derivatives of NSAIDs are represented by formulas as follows:

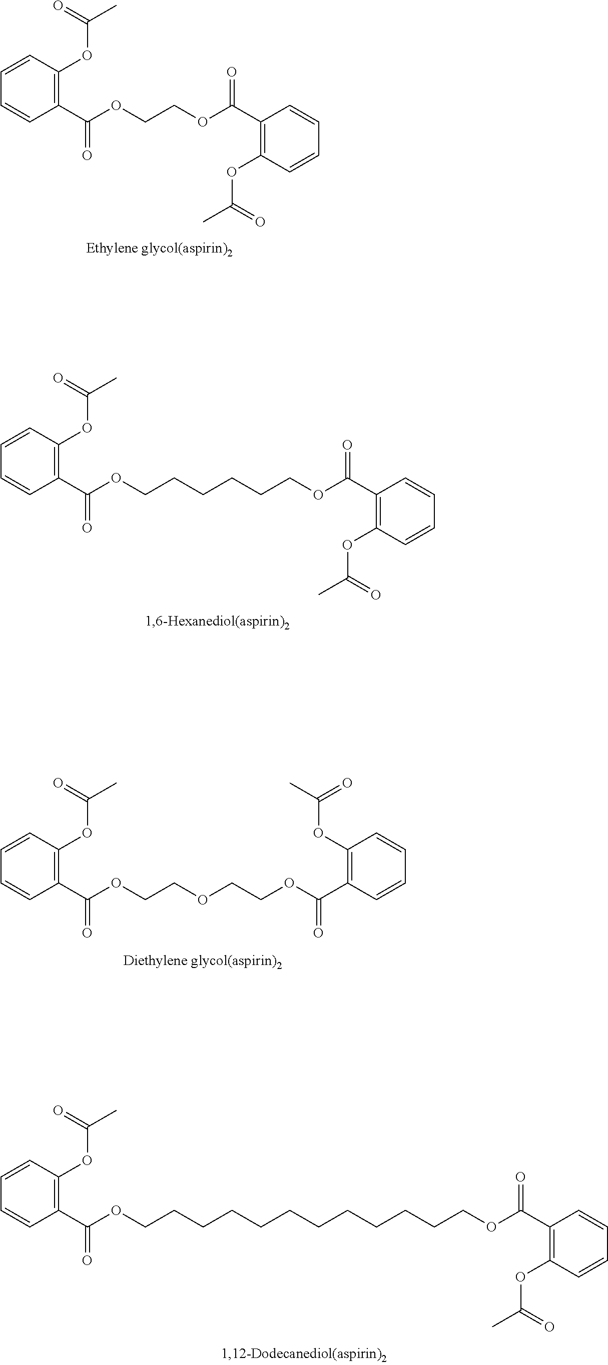 [SMILES: CC(=O)Oc1ccccc1C(=O)OCCOC(=O)c1ccccc1OC(C)=O ; CC(=O)Oc1ccccc1C(=O)OCCCCCCOC(=O)c1ccccc1OC(C)=O ; CC(=O)Oc1ccccc1C(=O)OCCOCCOC(=O)c1ccccc1OC(C)=O ; CC(=O)Oc1ccccc1C(=O)OCCCCCCCCCCCCOC(=O)c1ccccc1OC(C)=O]

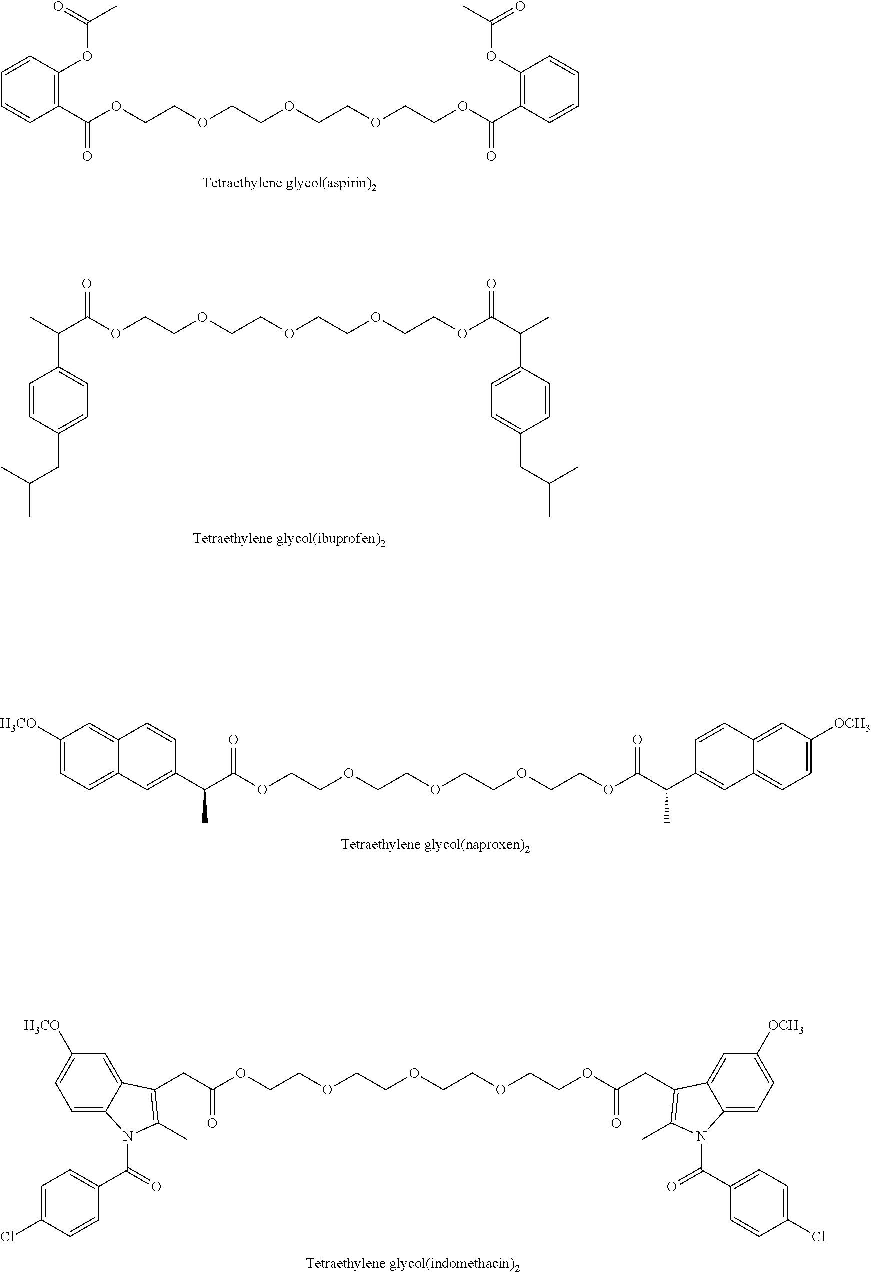 [SMILES: CC(=O)Oc1ccccc1C(=O)OCCOCCOCCOCCOC(=O)c1ccccc1OC(C)=O ; CC(C)Cc1ccc(C(C)C(=O)OCCOCCOCCOCCOC(=O)C(C)c2ccc(CC(C)C)cc2)cc1 ; COc1ccc2cc([C@H](C)C(=O)OCCOCCOCCOCCOC(=O)[C@@H](C)c3ccc4cc(OC)ccc4c3)ccc2c1 ; COc1ccc2c(c1)c(CC(=O)OCCOCCOCCOCCOC(=O)Cc1c(C)n(C(=O)c3ccc(Cl)cc3)c3ccc(OC)cc13)c(C)n2C(=O)c1ccc(Cl)cc1]

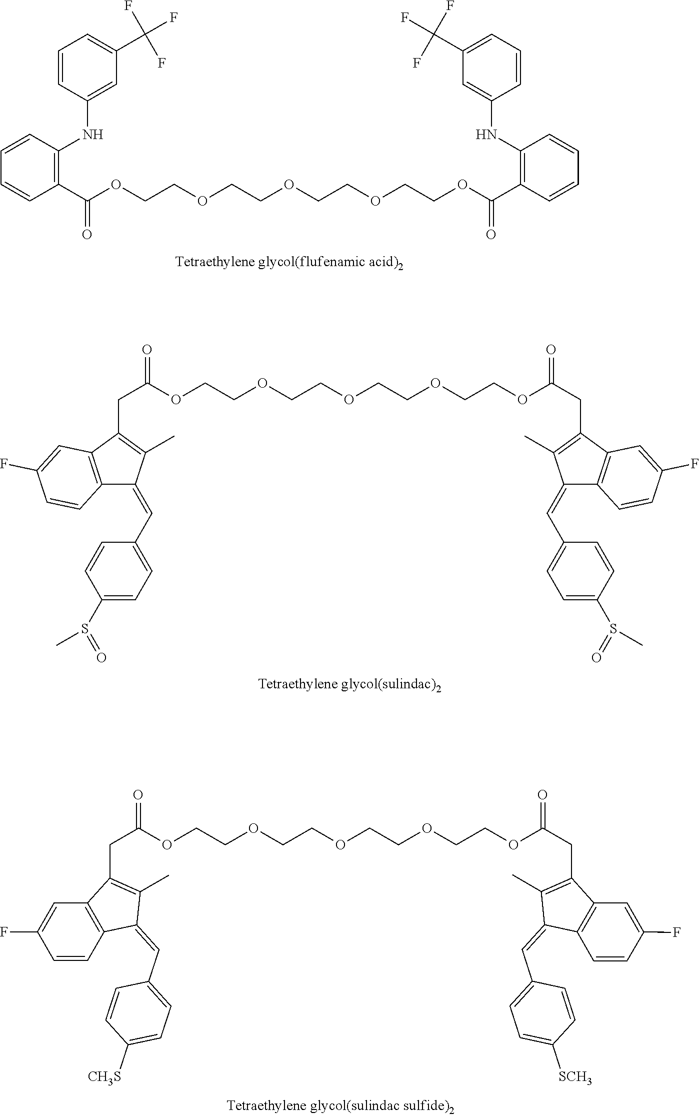

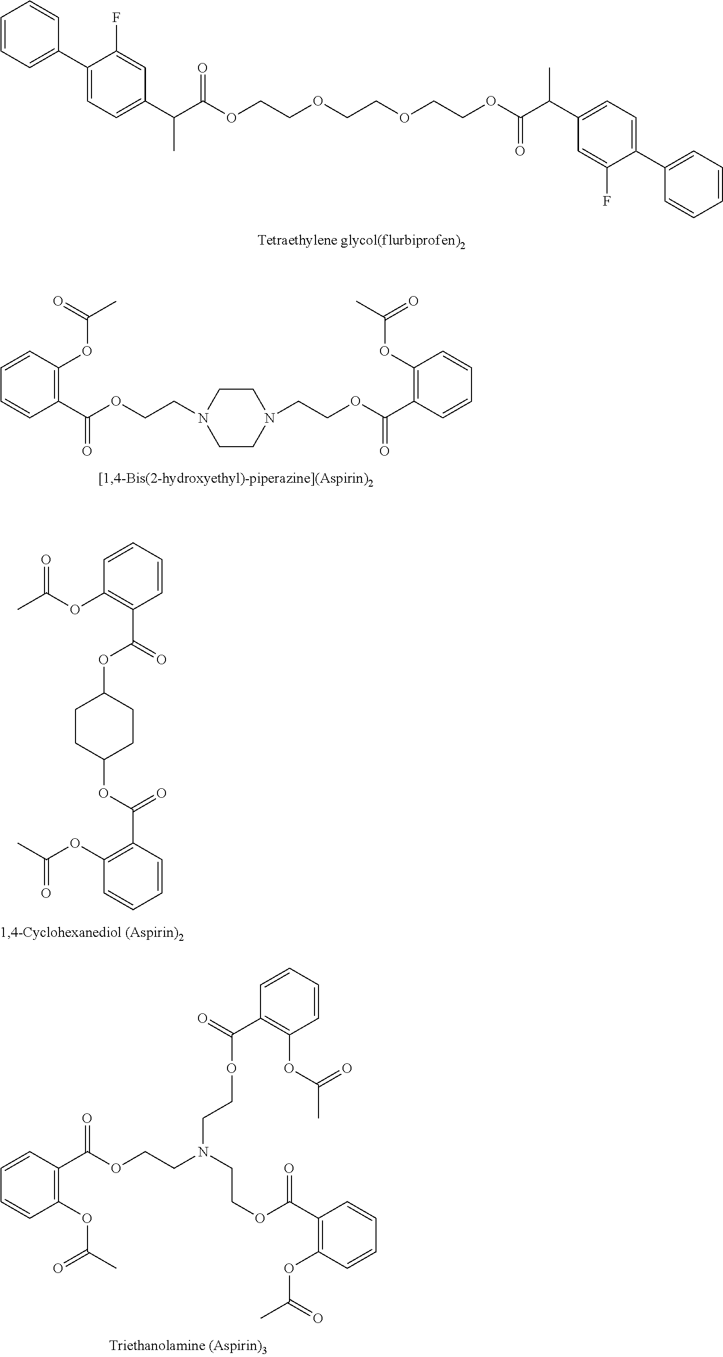 [SMILES: CC(C(=O)OCCOCCOCCOC(=O)C(C)c1ccc(-c2ccccc2)c(F)c1)c1ccc(-c2ccccc2)c(F)c1 ; CC(=O)Oc1ccccc1C(=O)OCCN1CCN(CCOC(=O)c2ccccc2OC(C)=O)CC1 ; CC(=O)Oc1ccccc1C(=O)OC1CCC(OC(=O)c2ccccc2OC(C)=O)CC1 ; CC(=O)Oc1ccccc1C(=O)OCCN(CCOC(=O)c1ccccc1OC(C)=O)CCOC(=O)c1ccccc1OC(C)=O]

A general scheme for the synthesis of the multiple NSAID-containing hydrophobic compounds and preparation of the NSAID nanospheres are described in the ensuing examples. The nanospheres showed sustained release of the free NSAIDs upon enzymatic hydrolysis by esterase.

### Antioxidant and Anti-Inflammatory Derivatives and Nanospheres

Various embodiments of the present invention use antioxidant and NSAID nanospheres. In one embodiment, antioxidant and NSAID nanospheres are capable of releasing the NSAIDs during a prolonged period of time.

Hydrophobic antioxidant and anti-inflammatory derivatives of an NSAID of the present invention may be represented by Formula B-II:

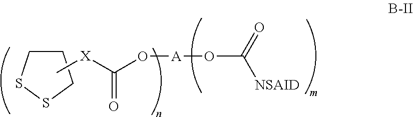

wherein X is selected from the group consisting of a substituted, unsubstituted, branched or unbranched chain of carbon atoms and may optionally contain a heteroatom; A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; n is an integer of at least one; and m is an integer of at least one. In one embodiment, X may be an unsubstituted, unbranched chain of 4 carbon atoms. In various embodiments, A is a moiety that is formed by esterification of at least two free esterifiable hydroxyl groups on a polyol. The polyol may be any polyol known in the art and as described above. The NSAID may be any NSAID known in the art and as described above

In one embodiment, the [1,2]-dithiolane moieties are from α-lipoic acid (“ALA”), and thus, the antioxidant and NSAID derivatives of the present invention may be represented by Formula B-III:

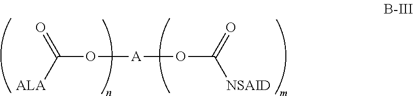

Accordingly, the antioxidant and NSAID nanospheres comprise a derivative of an NSAID and an α-lipoic acid.

Examples of particularly useful hydrophobic antioxidant and NSAID derivatives represented by formulas as follows:

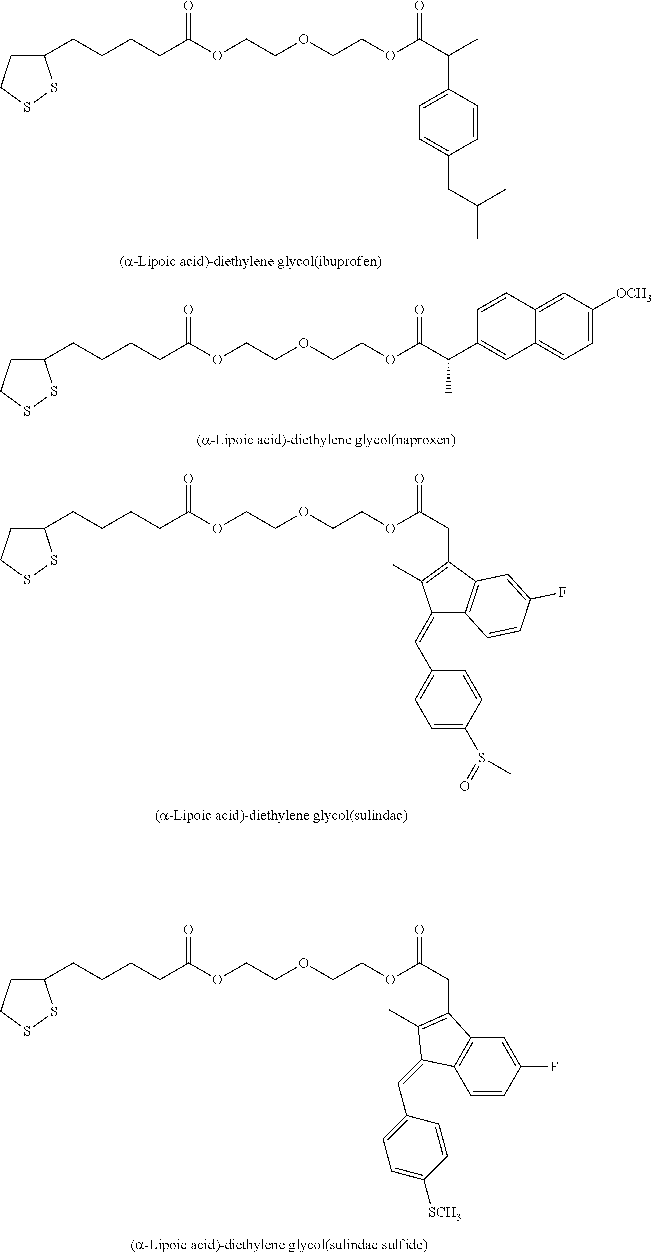 [SMILES: CC(C)Cc1ccc(C(C)C(=O)OCCOCCOC(=O)CCCCC2CCSS2)cc1 ; COc1ccc2cc([C@H](C)C(=O)OCCOCCOC(=O)CCCCC3CCSS3)ccc2c1 ; CC1=C(CC(=O)OCCOCCOC(=O)CCCCC2CCSS2)c2cc(F)ccc2/C1=C\c1ccc(S(C)=O)cc1 ; CSc1ccc(/C=C2/C(C)=C(CC(=O)OCCOCCOC(=O)CCCCC3CCSS3)c3cc(F)ccc32)cc1]

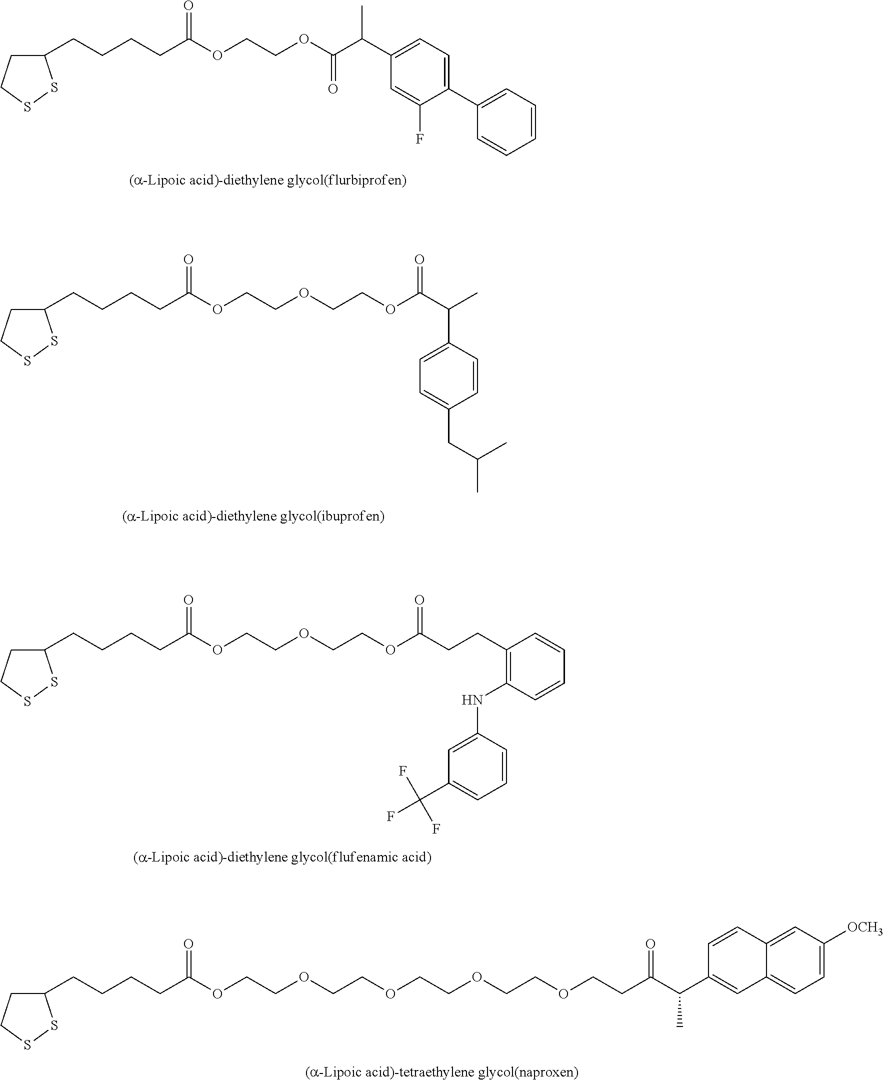 [SMILES: CC(C(=O)OCCOC(=O)CCCCC1CCSS1)c1ccc(-c2ccccc2)c(F)c1 ; CC(C)Cc1ccc(C(C)C(=O)OCCOCCOC(=O)CCCCC2CCSS2)cc1 ; O=C(CCCCC1CCSS1)OCCOCCOC(=O)CCc1ccccc1Nc1cccc(C(F)(F)F)c1 ; COc1ccc2cc([C@H](C)C(=O)CCOCCOCCOCCOCCOC(=O)CCCCC3CCSS3)ccc2c1]

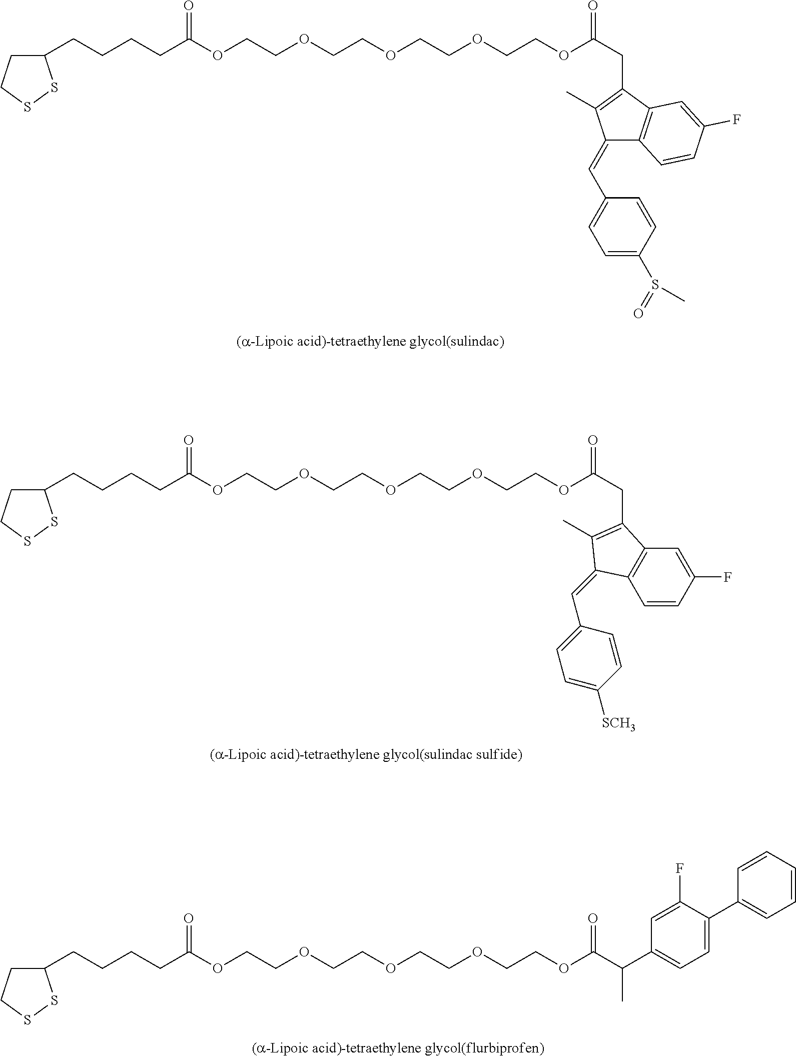 [SMILES: CC1=C(CC(=O)OCCOCCOCCOCCOC(=O)CCCCC2CCSS2)c2cc(F)ccc2/C1=C\c1ccc(S(C)=O)cc1 ; CSc1ccc(/C=C2/C(C)=C(CC(=O)OCCOCCOCCOCCOC(=O)CCCCC3CCSS3)c3cc(F)ccc32)cc1 ; CC(C(=O)OCCOCCOCCOCCOC(=O)CCCCC1CCSS1)c1ccc(-c2ccccc2)c(F)c1]

A general scheme for the synthesis of the α-lipoic acid and NSAID-containing hydrophobic compounds and preparation of the inventive antioxidant and NSAID nanospheres are described in the ensuing examples. The antioxidant activity of the nanospheres has been demonstrated by HOCl scavenging assay.

### CPT Nanospheres

In certain embodiments, the nanospheres are formed with antioxidant derivatives of camptothecin or antioxidant derivatives of captothecin analogs. Thus, in certain embodiments, the nanospheres comprise derivatives of camptothecin or antioxidant derivatives of captothecin analogs.

In one embodiment, an antioxidant derivative of camptothecin and/or an antioxidant derivative of a camptothecin analog may be represented by Formula C-II:

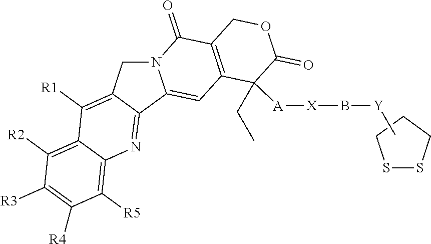

wherein A and B may be independently selected from the group consisting of —OC(O)—, —OC(O)O—, and —OC(O)N(R)—, wherein R may be a hydrogen atom, or a substituted, unsubstituted, branched or unbranched chain of carbon atoms and may contain heteroatoms (e.g., nitrogen, oxygen, sulfur, etc.); wherein X and Y may be each be a linker that may be a substituted, unsubstituted, branched or unbranched chain of carbon atoms and may contain heteroatoms (e.g., nitrogen, oxygen, sulfur, etc.); and wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

In one embodiment, an antioxidant derivative of camptothecin and/or antioxidant derivative of a camptothecin analog is prepared by the conjugation of a camptothecin or a camptothecin analog and an α-lipoic acid and is represented by Formula C-III:

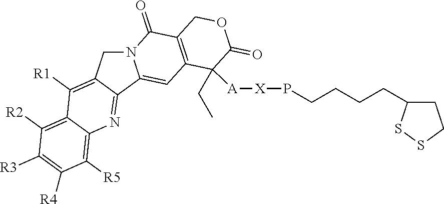

wherein A may be selected from the group consisting of —OC(O)—, —OC(O)O—, and —OC(O)N(R)—, wherein R may be a hydrogen atom, or a substituted, unsubstituted, branched or unbranched chain of carbon atoms and may contain heteroatoms (e.g., nitrogen, oxygen, sulfur, etc.); wherein P may be selected from the group consisting of —OC(O)—, and —N(R)C(O)—, wherein R may be a hydrogen atom, or a substituted, unsubstituted, branched or unbranched chain of carbon atoms and may contain heteroatoms (e.g., nitrogen, oxygen, sulfur, etc.); wherein X may be a linker that may be a substituted, unsubstituted, branched or unbranched chain of carbon atoms and may contain heteroatoms (e.g., nitrogen, oxygen, sulfur, etc.); and wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

In another embodiment, an antioxidant derivative of camptothecin and/or antioxidant derivative of a camptothecin analog is prepared by the conjugation of camptothecin or a camptothecin analog and α-lipoic acid via a diol and is represented Formula C-IV:

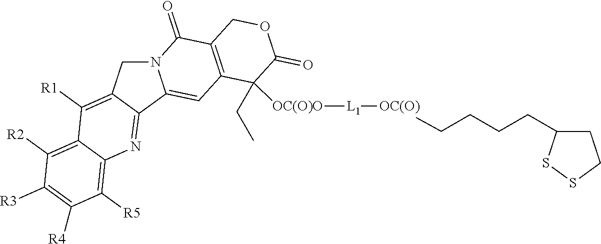

wherein L₁ may be a moiety formed by esterification of two free esterifiable hydroxyl groups on a diol; and wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

In various embodiments, diols that are useful in the present invention may be represented by the following formula:

HO—W—OH

wherein W may be a hydrocarbon group; for example, an alkyl, aryl, cycloaliphatic or aralkyl group; and may be saturated or unsaturated. W may also contain hetero atoms (e.g., nitrogen, oxygen, sulfur, etc.).

Additional examples of diols are those in Table 10. Further examples of diols that are useful in the present invention include, but are not limited to commercially available one as follows:

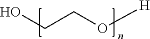

wherein n is an integer between 1 and 100.

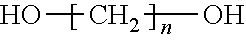

wherein n is an integer between 2 and 12.

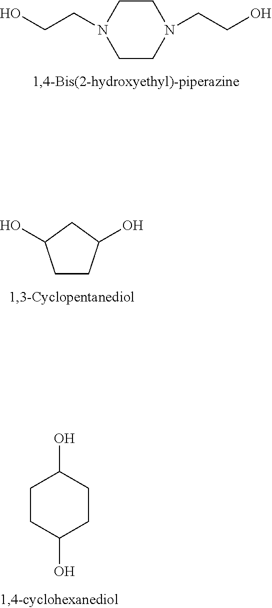 [SMILES: OCCN1CCN(CCO)CC1 ; OC1CCC(O)C1 ; OC1CCC(O)CC1]

Examples of particularly useful antioxidant derivatives of camptothecin and/or antioxidant derivatives of camptothecin analogs of this embodiment are represented by the following formulas:

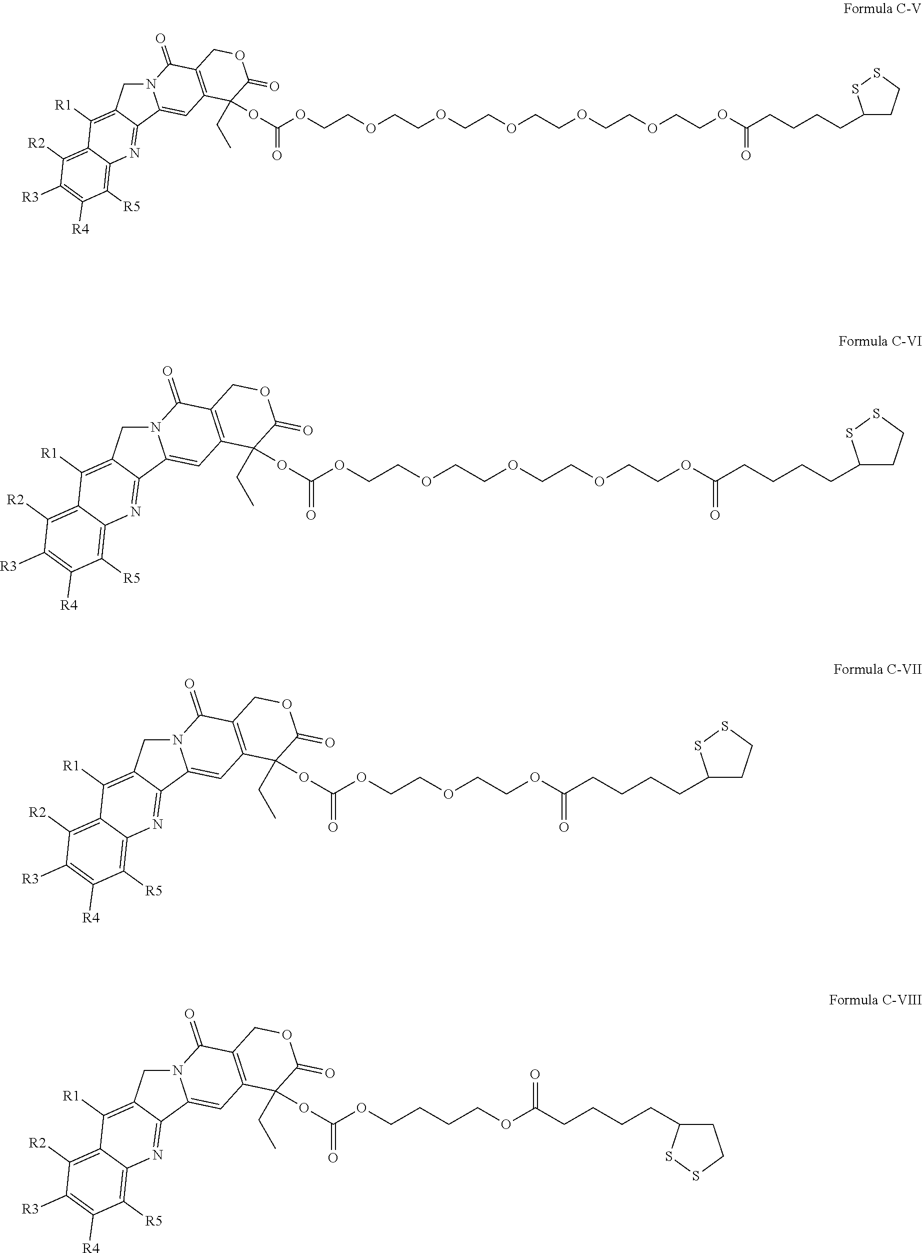

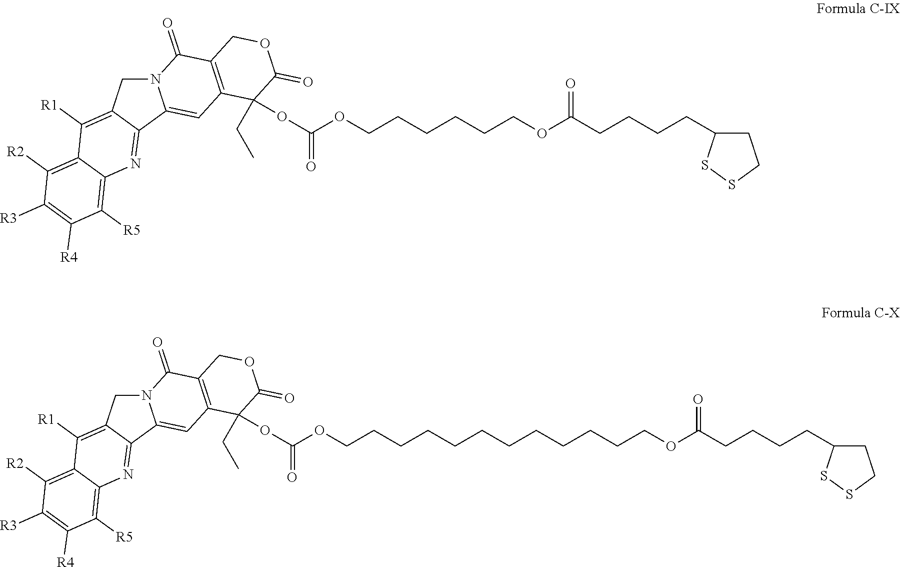

wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

One exemplary compound and its synthesis are shown below.

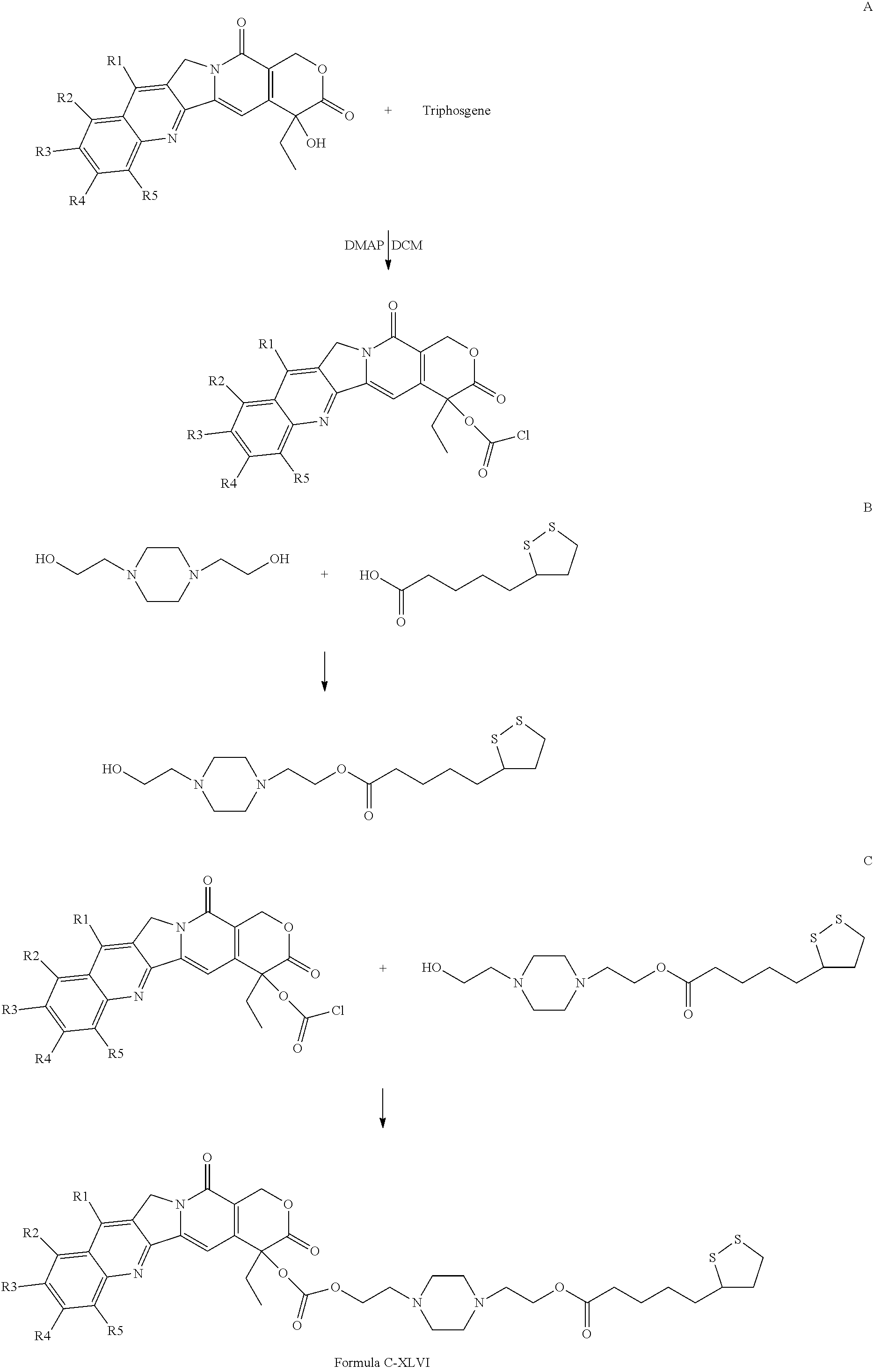

In another embodiment, an antioxidant derivative of a camptothecin and/or antioxidant derivative of a camptothecin analog is prepared by the conjugation of camptothecin or a camptothecin analog and an α-lipoic acid via a diamine and is represented by Formula C-XI:

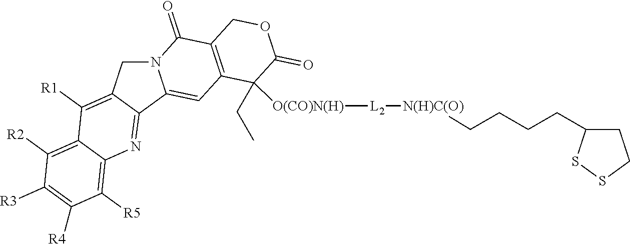

wherein L₂ may be a moiety formed by using a diamine as the linker in the process of producing the antioxidant camptothecin derivative or the antioxidant camptothecin analog derivative; and wherein R₁, R₂, R₃, R₄, R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

In one embodiment, diamines that are useful in the present invention may be represented by the following formula:

H₂N—X—NH,

wherein X may be a hydrocarbon group; for example, an alkyl, aryl, cycloaliphatic or aralkyl group; and may be saturated or unsaturated. X may also contain hetero atoms (e.g., nitrogen, oxygen, sulfur, etc.).

In other embodiments, diamines that are useful in the present inventive compounds include, but are not limited to commercially available ones as follows:

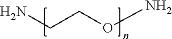

wherein n is an integer between 1 and 100.

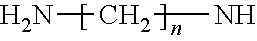

wherein n is an integer between 2 and 12.

Examples of particularly useful antioxidant derivatives of camptothecin and/or antioxidant derivatives of camptothecin analogs of this embodiment are represented by the following formulas:

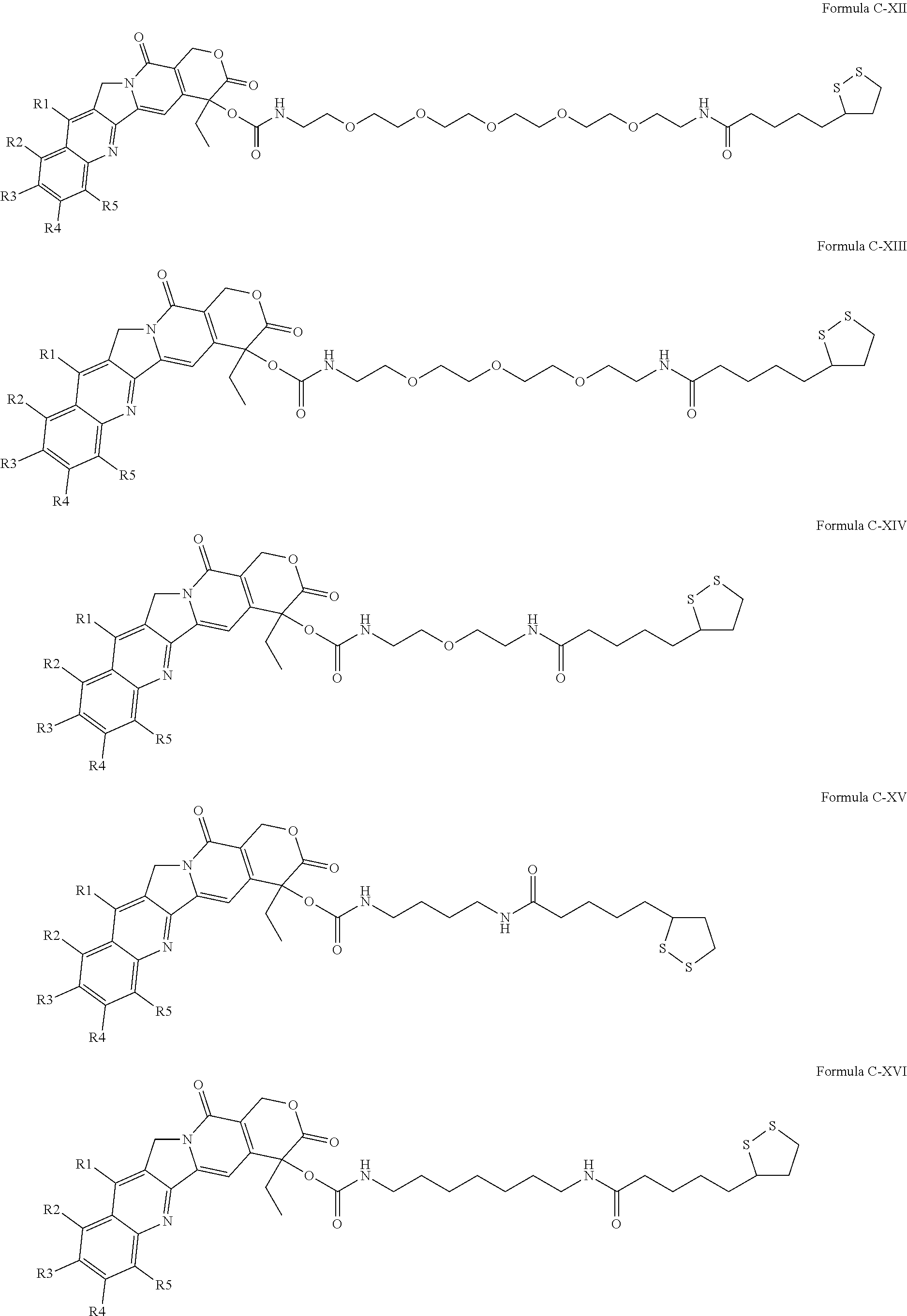

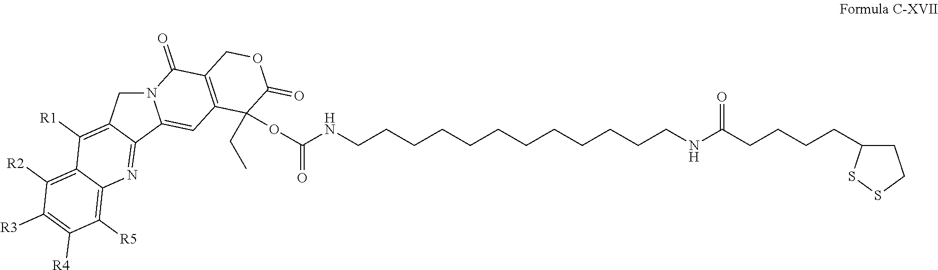

wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

One exemplary compound and its synthesis are shown below.

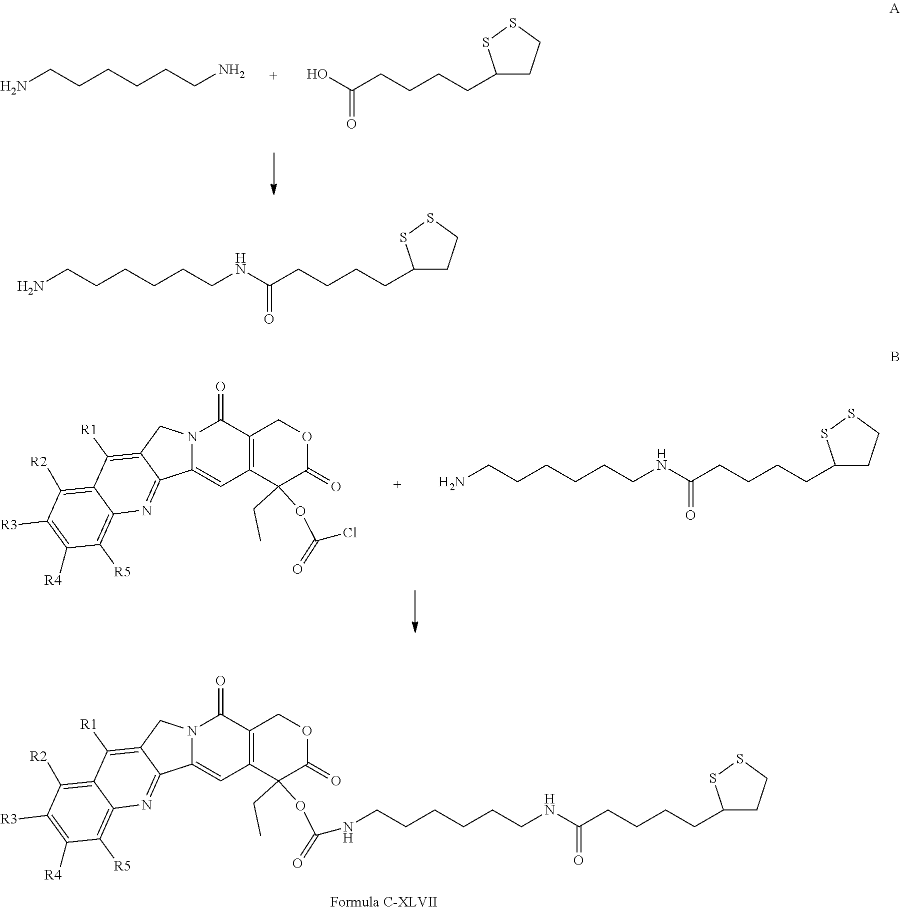

In another embodiment, an antioxidant derivative of camptothecin and/or antioxidant derivative of a camptothecin analog is prepared by the conjugation of camptothecin or a camptothecin analog and an α-lipoic acid via an aminoalcohol and is represented by Formula

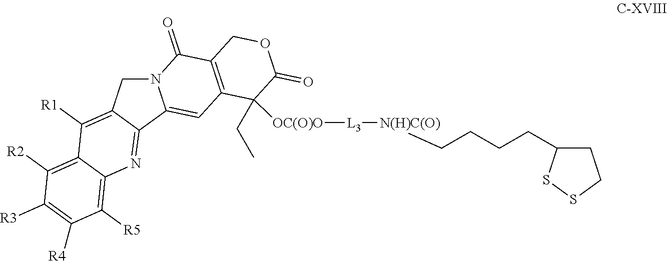

wherein L₃ may be a moiety formed by using an aminoalcohol as the linker in the process of producing the antioxidant camptothecin derivative or the antioxidant camptothecin analog derivative; and wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

Aminoalcohols that are useful in the present invention may be represented by the following formula:

H₂N—Y—OH

wherein Y may be a hydrocarbon group; for example, an alkyl, aryl, cycloaliphatic or aralkyl group; and may be saturated or unsaturated. Y may also contain hetero atoms (e.g., nitrogen, oxygen, sulfur, etc.).

Examples of aminoalcohols that are useful in the present inventive compounds include, but are not limited to commercially available one as follows:

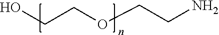

wherein n is an integer between 1 and 100.

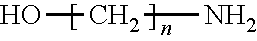

wherein n is an integer between 2 and 12.

Examples of particularly useful antioxidant derivatives of camptothecin and/or antioxidant derivatives of camptothecin analogs of this embodiment are represented by the following formulas:

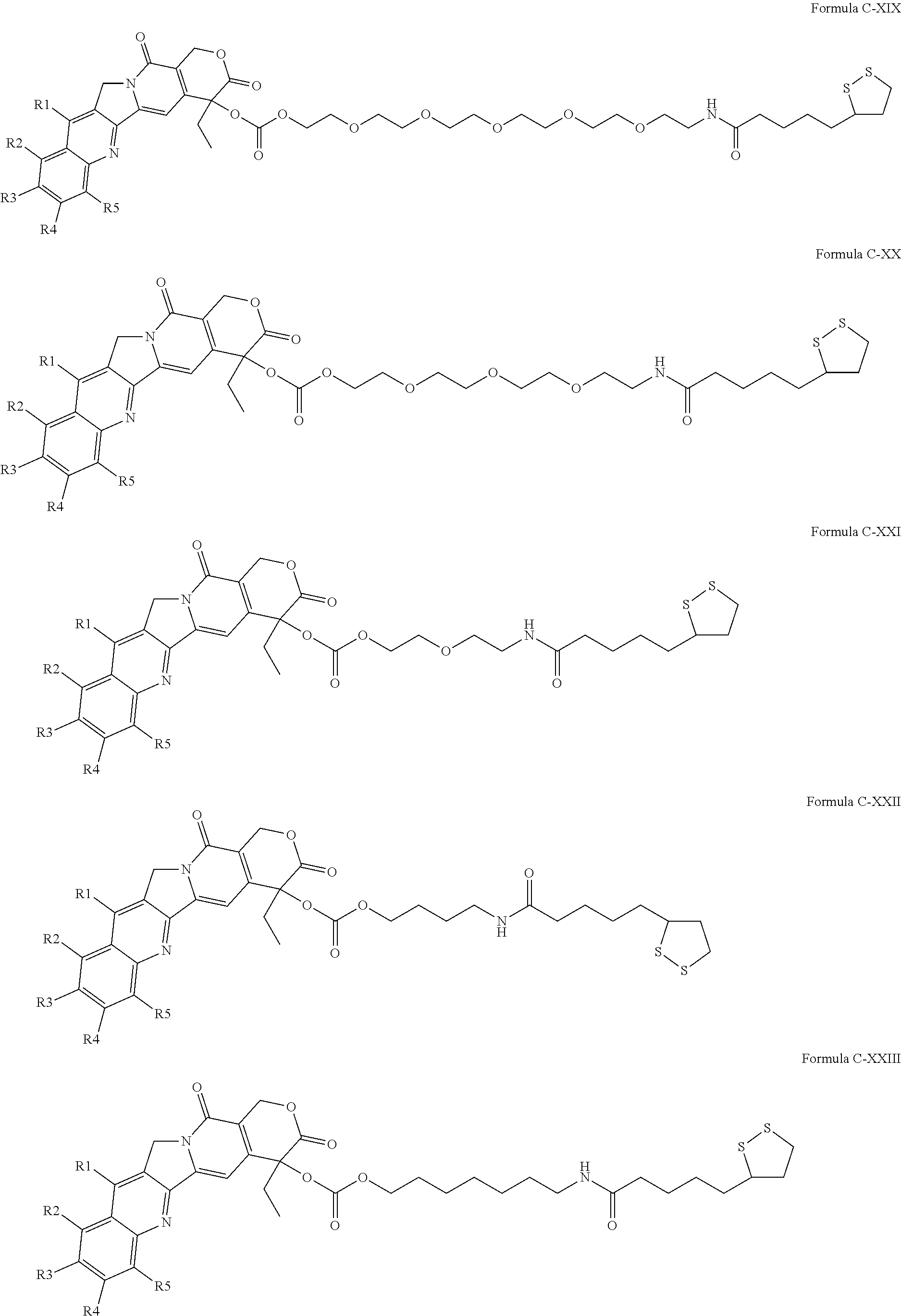

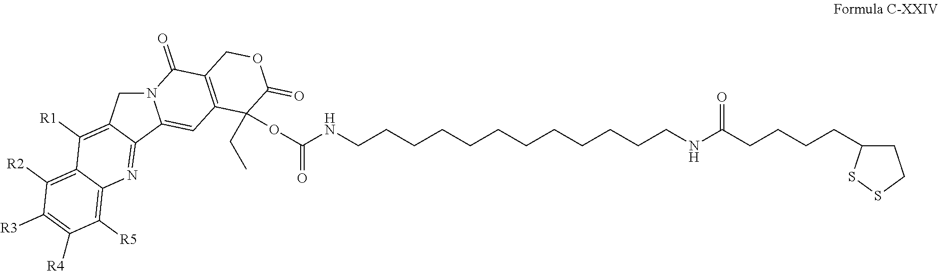

wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

One exemplary compound and its synthesis are shown below.

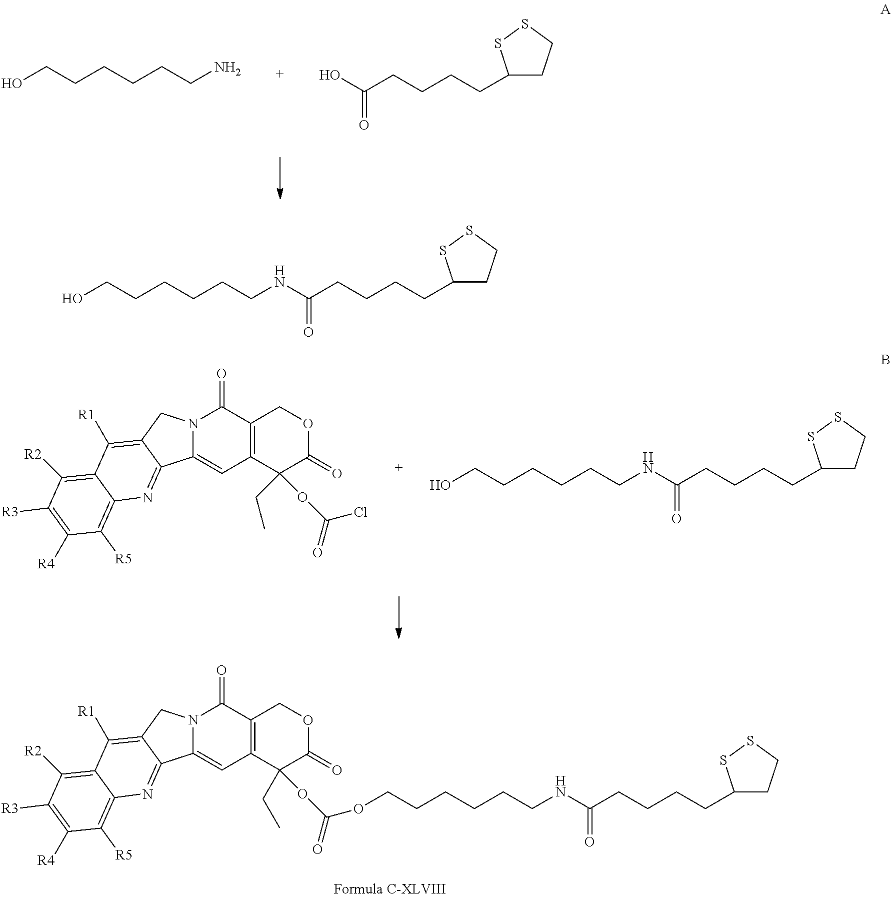

Additional embodiments of the present invention provide for the following compounds:

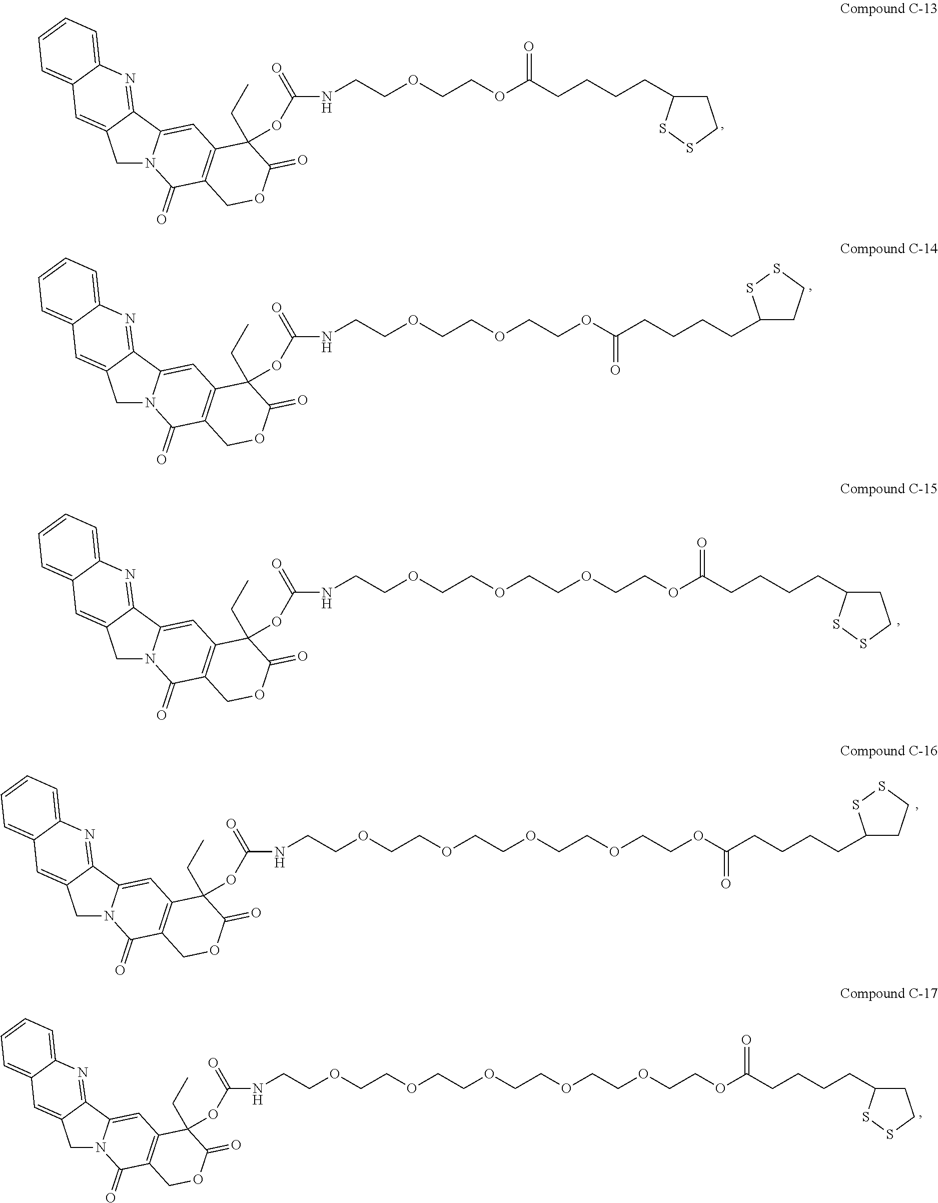 [SMILES: CCC1(OC(=O)NCCOCCOC(=O)CCCCC2CCSS2)C(=O)OCc2c1cc1n(c2=O)Cc2cc3ccccc3nc2-1 ; CCC1(OC(=O)NCCOCCOCCOC(=O)CCCCC2CCSS2)C(=O)OCc2c1cc1n(c2=O)Cc2cc3ccccc3nc2-1 ; CCC1(OC(=O)NCCOCCOCCOCCOC(=O)CCCCC2CCSS2)C(=O)OCc2c1cc1n(c2=O)Cc2cc3ccccc3nc2-1 ; CCC1(OC(=O)NCCOCCOCCOCCOCCOC(=O)CCCCC2CCSS2)C(=O)OCc2c1cc1n(c2=O)Cc2cc3ccccc3nc2-1 ; CCC1(OC(=O)NCCOCCOCCOCCOCCOCCOC(=O)CCCCC2CCSS2)C(=O)OCc2c1cc1n(c2=O)Cc2cc3ccccc3nc2-1]

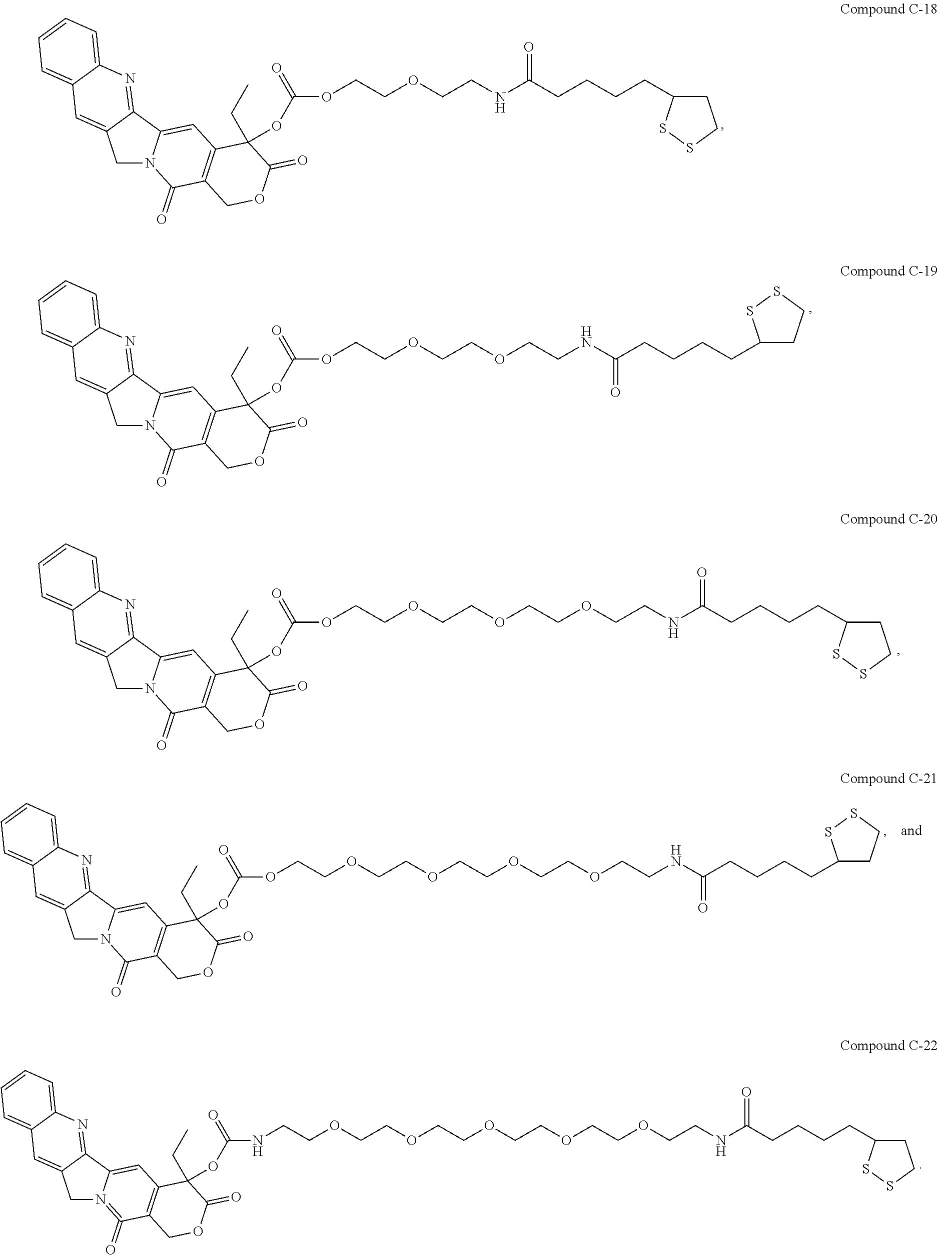

In another embodiment, the camptothecin analogs are modified by reaction with succinic anhydride or glutaric anhydride and an antioxidant derivative of camptothecin and/or antioxidant derivative of a camptothecin analog is prepared by the conjugation of an α-lipoic acid and the modified camptothecin or camptothecin analog. One exemplary compound and its synthesis are shown below.

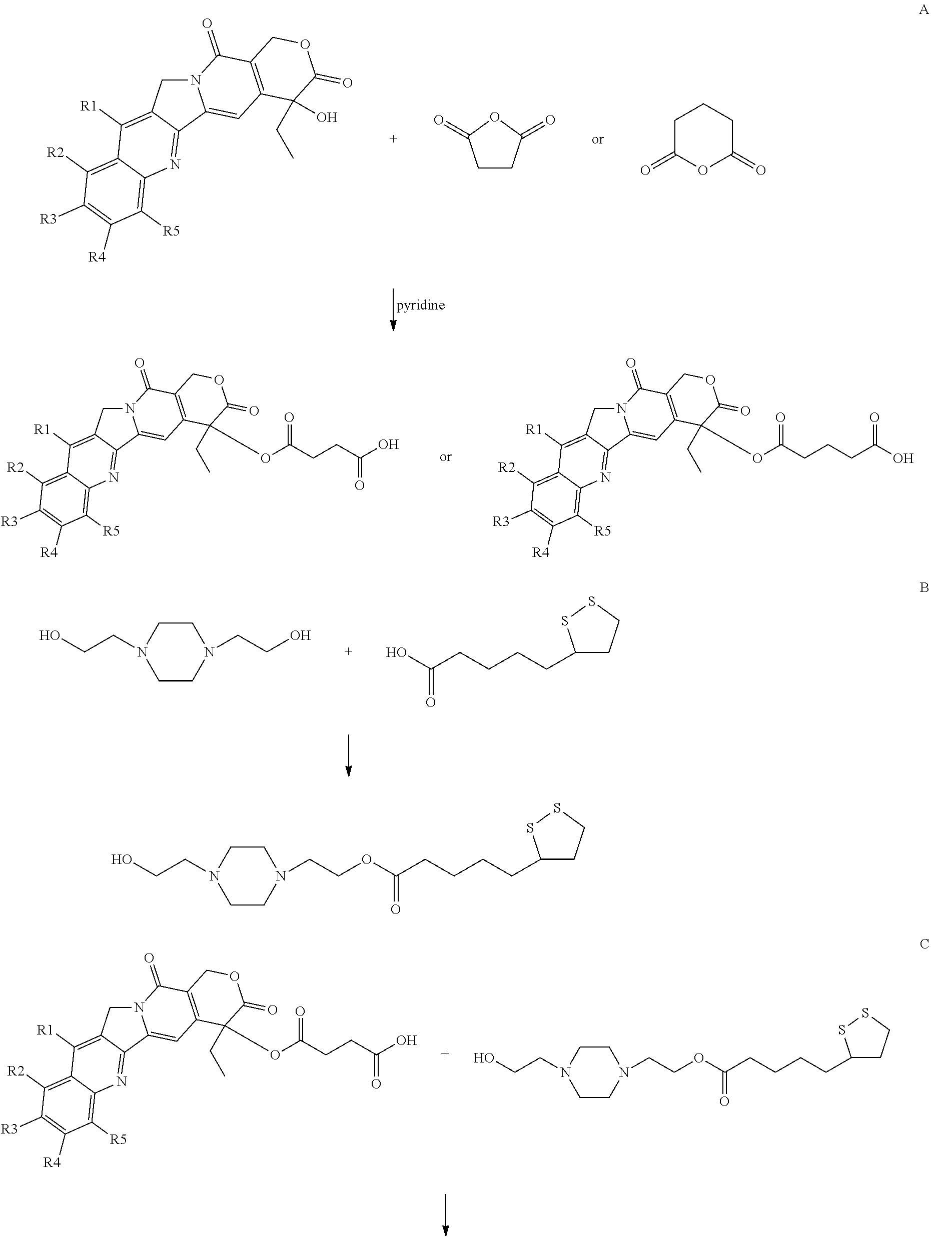

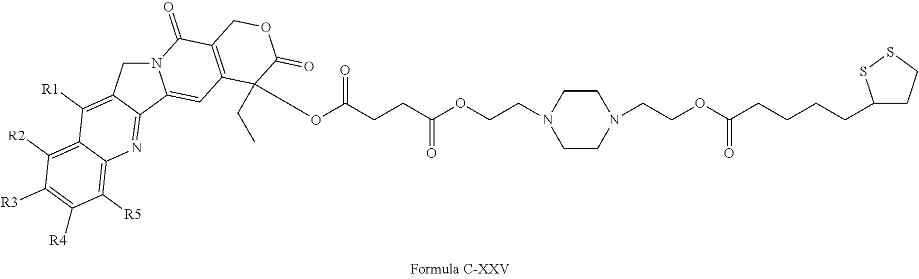

wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

Additional examples of particularly useful antioxidant derivatives of camptothecin and/or antioxidant derivatives of camptothecin analogs are represented by formulas as follows:

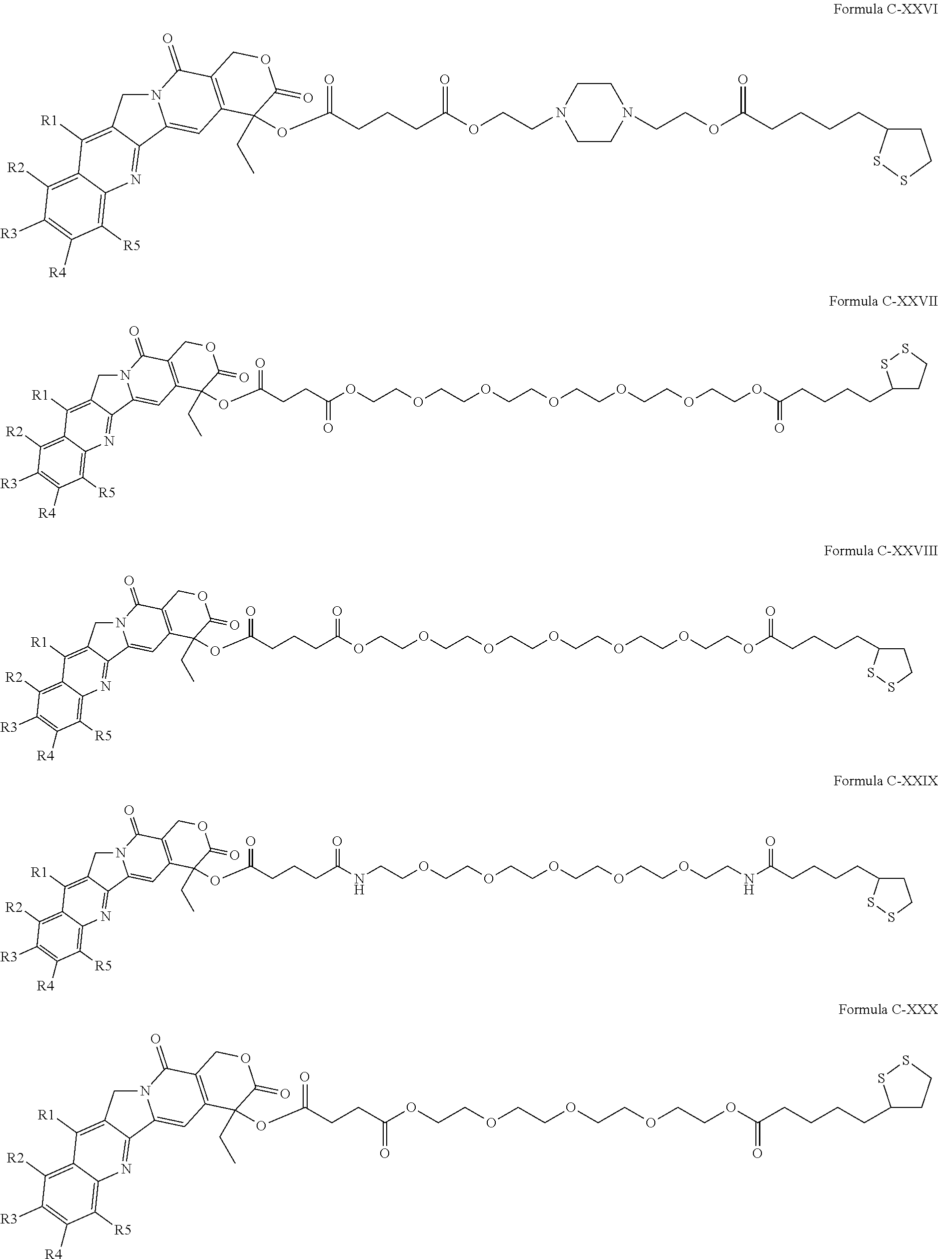

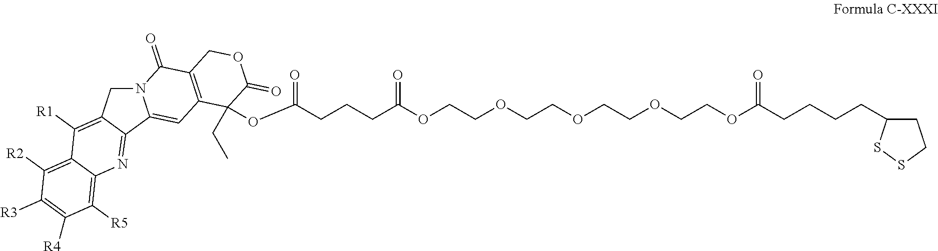

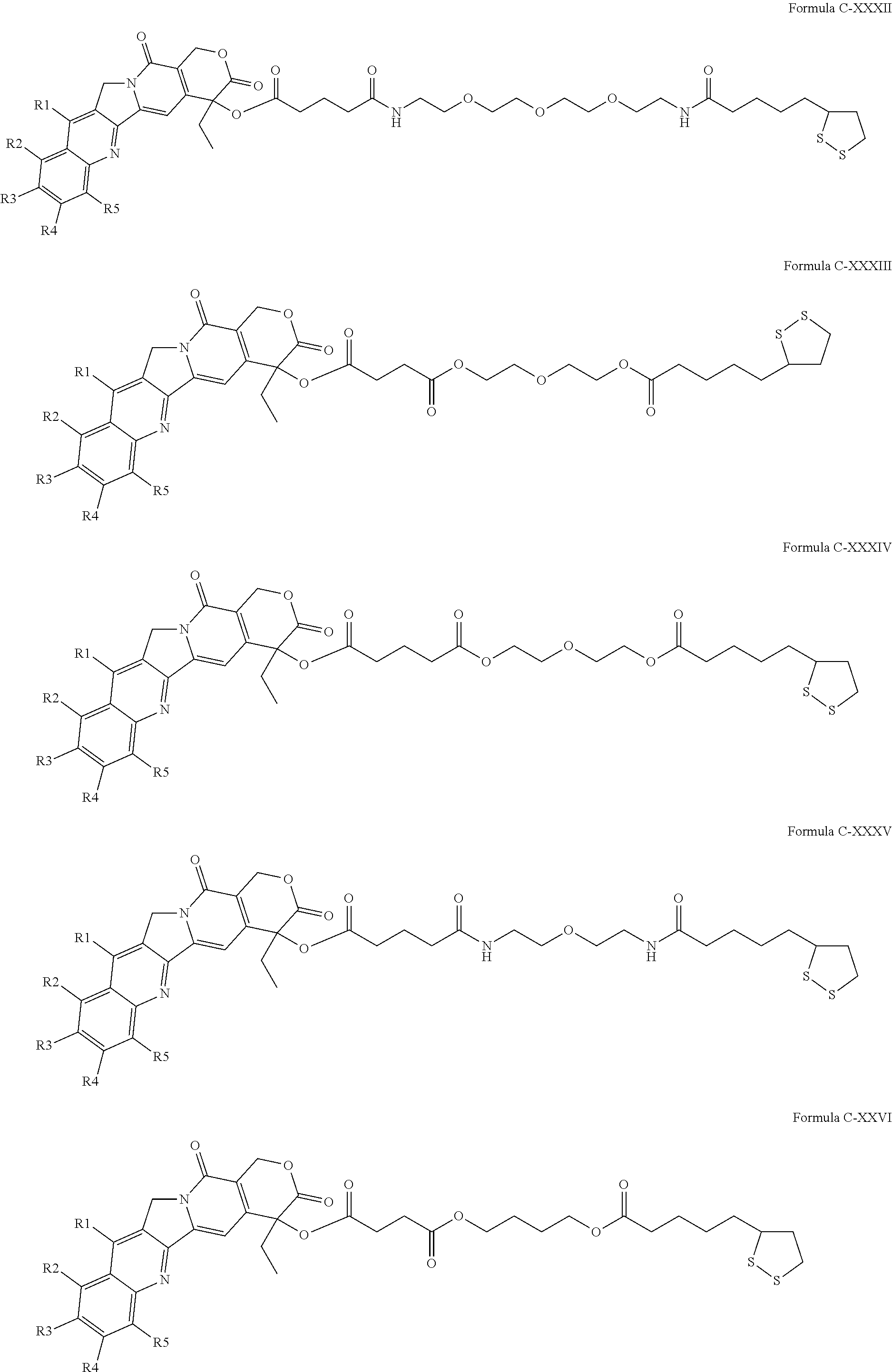

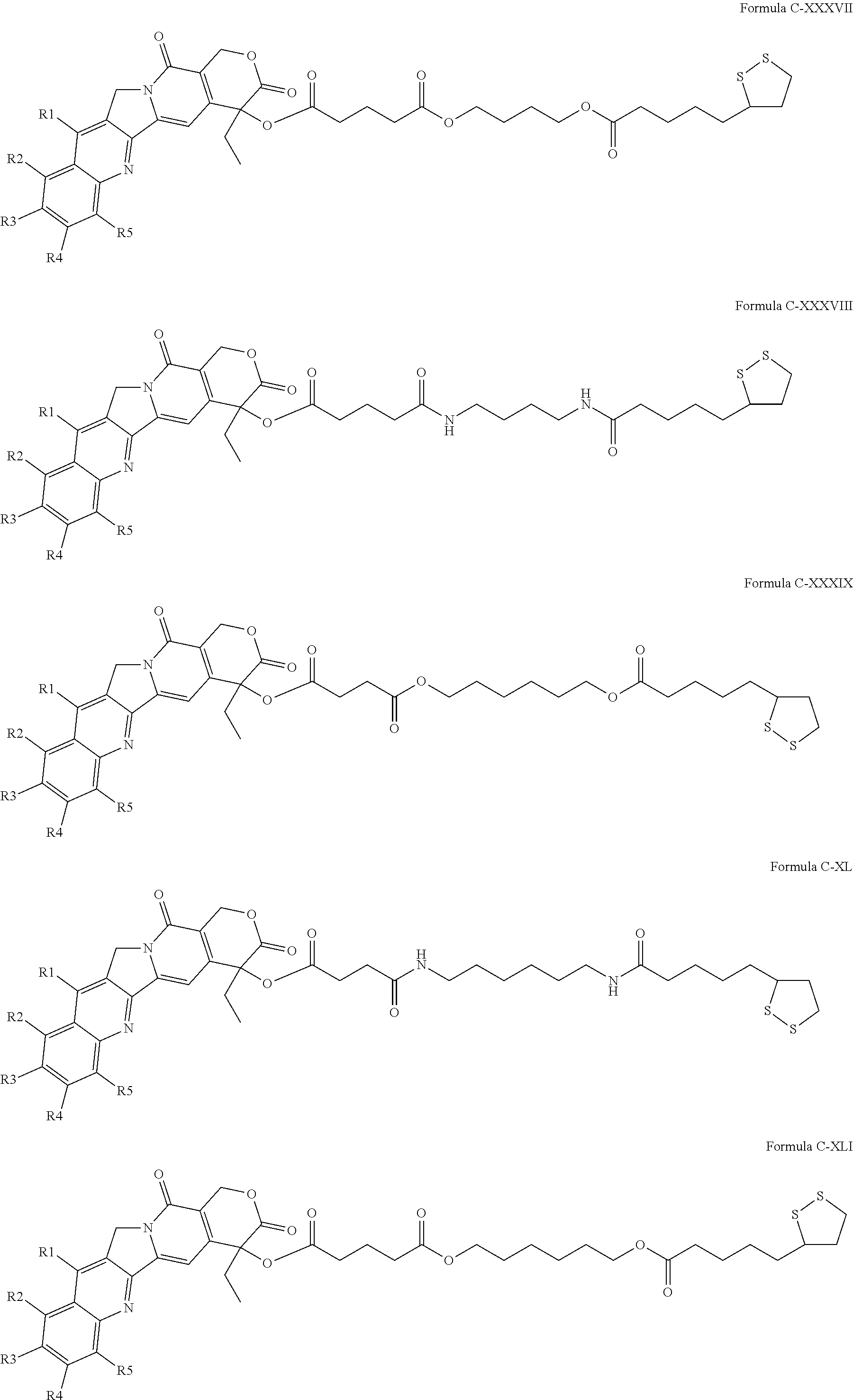

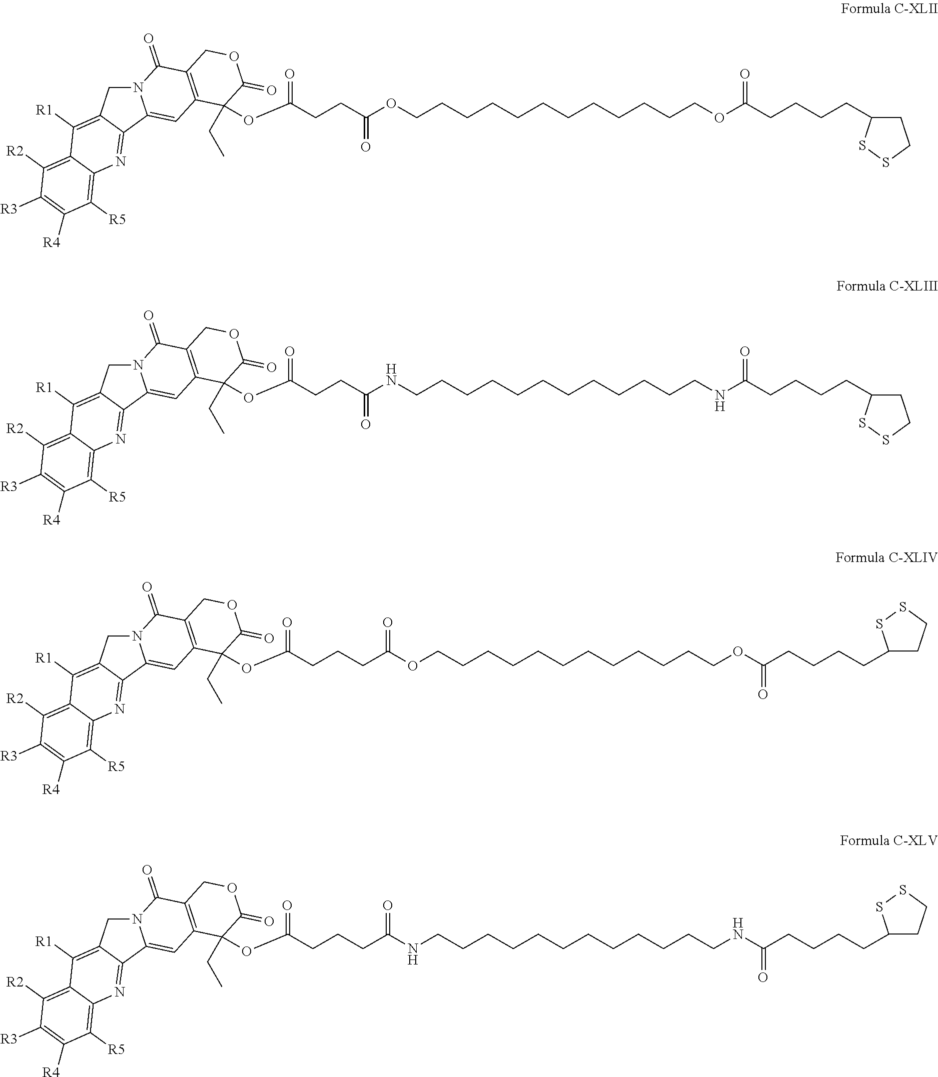

wherein R₁, R₂, R₃, R₄, and R₅ may each be independently selected from hydrogen or a substituent selected from an alkyl, aryl, cycloaliphatic, and aralkyl group, may be saturated or unsaturated, and may contain hetero atoms (e.g., nitrogen, oxygen, sulfur, halogens, etc).

In one particular embodiment, each of R₁ through R₅ of the formulas and/or compounds described above is H, and is shown below:

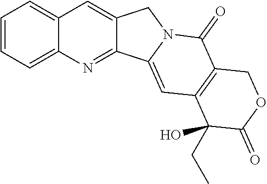 [SMILES: CC[C@@]1(O)C(=O)OCc2c1cc1n(c2=O)Cc2cc3ccccc3nc2-1]

A general scheme for the synthesis of the antioxidant derivatives of camptothecin and antioxidant derivatives of camptothecin analogs and preparation of the antioxidant-antineoplastic nanospheres are described in the ensuing examples. The synthetic procedure is both simple and versatile and leads to the synthesis of the antioxidant derivatives of camptothecin and antioxidant derivatives of camptothecin analogs varying in size and hydrophobicity.

### Statin Derivatives and Nanospheres

In certain embodiments, the nanospheres are formed with statin derivatives. Thus, in certain embodiments, the nanospheres comprise derivatives of statins

In one embodiment, a statin derivative may be represented by Formula D-I:

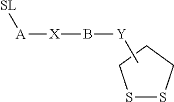

wherein A and B may be independently selected from the group consisting of —OC(O)—, —OC(O)O—, and —OC(O)N(R)—, wherein R may be a hydrogen atom, or a substituted, unsubstituted, branched or unbranched chain of carbon atoms and may contain heteroatoms (e.g., nitrogen, oxygen, sulfur, etc.); wherein X and Y may be each be a linker that may be a substituted, unsubstituted, branched or unbranched chain of carbon atoms and may contain heteroatoms (e.g., nitrogen, oxygen, sulfur, etc.); and wherein SL may be selected from the statin lactones from the group consisting of atorvastatin, fluvastatin, lovastatin, mevastatin, pitavastatin, rosuvastatin, and simvastatin.

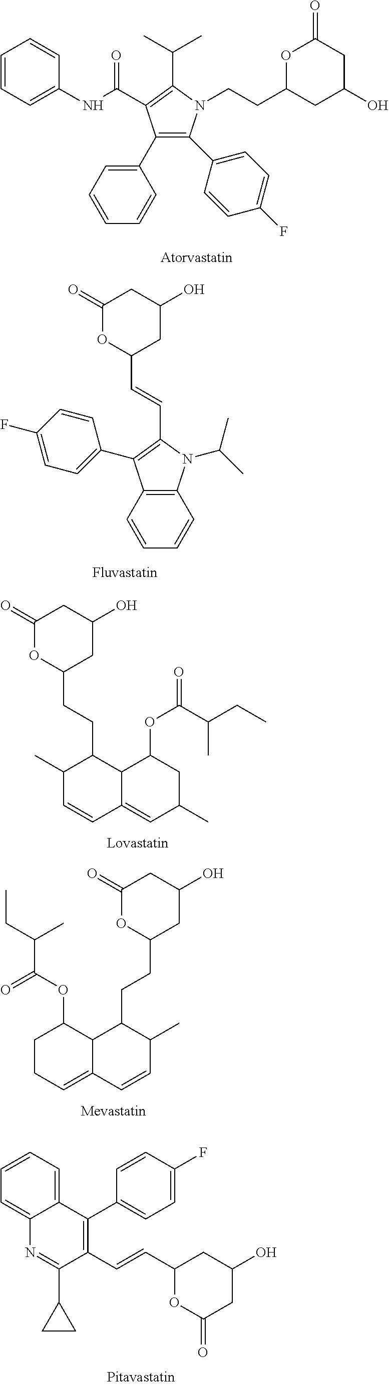 [SMILES: CC(C)c1c(C(=O)Nc2ccccc2)c(-c2ccccc2)c(-c2ccc(F)cc2)n1CCC1CC(O)CC(=O)O1 ; CC(C)n1c(/C=C/C2CC(O)CC(=O)O2)c(-c2ccc(F)cc2)c2ccccc21 ; CCC(C)C(=O)OC1CC(C)C=C2C=CC(C)C(CCC3CC(O)CC(=O)O3)C21 ; CCC(C)C(=O)OC1CCC=C2C=CC(C)C(CCC3CC(O)CC(=O)O3)C21 ; O=C1CC(O)CC(/C=C/c2c(C3CC3)nc3ccccc3c2-c2ccc(F)cc2)O1]

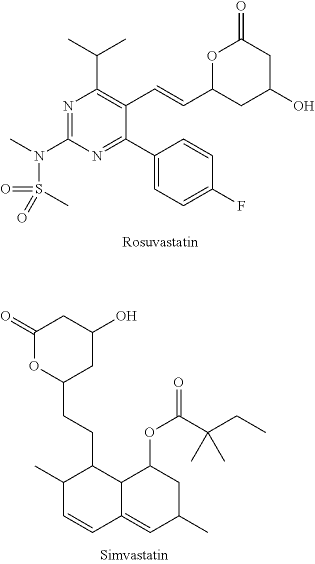 [SMILES: CC(C)c1nc(N(C)S(C)(=O)=O)nc(-c2ccc(F)cc2)c1/C=C/C1CC(O)CC(=O)O1 ; CCC(C)(C)C(=O)OC1CC(C)C=C2C=CC(C)C(CCC3CC(O)CC(=O)O3)C21]

In one embodiment, a statin derivative is prepared by the conjugation of a statin and an α-lipoic acid and is represented by Formula D-II:

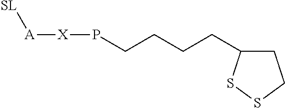

wherein A may be selected from the group consisting of —OC(O)—, —OC(O)O—, and —OC(O)N(R)—, wherein R may be a hydrogen atom, or a substituted, unsubstituted, branched or unbranched chain of carbon atoms and may contain heteroatoms (e.g., nitrogen, oxygen, sulfur, etc.); wherein P may be selected from the group consisting of —OC(O)—, and —N(R)C(O)—, wherein R may be a hydrogen atom, or a substituted, unsubstituted, branched or unbranched chain of carbon atoms and may contain heteroatoms (e.g., nitrogen, oxygen, sulfur, etc.); wherein X may be a linker that may be a substituted, unsubstituted, branched or unbranched chain of carbon atoms and may contain heteroatoms (e.g., nitrogen, oxygen, sulfur, etc.); and wherein SL may be selected from the statin lactones from the group consisting of atorvastatin, fluvastatin, lovastatin, mevastatin, pitavastatin, rosuvastatin, and simvastatin.

In another embodiment, an antioxidant derivative of statin is prepared by the conjugation of a statin lactone and α-lipoic acid via a diol and is represented Formula D-III:

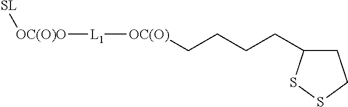

wherein L₁ may be a moiety formed by esterification of two free esterifiable hydroxyl groups on a diol; and wherein SL may be selected from the statin lactones from the group consisting of atorvastatin, fluvastatin, lovastatin, mevastatin, pitavastatin, rosuvastatin, and simvastatin.

In various embodiments, diols that are useful in the present invention may be represented by the following formula:

HO—W—OH

wherein W may be a hydrocarbon group; for example, an alkyl, aryl, cycloaliphatic or aralkyl group; and may be saturated or unsaturated. W may also contain hetero atoms (e.g., nitrogen, oxygen, sulfur, etc.).

Additional examples of diols are those in Table 10. Further, examples of diols that are useful in the present invention include, but are not limited to commercially available one as follows:

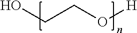

wherein n is an integer between 1 and 100.

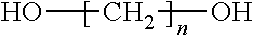

wherein n is an integer between 2 and 12.

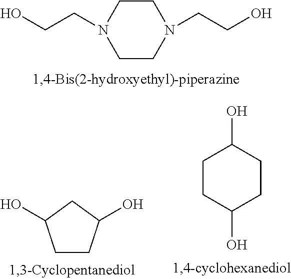 [SMILES: OCCN1CCN(CCO)CC1 ; OC1CCC(O)C1 ; OC1CCC(O)CC1]

Examples of particularly useful derivatives of statins of this embodiment are represented by the following formulas using lovastatin:

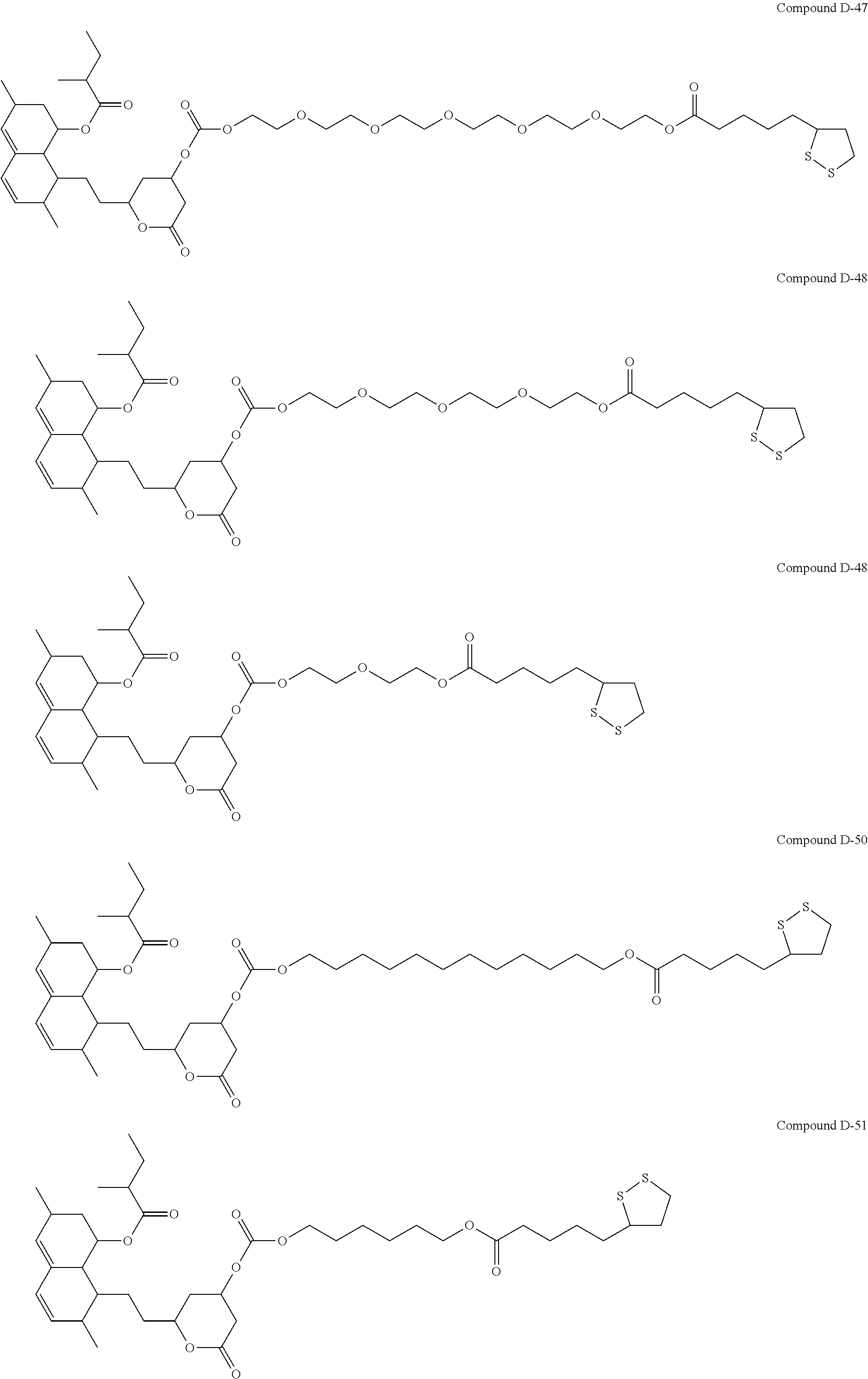 [SMILES: CCC(C)C(=O)OC1CC(C)C=C2C=CC(C)C(CCC3CC(OC(=O)OCCOCCOCCOCCOCCOCCOC(=O)CCCCC4CCSS4)CC(=O)O3)C21 ; CCC(C)C(=O)OC1CC(C)C=C2C=CC(C)C(CCC3CC(OC(=O)OCCOCCOCCOCCOC(=O)CCCCC4CCSS4)CC(=O)O3)C21 ; CCC(C)C(=O)OC1CC(C)C=C2C=CC(C)C(CCC3CC(OC(=O)OCCOCCOC(=O)CCCCC4CCSS4)CC(=O)O3)C21 ; CCC(C)C(=O)OC1CC(C)C=C2C=CC(C)C(CCC3CC(OC(=O)OCCCCCCCCCCCCOC(=O)CCCCC4CCSS4)CC(=O)O3)C21 ; CCC(C)C(=O)OC1CC(C)C=C2C=CC(C)C(CCC3CC(OC(=O)OCCCCCCOC(=O)CCCCC4CCSS4)CC(=O)O3)C21]

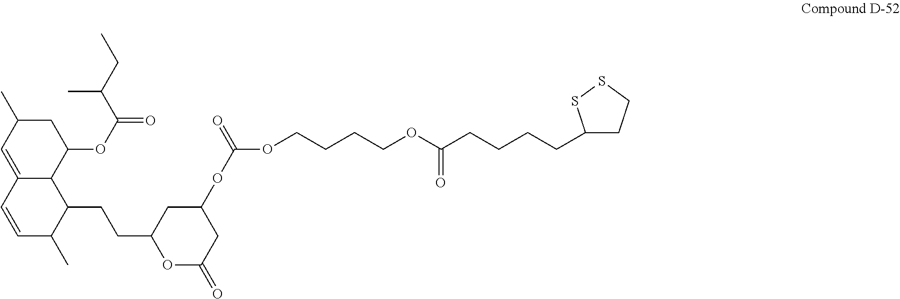 [SMILES: CCC(C)C(=O)OC1CC(C)C=C2C=CC(C)C(CCC3CC(OC(=O)OCCCCOC(=O)CCCCC4CCSS4)CC(=O)O3)C21]

In another embodiment, a statin derivative is prepared by the conjugation of a statin lactone and an α-lipoic acid via a diamine and is represented by Formula D-IV:

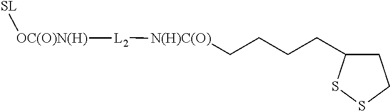

wherein L₂ may be a moiety formed by using a diamine as the linker in the process of producing the derivative of statin lactones, and wherein SL may be selected from the statin lactones consisting of atorvastatin, fluvastatin, lovastatin, mevastatin, pitavastatin, rosuvastatin, and simvastatin.

In one embodiment, diamines that are useful in the present invention may be represented by the following formula:

H₂N—X—NH₂

wherein X may be a hydrocarbon group; for example, an alkyl, aryl, cycloaliphatic or aralkyl group; and may be saturated or unsaturated. X may also contain hetero atoms (e.g., nitrogen, oxygen, sulfur, etc.).

In other embodiments, diamines that are useful in the present inventive compounds include, but are not limited to commercially available ones as follows:

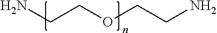

wherein n is an integer between 1 and 100.

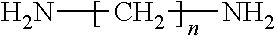

wherein n is an integer between 2 and 12.

Examples of particularly useful derivatives of statin lactones of this embodiment are represented by the following compounds using lovastatin:

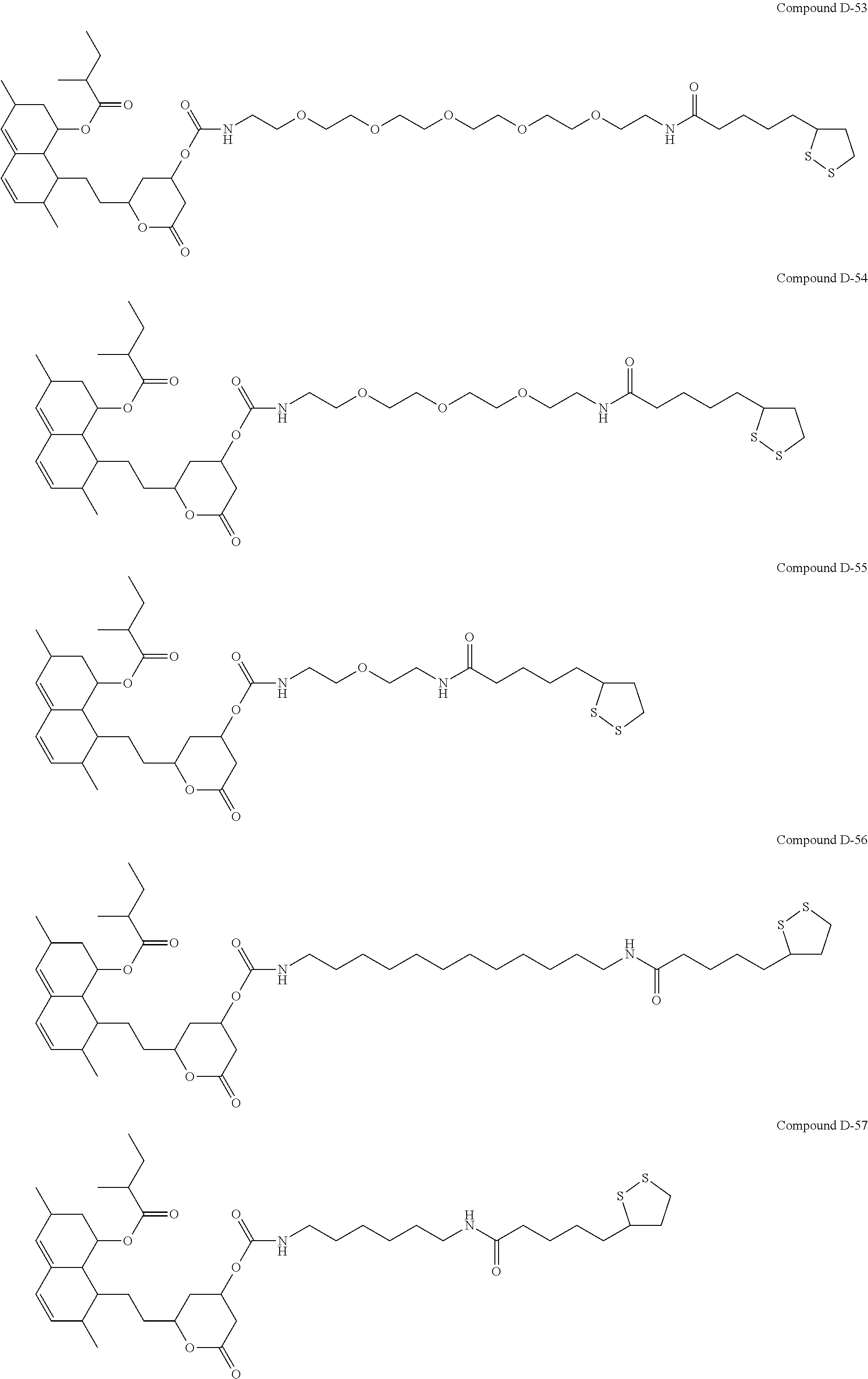 [SMILES: CCC(C)C(=O)OC1CC(C)C=C2C=CC(C)C(CCC3CC(OC(=O)NCCOCCOCCOCCOCCOCCNC(=O)CCCCC4CCSS4)CC(=O)O3)C21 ; CCC(C)C(=O)OC1CC(C)C=C2C=CC(C)C(CCC3CC(OC(=O)NCCOCCOCCOCCNC(=O)CCCCC4CCSS4)CC(=O)O3)C21 ; CCC(C)C(=O)OC1CC(C)C=C2C=CC(C)C(CCC3CC(OC(=O)NCCOCCNC(=O)CCCCC4CCSS4)CC(=O)O3)C21 ; CCC(C)C(=O)OC1CC(C)C=C2C=CC(C)C(CCC3CC(OC(=O)NCCCCCCCCCCCCNC(=O)CCCCC4CCSS4)CC(=O)O3)C21 ; CCC(C)C(=O)OC1CC(C)C=C2C=CC(C)C(CCC3CC(OC(=O)NCCCCCCNC(=O)CCCCC4CCSS4)CC(=O)O3)C21]

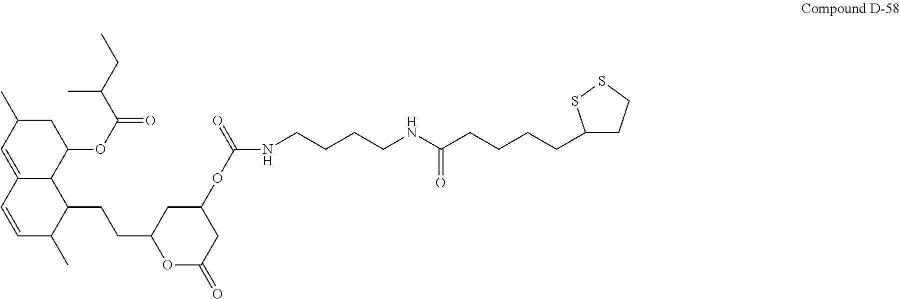 [SMILES: CCC(C)C(=O)OC1CC(C)C=C2C=CC(C)C(CCC3CC(OC(=O)NCCCCNC(=O)CCCCC4CCSS4)CC(=O)O3)C21]

In another embodiment, a derivative of statin lactone is prepared by the conjugation of a statin lactone and an α-lipoic acid via an aminoalcohol and is represented by Formula D-V:

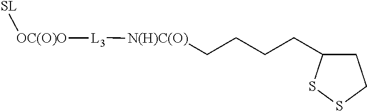

wherein L₃ may be a moiety formed by using an aminoalcohol as the linker in the process of producing the statin lactone derivative; and wherein SL may be selected from the statin lactones from the group consisting of atorvastatin, fluvastatin, lovastatin, mevastatin, pitavastatin, rosuvastatin, and simvastatin.

Aminoalcohols that are useful in the present invention may be represented by the following formula:

H₂N—Y—OH

wherein Y may be a hydrocarbon group; for example, an alkyl, aryl, cycloaliphatic or aralkyl group; and may be saturated or unsaturated. Y may also contain hetero atoms (e.g., nitrogen, oxygen, sulfur, etc.).

Examples of aminoalcohols that are useful in the present inventive compounds include, but are not limited to commercially available one as follows:

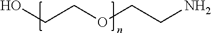

wherein n is an integer between 1 and 100.

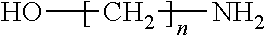

wherein n is an integer between 2 and 12.

Examples of particularly useful derivatives of statin lactones of this embodiment are represented by the following compounds:

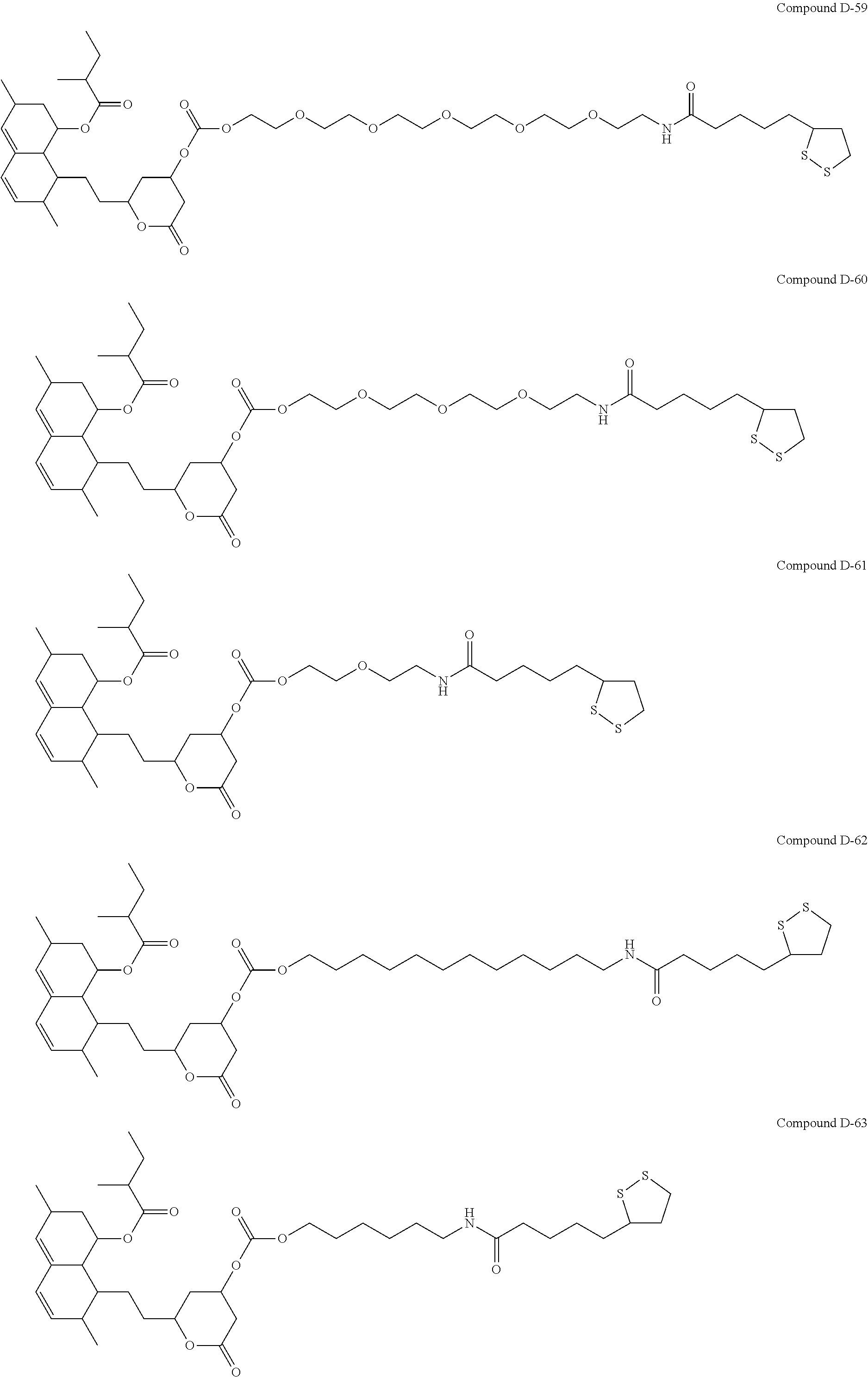 [SMILES: CCC(C)C(=O)OC1CC(C)C=C2C=CC(C)C(CCC3CC(OC(=O)OCCOCCOCCOCCOCCOCCNC(=O)CCCCC4CCSS4)CC(=O)O3)C21 ; CCC(C)C(=O)OC1CC(C)C=C2C=CC(C)C(CCC3CC(OC(=O)OCCOCCOCCOCCNC(=O)CCCCC4CCSS4)CC(=O)O3)C21 ; CCC(C)C(=O)OC1CC(C)C=C2C=CC(C)C(CCC3CC(OC(=O)OCCOCCNC(=O)CCCCC4CCSS4)CC(=O)O3)C21 ; CCC(C)C(=O)OC1CC(C)C=C2C=CC(C)C(CCC3CC(OC(=O)OCCCCCCCCCCCCNC(=O)CCCCC4CCSS4)CC(=O)O3)C21 ; CCC(C)C(=O)OC1CC(C)C=C2C=CC(C)C(CCC3CC(OC(=O)OCCCCCCNC(=O)CCCCC4CCSS4)CC(=O)O3)C21]

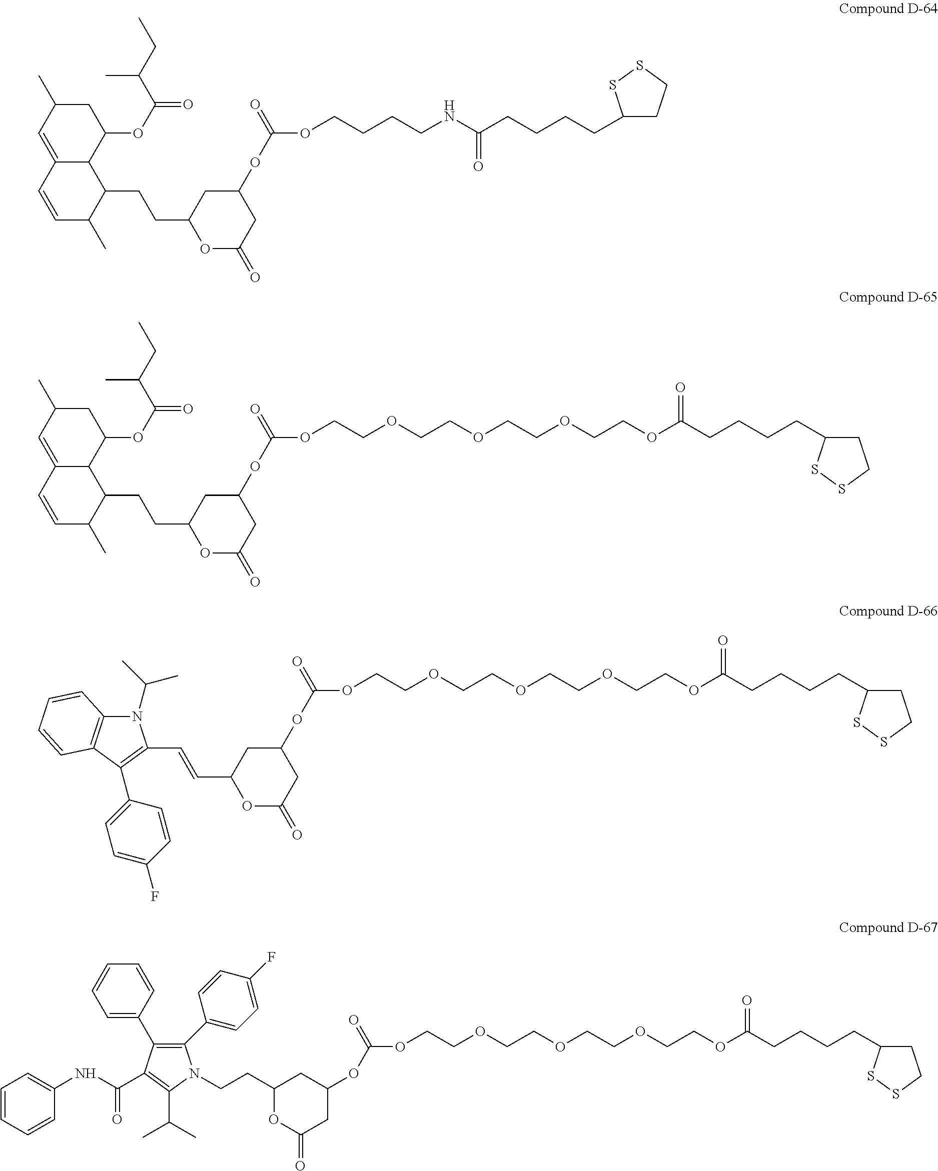 [SMILES: CCC(C)C(=O)OC1CC(C)C=C2C=CC(C)C(CCC3CC(OC(=O)OCCCCNC(=O)CCCCC4CCSS4)CC(=O)O3)C21 ; CCC(C)C(=O)OC1CC(C)C=C2C=CC(C)C(CCC3CC(OC(=O)OCCOCCOCCOCCOC(=O)CCCCC4CCSS4)CC(=O)O3)C21 ; CC(C)n1c(/C=C/C2CC(OC(=O)OCCOCCOCCOCCOC(=O)CCCCC3CCSS3)CC(=O)O2)c(-c2ccc(F)cc2)c2ccccc21 ; CC(C)c1c(C(=O)Nc2ccccc2)c(-c2ccccc2)c(-c2ccc(F)cc2)n1CCC1CC(OC(=O)OCCOCCOCCOCCOC(=O)CCCCC2CCSS2)CC(=O)O1]

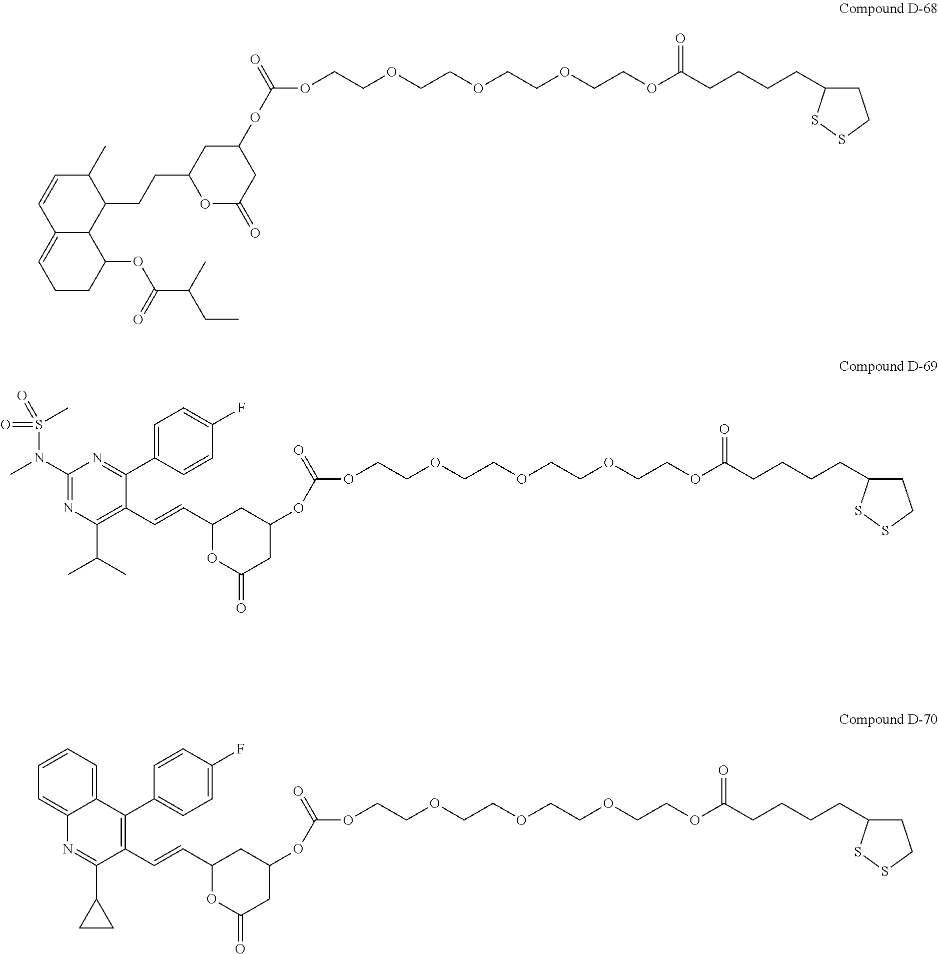 [SMILES: CCC(C)C(=O)OC1CCC=C2C=CC(C)C(CCC3CC(OC(=O)OCCOCCOCCOCCOC(=O)CCCCC4CCSS4)CC(=O)O3)C21 ; CC(C)c1nc(N(C)S(C)(=O)=O)nc(-c2ccc(F)cc2)c1/C=C/C1CC(OC(=O)OCCOCCOCCOCCOC(=O)CCCCC2CCSS2)CC(=O)O1 ; O=C(CCCCC1CCSS1)OCCOCCOCCOCCOC(=O)OC1CC(=O)OC(/C=C/c2c(C3CC3)nc3ccccc3c2-c2ccc(F)cc2)C1]

Additional embodiments of the present invention provide for the following compounds:

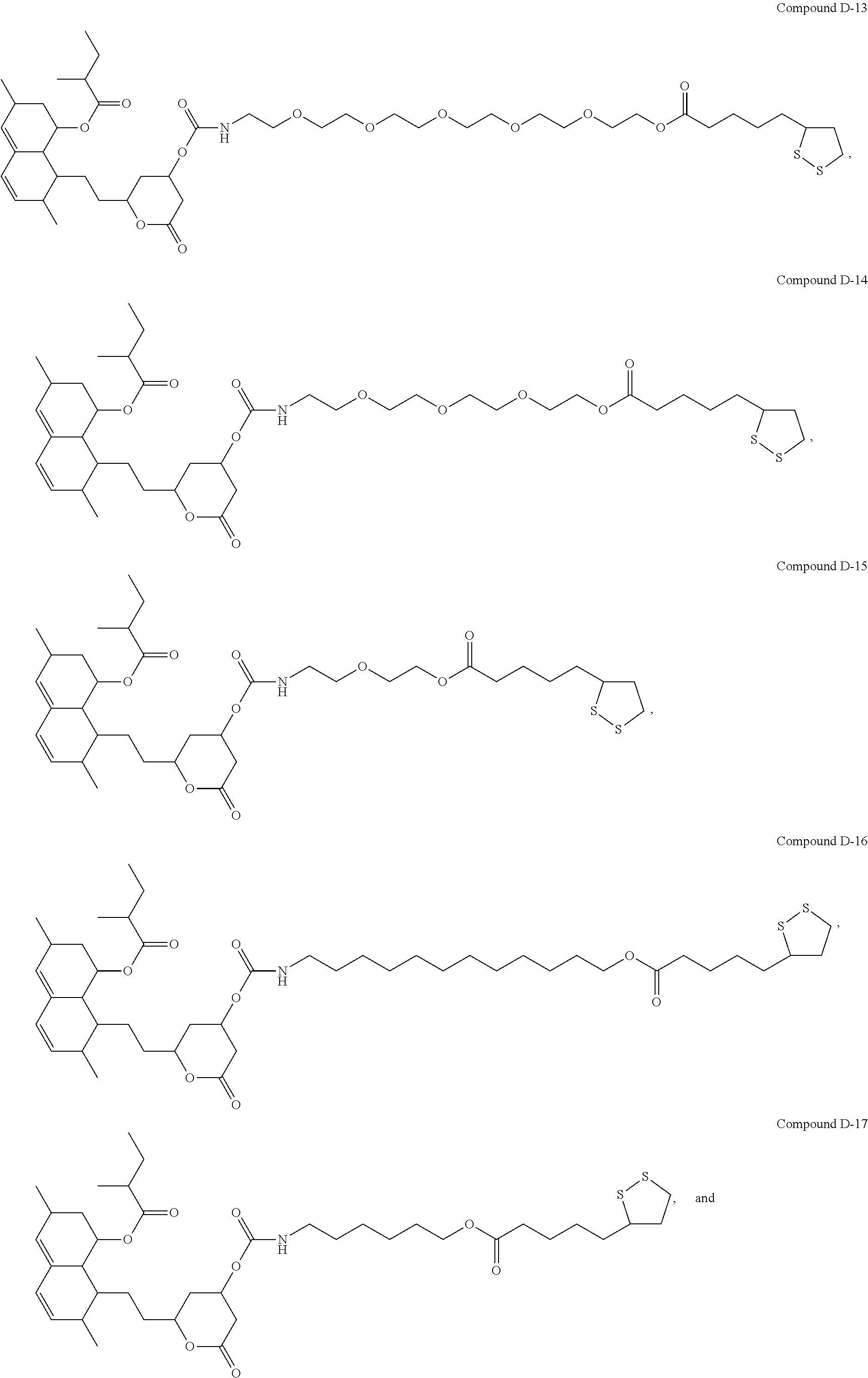 [SMILES: CCC(C)C(=O)OC1CC(C)C=C2C=CC(C)C(CCC3CC(OC(=O)NCCOCCOCCOCCOCCOCCOC(=O)CCCCC4CCSS4)CC(=O)O3)C21 ; CCC(C)C(=O)OC1CC(C)C=C2C=CC(C)C(CCC3CC(OC(=O)NCCOCCOCCOCCOC(=O)CCCCC4CCSS4)CC(=O)O3)C21 ; CCC(C)C(=O)OC1CC(C)C=C2C=CC(C)C(CCC3CC(OC(=O)NCCOCCOC(=O)CCCCC4CCSS4)CC(=O)O3)C21 ; CCC(C)C(=O)OC1CC(C)C=C2C=CC(C)C(CCC3CC(OC(=O)NCCCCCCCCCCCCOC(=O)CCCCC4CCSS4)CC(=O)O3)C21 ; CCC(C)C(=O)OC1CC(C)C=C2C=CC(C)C(CCC3CC(OC(=O)NCCCCCCOC(=O)CCCCC4CCSS4)CC(=O)O3)C21]

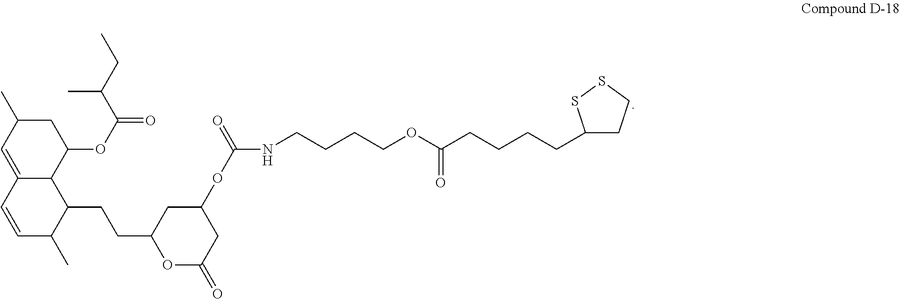 [SMILES: CCC(C)C(=O)OC1CC(C)C=C2C=CC(C)C(CCC3CC(OC(=O)NCCCCOC(=O)CCCCC4CCSS4)CC(=O)O3)C21]

In another embodiment, a statin derivative is prepared by the conjugation of statin lactones and a spacer molecule and is represented by Formula D-VI:

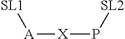

wherein A and P may be selected independently from the group consisting of —OC(O)—, —OC(O)O—, and —OC(O)N(R)—, wherein R may be a hydrogen atom, or a substituted, unsubstituted, branched or unbranched chain of carbon atoms and may contain heteroatoms (e.g., nitrogen, oxygen, sulfur, etc.); wherein X may be a linker that may be a substituted, unsubstituted, branched or unbranched chain of carbon atoms and may contain heteroatoms (e.g., nitrogen, oxygen, sulfur, etc.); and wherein SL1 and SL2 may be selected independently from the statin lactones from the group consisting of atorvastatin, fluvastatin, lovastatin, mevastatin, pitavastatin, rosuvastatin, and simvastatin.

Examples of particularly useful derivatives of statin lactones of this embodiment are represented by the following formulas:

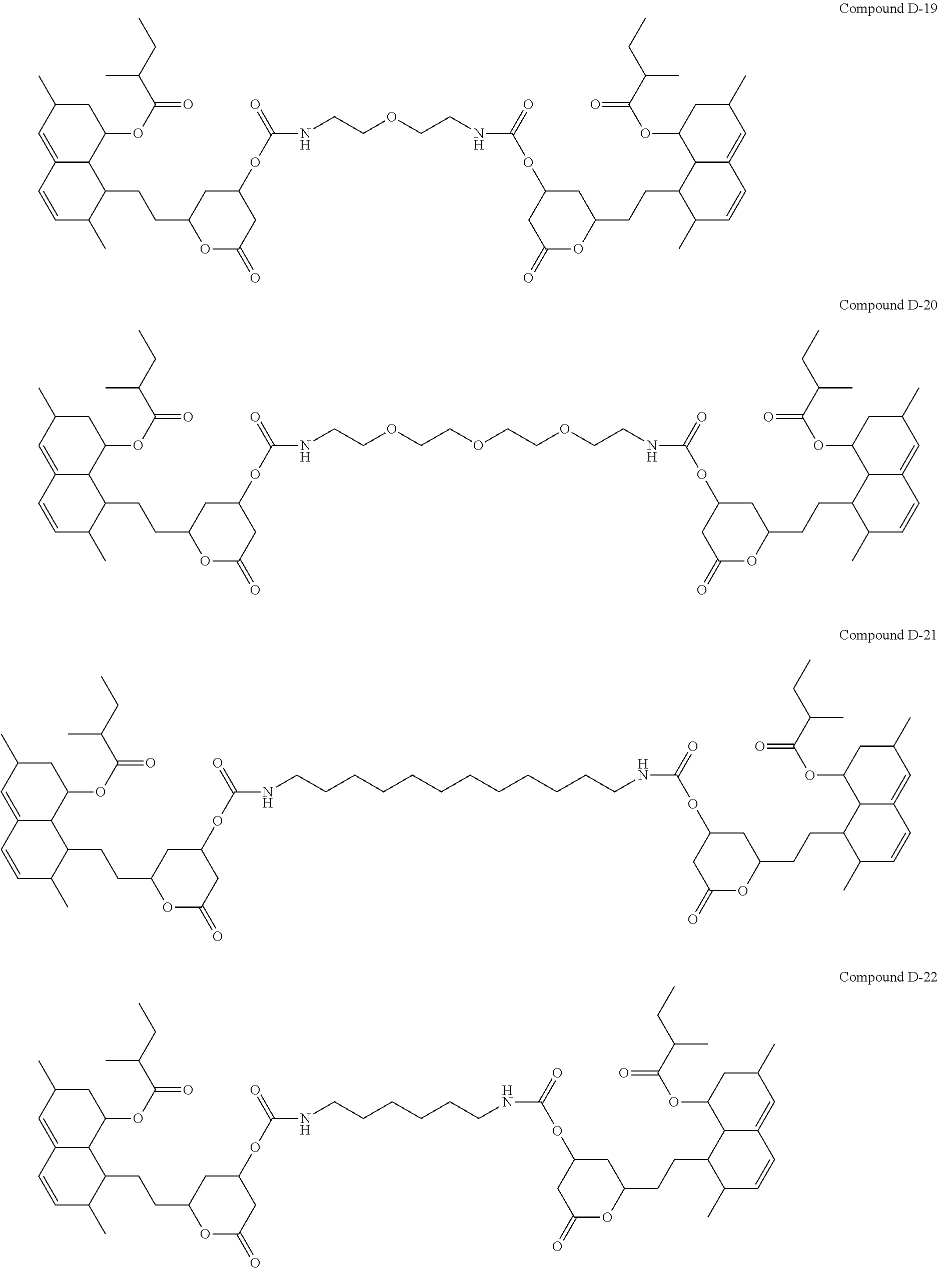

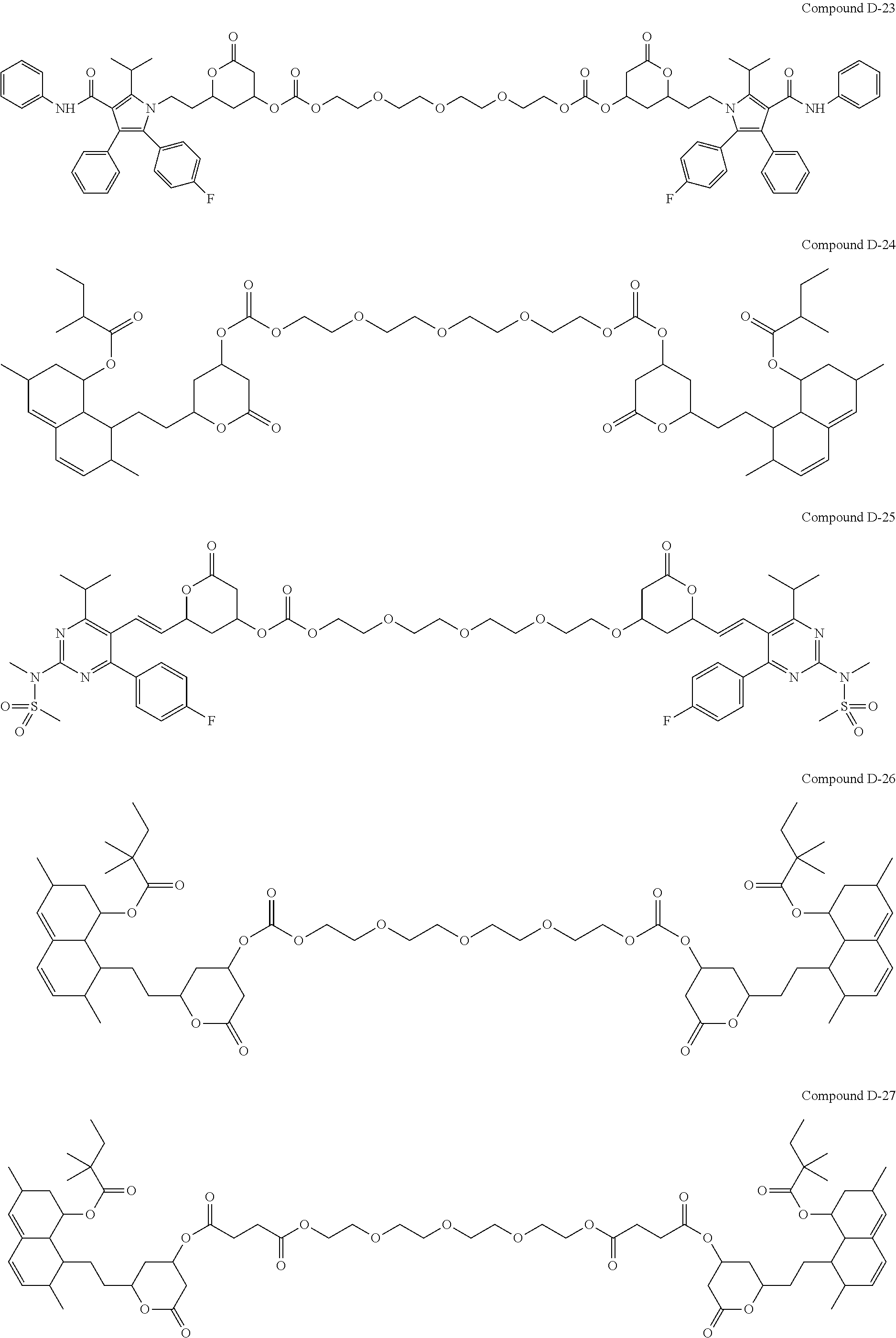

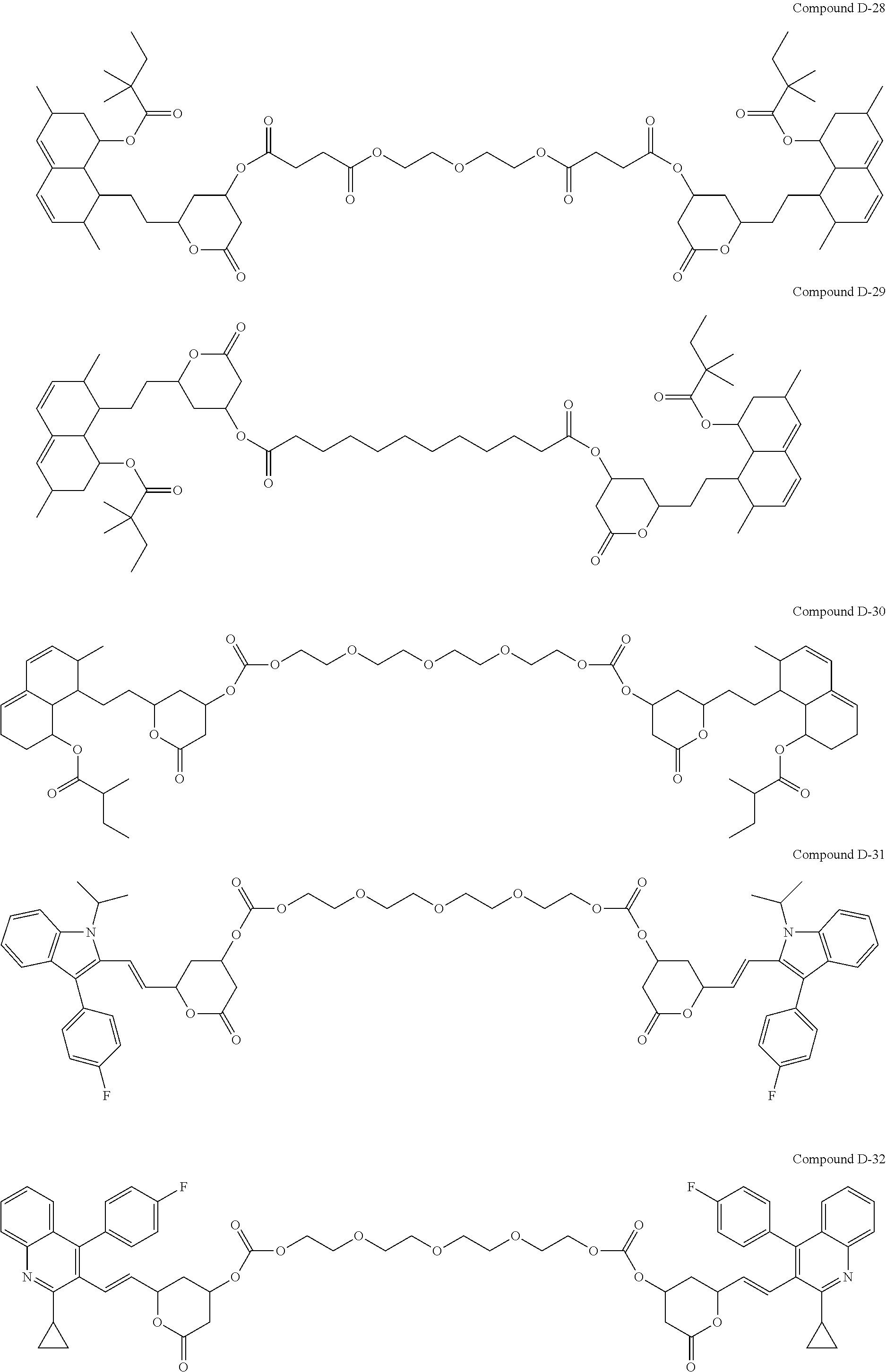

A general scheme for the synthesis of the derivatives of statin and preparation of the nanospheres are described in the ensuing examples. The synthetic procedure is both simple and versatile and leads to the synthesis of the derivatives of statins varying in size and hydrophobicity.

### Therapeutic Agents

In various embodiments, the therapeutic agent is a chemotherapeutic agent or a statin. The chemotherapeutic agent can be selected from the group consisting of paclitaxel, doxorubicin, temozolomide, 5-fluorouracil, camptothecin, and combinations thereof, and the statin can be selected from the statin lactones consisting of atorvastatin, fluvastatin, lovastatin, mevastatin, pitavastatin, rosuvastatin, and simvastatin. In various embodiments, the therapeutic agent is selected from the group consisting of a peptide, antisense nucleic acid, DNA, RNA, protein, and combinations thereof. In a specific embodiment for the treatment of traumatic brain injury, the therapeutic agent is selected from the group consisting of a NSAID, statin, erythropoietin, and combinations thereof.

### Amphiphilic Spacer

A hydrophilic or hydrophobic spacer used in the present disclosure is a molecule that comprises hydrophilic or hydrophobic parts in one molecule, and further comprises chemically active functional group on one end or both ends which can be used as a carrier for a therapeutic agent, diagnostic agent, or another spacer by conjugating it with the therapeutic agent, diagnostic agent, or another spacer molecule.

An amphiphilic spacer used in the present disclosure is a molecule that comprises both hydrophilic and hydrophobic parts in one molecule, and the hydrophilic part further comprises chemically active functional group which can be used as a carrier for a therapeutic or diagnostic agent by conjugating it with the therapeutic agent or diagnostic agent. In various embodiments, the chemically active functional group can be selected from the group consisting of thiol, amine, carboxylic acid, carboxylic acid NHS ester, maleimide, hydrazine, ketone, and aldehyde. An amphiphilic spacer used in the present disclosure also can be made by conjugating a hydrophilic spacer with a hydrophobic spacer. The end of the hydrophilic part further comprises chemically active functional group which can be used as a carrier for a therapeutic or diagnostic agent by conjugating it with the therapeutic agent or diagnostic agent.

In various embodiments, the amphiphilic spacer comprises a hydrophobic part and hydrophilic part. In various embodiments, the hydrophobic part of amphiphilic spacer is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups, and combinations thereof.

In various embodiments, the hydrophilic part of amphiphilic spacer comprises a molecule selected from the group consisting of heteroatom-containing branched and unbranched alkenyl, heteroatom-containing branched and unbranched alkynyl, aryl, cyclic aliphatic, cyclic aromatic, heterocyclic, and aromatic heterocyclic groups, and a chemically active group selected from the group consisting of thiol, amine, carboxylic acid, carboxylic acid NHS ester, maleimide, hydrazine, ketone, aledehyde, and combinations thereof.

### Amphiphilic Polymer

In various embodiments, the amphiphilic polymer comprises a polymer backbone, a hydrophilic part of the polymer and a hydrophobic part of the polymer. In various embodiments, the polymer backbone can natural polymer, modified natural polymer, synthetic polymer, and combinations thereof.

In various embodiments, the polymer backbone is selected from the group consisting of a polyanhydride, polyester, polyorthoester, polyesteramide, polyacetal, polyketal, polycarbonate, polyphosphoester, polyphosphazene, polyvinylpyrrolidone, polydioxanone, poly(malic acid), poly(amino acid), polymer of N-2-(hydroxypropyl)methacrylamide (HPMA), polymer of N-isopropyl acrylamide (NIPAAm), polyglycolide, polylactide, copolymer of glycolide and lactide, and combinations thereof.

In various embodiments, the hydrophobic part of amphiphilic polymer is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups, and combinations thereof.

In various embodiments, the hydrophilic part of amphiphilic polymer comprises a molecule selected from the group consisting of heteroatom-containing branched and unbranched alkenyl, heteroatom-containing branched and unbranched alkynyl, aryl, cyclic aliphatic, cyclic aromatic, heterocyclic, and aromatic heterocyclic groups, and a chemically active group selected from the group consisting of thiol, amine, carboxylic acid, carboxylic acid NHS ester, maleimide, hydrazine, ketone, aledehyde, and combinations thereof

Accordingly, in various embodiments, the nanospheres comprise tocopherol and a therapeutic agent or a diagnostic agent conjugated to a hydrophilic, hydrophobic, or amphiphilic spacer.

In certain embodiments, the nanospheres comprise tocopherol and an antioxidant α-lipoic acid-containing hydrophobic compound and therapeutic agent or a diagnostic agent conjugated to a hydrophilic, hydrophobic, or amphiphilic spacer.

In certain embodiments, the nanospheres comprise tocopherol and a hydrophobic NSAID derivative and a therapeutic agent or a diagnostic agent conjugated to an amphiphilic spacer. In certain embodiments, the nanospheres comprise tocopherol and a hydrophobic antioxidant and anti-inflammatory derivative of an NSAID and a therapeutic agent or a diagnostic agent conjugated to a hydrophilic, hydrophobic, or amphiphilic spacer.

In certain embodiments, the nanospheres comprise tocopherol and derivatives of statin lactones and a therapeutic agent or a diagnostic agent conjugated to a hydrophilic, hydrophobic, or amphiphilic spacer.

In certain embodiments, the nanospheres comprise tocopherol and antioxidant derivatives of camptothecin and/or antioxidant derivatives of camptothecin analogs and a therapeutic agent or a diagnostic agent conjugated to a hydrophilic, hydrophobic, or amphiphilic spacer.

In various embodiments, the nanospheres comprise tocopherol and a therapeutic agent or a diagnostic agent conjugated to an amphiphilic polymer.

In certain embodiments, the nanospheres comprise tocopherol and an antioxidant α-lipoic acid-containing hydrophobic compound and therapeutic agent or a diagnostic agent conjugated to an amphiphilic polymer.

In certain embodiments, the nanospheres comprise tocopherol and a hydrophobic NSAID derivative and a therapeutic agent or a diagnostic agent conjugated to an amphiphilic polymer. In certain embodiments, the nanospheres comprise tocopherol and a hydrophobic antioxidant and anti-inflammatory derivative of an NSAID and a therapeutic agent or a diagnostic agent conjugated to an amphiphilic polymer.

In certain embodiments, the nanospheres comprise tocopherol and derivatives of statin lactones and a therapeutic agent or a diagnostic agent conjugated to an amphiphilic polymer.

In certain embodiments, the nanospheres comprise tocopherol and antioxidant derivatives of camptothecin and/or antioxidant derivatives of camptothecin analogs and a therapeutic agent or a diagnostic agent conjugated to an amphiphilic polymer.

Various embodiments provide for methods of treating cancer. The method may comprise providing a nanosphere of the present invention wherein a therapeutic agent is conjugated to a hydrophilic spacer, a hydrophobic spacer, an amphiphilic spacer, or an amphiphilic polymer; and administering the nanosphere to a subject in need thereof.

Various embodiments provide for methods of imaging and diagnosing cancer. The method may comprise providing a cancer-targeted nanosphere of the present invention wherein a imaging and/or diagnostic agent is conjugated to a hydrophilic spacer, a hydrophobic spacer, an amphiphilic spacer, or an amphiphilic polymer; administering the nanosphere to a subject in need thereof; and imaging the subject to detect the cancer. In various embodiments, the imaging and/or diagnostic agents can include, but are not limited to fluorescent dyes and antibodies against proteins overexpressed in cancer, such as growth factors (including but not limited endothelial growth factors and fibroblast growth factors, placenta growth factors and keratinocyte growth factors) and growth factor receptors (including but not limited endothelial growth factor receptors (EGFR) and receptor tyrosine kinases such as HER-2 and Platelet-derived growth factor receptors.

In various embodiments, the present invention provides pharmaceutical compositions including a pharmaceutically acceptable excipient along with a therapeutically effective amount of the nanospheres of the present invention. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, or ocular. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective amount of the antioxidant derivatives of camptothecin and/or antioxidant derivatives of camptothecin analogs, or the camptothecin nanosphere prodrugs can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

The present invention is also directed to a kit to treat cancer. The kit is an assemblage of materials or components, including at least one of the inventive compositions. Thus, in some embodiments the kit contains a composition including the nanospheres of the present invention as described above.

The exact nature of the components configured in the inventive kit depends on its intended purpose. For example, some embodiments are configured for the purpose of treating cancer. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals. In other embodiments, the kit is configured particularly for diagnostic purposes; for example, diagnosing cancer.

Instructions for use may be included in the kit. “Instructions for use” typically include a tangible expression describing the technique to be employed in using the components of the kit to effect a desired outcome, such as to treat cancer. Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive nanospheres comprising a therapeutic agent or an imaging agent conjugated to a hydrophilic spacer, a hydrophobic spacer, an amphiphilic spacer, or an amphiphilic polymer. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

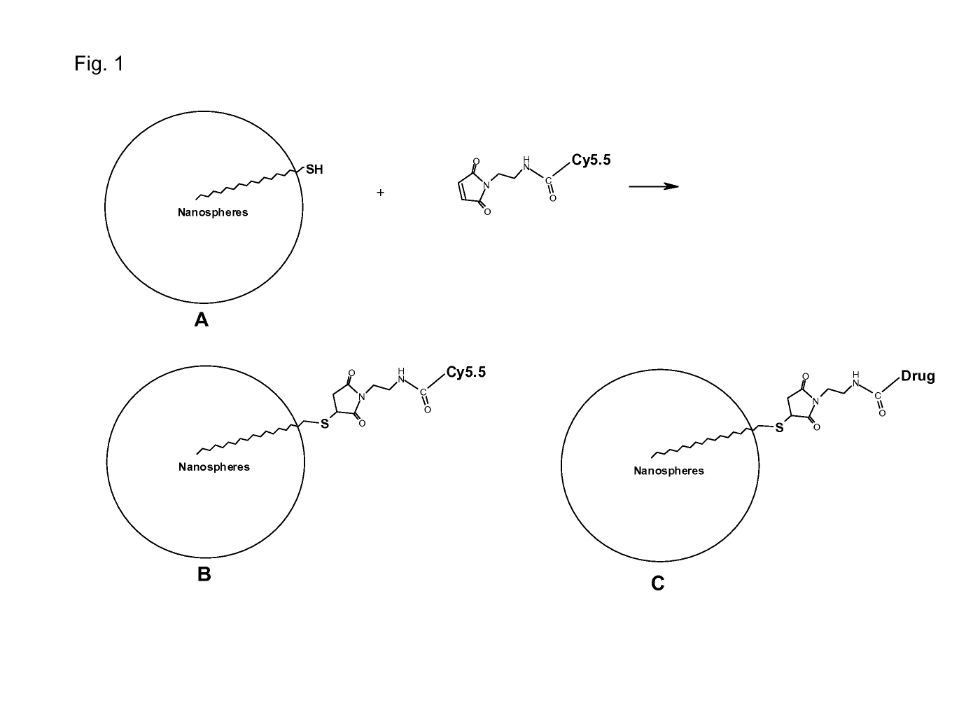

To prepare cy3/cy5/cy5.5-labeled antioxidant-antineoplastic nanospheres, antioxidant-antineoplastic nanospheres were prepared using identical procedure as described in Example A-Example D below except that 0.1-2 mg of 1-octadecanethiol (Aldrich, code 01858) was added to the organic phase prior to spontaneous emulsification (A in FIGS. 1).

To 3 mL of the suspension of 1-octadecanethiol-containing antioxidant-antineoplastic nanospheres 500 μL of 10×PBS and 1.5 molar equivalent of Cy3/Cy5/Cy5.5 maleimide were added (B in FIG. 1). As C in FIG. 1 shows, this intermediate can be used to carrier drugs that are modified to have maleimide group.

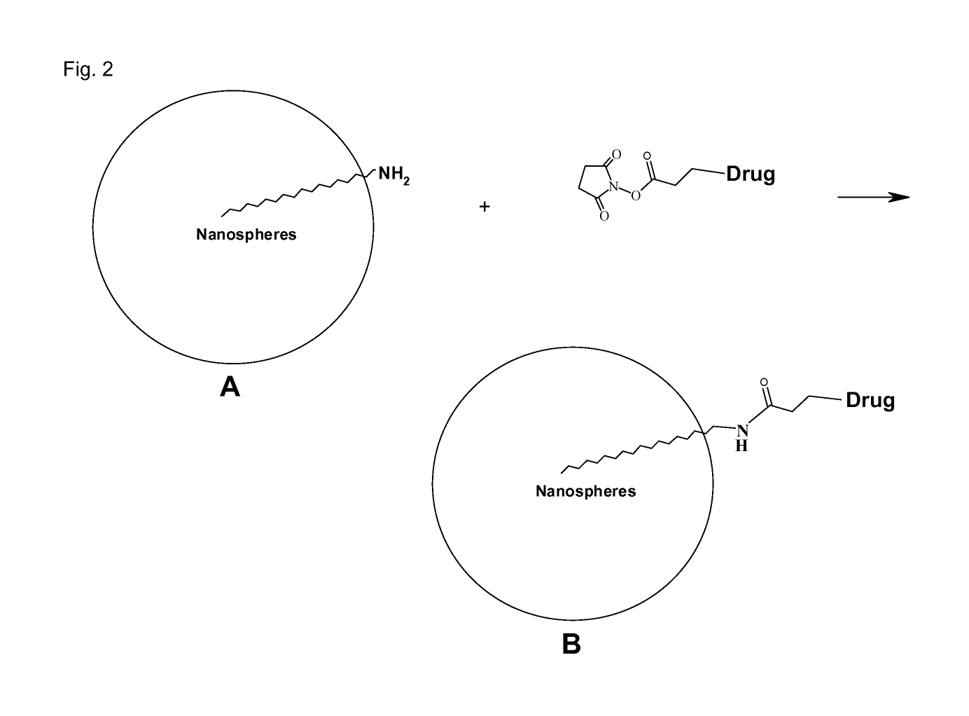

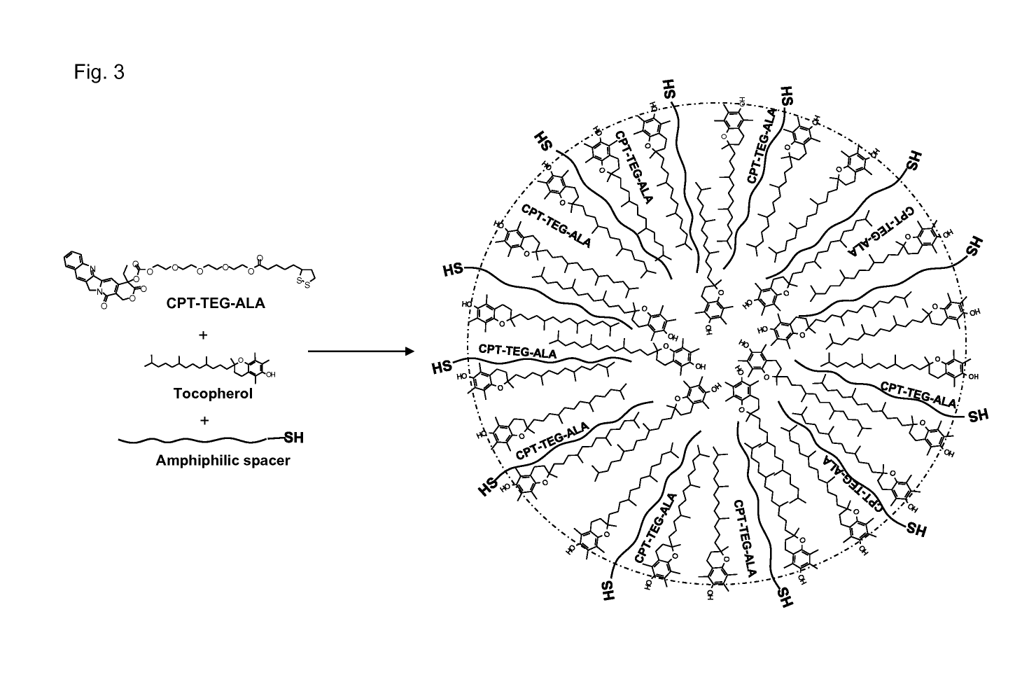

As depicted in FIG. 2, the SH-maleimide pair can be replaced by NH₂—NHS pair or others.

### Example A

### Preparation of the Antioxidant-Antineoplastic Nanospheres

Nanospheres were prepared according to the method using spontaneous emulsification with slight modification. Briefly, 15 mg of the compounds (mixture of camptothecin derivatives and ALA₂(1,12-dodecanediol) were dissolved in acetone (5 mL, 0.1% polysorbate 80). The organic solution was poured under moderate stirring on a magnetic plate into an aqueous phase prepared by dissolving 25 mg of Pluronic F68 in 10 mL bidistilled water (0.25% w/v). Following 15 min of magnetic stirring, the acetone was removed under reduced pressure at room temperature. The nanospheres were filtered through 0.8 μm hydrophilic syringe filter and stored at 4° C. The hydrodynamic size measurement and size distribution of the nanospheres was performed by the dynamic light scattering (DLS) using a Coulter N4-Plus Submicron Particle Sizer (Coulter Corporation, Miami, Fla.).

Additionally, 25 mg of the compounds (mixture of the antioxidant camptothecin derivatives, multiple α-lipoic acid containing compounds and α-tocopherol) were dissolved in acetone (5 mL, 0.1% polysorbate 80). The organic solution was poured under moderate stirring on a magnetic plate into an aqueous phase prepared by dissolving 25 mg of Pluronic F68 in 10 mL bidistilled water (0.25% w/v). Following 15 min of magnetic stirring, the acetone was removed under reduced pressure at room temperature. The nanospheres were filtered through 0.8 μm hydrophilic syringe filter and stored at 4° C. The hydrodynamic size measurement and size distribution of the nanospheres was performed by the dynamic light scattering (DLS) using a Coulter N4-Plus Submicron Particle Sizer (Coulter Corporation, Miami, Fla.). Control nanosphere was prepared from multiple α-lipoic acid containing compounds and α-tocopherol in the absence of camptothecin derivatives.

TABLE 1
Size and Polydispersity Index (P.I.):
Antioxidant-Antineoplastic Nanosphere I

| ALA₂(1,12- | α-Tocopherol | Compound | Size | |
|---|---|---|---|---|
| dodecanediol) (mg) | (mg) | C-10 (mg) | (nm) | P.I. |
| 25 | 5 | 0 | 124 ± 32 | 0.09 |
| 25 | 5 | 1 | 132 ± 39 | 0.13 |

### Example B

### Preparation of the Antioxidant-Antineoplastic Nanospheres

Nanospheres were prepared according to the method described in Example 5 using spontaneous emulsification from 25 mg of the compounds (mixture of camptothecin derivatives and α-tocopherol). Control nanosphere was prepared from α-tocopherol or Ibu₂TEG in the absence of camptothecin derivatives.

TABLE 2
Size and Polydispersity Index (P.I.): Antioxidant-
Antineoplastic Nanosphere II

| α-Tocopherol | Compound | Size | |
|---|---|---|---|
| (mg) | C-10 (mg) | (nm) | P.I. |
| 25 | 1 | 128 ± 45 | 0.25 |
| 25 | 0 | 121 ± 40 | 0.20 |

### Example C

### Preparation of the Anti-Inflammatory-Antineoplastic Nanosphere

Nanospheres were prepared according to the method described in Example 5 using spontaneous emulsification from 25 mg of the compounds (mixture of camptothecin derivatives, derivatives of non-steroidal anti-inflammatory drugs (NSAIDs) and α-tocopherol). Control nanosphere was prepared from α-tocopherol or a mixture of α-tocopherol and derivatives of NSAIDs in the absence of camptothecin derivatives.

TABLE 3
Size and Polydispersity Index (P.I.): Anti-
inflammatory-Antineoplastic Nanosphere

| Ibu₂TEG | α-Tocopherol | Compound | Size | |
|---|---|---|---|---|
| (mg) | (mg) | C-10 (mg) | (nm) | P.I. |
| 25 | 5 | 1 | 124 ± 32 | 0.09 |
| 25 | 5 | 0 | 130 ± 34 | 0.09 |

### Example D

### Anticancer and Antiproliferative Effects of the Nanospheres Comprising Camptothecin Derivatives

The U87-MG human glioma cell line was obtained from American Type Culture Collection (ATCC) (Rockville, Md., USA). The cells were grown and maintained in Minimum Essential Medium (MEM) (Invitrogen) containing antibiotics 100 U/mL penicillin (Invitrogen) and 100 μg/mL streptomycin (Invitrogen), and supplemented with 10% fetal bovine serum (FBS) (Invitrogen). Cells were kept at 37° C. in a humidified atmosphere including 5% CO₂.

Nanospheres were prepared from the mixture of Compound C-10 (1 mg), α-tocopherol (25 mg), and multiple α-lipoic acid containing compound (ALA)₃Glycerol; or Compound C-10 (1 mg) and α-tocopherol (25 mg); or Compound C-10 (1 mg), α-tocopherol (25 mg), and NSAID derivative Ibu₂TEG, and dialyzed in phosphate buffered saline (PBS) overnight. The human glioma cells (U87-MG) were seeded in a 6-well flask at 10⁵ cells/well and allowed to grow for 24 h. The medium was changed and the cells were treated with nanospheres at final concentration ranging from 0.1 to 2 μM for the Compound C-10. After a 4-day treatment, the medium was remove, cells were washed with PBS and 1 mL of 0.25% trypsin/EDTA (Gibco) was added to detach the cells. The cells were counted immediately in a hemacytometer. Control culture was grown in the absence of nanospheres.

### Example 2

### Synthesis of α-Lipoic Acid Derivative ALA₂(1,12-dodecanediol)

α-Lipoic acid (2.48 g, 12 mmol, 1.2 equiv.) and 1,12-dodecanediol (10 mmol OH, 1.0 equiv.) in 20 mL of anhydrous dichloromethane (DCM) were reacted with 4-(dimethylamino)-pyridine (DMAP, 1.47 g, 12 mmol, 1.2 equiv.) in the presence of molecular sieve (60 Å, 10-20 mesh beads) for 10 min at room temperature. N-(3-Dimethylaminopropyl)-N-ethylcarbodiimide hydrochloride (EDCI, 2.3 g, 12 mmol, 1.2 equiv.) was added portionwise over 10 min and the reaction mixture was stirred for 12 h at room temperature in the dark, filtered, and then concentrated under vacuum to reduce the volume. The resulting reaction mixture was purified using silica gel by direct loading onto the column without further preparation. The solvent was removed under reduced pressure to give the products. ¹H NMR and ¹³C NMR spectra of the compound are provided.

U.S. Provisional Application Ser. No. 61/018,749, filed Jan. 3, 2008, and International Application Publication No. WO 2009/086547, filed Dec. 30, 2008, herein incorporated by reference in their entirety as though fully set forth, provide additional examples of synthesizing α-lipoic acid derivatives that are used in the present invention.

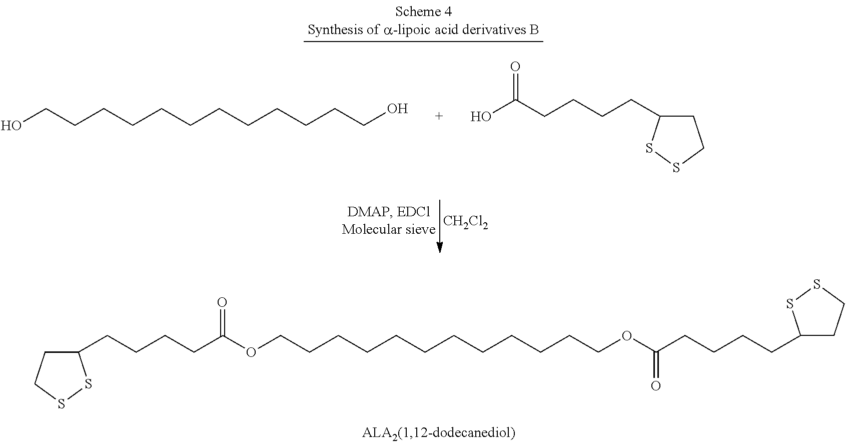 [SMILES: OCCCCCCCCCCCCO ; O=C(O)CCCCC1CCSS1 ; "CH2Cl2" ; O=C(CCCCC1CCSS1)OCCCCCCCCCCCCOC(=O)CCCCC1CCSS1]

### Example 3

### Synthesis of Bifunctional Derivatives of α-Lipoic Acid and NSAIDs

α-Lipoic acid (ALA, 10 mmol) and tetraethylene glycol (TEG, 30 mmol) in 50 ml of anhydrous dichloromethane (DCM) were reacted with 4-(dimethylamino)-pyridine (DMAP, 15 mmol) in the presence of a molecular sieve (Fluka, 3 Å, 10-20 mesh beads) for 10 min at room temperature. N-(3-Dimethylaminopropyl)-N-ethylcarbodiimide hydrochloride (EDCI, 10 mmol) was added portionwise over 10 min and the reaction mixture was stirred for 5 h at room temperature in the dark, filtered, and then concentrated under vacuum to reduce the volume. The product ALA-TEG-OH and dimeric byproduct ALA-TEG-ALA were purified using column chromatography by loading the concentrated reaction mixture on the column without prior preparation and characterized as described above. Mono-ALA derivatives of TEG (3.8 mmol) and NSAIDs (4.1 mmol, indomethacin: Ind, ibuprofen: Ibu, naproxen: Npx) in 20 ml of anhydrous DCM were reacted with DMAP (4.1 mmol) in the presence of molecular sieve for 10 min at room temperature. EDCI (4.1 mmol) was added portionwise over 10 min and the reaction mixture was stirred for 5 h at room temperature in the dark, filtered, and then concentrated under vacuum at room temperature. The products were purified using column chromatography and characterized as described above.

### ALA-TEG-OH:

The column chromatography on silica gel (CHCl₃:MeOH 50:1) gave the compound as a yellow oil (63%). TLC (CHCl₃:MeOH 50:0.5) R_{f} 0.19; ¹H NMR (400 MHz, CDCl₃): δ=1.47 (m, 2×H, Hₐ), 1.68 (m, 4×H, H_{b}), 1.91 (m, 1×H, H_{c}), 2.36 (t, 2×H, H_{d}), 2.46 (m, 1×H, Hₑ), 2.61 (s, 1×H, —OH), 3.11 (m, 1×H, H_{f}), 3.18 (m, 1×H, H_{g}), 3.56 (m, 1×H, Hₕ), 3.61 (m, 2×H, H_{E}), 3.67 (s, 8×H, H_{A}), 3.71 (m, 4×H, H_{B}), 4.24 (m, 2×H, H_{D}). ¹³C NMR (100 MHz, CDCl₃): δ=24.55, 28.67, 33.86, 34.54, 38.45, 40.19, 56.31, 61.6, 63.37, 69.11, 70.19, 70.43, 70.45, 70.56, 173.47.

### ALA-TEG-ALA:

The column chromatography on silica gel (CHCl₃:MeOH 90:1) gave the compound as a yellow oil. TLC (CHCl₃:MeOH 100:0.5) R_{f} 0.12; ¹H NMR (400 MHz, CDCl₃): δ=1.47 (m, 4H, 2×Ha), 1.68 (m, 8H, 2×Hb), 1.91 (m, 2H, 2×Hc), 2.35 (t, J=7.5 Hz, 4H, 2×Hd), 2.46 (m, 2H, 2×He), 3.15 (m, 4H, 2×Hf+Hg), 3.57 (m, 2H, 2×Hh), 3.65 (s, 8H, O—CH₂—CH₂—O), 3.70 (t, J=4.8 Hz, 4H, 2×O—CH₂—CH₂—OCO), 4.23 (t, J=4.8 Hz, 4H, 2×CO—O—CH₂—). ¹³C NMR (100 MHz, CDCl₃): δ=24.56, 28.71, 33.94, 34.56, 38.5, 40.22, 56.33, 63.44, 69.16, 70.56, 173.36.

### ALA-TEG-Ind:

The column chromatography on silica gel (CHCl₃:MeOH 100:0.5) gave the compound as a yellow oil (73%). TLC (CHCl₃:MeOH 50:0.5) R_{f} 0.33; ¹H NMR (400 MHz, CDCl₃): δ=1.48 (m, 2×H, Hₐ), 1.69 (m, 4×H, H_{b}), 1.92 (m, 1×H, H_{c}), 2.33-2.43 (m, 5×H, H₈+H_{d}), 2.47 (m, 1×H, Hₑ), 3.15 (m, 2×H, H_{f}+H_{g}), 3.54-3.75 (m, 15×H, H₇+H_{A}+H_{B}+Hₕ), 3.86 (s, 3×H, H₆), 4.27 (m, 4×H, H_{D}+H_{E}), 6.68 (q, 1×H, H₅), 6.95 (d, 1×H, H₄), 6.99 (d, 1×H, H₃), 7.49 (m, 2×H, H₂), 7.68 (m, 2×H, H₁). ¹³C NMR (100 MHz, CDCl₃): δ=13.4, 24.6, 28.7, 30.2, 33.9, 34.6, 38.5, 40.2, 55.71, 56.3, 63.4, 64.1, 69.1, 69.16, 70.53, 70.58, 101.39, 111.59, 112.50, 114.92, 129.12, 130.65, 130.78, 131.18, 133.91, 135.98, 139.20, 156.03, 168.24, 170.77, 173.41.

### ALA-TEG-Ibu:

The column chromatography on silica gel (CHCl₃:MeOH 100:0.5) gave the compound as a yellow oil (69%). TLC (CHCl₃:MeOH 50:0.5) R_{f} 0.37; ¹H NMR (400 MHz, CDCl₃): δ=0.86 (d, 6×H, H₇), 1.37-1.48 (m, 5×H, H₆+Hₐ), 1.64 (m, 4×H, H_{b}), 1.85-1.95 (m, 2×H, H₅+H_{c}), 2.32 (t, 2×H, H_{d}), 2.38-2.45 (m, 3×H, H₄+Hₑ), 3.04-3.18 (m, 2×H, H_{g}+H_{f}) 3.50-3.73 (m, 14×H, H₃+H_{A}+H_{B}+Hₕ), 4.20 (m, 4×H, H_{D}+H_{E}), 7.05 (d, 2×H, H₂), 7.18 (d, 2×H, H₁). ¹³C NMR (100 MHz, CDCl₃): δ=18.59, 22.41, 24.6, 28.71, 30.16, 33.91, 34.58, 38.47, 40.20, 44.98, 45.01, 56.31, 63.43, 63.85, 69.05, 69.16, 70.54, 70.59, 127.18, 129.27, 137.67, 140.44, 173.38, 174.62.

### ALA-TEG-Npx:

The column chromatography on silica gel (CHCl₃:MeOH 100:0.5) gave the compound as a yellow oil (65%). TLC (CHCl₃:MeOH 50:0.5) R_{f} 0.33; ¹H NMR (400 MHz, CDCl₃): δ=1.44 (m, 2×H, Hₐ), 1.54-1.71 (m, 7×H, H₅+H_{b}), 1.88 (m, 1×H, Hₑ), 2.33 (t, 2×H, H_{d}), 2.43 (m, 1×H, Hₑ), 3.05-3.19 (m, 2×H, H_{f}+H_{g}), 3.39-3.67 (m, 13×H, H_{A}+H_{B}+Hₕ), 3.88 (m, 4×H, H₄), 4.21 (m, 4×H, H_{D}+H_{E}), 7.12 (m, 2×H, H₃), 7.40 (q, 1×H, H₂), 7.68 (m, 3×H, H₁). ¹³C NMR (100 MHz, CDCl₃): δ=18.57, 24.61, 28.73, 33.93, 34.57, 38.48, 40.12, 45.33, 55.32, 56.33, 63.44, 63.96, 69.03, 69.14, 70.53, 105.57, 118.97, 125.99, 126.28, 127.11, 128.91, 129.28, 133.68, 135.63, 157.63, 173.44, 174.59.

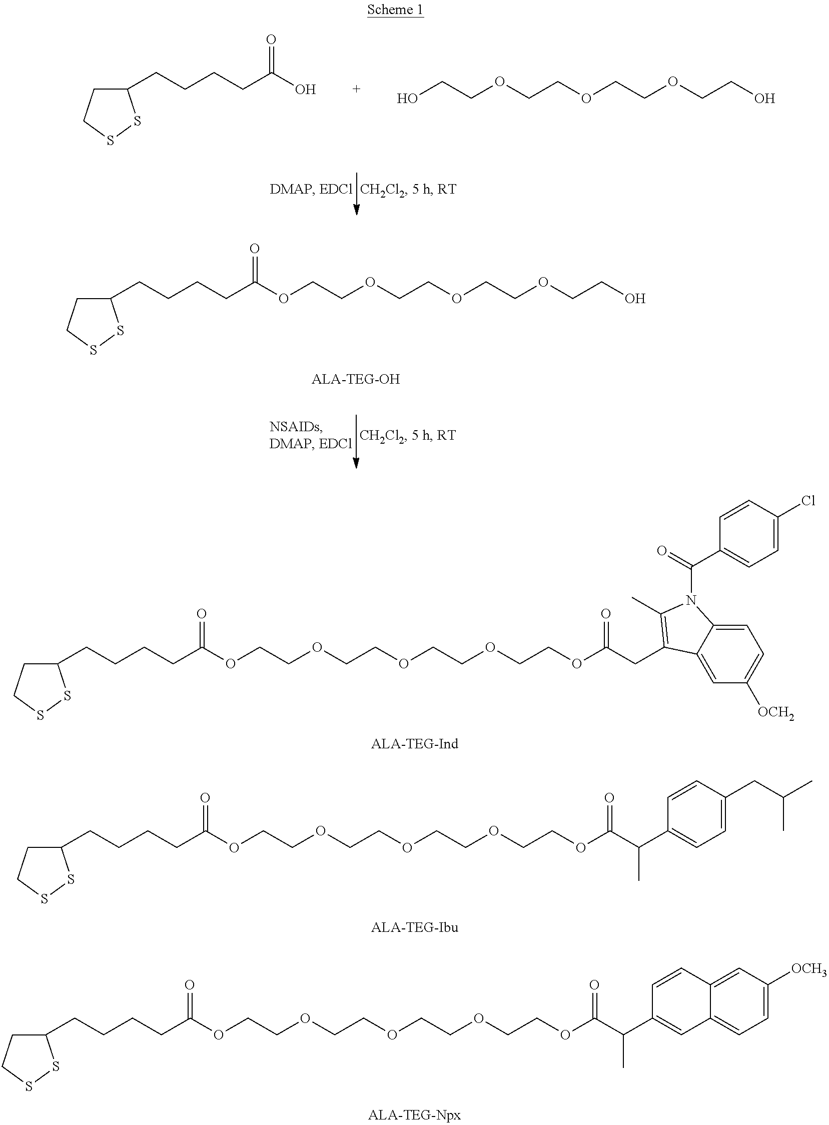

The same procedure, except that diethylene glycol was used instead of tetraethylene glycol, was used for the synthesis of the following compounds:

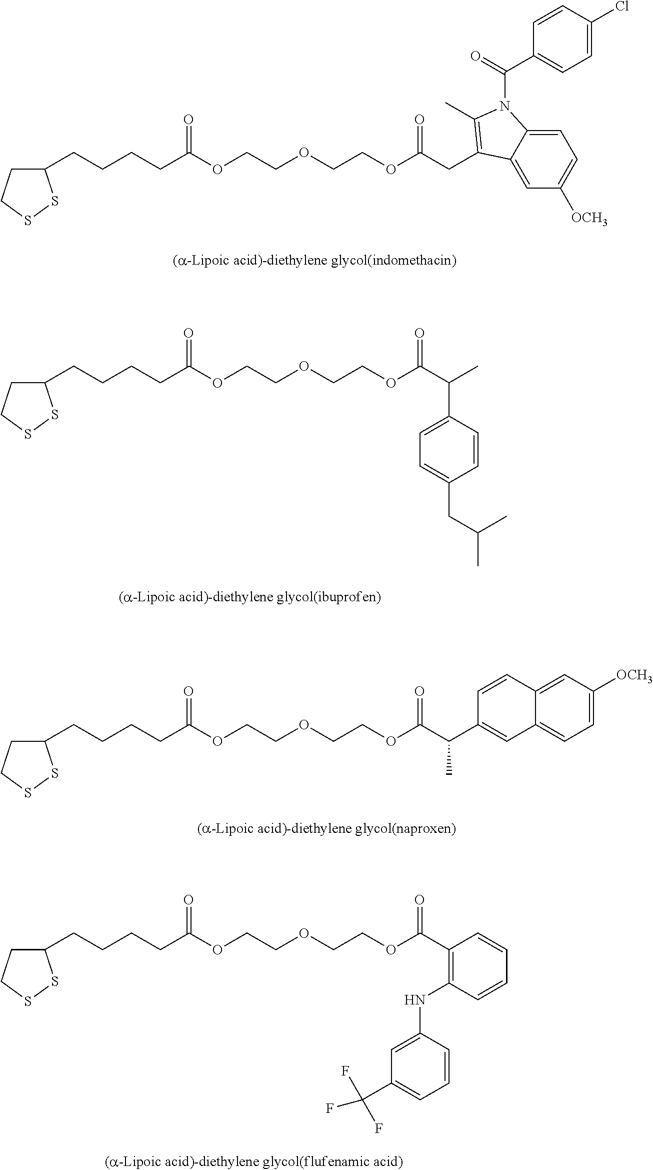 [SMILES: COc1ccc2c(c1)c(CC(=O)OCCOCCOC(=O)CCCCC1CCSS1)c(C)n2C(=O)c1ccc(Cl)cc1 ; CC(C)Cc1ccc(C(C)C(=O)OCCOCCOC(=O)CCCCC2CCSS2)cc1 ; COc1ccc2cc([C@H](C)C(=O)OCCOCCOC(=O)CCCCC3CCSS3)ccc2c1 ; O=C(CCCCC1CCSS1)OCCOCCOC(=O)c1ccccc1Nc1cccc(C(F)(F)F)c1]

### Example 4

### Synthesis of Dimeric Derivatives of NSAIDs

NSAIDs (6 mmol) and TEG (2.5 mmol) in 40 ml of anhydrous DCM were reacted with DMAP (6 mmol) in the presence of molecular sieve for 10 min at room temperature. EDCI (6 mmol) was added portionwise over 10 min and the reaction mixture was stirred for 5 h at room temperature in the dark, filtered, and then concentrated under vacuum. The products were purified (column chromatography, 100:0.5 CH₃Cl: MeOH) and characterized as described above.

### Ind₂TEG:

The column chromatography on silica gel (CHCl₃:MeOH 100:0.5) gave the compound as a yellow oil (78%). TLC (CHCl₃:MeOH 50:0.5) R_{f} 0.25; ¹H NMR (400 MHz, CDCl₃): δ=2.35 (s, 6×H, Hₛ), 3.56 (m, 8×H, H_{A}), 3.64-3.70 (m, 8×H, H₇+H_{B}), 3.80 (s, 6×H, H₆), 4.25 (t, 4×H, H_{D}+H_{E}), 6.64 (q, 2×H, H₅), 6.86 (d, 2×H, H₄), 6.95 (d, 2×H, H₃), 7.43 (m, 4×H, H₂), 7.62 (m, 4×H, H₁). ¹³C NMR (100 MHz, CDCl₃): δ=13.4, 30.19, 55.69, 64.13, 69.07, 70.52, 70.57, 101.4, 111.58, 112.51, 114.93, 129.11, 130.66, 130.79, 131.18, 133.93, 135.98, 139.18, 156.04, 168.22, 170.77.

### Ibu₂TEG:

The column chromatography on silica gel (CHCl₃:MeOH 100:0.5) gave the compound as a colorless oil (83%). TLC (CHCl₃:MeOH 50:0.5) R_{f} 0.54; ¹H NMR (400 MHz, CDCL₃): δ=0.90 (d, 12×H, H₇), 1.49 (d, 6×H, H₆), 1.84 (m, 2×H, H₅), 2.44 (d, 4×H, H₄), 3.55 (m, 8×H, H_{A}), 3.63 (m, 4×H, H_{B}), 3.73 (q, 2×H, H₃), 4.22 (m, 4×H, H_{D}+H_{E}), 7.08 (m, 4×H, H₂), 7.21 (m, 4×H, H₁). ¹³C NMR (100 MHz, CDCl₃): δ=18.60, 22.42, 30.19, 45.02, 45.04, 63.87, 69.08, 70.57, 70.61, 127.20, 129.29, 137.70, 140.48, 174.67.

### Npx₂TEG:

The column chromatography on silica gel (CHCl₃:MeOH 100:0.5) gave the compound as a colorless oil (75%). TLC (CHCl₃:MeOH 50:0.5) R_{f} 0.46; ¹H NMR (400 MHz, CDCl₃): δ=1.58 (d, 6×H, H₅), 3.44 (m, 8×H, H_{A}), 3.60 (m, 4×H, H_{B}), 3.90 (m, 8×H, H₄), 4.22 (m, 4×H, H_{D}+H_{E}), 7.12 (m, 4×H, H₃), 7.41 (q, 2×H, H₂), 7.68 (m, 6×H, H₁). ¹³C NMR (100 MHz, CDCl₃): δ=18.56, 45.33, 55.29, 63.95, 69.02, 70.44, 70.47, 105.56, 118.96, 125.96, 126.27, 127.11, 128.91, 129.27, 133.68, 135.62, 157.63, 174.60.

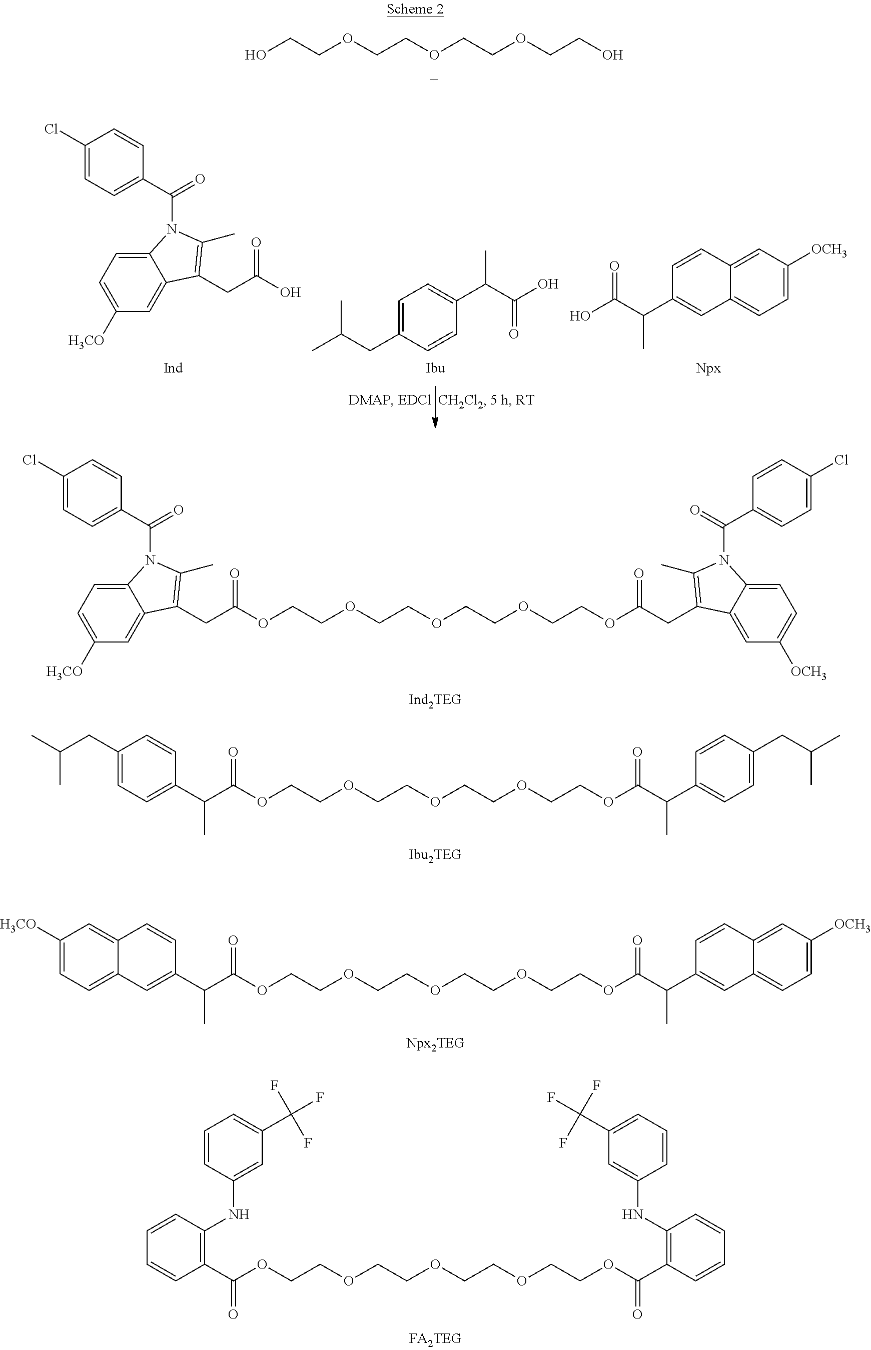

### Example 5

### Spontaneous Emulsification

Nanoprodrugs were prepared according to the method using spontaneous emulsification (Bouchemal et al., 2004b). Briefly, 25 mg of the compounds were dissolved in acetone (5 ml) containing polysorbate 80 (0.1% w/v). The organic solution was poured under moderate stirring on a magnetic plate into an aqueous phase prepared by dissolving 25 mg of Pluronic F68 in 10 ml distilled water (0.25% w/v). Following 15 min of magnetic stirring, the acetone was removed under reduced pressure at room temperature. The suspensions were filtered through 0.8 μm hydrophilic syringe filter (Corning, Part No. 431221, Fisher Scientific Co., Pittsburgh, Pa., USA) and stored at 4° C.

### Example 6

### Synthesis of Antioxidant Compounds

α-Lipoic acid (2.48 g, 12 mmol, 1.2 equiv.) and the compounds containing two hydroxyl groups (1,12-dodecanediol (“1,12-DD”)) (10 mmol OH, 1.0 equiv.) in 20 mL of anhydrous dichloromethane (DCM) were reacted with 4-(dimethylamino)-pyridine (DMAP, 1.47 g, 12 mmol, 1.2 equiv.) in the presence of molecular sieve (60 Å, 10-20 mesh beads) for 10 min at room temperature. N-(3-Dimethylaminopropyl)-N-ethylcarbodiimide hydrochloride (EDCI, 2.3 g, 12 mmol, 1.2 equiv.) was added portionwise over 10 min and the reaction mixture was stirred for 12 h at room temperature in the dark, filtered, and then concentrated under vacuum to reduce the volume. The resulting reaction mixture was purified using silica gel by direct loading onto the column without further preparation. The solvent was removed under reduced pressure to give the products. See also International Application No. PCT/US08/88541, which is incorporated herein by reference in its entirety as though fully set forth.

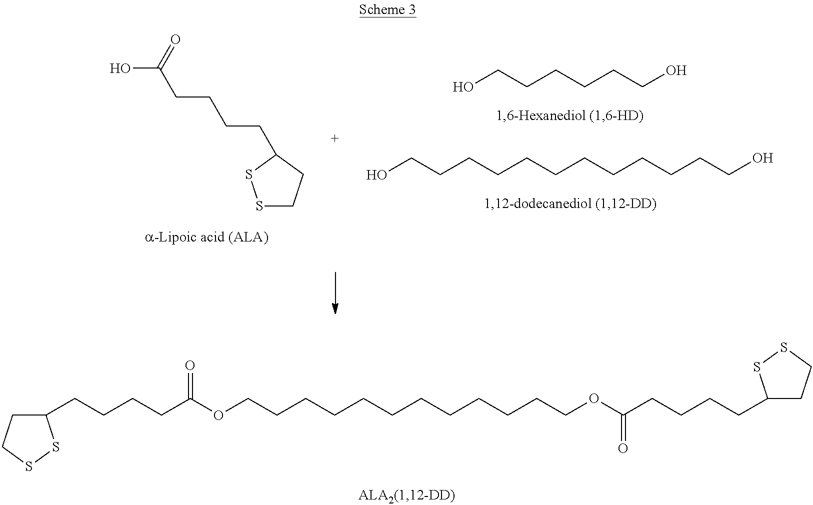 [SMILES: O=C(O)CCCCC1CCSS1 ; OCCCCCCCCCCCCO ; OCCCCCCO ; O=C(CCCCC1CCSS1)OCCCCCCCCCCCCOC(=O)CCCCC1CCSS1]

### Example 7

### Preparation of Nanospheres from the Mixture of the Hydrophobic NSAIDs Derivatives and Poly(Lactide-Co-Glycolide)(PLGA)

Nanospheres were prepared according to the method described above using spontaneous emulsification from a mixture of the hydrophobic derivatives of NSAIDs (25 mg) with PLGA (100 mg) (Sigma, P2191, lactide: glycolide 50:50, mol. wt 40,000-75,000), α-tocopherol (25 mg).

TABLE 5
Size and Polydispersity Index (P.I.)

| NSAID-containing | Second | Size | |
|---|---|---|---|
| hydrophobic compounds | components | (nm) | P.I. |
| 25 mg Tetraethylene | 100 mg | 155 ± 48 | 0.16 |
| glycol(ibuprofen)₂ | PLGA | | |
| 25 mg Tetraethylene | 25 mg | 189 ± 55 | 0.13 |
| glycol(ibuprofen)₂ | α-tocopherol | | |
| 20 mg Tetraethylene | 50 mg | 204 ± 62 | 0.145 |
| glycol(ibuprofen)₂ | ALA₂(1,12-DD) | | |

### Example 8

### Preparation of the Antioxidant-Antineoplastic Nanospheres Using Multiple-Step Spontaneous Emulsification to Increase Concentration

To prepare the antioxidant-antineoplastic nanospheres with a higher concentration, a multiple-step spontaneous emulsification was applied. Generally, 25-100 mg of the compounds (mixture of the antioxidant camptothecin derivatives, multiple α-lipoic acid containing compounds, derivatives of non-steroidal anti-inflammatory drugs (NSAIDs) and α-tocopherol) were dissolved in acetone (5 mL, 0.1% polysorbate 80). The organic solution was poured under moderate stirring on a magnetic plate into an aqueous phase prepared by dissolving 25 mg of Pluronic F68 in 10 mL bidistilled water (0.25% w/v). Following 15 min of magnetic stirring, the acetone was removed under reduced pressure at room temperature. The combined process of spontaneous emulsification and removal of acetone was repeated up to five times using the same aqueous suspension.

The suspension was dialyzed in cellulose membrane tube (Sigma, code D9777) overnight in distilled water and filtered through 0.45 μm hydrophilic syringe filter (Sigma, code CLS431220) and stored at 4° C. The hydrodynamic size measurement and size distribution of the nanospheres was performed by the dynamic light scattering (DLS) using a Coulter N4-Plus Submicron Particle Sizer (Coulter Corporation, Miami, Fla.).

### Example 9

### Preparation of the Fluorescent-Labeled Antioxidant-Antineoplastic Nanospheres

To demonstrate intracellular uptake in vitro cell culture, distribution in animal body, and intra-tumoral accumulation of the antioxidant-antineoplastic nanospheres, we prepared antioxidant-antineoplastic nanospheres labeled with a hydrophobic dye Coumarin 6 (Sigma, code 442631) or with a hydrophilic dye cy3/cy5/cy5.5 (GE Healthcare Life Sciences). Coumarin 6-labeled antioxidant-antineoplastic nanospheres were prepared using identical procedure as described in Example 5-Example 8 except that 50 μg of the dye was added to the organic phase prior to spontaneous emulsification. The incorporated Coumarin 6 remains associated with antioxidant-antineoplastic nanospheres during dialysis overnight.

To prepare cy3/cy5/cy5.5-labeled antioxidant-antineoplastic nanospheres, antioxidant-antineoplastic nanospheres were prepared using identical procedure as described in Example 5-Example 8 except that 0.1-2 mg of 1-octadecanethiol (Aldrich, code O1858) was added to the organic phase prior to spontaneous emulsification. The antioxidant-antineoplastic nanospheres were dialyzed overnight, and the concentration of thiol groups was determined as follows: Aldrithiol-2 (Sigma, code143049) was dissolved in ethanol (100 mM) and 10 μL of the solution was added to the suspension of antioxidant-antineoplastic nanospheres (80 μL). After addition of 10 μL of 10×PBS the mixture was incubated for 30 min at 37° C. The released 2-thiopyridone was separated using RP-HPLC with 50% acetonitrile as described in Example 1 and detected with UV detector at 341 nm. A standard curve for the determination of the released 2-thiopyridone was generated by measuring 2-thiopyridone generated from the reaction of known amount of Aldrithiol-2 and DTT.

To 3 mL of the suspension of 1-octadecanethiol-containing antioxidant-antineoplastic nanospheres 500 μL of 10×PBS and 1.5 molar equivalent of Cy3/Cy5/Cy5.5 maleimide were added. The reaction mixture was incubated overnight at room temperature and dialyzed at least 6 h to remove unbound cy5.5 maleimide from the suspension and filtered through 0.45 μm hydrophilic syringe filter (Sigma, code CLS431220) and stored at 4° C.

### Example 10

### Synthesis of the Statin Lactone and α-Lipoic Acid Derivatives A

α-Lipoic acid (ALA, 2.06 g, 10 mmol.) and a diol compound (tetraethylene glycol, TEG) (30 mmol) in 50 mL of anhydrous dichloromethane (DCM) were reacted with 4-(dimethylamino)-pyridine (DMAP, 15 mmol) in the presence of a molecular sieve (60 Å, 10-20 mesh beads) for 10 min at room temperature. N-(3-Dimethylaminopropyl)-N-ethylcarbodiimide hydrochloride (EDCI, 2.3 g, 12 mmol) was added portionwise over 10 min and the reaction mixture was stirred for 5 h at room temperature in the dark, filtered, and then concentrated under vacuum to reduce the volume. The resulting reaction mixture was purified using silica gel by direct loading onto the column without further preparation. The solvent was removed under reduced pressure to give the products.

A statin lactone (0.4 mmol), triphosgene (0.15 mmol), and DMAP (1.3 mmol) in anhydrous DCM was stirred for 10 min. The mono-ALA-TEG (0.4 mmol) was added and the reaction mixture was stirred for 24 h. The reaction mixture was concentrated under vacuum to reduce the volume. The resulting reaction mixture was purified using silica gel by direct loading onto the column without further preparation. The solvent was removed under reduced pressure to give the products. (See Scheme 5).

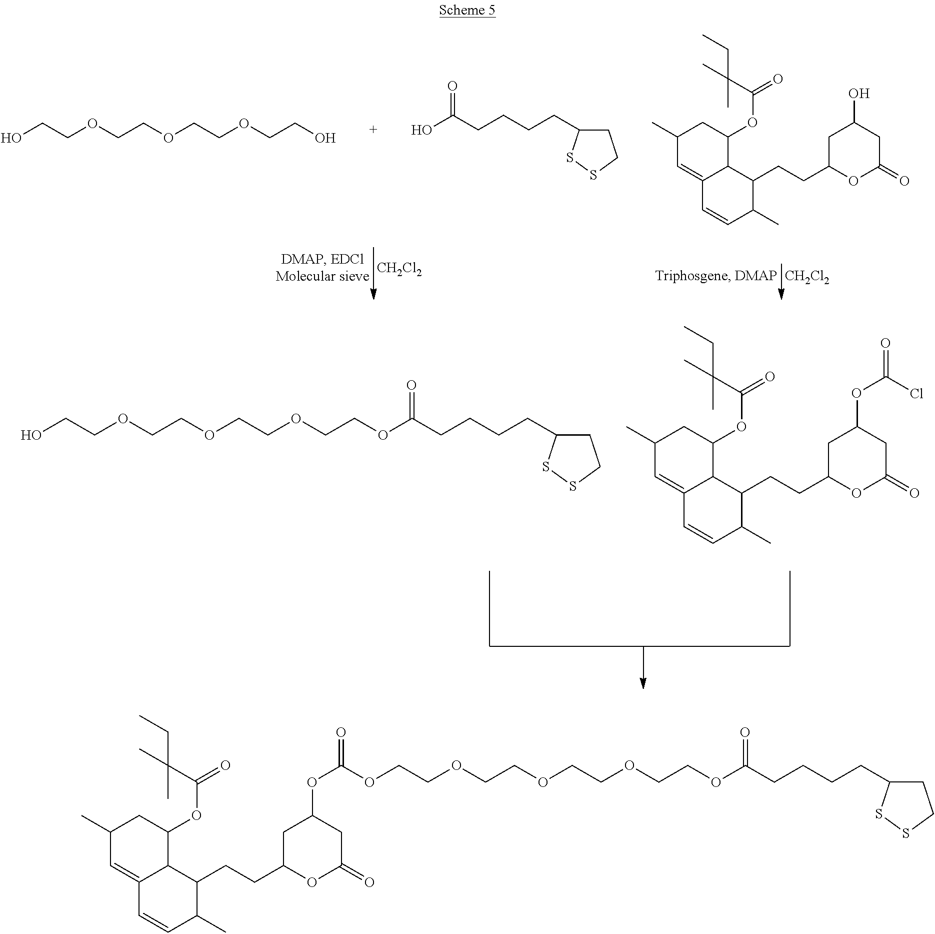

### Example 11

### Synthesis of the Statin Lactone and α-Lipoic Acid Derivatives B

A statin lactone (0.4 mmol), triphosgene (0.15 mmol), and DMAP (1.3 mmol) in anhydrous DCM was stirred for 10 min. TEG (0.2 mmol) was added and the reaction mixture was stirred for 24 h. The reaction mixture was concentrated under vacuum to reduce the volume. The resulting reaction mixture was purified using silica gel by direct loading onto the column without further preparation. The solvent was removed under reduced pressure to give the products. (See Scheme 6).

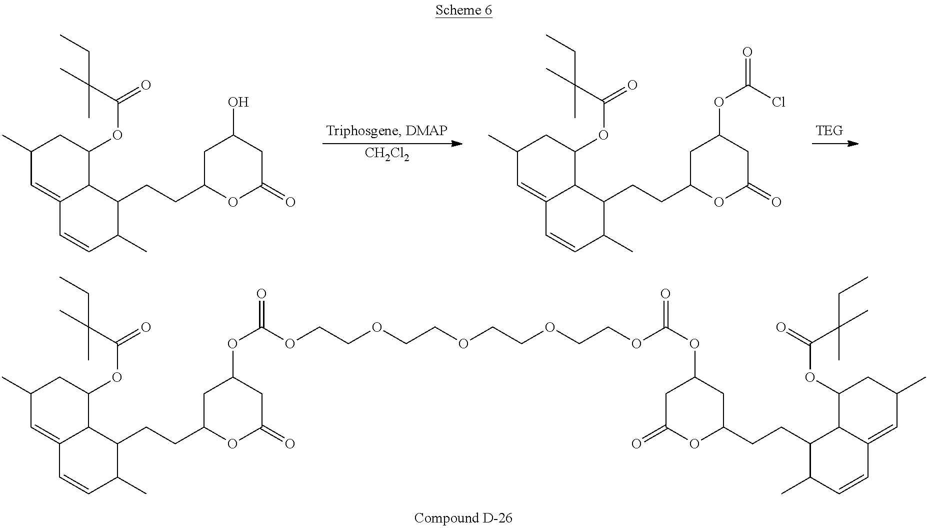

### Example 12

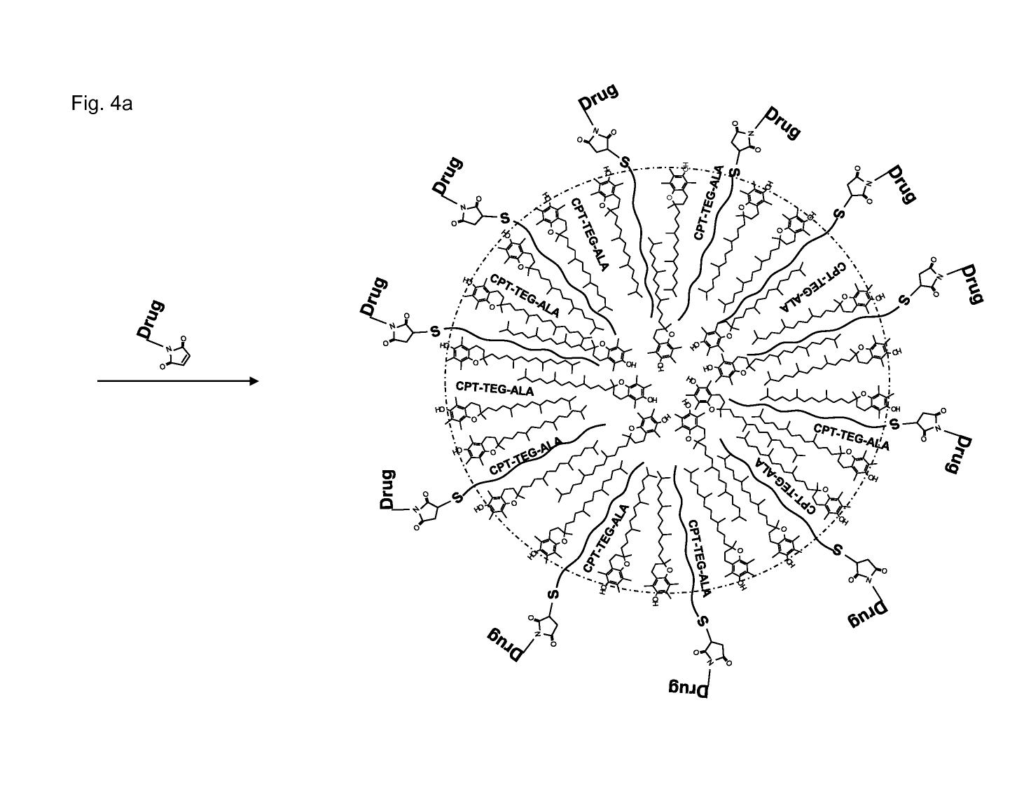

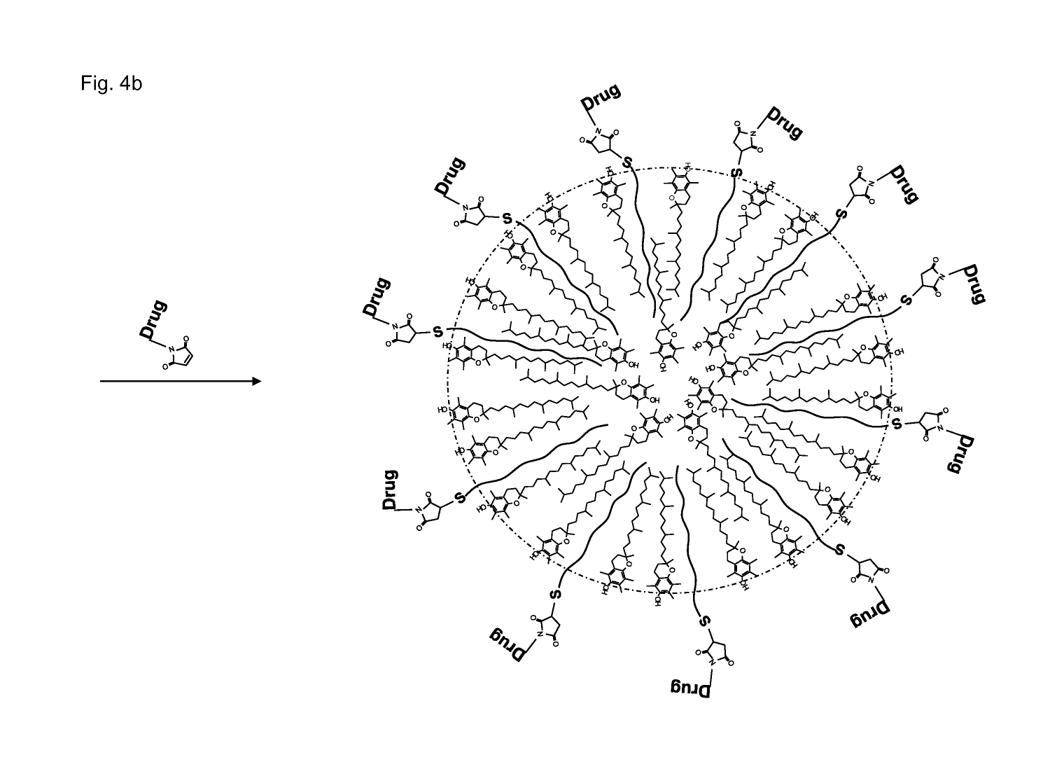

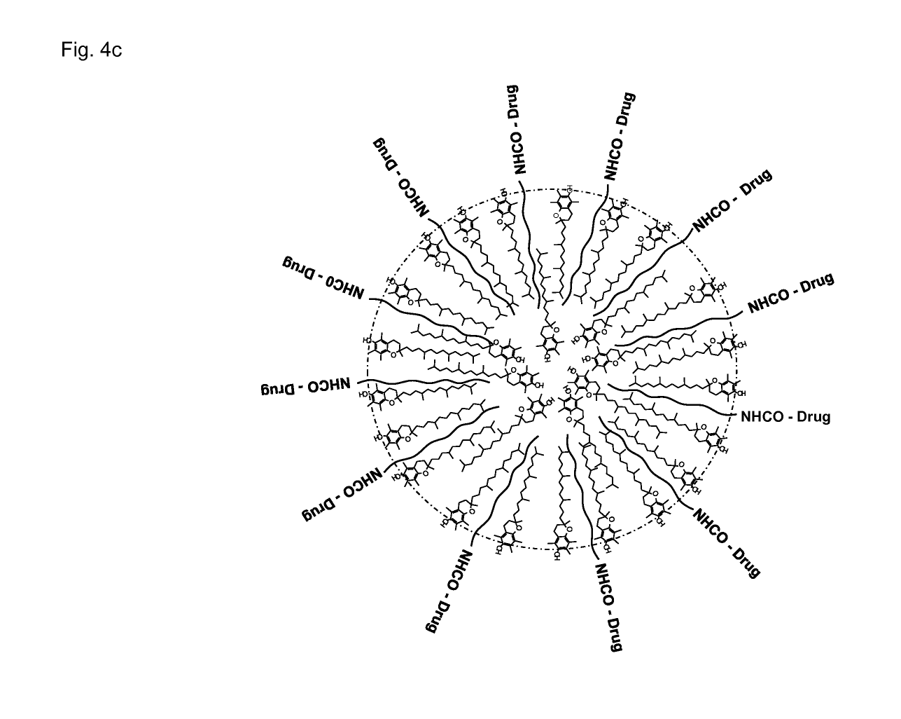

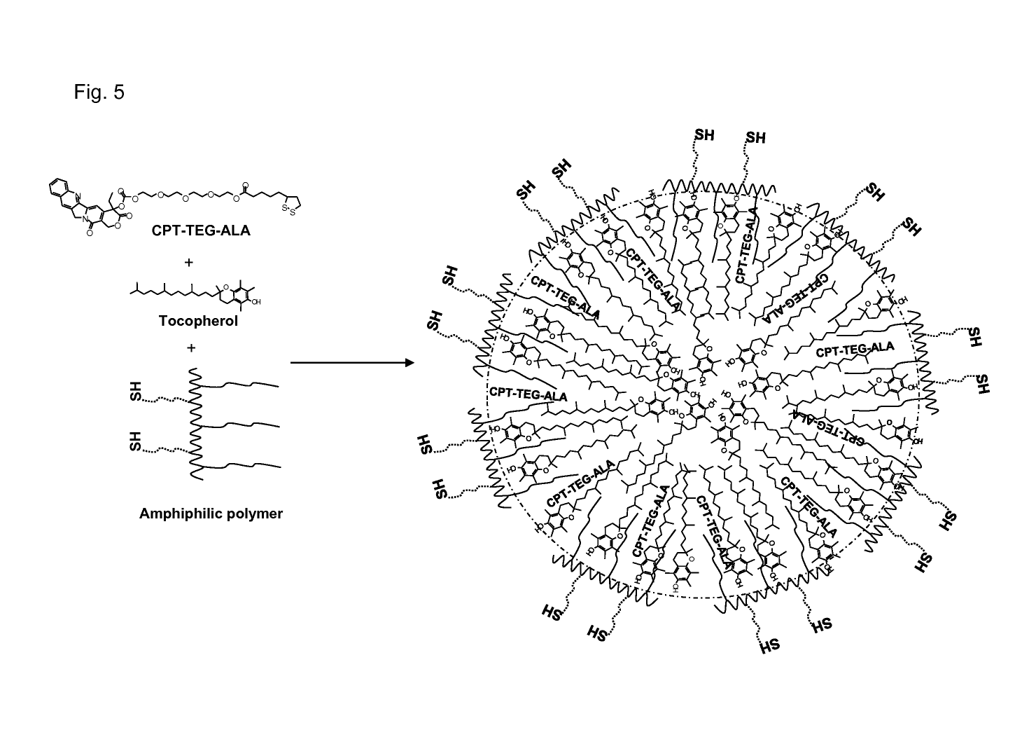

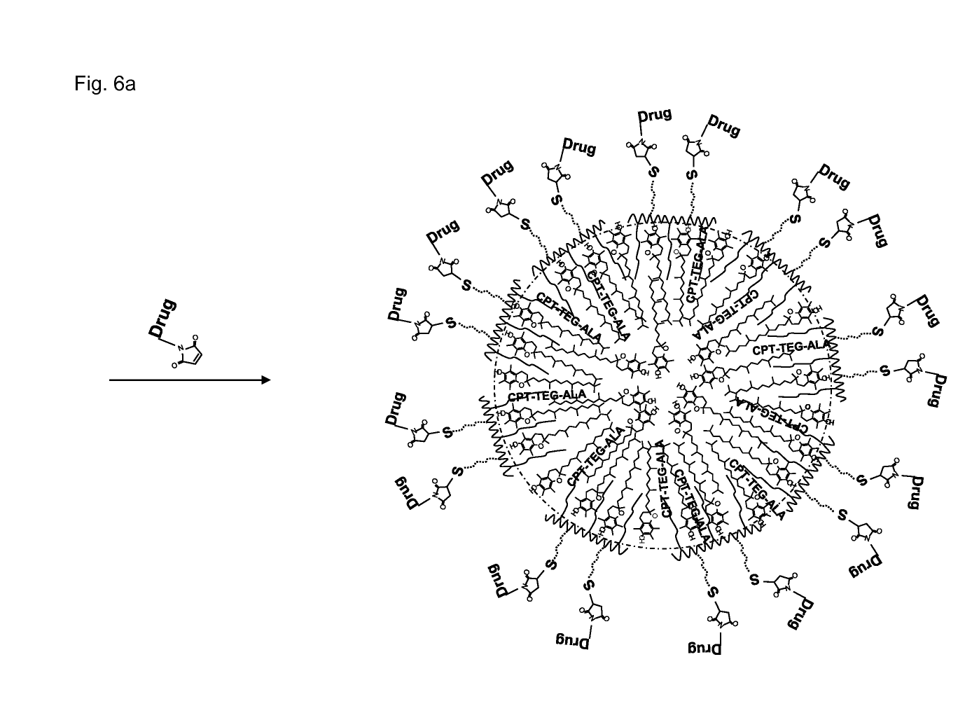

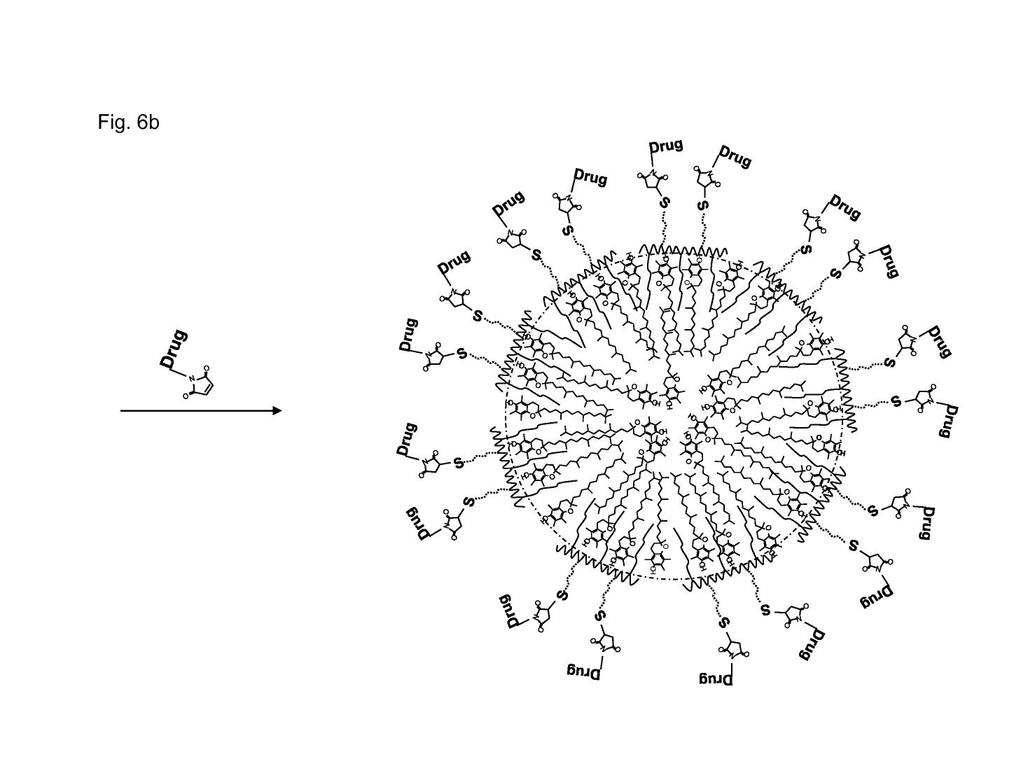

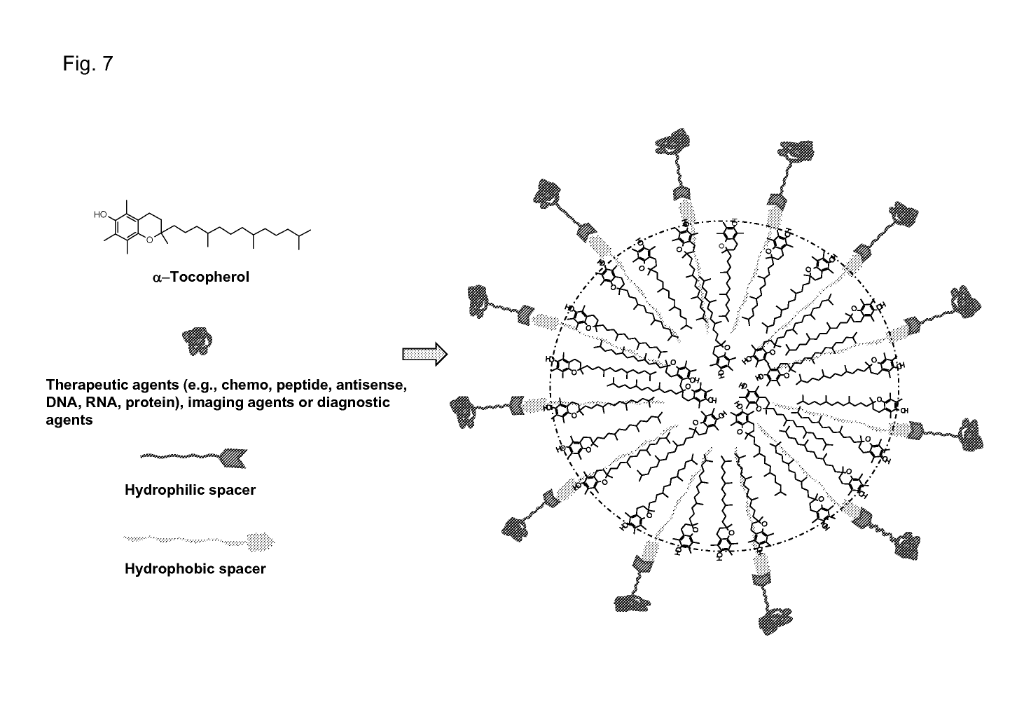

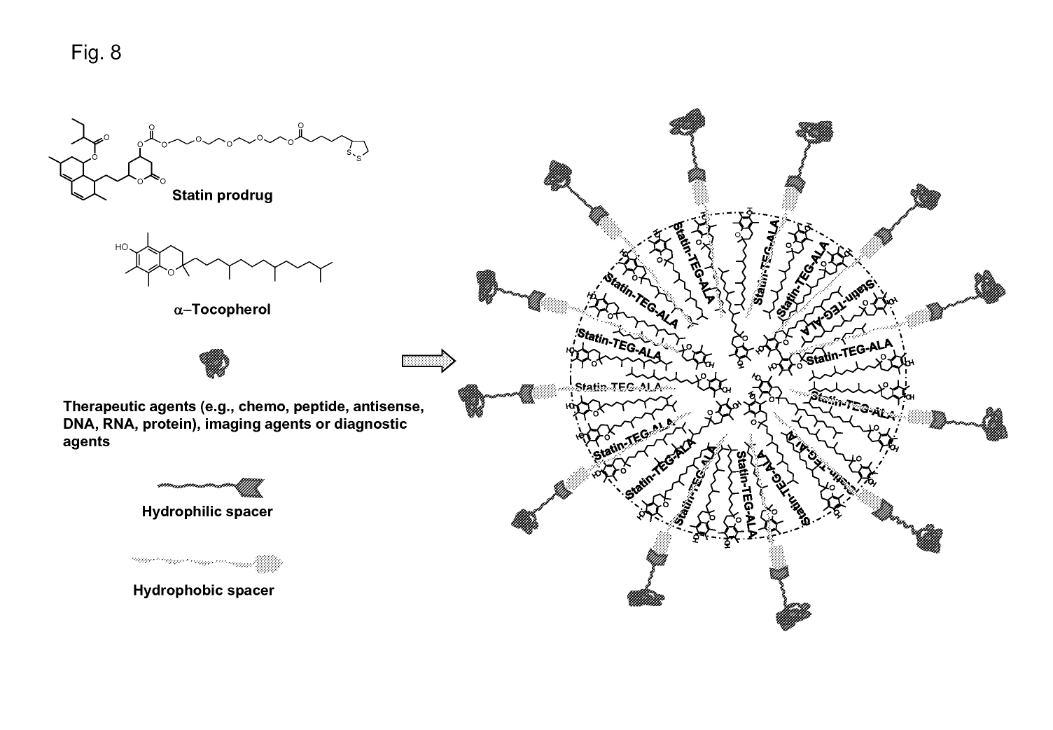

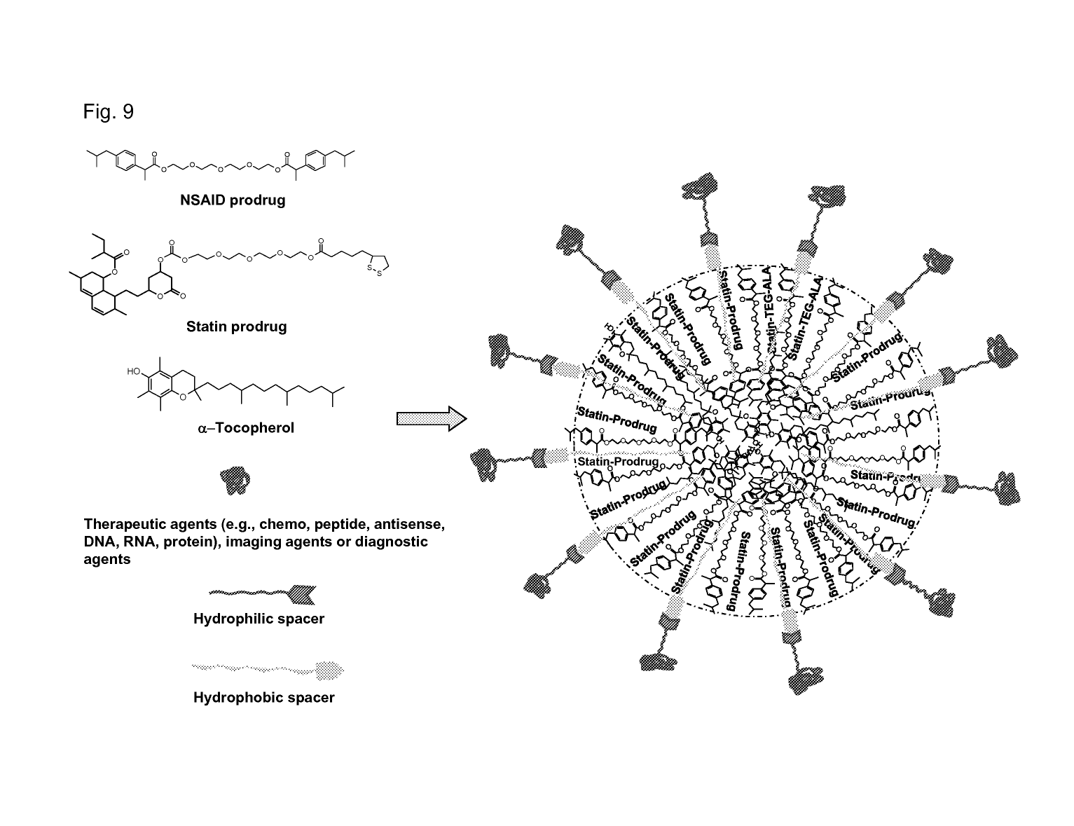

To prepare antioxidant-antineoplastic nanospheres carrying therapeutic agents on the surface, antioxidant-antineoplastic nanospheres surface-modified with amphiphilic spacer or amphiphilic polymer were prepared using identical procedure as described in Example A-Example D above except that 0.1-5 mg of amphiphilic spacer or amphiphilic polymer was added to the organic phase prior to spontaneous emulsification (FIGS. 3 and 5). The hydrophilic, reactive chemical groups of the amphiphilic spacer or amphiphilic polymer are directed to the surface of the nanospheres. As FIGS. 4 and 6 shows, the surface-modified nanospheres can be used to carrier therapeutic agents containing chemical groups that react with the chemical groups of the amphiphilic spacer or amphiphilic polymer on the surface of the nanospheres.

As depicted in FIGS. 1 and 2, the SH-maleimide pair can be replaced by NH₂—NHS pair or others.

### Example 13

### Synthesis of Camptothecin Prodrug and Nanoprodrug Preparation

Camptothecin prodrug CPT-TEG-ALA was synthesized by introducing biodegradable ester and carbonate bonds as described¹⁴. Nanoprodrug was prepared according to the method using spontaneous emulsification^{14,17} with multi-step modification. For Single step procedure, 7 mg of CPT-TEG-ALA and 50 mg α-tocopherol were dissolved in acetone (5 ml) containing polysorbate 80 (0.1% w/v). The organic solution was poured under moderate stirring on a magnetic plate into an aqueous phase prepared by dissolving 25 mg of Pluronic F68 in 10 ml distilled water (0.25% w/v). Following 15 min of stirring, acetone was removed under reduced pressure. For Multi-step procedure, the emulsification/evaporation cycle was repeated three times. Nanoprodrug (CPT-TEG-ALA/Toco) suspension obtained from the first cycle was used as aqueous phase for the second emulsification, and so forth. The suspension was dialyzed in cellulose membrane tube (Sigma) overnight in distilled water and filtered consecutively through 0.8, 0.45, and 0.2 μm hydrophilic syringe filter (Corning) and stored at 4° C. α-Tocopherol control nanosuspension was prepared using the same procedure except for the omission of camptothecin prodrug. For the visualization of the nanoprodrugs nanoparticle tracking analysis (NTA) was performed using a digital microscope LM10 system¹⁶.

### Example 14

### Fluorescent Labeling

Cy5.5 was incorporated into the nanoprodrug for fluorescent imaging. Cy5.5-labeled nanoprodrug was prepared using single step procedure as described above except that 2 mg of 1-octadecanethiol (Aldrich) was added to the organic phase prior to spontaneous emulsification. To 2 mL of the suspension of 1-octadecanethiol-containing nanoprodrugs 500 μL of 10×PBS and molar equivalent of Cy5.5 maleimide (GE Healthcare) were added. The reaction mixture was incubated overnight at room temperature under light protection. To remove unbound Cy5.5 maleimide, the suspension was purified on a G-25 Sephadex column (GE Healthcare) equilibrated with 20 mM sodium citrate buffer with 0.15 M NaCl³³. The labeled nanoprodrug was filtered and stored as described above. The concentration of the bound Cy5.5 was determined as follows: 200 μL of nanoprodrug suspention was mixed with 800 μL acetonitrile and the absorbance was measured at 675 nm. The concentration was calculated using a standard curve generated with Cy5.5 maleimide.

### Example 15

### In Vitro Cell Culture and Cellular Uptake of Nanoprodrugs

The human glioblastoma cell line U87 MG was obtained from American Type Culture Collection (ATCC). The cells were grown at 37° C. at an atmosphere of 5% CO₂ in humidified air in Minimum Essential Medium (MEM, Invitrogen) containing antibiotics penicillin (100 U/mL) and streptomycin (100 μg/mL) and supplemented with 10% fetal bovine serum (FBS, Invitrogen). To demonstrate intracellular uptake and degradation of the nanoprodrugs, cells were grown in 75 cm² culture flask up to ˜70% confluent density and treated with CPT-TEG-ALA/Toco nanoprodrug (5 μM) for 3 days. Cells were washed three times with PBS to remove free nanoprodrugs and trypsinized. Cells were collected by centrifugation and the pellet was resuspended in PBS. After three resuspension/cenbtrifugation cycles, approximately 80 million cells were treated with 1 mL of lysis buffer (1% of Triton X-100, 10 mM Tris-HCl, pH 7.4) for 15 min at 37° C. The lysate was mixed with acetonitrile (3 mL) and centrifuged for 10 min at 10,000×g. The supernatant was collected and evaporated to dryness. The residue was dissolved in 500 μL acetonitrile and centrifuged for 15 min at 20,000×g. The supernatant was analyzed with RP-HPLC as described¹⁴. To demonstrate intracellular uptake of the fluorescent nanoprodrugs, cells were incubated in the presence of fluorescent-labeled nanoprodrugs. Four chamber culture slides (BD Biosciences) were seeded with U87 MG cells, and the cells were allowed to attach for 24 h. The medium was replaced with 1.0 mL of freshly prepared suspension of the fluorescent-labeled nanoprodrugs (1 μM Cy5.5) in medium, and the chamber slides were incubated for 5 h. Cells were washed three times with PBS to remove free nanoprodrugs, one drop of mounting medium with 4′,6-diamidino-2-phenylindole (DAPI) (Prolong Gold, Invitrogen) was added and then cover slides were placed. For microscopic analysis, a confocal laser-scanning microscope (Leica Microsystem SP5) equipped with digital camera with fluorescent microscope (Model Upright Zeiss) was used.

### Example 16

### Animal Model

All animal studies were performed according to Cedars-Sinai Medical Center Institutional Animal Care and Use Committee protocols. Female 6- to 8-week-old athymic nu/nu mice (Charles River Laboratories) were used for all experiments. For subcutaneous tumor model, mice were injected in the right flank with 10⁷ U87 MG human glioma cells suspended in PBS (100 uL). For Intracranial tumor model, mice underwent intracranial stereotactic implantation of U87 MG cells. Mice were anesthetized using a ketamine and dexmedetomidine combination as a single intraperitoneal injection. 10⁵ U87 MG cells suspended in 2 μl of PBS were implanted in the right frontal region of the brain using a Hamilton syringe. The animals received intraperitoneal injection of atipamezole to reverse the dexmedetomidine effect. A single subcutaneous injection of buprenorphine was administered for pain relief.

### Example 17

### In Vivo Anti-Tumor Efficacy of Nanoprodrug

The anti-tumor effect of the CPT-TEG-ALA/Toco nanoprodrug was tested on subcutaneous and intracranial xenografts of U87 MG tumors in mice. In the subcutaneous model, treatment was started when the tumor size reached approx. 0.5-1.0 cm in diameter. The animals (n=6) received intravenous (tail vein) injection of nanoprodrugs on a daily basis for five days (4 mg/kg/day CPT-TEG-ALA). Two perpendicular diameters of the tumor are measured, and the volume is calculated according to the equation: V (mm³)=L (mm)×W² (mm²)/2, where L is the longest diameter and W is the diameter perpendicular to L. In the intracranial model, the animals (n=8) received intravenous (tail vein) injection of nanoprodrugs (16 mg/kg/day CPT-TEG-ALA) beginning 7 days after tumor implantation every three days for 4 weeks. When the animals manifested severe hemiparesis, or exhibited inability to access food, water, seizure activity, weakness, paralysis, the animals were sacrificed. As control, animals received injection of irinotecan, α-tocopherol nanosuspension, and saline

### Example 18

### Optical Imaging

In the subcutaneous model, 100 μL fluorescent nanoprodrug (10 μM Cy5.5) were injected via tail vein injection after the tumor sized reached >1 cm. In the intracranial model, fluorescent nanoprodrug was injected when there were signs of significant neurological impairment. Fluorescent imaging of the living animals and harvested organs were performed using Xenogen 200 Imaging System (Caliper Life Sciences). Organs (brain, heart, liver, kidney, spleen, and lung) were harvested from the animals and imaged immediately to determine the accumulation of nanoprodrug. Imaging was made on whole body (subcutaneous model only) and on isolated organs and tumor sections embedded and frozen in OCT compound. For fluorescent confocal microscopy, tumors were cryosectioned (10 μm), one drop of mounting medium with DAPI (Prolong Gold, Invitrogen) was added and then cover slides were placed. For microscopic analysis, a confocal laser-scanning microscope (Leica Microsystem SP5) equipped with digital camera with fluorescent microscope (Model Upright Zeiss) was used.

### Example 19

### Histology and Immunohistochemistry

Whole brains were harvested immediately after the animals were sacrificed, frozen in OCT compound, cryosectioned (10 μm), and stained with hematoxylin and eosin. For immunohistochemistry, sections were fixed in 4% PFA for 5 min. To demonstrate tumor angiogenesis, the frozen sections were treated with rat anti-mouse CD31 (1:100; BD Biosciences) and then FITC-conjugated goat anti-rat IgG (Sigma). To detect the proliferative activity, sections were treated with rabbit anti-human Ki-67 and then FITC-conjugated goat anti-rabbit IgG (Sigma). All sections were counterstained with DAPI by adding one drop of mounting medium with DAPI (Prolong Gold, Invitrogen). Confocal microscopic analysis was performed as described above.

### Example 20

### Statistical Analysis

Other than survival study, the results were analyzed and expressed as mean±standard deviation (S.D.). Statistical analysis of the results was carried out using Student's t-test. For mouse survival study, log-rank statistical analysis was performed. For all tests, differences were considered statistically significant at P<0.05.

### REFERENCES

1. Krex, D. et al. Long-term survival with glioblastoma multiforme. Brain 130, 2596-2606 (2007).
2. Yang, I. & Aghi, M. K. New advances that enable identification of glioblastoma recurrence. Nat. Rev. Clin. Oncol. 6, 648-657 (2009).
3. Cecchelli, R. et al. Modelling of the blood-brain barrier in drug discovery and development. Nat. Rev. Drug Disc. 6, 650-661 (2007).
4. Hawkins, B. T. & Davis, T. P. The blood-brain barrier/neurovascular unit in health and disease. Pharmacol. Rev. 57, 173-185 (2005).
5. Juillerat-Jeanneret, L. The targeted delivery of cancer drugs across the blood-brain barrier: chemical modifications of drugs or drug-nanoparticles? Drug Discov. Today 13, 1099-1106 (2008).
6. Wolburg, H. et al. Localization of claudin-3 in tight junctions of the blood-brain barrier is selectively lost during experimental autoimmune encephalomyelitis and human glioblastoma multiforme. Acta Neuropathol. 105, 586-592 (2003).
7. Zlokovic, B. V. The blood-brain barrier in health and chronic neurodegenerative disorders. Neuron 57, 178-201 (2008).
8. Shlosberg, D., Benifla, M., Kaufer, D. & Friedman, A. Blood-brain barrier breakdown as a therapeutic target in traumatic brain injury. Nat. Rev. Neurol. 6, 393-403 (2010).
9. Iyer, A. K., Khaled, G., Fang, J. & Maeda, H. Exploiting the enhanced permeability and retention effect for tumor targeting. Drug Discov. Today 11, 812-818 (2006).
10. Maeda, H. The enhanced permeability and retention (EPR) effect in tumor vasculature: the key role of tumor-selective macromolecular drug targeting. Adv. Enzyme Regul. 41, 189-207 (2001).
11. Ferrari, M. Cancer nanotechnology: opportunities and challenges. Nat. Rev. Cancer 5, 161-171 (2005).
12. Kuo, F. et al. Nanoemulsions of an anti-oxidant synergy formulation containing gamma tocopherol have enhanced bioavailability and anti-inflammatory properties. Int. J. Pharm. 363, 206-213 (2008).
13. Pizzolato, J. F. & Saltz, L. B. The camptothecins. The Lancet. 361, 2235-2242 (2003).
14. Lee, B. S. et al. Oxidative stimuli-responsive nanoprodrug of camptothecin kills glioblastoma cells. Bioorg. Med. Chem. Lett. 20, 5262-5268 (2010).
15. Shafiq, S. et al. Development and bioavailability assessment of ramipril nanoemulsion formulation. Eur. J. Pharma. Biopharm. 66, 227-243 (2007).
16. Filipe, V., Hawe, A. & Jiskoot, W. Critical evaluation of nanoparticle tracking analysis (NTA) by NanoSight for the measurement of nanoparticles and protein aggregates. Pharm. Res. 27, 796-810 (2010).
17. Lee, B. S. et al. Stimuli-responsive antioxidant nanoprodrugs of NSAIDs. Int. J. Pharm. 372, 112-124 (2009).
18. Ohshima, H. & Bartsch, H. Chronic infections and inflammation process as cancer risk factors: possible role of nitric oxide in carcinogenesis. Mutat. Res. 305, 253-264 (1994).
19. Weitzman, S. A. & Gordon, L. I. Inflammation and cancer: role of phagocyte-generated oxidants in carcinogenesis. Blood 76, 655-663 (1990).
20. Trush, M. A. & Kensler, T. W. An overview of the relationship between oxidative stress and chemical carcinogenesis. Free Radic. Biol. Med. 10, 201-209 (1991).
21. Cerutti, P. A. Prooxidant states and tumor promotion. Science 227, 375-381 (1985).
22. Bunt, S. K. et al. Reduced inflammation in the tumor microenvironment delays the accumulation of myeloid-derived suppressor cells and limits tumor progression. Cancer Res. 67, 10019-10026 (2007).
23. Henson, J. W., Cordon-Cardo, C. & Posner, J. B. P-glycoprotein expression in brain tumors. J Neurooncol. 14, 37-43 (1992).
24. Sawada, T., Kato, Y., Sakayori, N., Takekawa, Y. & Kobayashi, M. Expression of the multidrugresistance P-glycoprotein (Pgp, MDR-1) by endothelial cells of the neovasculature in central nervous system tumors. Brain Tumor Pathol. 16, 23-27 (1999).
25. Bredel, M. & Zentner, J. Brain-tumour drug resistence: the bare essentials. Lancet Oncol. 3, 397-406 (2002).
26. Shapiro, A. B., Corder, A. B., & Ling, V. P-glycoprotein-mediated Hoechst 33342 transport out of the lipid bilayer. Eur. J. Biochem. 250, 115-121 (1997).
27. Iyer, L. et al. Biliary transport of irinotecan and metabolites in normal and P-glycoprotein-deficient mice. Cancer Chemother. Pharmacol. 49, 336-41 (2002).
28. Sen, W. J. et al. CPT-11 sensitivity in relation to the expression of P170-glycoprotein and multidrug resistance-associated protein. Br. J. Cancer 77, 359-65 (1998).
29. Wang, M., Wang, T., Liu, S., Yoshida, D. & Teramoto, A. The expression of matrix metalloproteinase-2 and -9 in human gliomas of different pathological grades. Brain Tumor Pathol. 20, 65-72 (2003).
30. Gilbertson, R. J. & Rich, J. N. Making a tumour's bed: glioblastoma stem cells and the vascular niche. Nat. Rev. Cancer 7, 733-736 (2007).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A nanosphere comprising:
tocopherol and
a therapeutic agent or an imaging agent conjugated to a hydrophilic spacer, a hydrophobic spacer, an amphiphilic spacer, or an amphiphilic polymer.

2. A method of treating cancer in a subject in need thereof, comprising:
providing a nanosphere of claim 1; and
administering a therapeutically effective amount of the nanosphere to the subject to treat the cancer.

3. A method of detecting or diagnosing cancer in a subject in need thereof comprising:
providing a nanosphere of claim 1;
administering an effective amount of the nanosphere to the subject; and
imaging the subject to detect or diagnose the cancer.

4. The nanosphere of claim 1, further comprising:
an antioxidant α-lipoic acid-containing hydrophobic compound having Formula A-Ia: 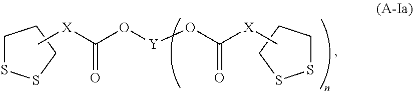
wherein X is selected from the group consisting of a substituted, unsubstituted, branched or unbranched chain of carbon atoms, and may optionally contain a heteroatom; Y is selected from the group consisting of a branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic group; and n is an integer of at least one.

5. The nanosphere of claim 4, wherein the dithiolane moiety in Formula Ia is an α-lipoic acid and is represented by Formula A-IIa:

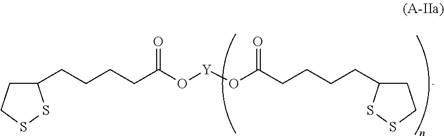

6. A method of treating cancer in a subject in need thereof, comprising:
providing the nanosphere of claim 4; and
administering a therapeutically effective amount of the nanosphere to the subject to treat the cancer.

7. A method of detecting or diagnosing cancer in a subject in need thereof comprising:
providing the nanosphere of claim 4;
administering an effective amount of the nanosphere to the subject; and
imaging the subject to detect or diagnose the cancer.

8. The nanosphere of claim 1, further comprising:
a hydrophobic nonsteroidal anti-inflammatory drug (NSAID) derivative having Formula B-I: 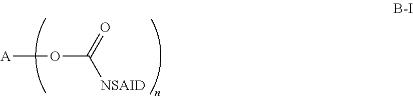
wherein the A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; and n is an integer of at least two.

9. A method of treating cancer in a subject in need thereof, comprising:
providing the nanosphere of claim 8; and
administering a therapeutically effective amount of the nanosphere to the subject to treat the cancer.

10. A method of detecting or diagnosing cancer in a subject in need thereof comprising:
providing the nanosphere of claim 8;
administering an effective amount of the nanosphere to the subject; and
imaging the subject to detect or diagnose the cancer.

11. The nanosphere of claim 1, further comprising:
a hydrophobic antioxidant and anti-inflammatory derivative of an nonsteroidal anti-inflammatory drug (NSAID) having Formula B-II: 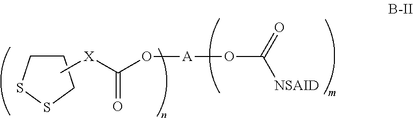
wherein X is selected from the group consisting of a substituted, unsubstituted, branched or unbranched chain of carbon atoms and may optionally contain a heteroatom; A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; n is an integer of at least one; and m is an integer of at least one.

12. The nanosphere of claim 11, wherein the hydrophobic antioxidant and anti-inflammatory derivative of an NSAID is Formula B-III: 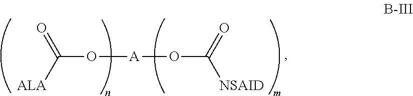
wherein ALA represents α-lipoic acid.

13. A method of treating cancer in a subject in need thereof, comprising:
providing the nanosphere of claim 11; and
administering a therapeutically effective amount of the nanosphere to the subject to treat the cancer.

14. A method of detecting diagnosing cancer in a subject in need thereof comprising:
providing the nanosphere of claim 11;
administering an effective amount of the nanosphere to the subject; and
imaging the subject to detect or diagnose the cancer.

15. The nanosphere of claim 1, further comprising:
a statin lactone derivative having Formula D-I, D-II, D-III, D-IV, D-V or D-VI: 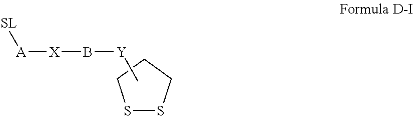
wherein A and B are independently selected from the group consisting of —OC(O)—, —OC(O)O—, and —OC(O)N(R)—, wherein R is a hydrogen atom, or a substituted, unsubstituted, branched or unbranched chain of carbon atoms and may optionally comprise heteroatoms; X and Y are linkers, each independently comprises substituted, unsubstituted, branched or unbranched chain of carbon atoms and may optionally comprise heteroatoms; and SL is selected from statin lactones from the group consisting of atorvastatin, fluvastatin, lovastatin, mevastatin, pitavastatin, rosuvastatin, and simvastatin, 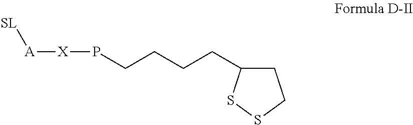
wherein A is selected from the group consisting of —OC(O)—, —OC(O)O—, and —OC(O)N(R)—, wherein R is a hydrogen atom, or a substituted, unsubstituted, branched or unbranched chain of carbon atoms and may optionally comprise heteroatoms; P is selected from the group consisting of —OC(O)—, and —N(R)C(O)—, wherein R is a hydrogen atom, or a substituted, unsubstituted, branched or unbranched chain of carbon atoms and may optionally comprise heteroatoms; X is a linker comprising a substituted, unsubstituted, branched or unbranched chain of carbon atoms and may optionally comprise heteroatoms; and SL is selected from statin lactones from the group consisting of atorvastatin, fluvastatin, lovastatin, mevastatin, pitavastatin, rosuvastatin, and simvastatin, 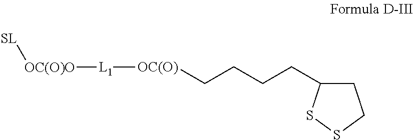
wherein L₁ is a moiety formed by esterification of two free esterifiable hydroxyl groups on a diol; and SL is selected from statin lactones from the group consisting of atorvastatin, fluvastatin, lovastatin, mevastatin, pitavastatin, rosuvastatin, and simvastatin, 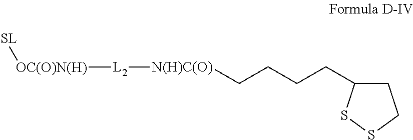
wherein L₂ is a moiety formed by using a diamine as the linker in the process of producing the statin lactone derivative, and SL is selected from statin lactones from the group consisting of atorvastatin, fluvastatin, lovastatin, mevastatin, pitavastatin, rosuvastatin, and simvastatin, 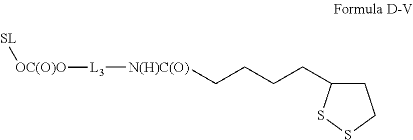
wherein L₃ may be a moiety formed by using an aminoalcohol as a linker in the process of producing the statin lactone derivative; and SL is selected from statin lactones from the group consisting of atorvastatin, fluvastatin, lovastatin, mevastatin, pitavastatin, rosuvastatin, and simvastatin, 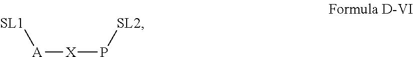
wherein A and P are selected independently from the group consisting of —OC(O)—, —OC(O)O—, and —OC(O)N(R)—, wherein R is a hydrogen atom, or a substituted, unsubstituted, branched or unbranched chain of carbon atoms and may optionally comprise heteroatoms; X is a linker comprising a substituted, unsubstituted, branched or unbranched chain of carbon atoms and may optionally comprise heteroatoms; and SL1 and SL2 are selected independently from the statin lactones from the group consisting of atorvastatin, fluvastatin, lovastatin, mevastatin, pitavastatin, rosuvastatin, and simvastatin.

16. The nanosphere of claim 15, wherein the statin lactone derivative is selected from the group consisting of:

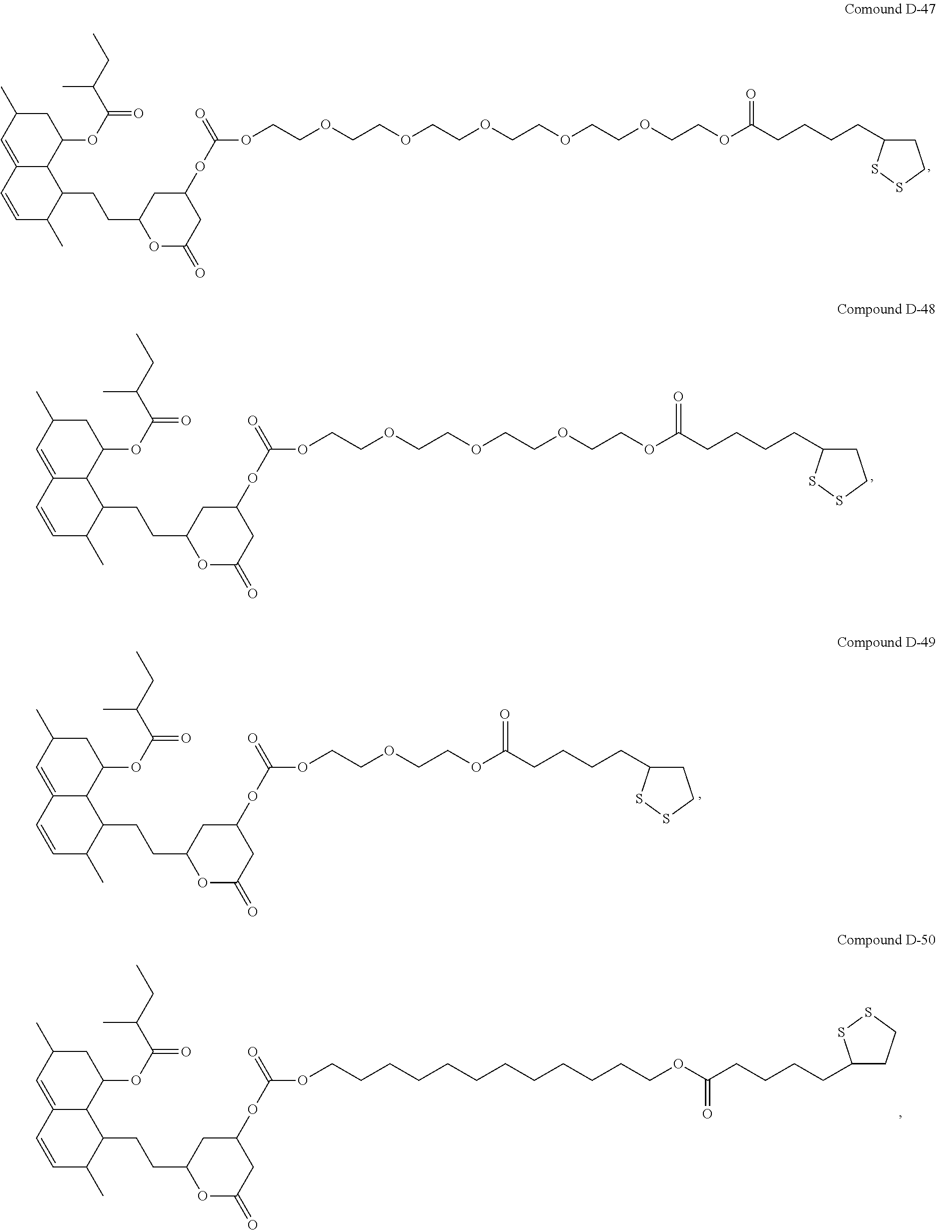 [SMILES: CCC(C)C(=O)OC1CC(C)C=C2C=CC(C)C(CCC3CC(OC(=O)OCCOCCOCCOCCOCCOCCOC(=O)CCCCC4CCSS4)CC(=O)O3)C21 ; CCC(C)C(=O)OC1CC(C)C=C2C=CC(C)C(CCC3CC(OC(=O)OCCOCCOCCOCCOC(=O)CCCCC4CCSS4)CC(=O)O3)C21 ; CCC(C)C(=O)OC1CC(C)C=C2C=CC(C)C(CCC3CC(OC(=O)OCCOCCOC(=O)CCCCC4CCSS4)CC(=O)O3)C21 ; CCC(C)C(=O)OC1CC(C)C=C2C=CC(C)C(CCC3CC(OC(=O)OCCCCCCCCCCCCOC(=O)CCCCC4CCSS4)CC(=O)O3)C21]

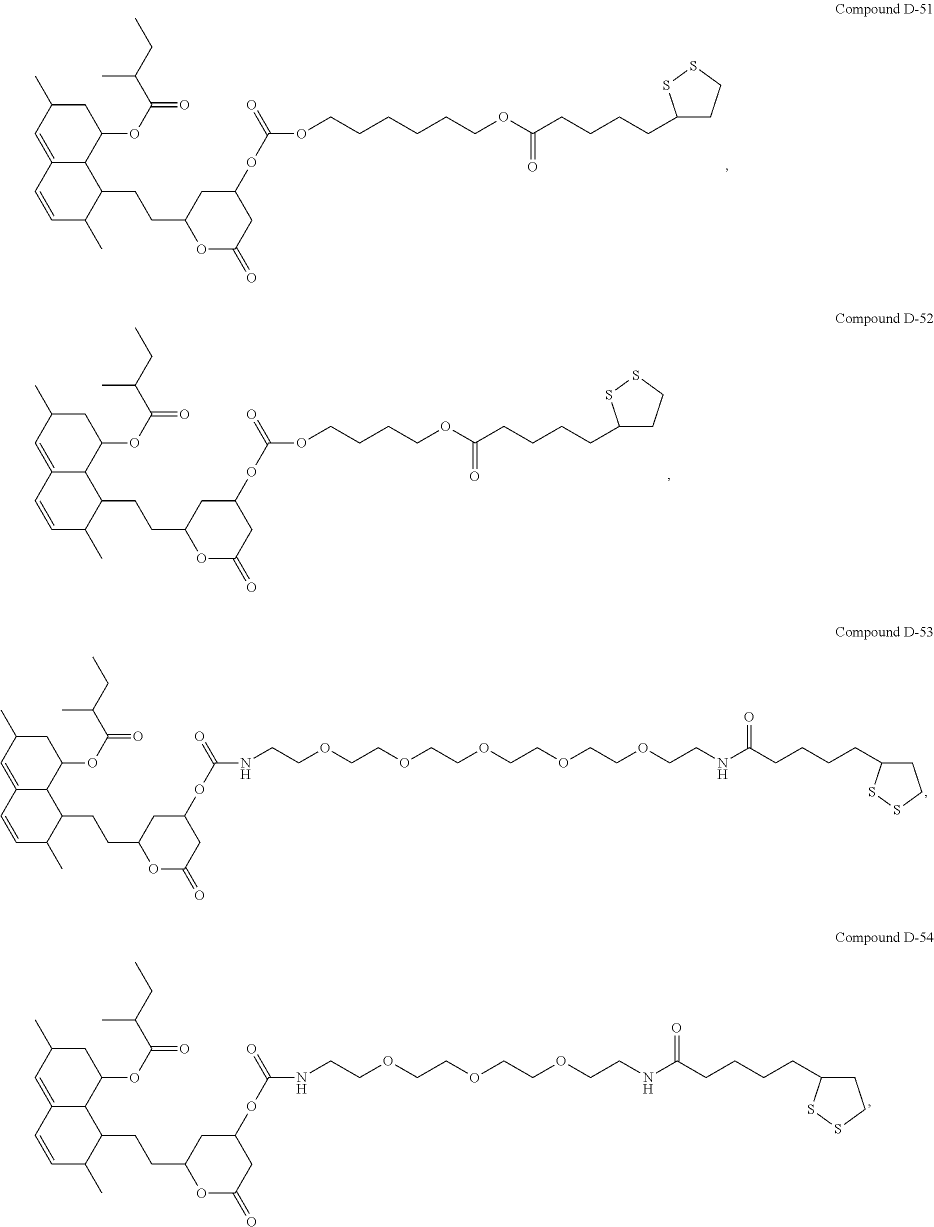 [SMILES: CCC(C)C(=O)OC1CC(C)C=C2C=CC(C)C(CCC3CC(OC(=O)OCCCCCCOC(=O)CCCCC4CCSS4)CC(=O)O3)C21 ; CCC(C)C(=O)OC1CC(C)C=C2C=CC(C)C(CCC3CC(OC(=O)OCCCCOC(=O)CCCCC4CCSS4)CC(=O)O3)C21 ; CCC(C)C(=O)OC1CC(C)C=C2C=CC(C)C(CCC3CC(OC(=O)NCCOCCOCCOCCOCCOCCNC(=O)CCCCC4CCSS4)CC(=O)O3)C21 ; CCC(C)C(=O)OC1CC(C)C=C2C=CC(C)C(CCC3CC(OC(=O)NCCOCCOCCOCCNC(=O)CCCCC4CCSS4)CC(=O)O3)C21]

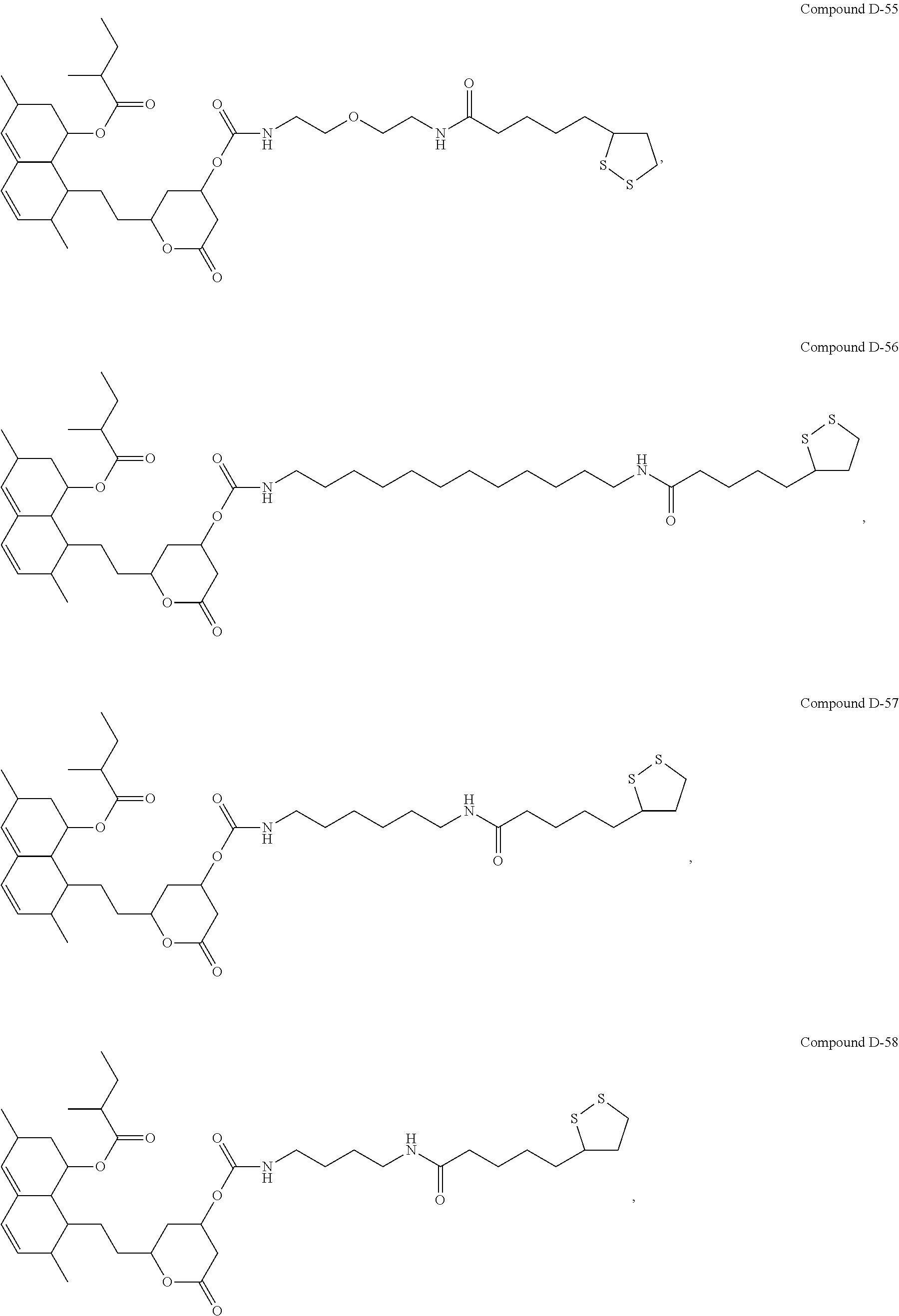 [SMILES: CCC(C)C(=O)OC1CC(C)C=C2C=CC(C)C(CCC3CC(OC(=O)NCCOCCNC(=O)CCCCC4CCSS4)CC(=O)O3)C21 ; CCC(C)C(=O)OC1CC(C)C=C2C=CC(C)C(CCC3CC(OC(=O)NCCCCCCCCCCCCNC(=O)CCCCC4CCSS4)CC(=O)O3)C21 ; CCC(C)C(=O)OC1CC(C)C=C2C=CC(C)C(CCC3CC(OC(=O)NCCCCCCNC(=O)CCCCC4CCSS4)CC(=O)O3)C21 ; CCC(C)C(=O)OC1CC(C)C=C2C=CC(C)C(CCC3CC(OC(=O)NCCCCNC(=O)CCCCC4CCSS4)CC(=O)O3)C21]

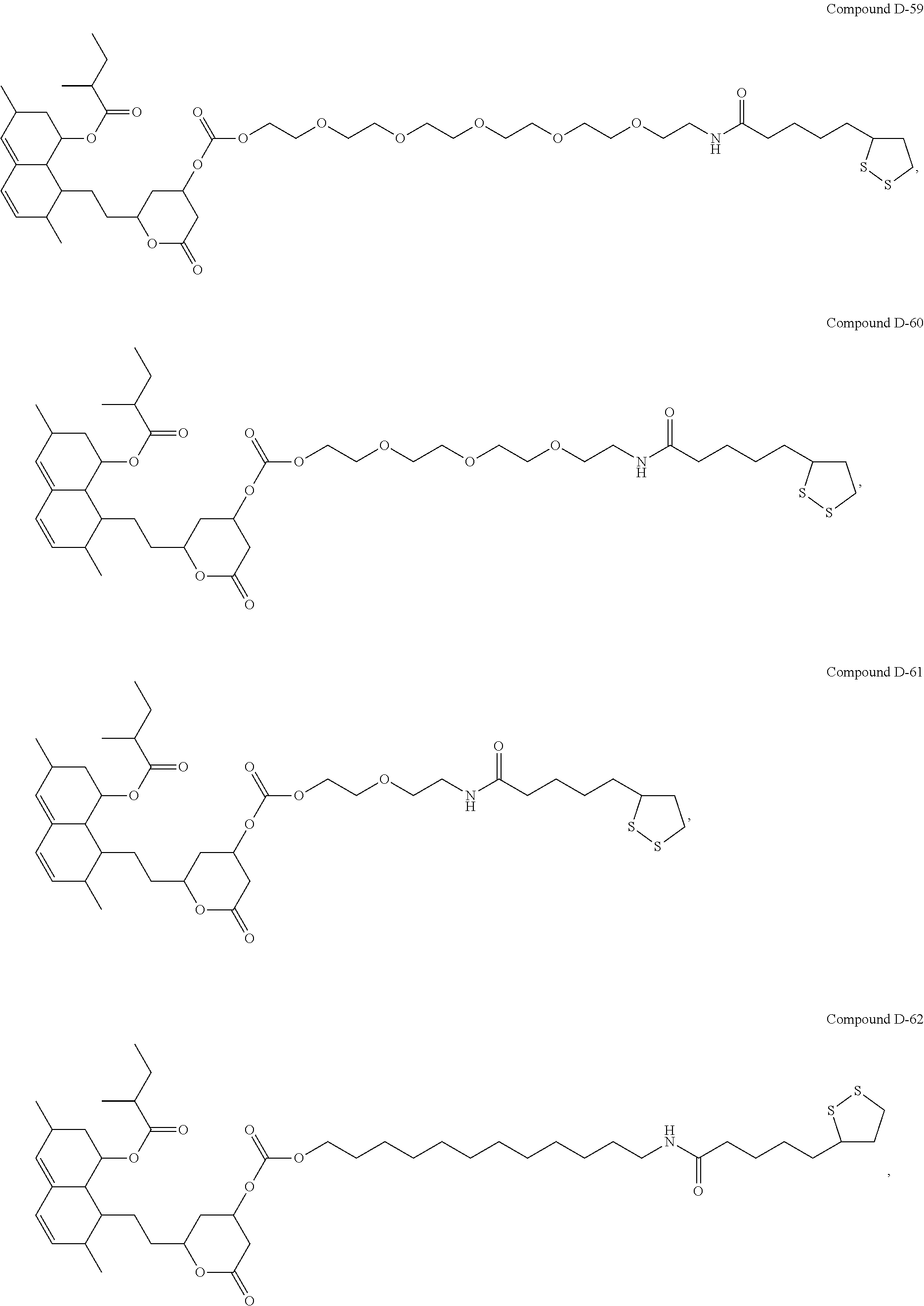 [SMILES: CCC(C)C(=O)OC1CC(C)C=C2C=CC(C)C(CCC3CC(OC(=O)OCCOCCOCCOCCOCCOCCNC(=O)CCCCC4CCSS4)CC(=O)O3)C21 ; CCC(C)C(=O)OC1CC(C)C=C2C=CC(C)C(CCC3CC(OC(=O)OCCOCCOCCOCCNC(=O)CCCCC4CCSS4)CC(=O)O3)C21 ; CCC(C)C(=O)OC1CC(C)C=C2C=CC(C)C(CCC3CC(OC(=O)OCCOCCNC(=O)CCCCC4CCSS4)CC(=O)O3)C21 ; CCC(C)C(=O)OC1CC(C)C=C2C=CC(C)C(CCC3CC(OC(=O)OCCCCCCCCCCCCNC(=O)CCCCC4CCSS4)CC(=O)O3)C21]

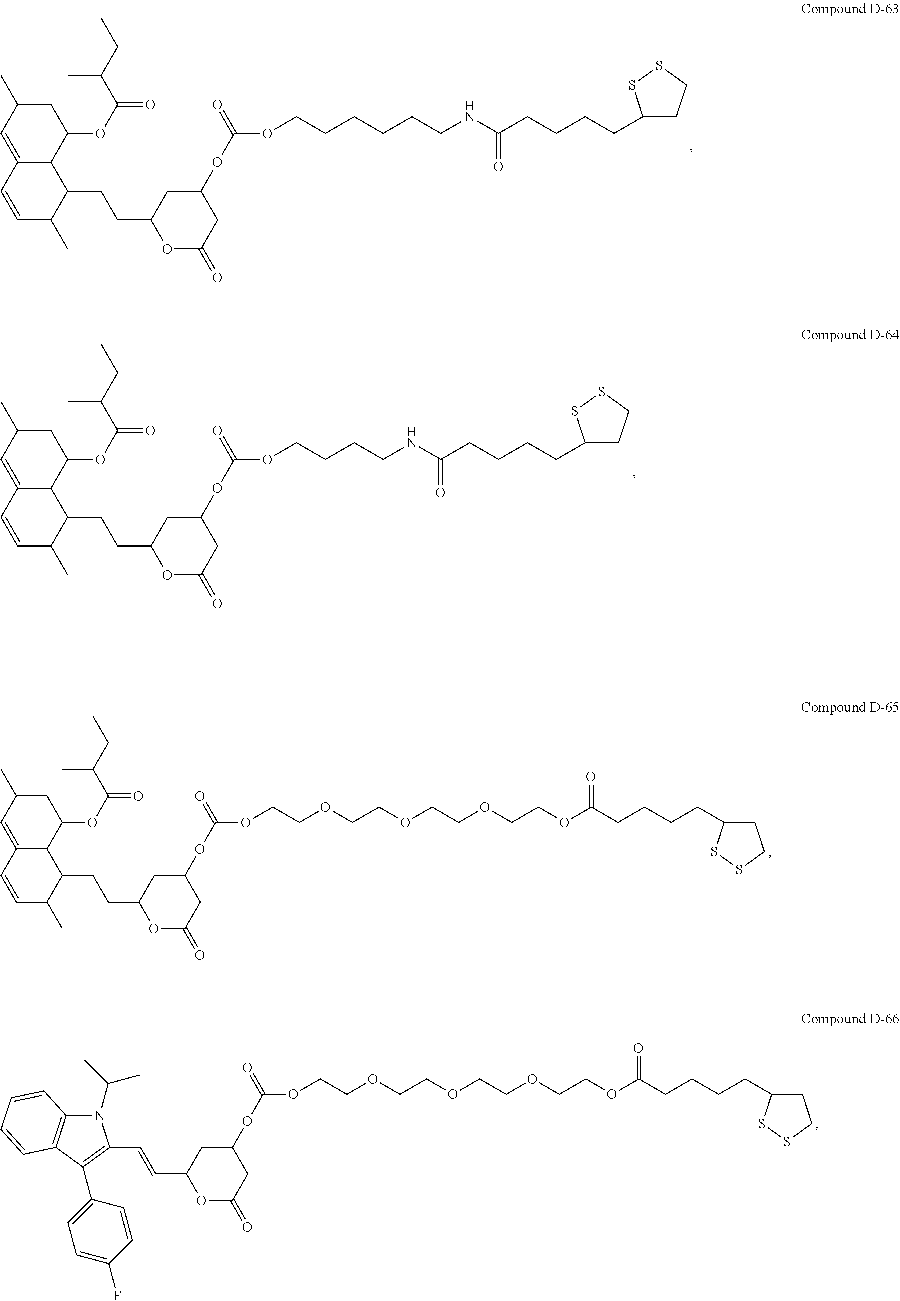 [SMILES: CCC(C)C(=O)OC1CC(C)C=C2C=CC(C)C(CCC3CC(OC(=O)OCCCCCCNC(=O)CCCCC4CCSS4)CC(=O)O3)C21 ; CCC(C)C(=O)OC1CC(C)C=C2C=CC(C)C(CCC3CC(OC(=O)OCCCCNC(=O)CCCCC4CCSS4)CC(=O)O3)C21 ; CCC(C)C(=O)OC1CC(C)C=C2C=CC(C)C(CCC3CC(OC(=O)OCCOCCOCCOCCOC(=O)CCCCC4CCSS4)CC(=O)O3)C21 ; CC(C)n1c(/C=C/C2CC(OC(=O)OCCOCCOCCOCCOC(=O)CCCCC3CCSS3)CC(=O)O2)c(-c2ccc(F)cc2)c2ccccc21]

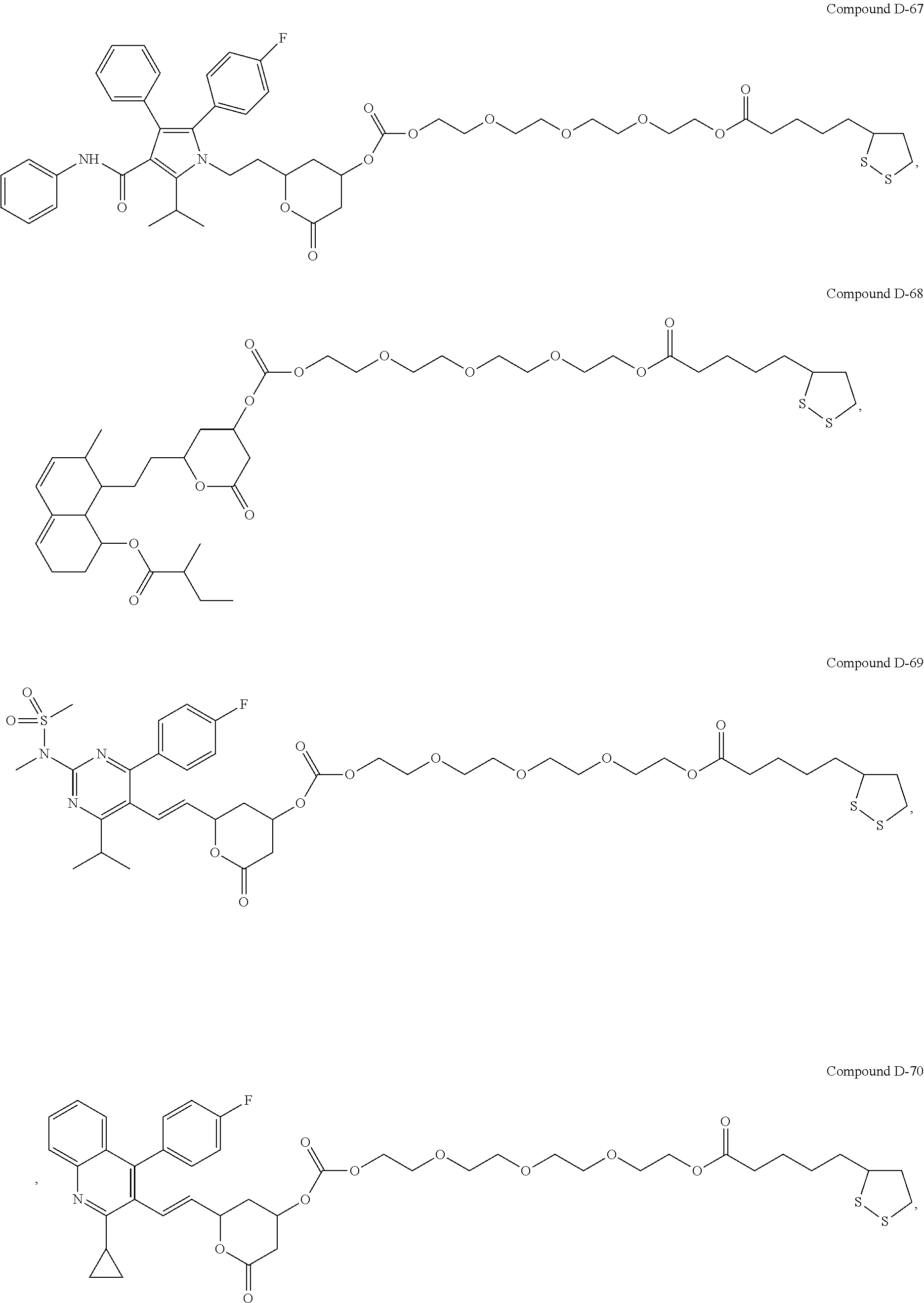 [SMILES: CC(C)c1c(C(=O)Nc2ccccc2)c(-c2ccccc2)c(-c2ccc(F)cc2)n1CCC1CC(OC(=O)OCCOCCOCCOCCOC(=O)CCCCC2CCSS2)CC(=O)O1 ; CCC(C)C(=O)OC1CCC=C2C=CC(C)C(CCC3CC(OC(=O)OCCOCCOCCOCCOC(=O)CCCCC4CCSS4)CC(=O)O3)C21 ; CC(C)c1nc(N(C)S(C)(=O)=O)nc(-c2ccc(F)cc2)c1/C=C/C1CC(OC(=O)OCCOCCOCCOCCOC(=O)CCCCC2CCSS2)CC(=O)O1 ; O=C(CCCCC1CCSS1)OCCOCCOCCOCCOC(=O)OC1CC(=O)OC(/C=C/c2c(C3CC3)nc3ccccc3c2-c2ccc(F)cc2)C1]

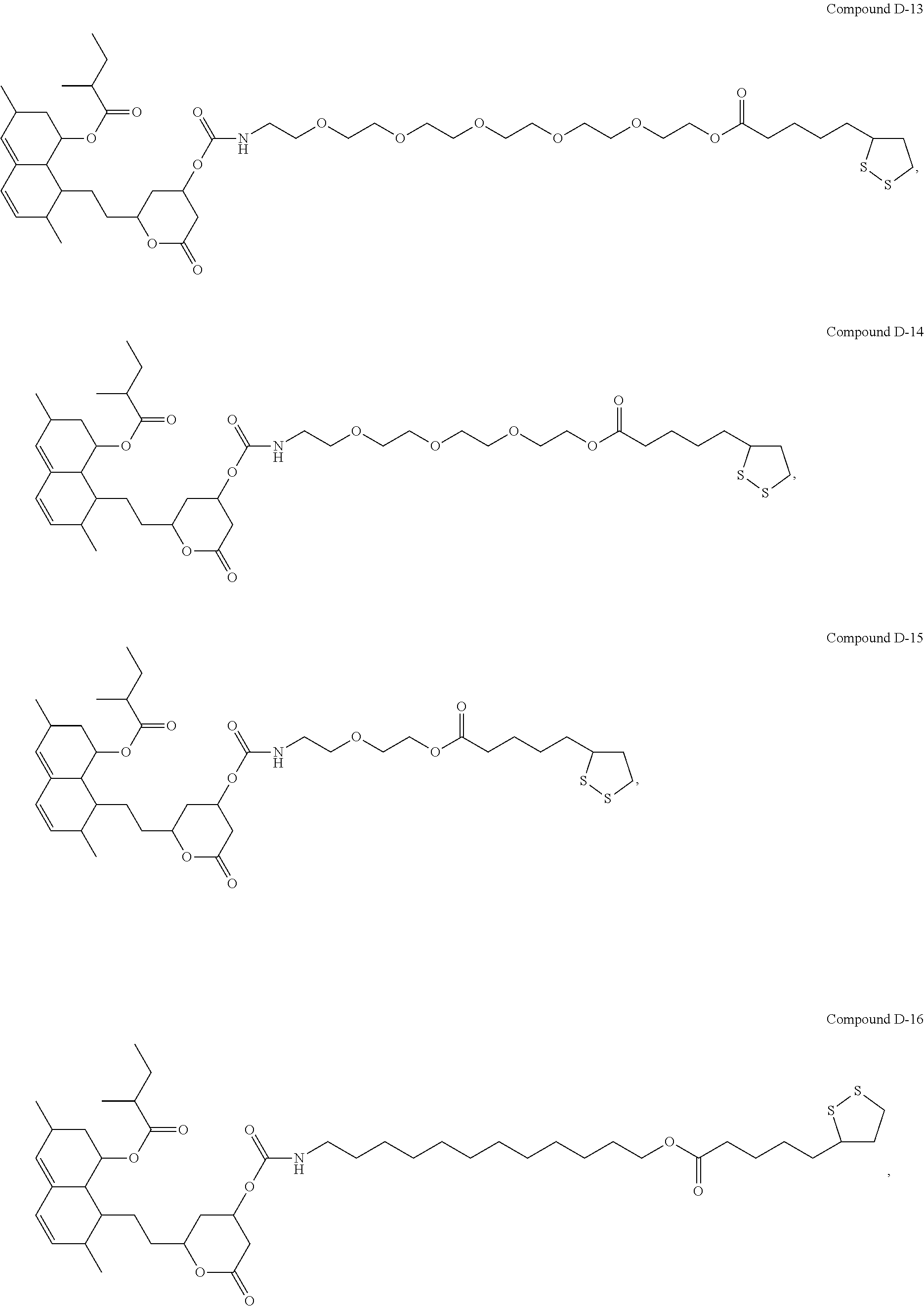 [SMILES: CCC(C)C(=O)OC1CC(C)C=C2C=CC(C)C(CCC3CC(OC(=O)NCCOCCOCCOCCOCCOCCOC(=O)CCCCC4CCSS4)CC(=O)O3)C21 ; CCC(C)C(=O)OC1CC(C)C=C2C=CC(C)C(CCC3CC(OC(=O)NCCOCCOCCOCCOC(=O)CCCCC4CCSS4)CC(=O)O3)C21 ; CCC(C)C(=O)OC1CC(C)C=C2C=CC(C)C(CCC3CC(OC(=O)NCCOCCOC(=O)CCCCC4CCSS4)CC(=O)O3)C21 ; CCC(C)C(=O)OC1CC(C)C=C2C=CC(C)C(CCC3CC(OC(=O)NCCCCCCCCCCCCOC(=O)CCCCC4CCSS4)CC(=O)O3)C21]

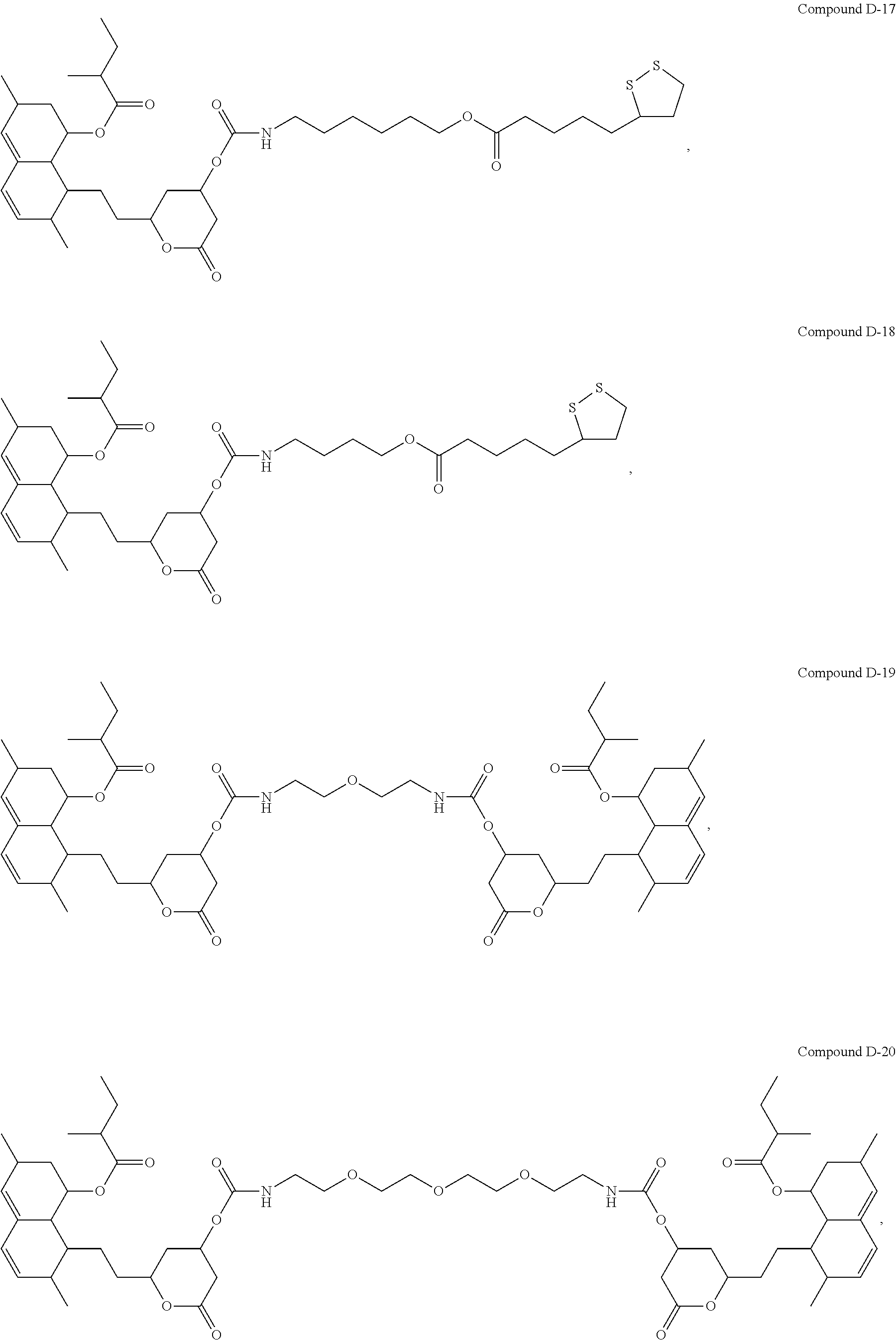 [SMILES: CCC(C)C(=O)OC1CC(C)C=C2C=CC(C)C(CCC3CC(OC(=O)NCCCCCCOC(=O)CCCCC4CCSS4)CC(=O)O3)C21 ; CCC(C)C(=O)OC1CC(C)C=C2C=CC(C)C(CCC3CC(OC(=O)NCCCCOC(=O)CCCCC4CCSS4)CC(=O)O3)C21 ; CCC(C)C(=O)OC1CC(C)C=C2C=CC(C)C(CCC3CC(OC(=O)NCCOCCNC(=O)OC4CC(=O)OC(CCC5C(C)C=CC6=CC(C)CC(OC(=O)C(C)CC)C65)C4)CC(=O)O3)C21 ; CCC(C)C(=O)OC1CC(C)C=C2C=CC(C)C(CCC3CC(OC(=O)NCCOCCOCCOCCNC(=O)OC4CC(=O)OC(CCC5C(C)C=CC6=CC(C)CC(OC(=O)C(C)CC)C65)C4)CC(=O)O3)C21]

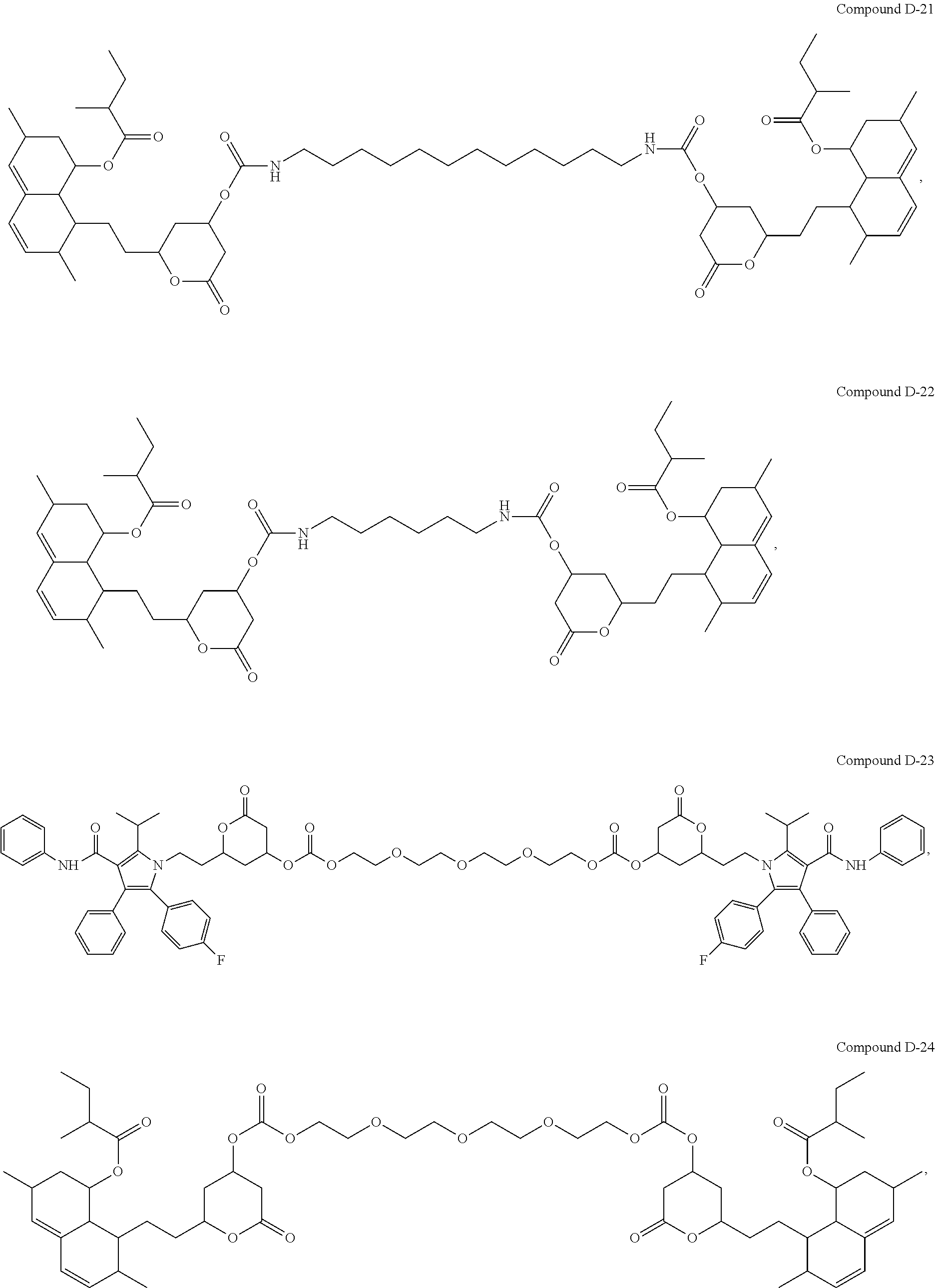

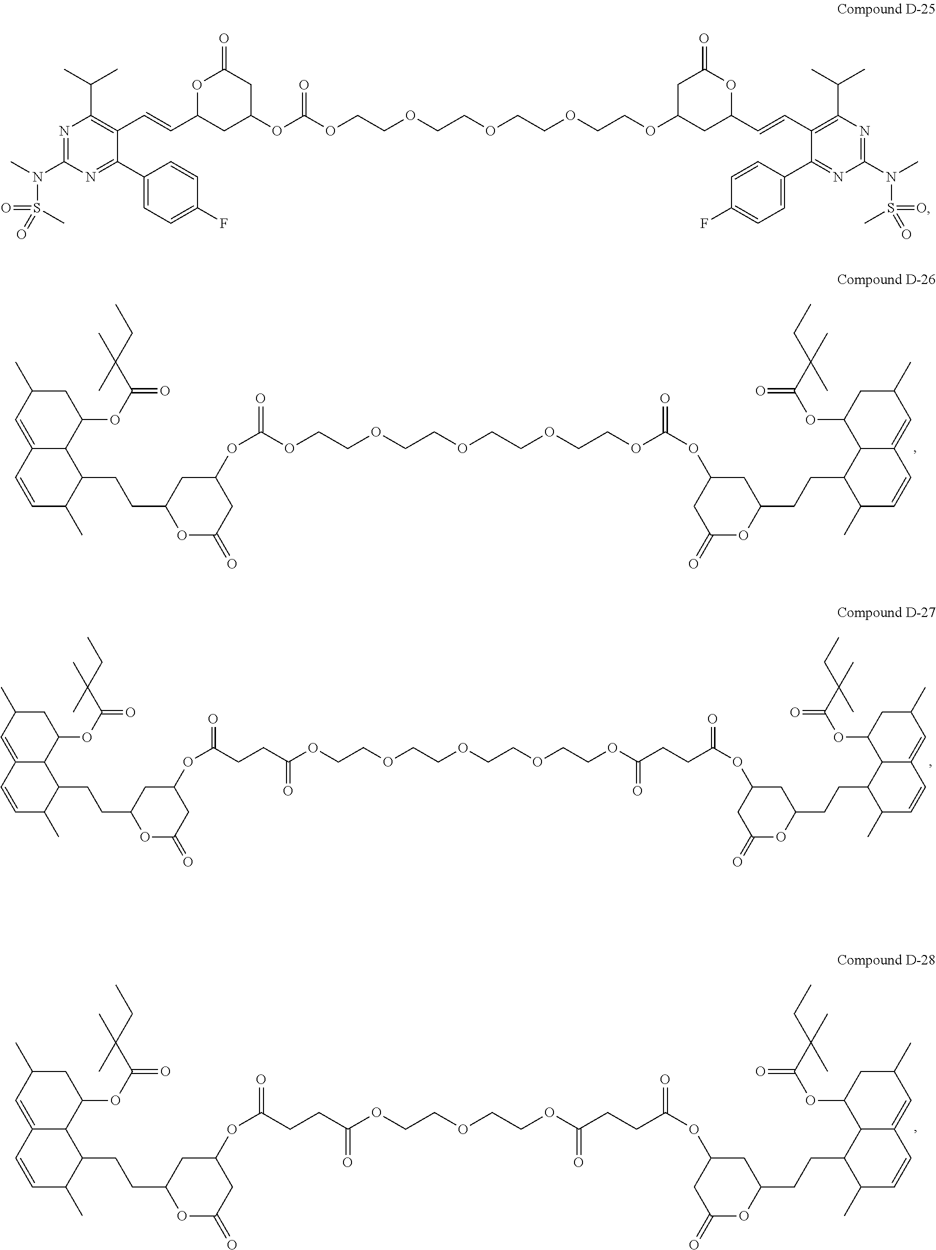

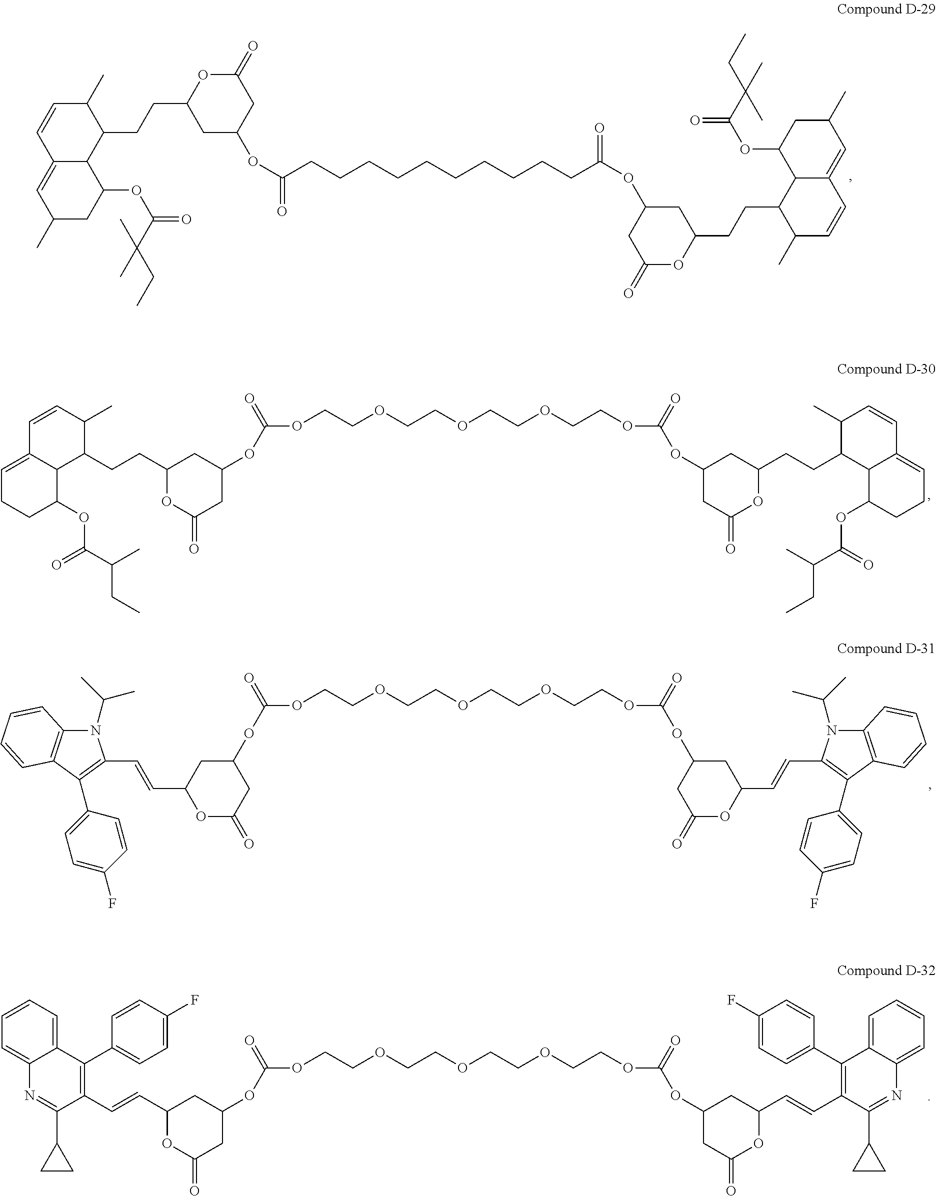

17. A method of lowering cholesterol levels, lowering the likelihood of cardiovascular disease, or treating cardiovascular disease in a subject in need thereof, comprising:
providing the nanosphere of claim 15; and
administering a therapeutically effective amount of the nanosphere to the subject to lower the cholesterol levels, lower the likelihood of cardiovascular disease, or treat cardiovascular disease.

18. A method of detecting or diagnosing cancer in a subject in need thereof comprising:
providing the nanosphere of claim 15;
administering an effective amount of the nanosphere to the subject; and
imaging the subject to detect or diagnose the cancer.

19. The nanosphere of claim 1, further comprising:
an antioxidant derivative of camptothecin and/or an antioxidant derivative of a camptothecin analog.

20. The nanosphere of claim 19, wherein the antioxidant derivative of camptothecin and/or an antioxidant derivative of a camptothecin analog is 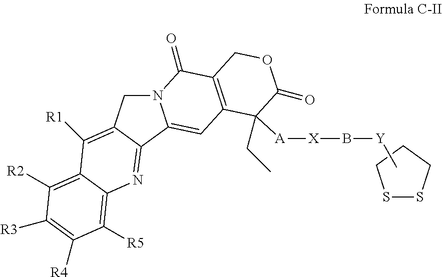
wherein A and B are independently selected from the group consisting of —OC(O)—, —OC(O)O—, and —OC(O)N(R)—, wherein R is a hydrogen atom, or a substituted, unsubstituted, branched or unbranched chain of carbon atoms;
X and Y are linkers, each independently comprising a substituted, unsubstituted, branched or unbranched chain of carbon atoms and may optionally contain a heteroatom; and
R₁, R₂, R₃, R₄, and R₅ are each independently selected from the group consisting of hydrogen, alkyl, aryl, cycloaliphatic, and aralkyl and may each optionally contain a hetero atom.

21. The nanosphere of claim 20, wherein the antioxidant derivative of camptothecin and/or an antioxidant derivative of a camptothecin analog is: 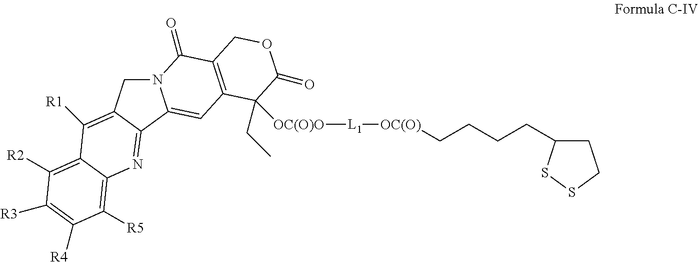
wherein L₁ is a moiety formed by esterification of two free esterifiable hydroxyl groups on a diol.

22. The nanosphere of claim 21, wherein the antioxidant derivative of camptothecin and/or an antioxidant derivative of a camptothecin analog is selected from the group consisting of:

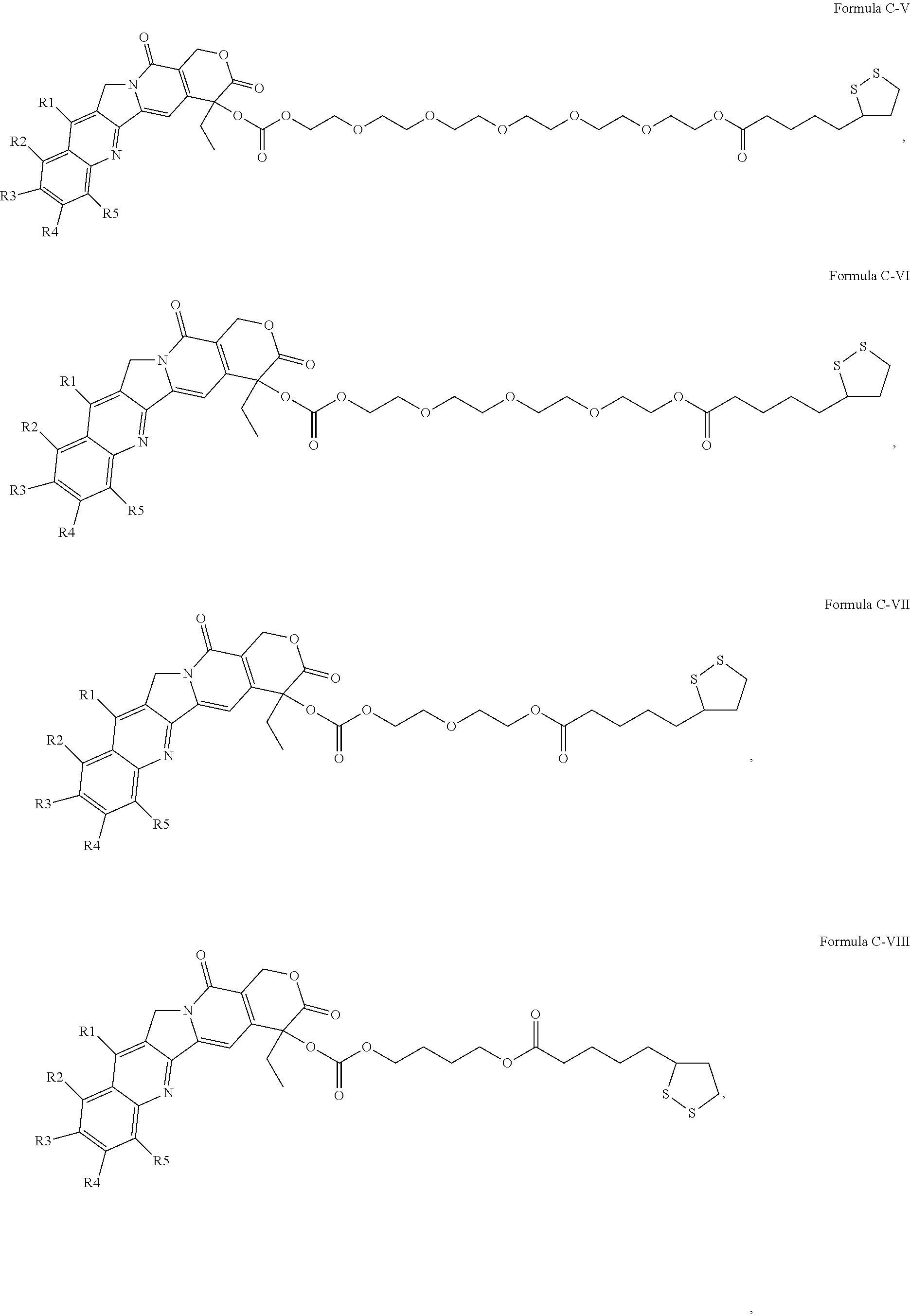

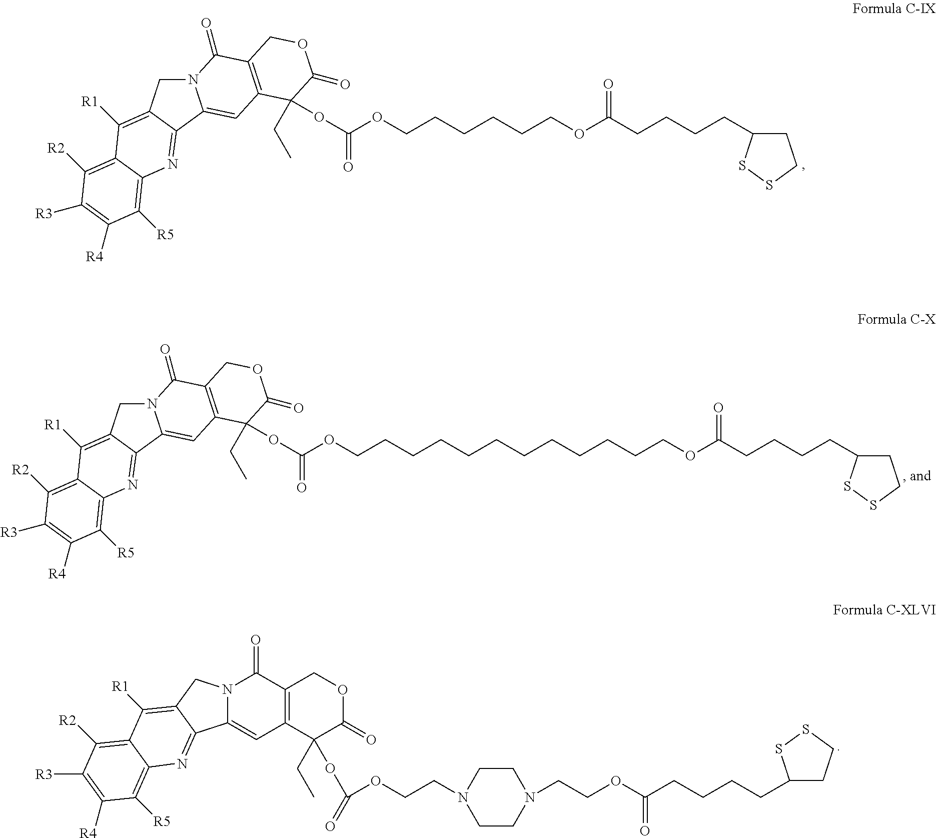

23. The nanosphere of claim 20, wherein the antioxidant derivative of camptothecin and/or an antioxidant derivative of a camptothecin analog is: 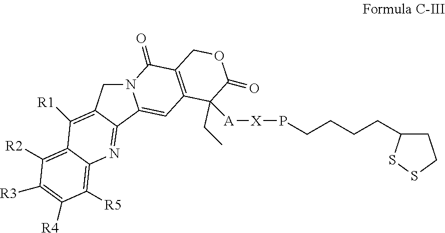
wherein A is selected from the group consisting of —OC(O)—, —OC(O)O—, and —OC(O)N(R)—, wherein R is a hydrogen atom, or a substituted, unsubstituted, branched or unbranched chain of carbon atoms;
P is selected from the group consisting of —OC(O)—, and —N(R)C(O)—, wherein R is a hydrogen atom, or a substituted, unsubstituted, branched or unbranched chain of carbon atoms;
X is a linker comprising a substituted, unsubstituted, branched or unbranched chain of carbon atoms and may optionally contain a heteroatom; and
R₁, R₂, R₃, R₄, and R₅ are each independently selected from the group consisting of hydrogen, alkyl, aryl, cycloaliphatic, and aralkyl, and may each optionally contain a hetero atom.

24. The nanosphere of claim 23, wherein the antioxidant derivative of camptothecin and/or an antioxidant derivative of a camptothecin analog is Formula C-XI 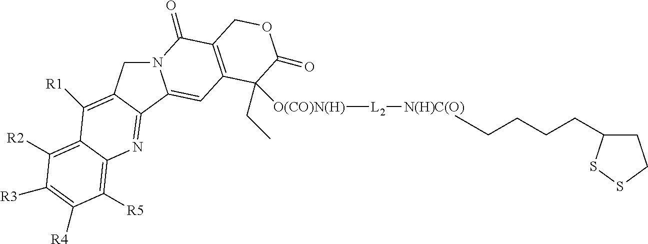
wherein L₂ is a moiety formed by using a diamine as the linker in the process of producing the compound.

25. The nanosphere of claim 23, wherein the antioxidant derivative of camptothecin and/or an antioxidant derivative of a camptothecin analog is selected from the group consisting of:

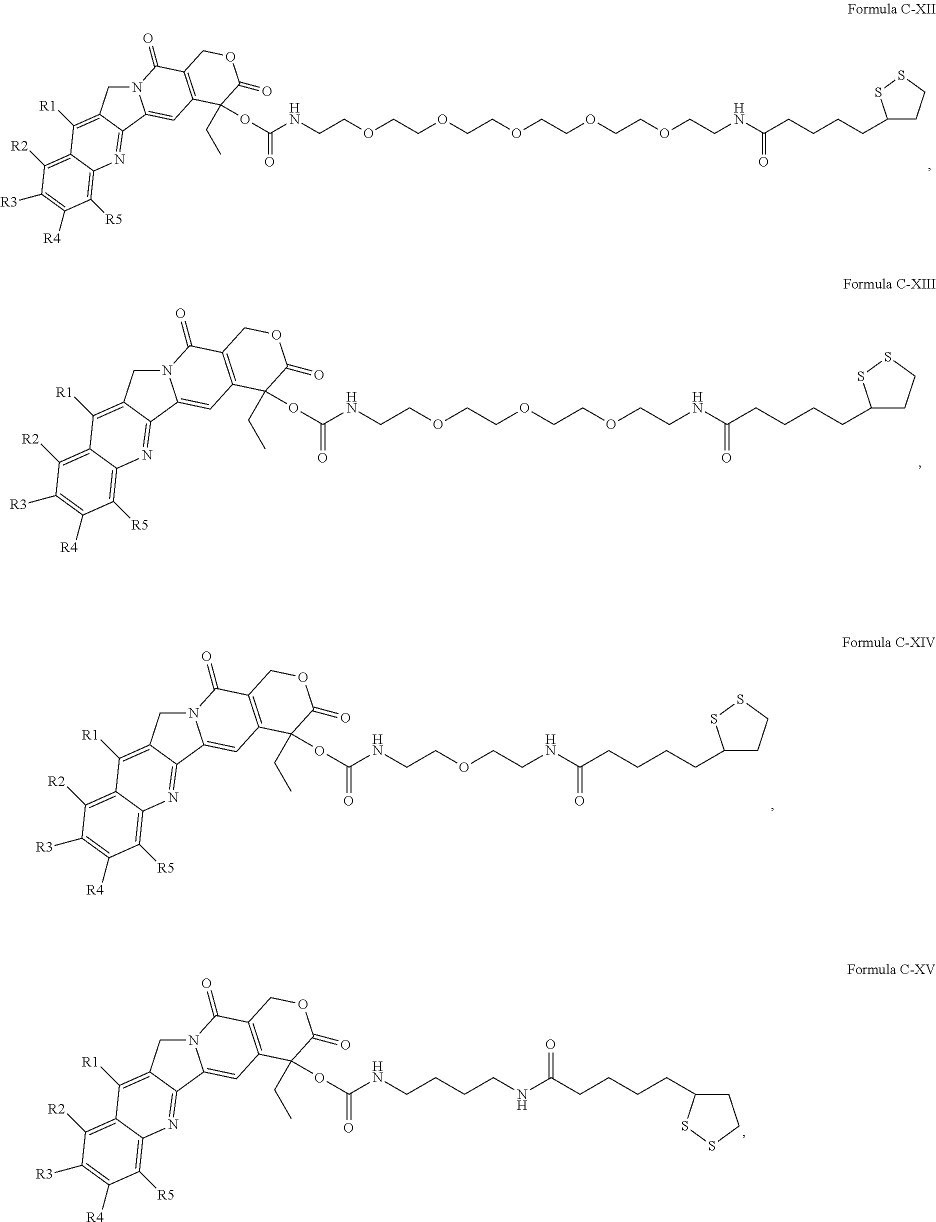

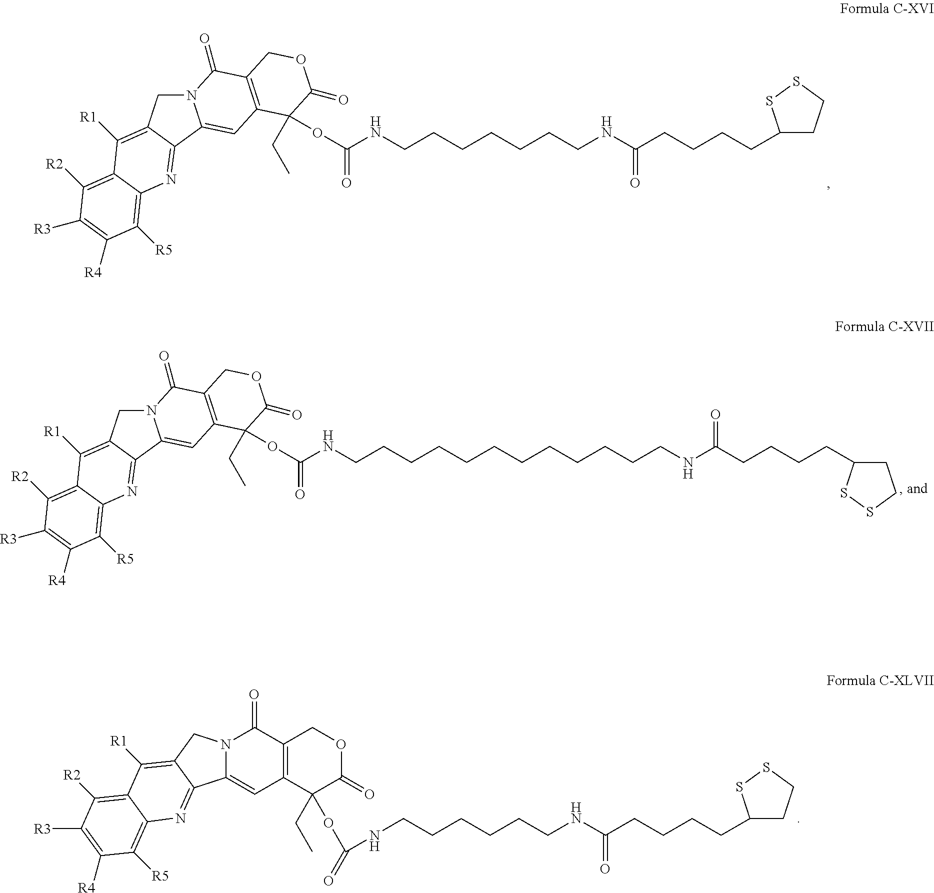

26. The nanosphere of claim 23, wherein the antioxidant derivative of camptothecin and/or an antioxidant derivative of a camptothecin analog is Formula C-XVIII: 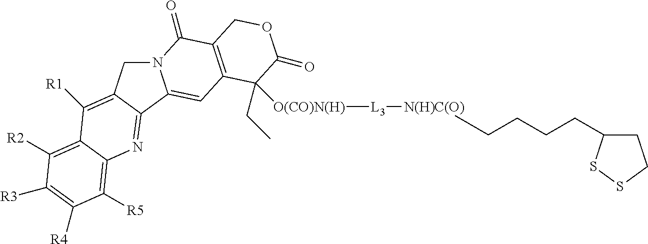
wherein L₃ is a moiety formed by using an aminoalcohol as the linker in the process of producing the compound.

27. The nanosphere of claim 26, wherein the antioxidant derivative of camptothecin and/or an antioxidant derivative of a camptothecin analog is selected from the group consisting of:

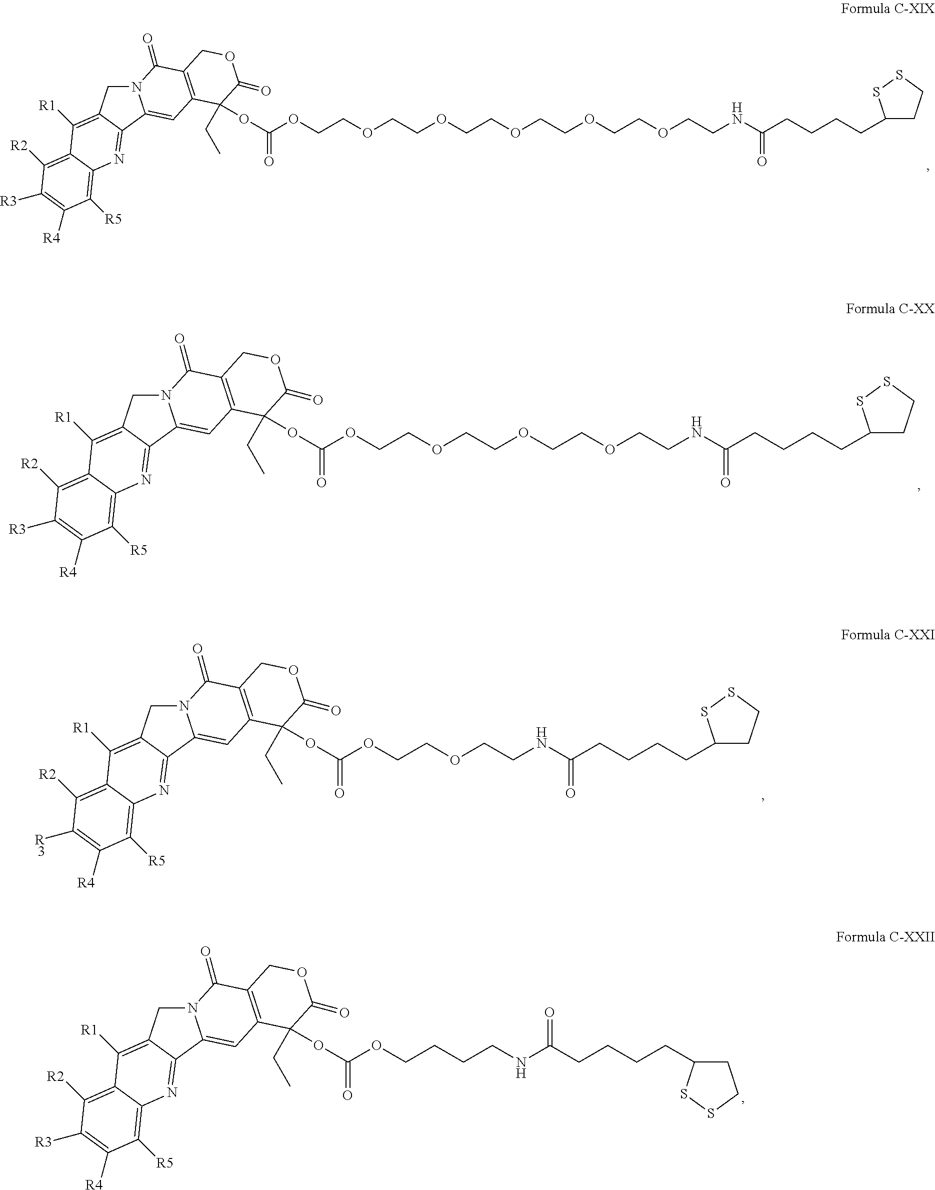

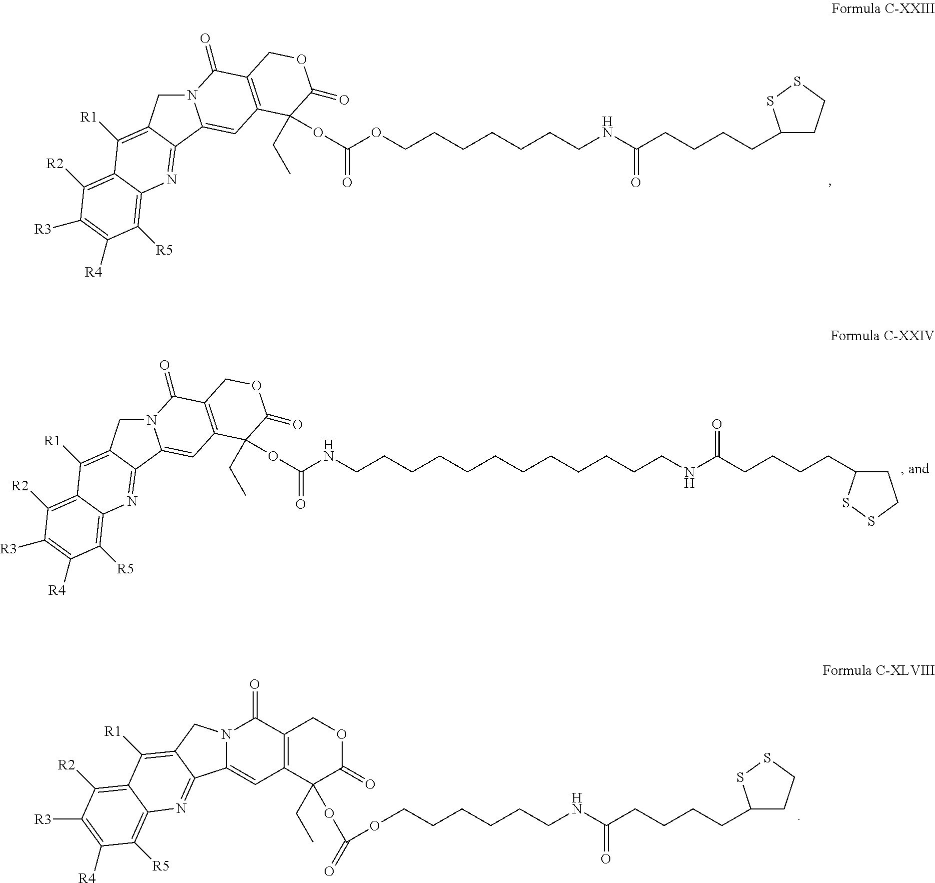

28. The nanosphere of claim 19, wherein the antioxidant derivative of camptothecin and/or an antioxidant derivative of a camptothecin analog is a compound produced by conjugation of an α-lipoic acid and camptothecin or a camptothecin analog modified by reacting with succinic anhydride or glutaric anhydride, wherein the camptothecin analog is represented by Formula C-I 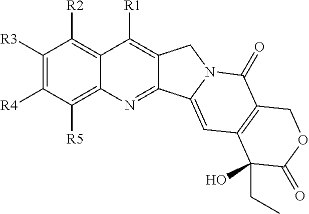
wherein R₁, R₂, R₃, R₄, and R₅ are each independently selected from the group consisting of hydrogen, alkyl, aryl, cycloaliphatic, and aralkyl, and may optionally contain a hetero atom.

29. The nanosphere of claim 19, wherein the antioxidant derivative of camptothecin and/or an antioxidant derivative of a camptothecin analog is selected from the group consisting of: 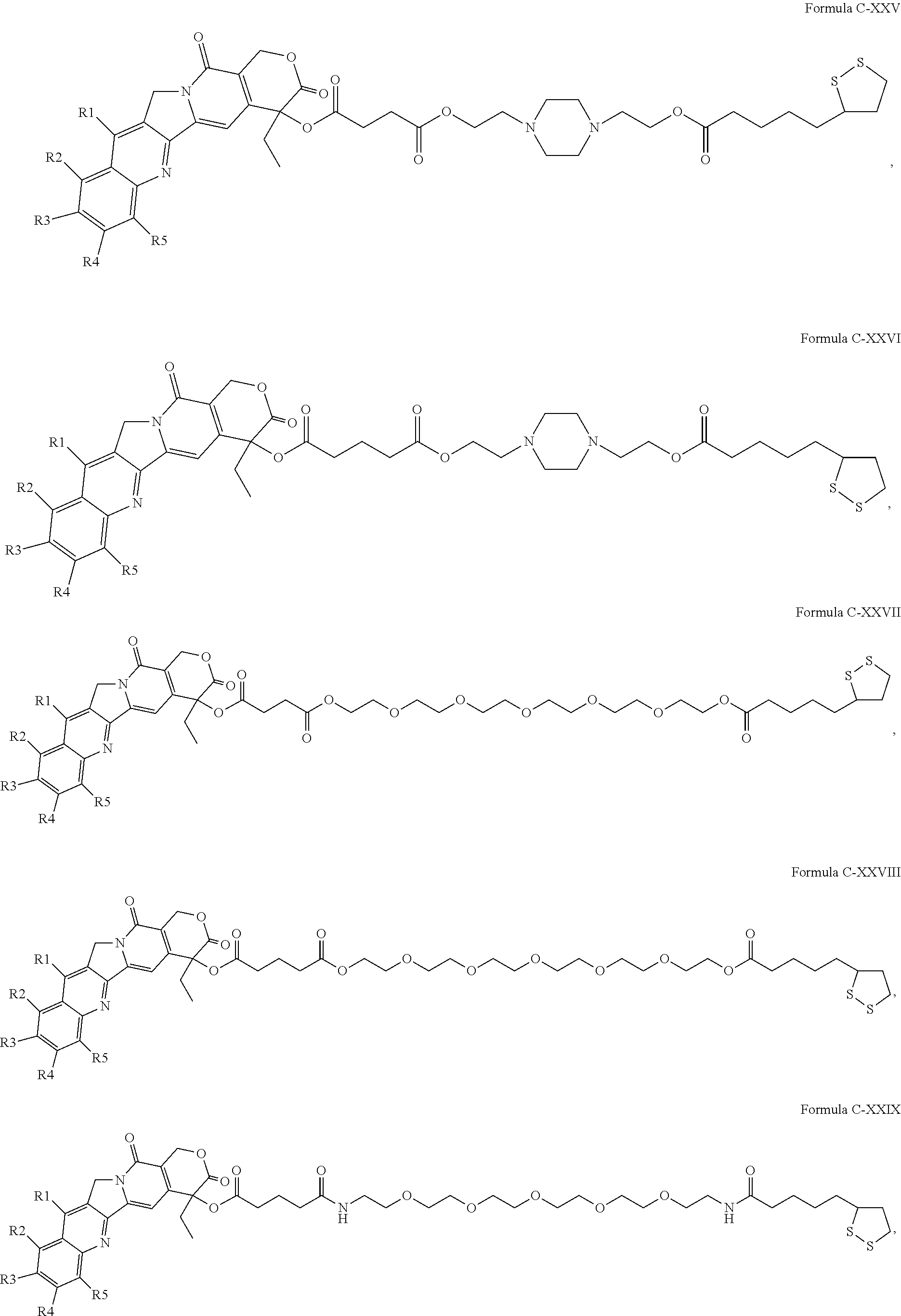 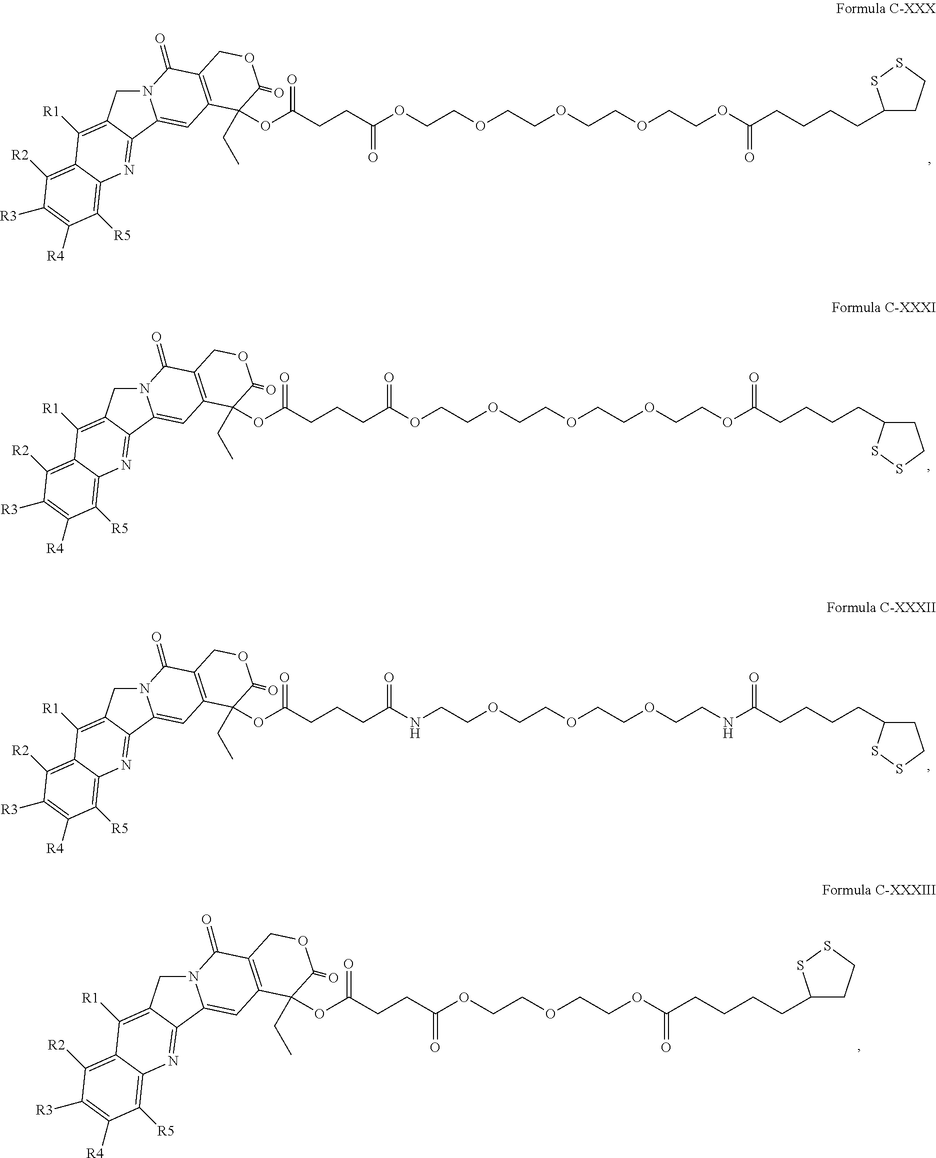 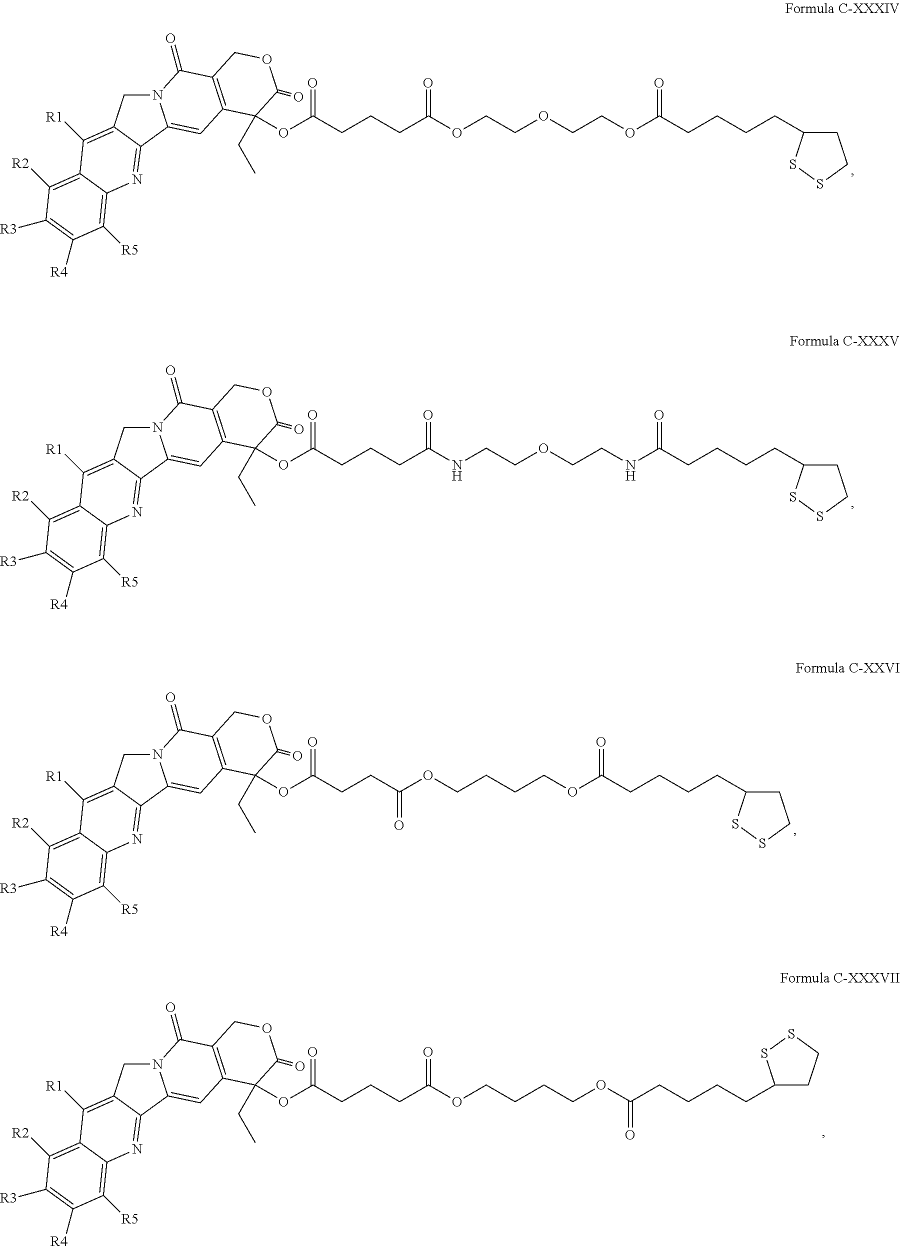 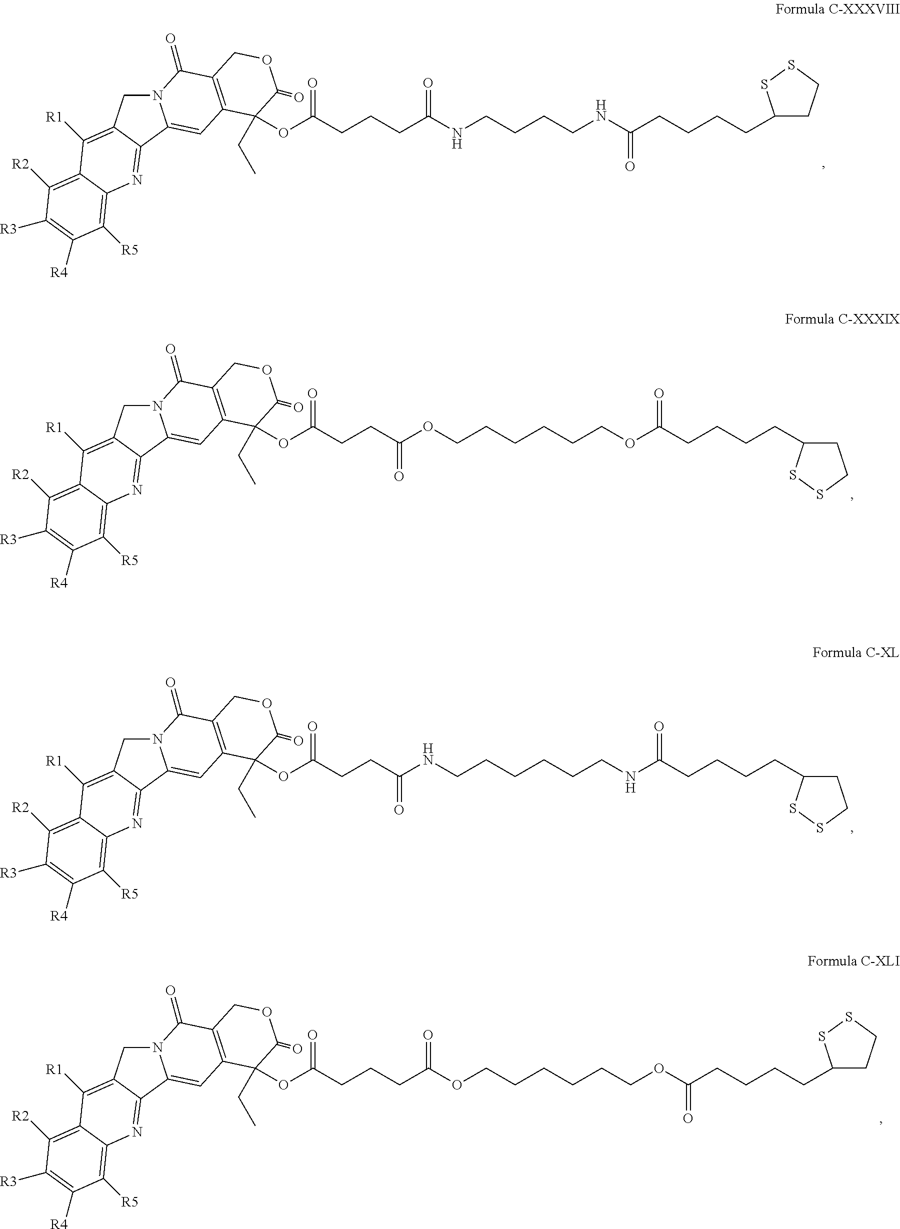 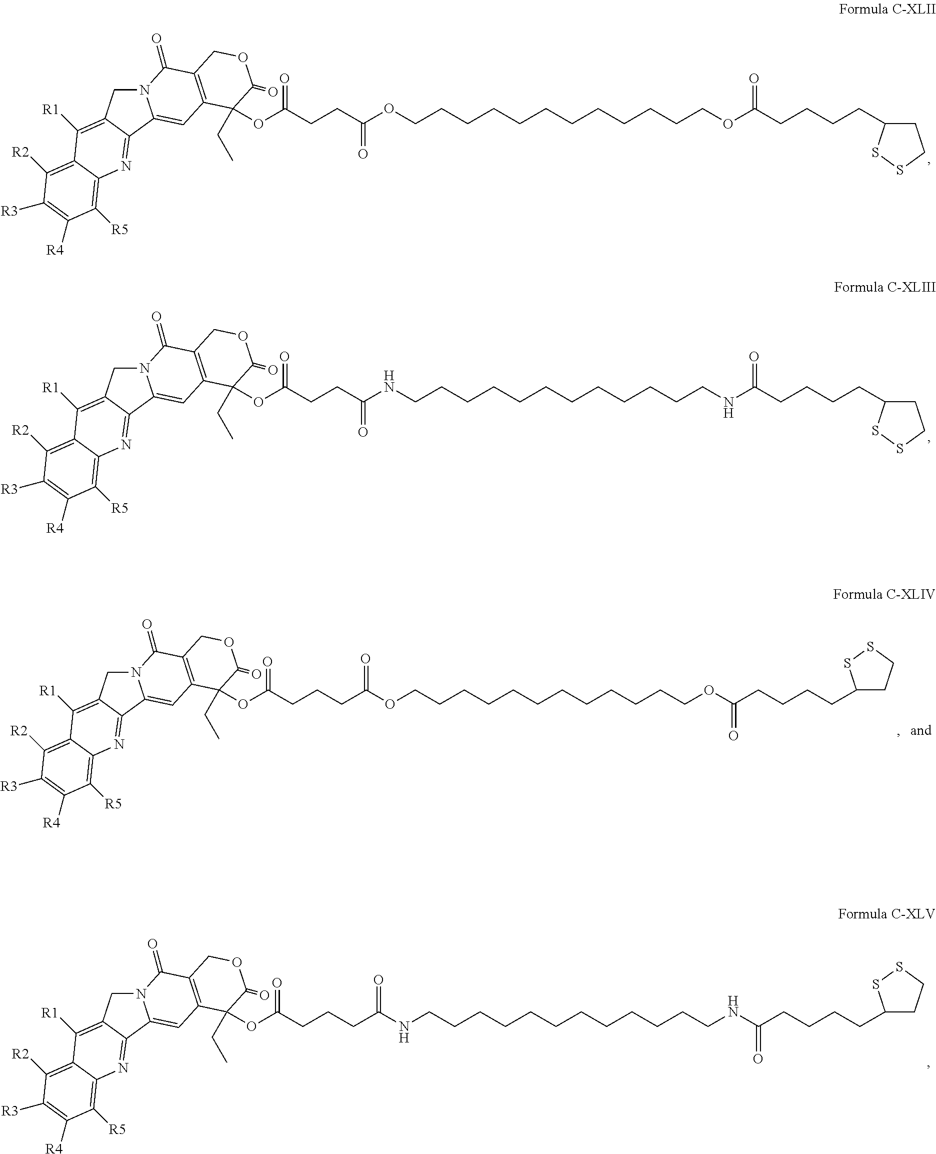
wherein R₁, R₂, R₃, R₄, and R₅ are each independently selected from the group consisting of hydrogen, alkyl, aryl, cycloaliphatic, and aralkyl and may each optionally contain a hetero atom.

30. The nanosphere of claim 19, wherein the antioxidant derivative of camptothecin and/or an antioxidant derivative of a camptothecin analog is selected from the group consisting of:

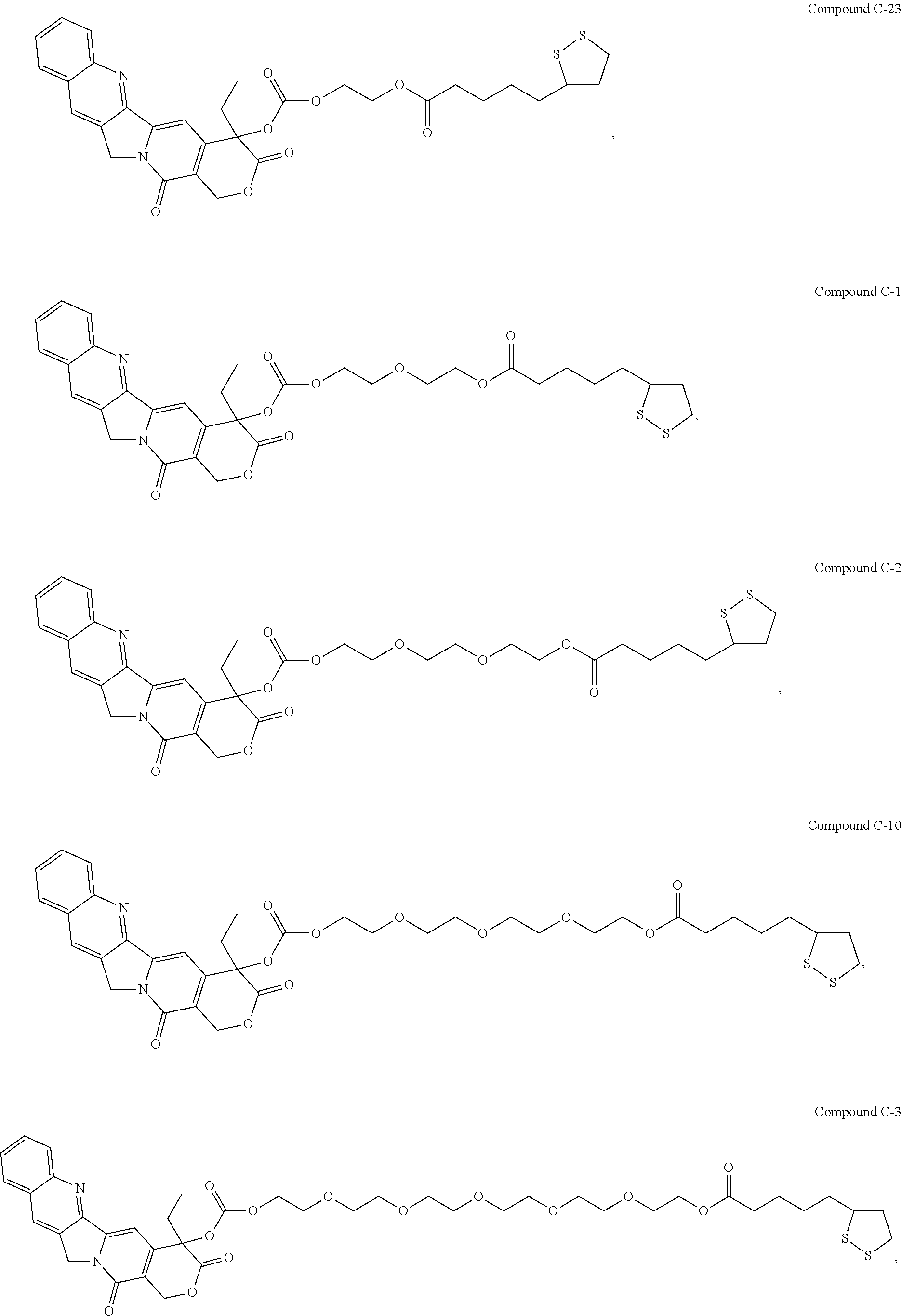 [SMILES: CCC1(OC(=O)OCCOC(=O)CCCCC2CCSS2)C(=O)OCc2c1cc1n(c2=O)Cc2cc3ccccc3nc2-1 ; CCC1(OC(=O)OCCOCCOC(=O)CCCCC2CCSS2)C(=O)OCc2c1cc1n(c2=O)Cc2cc3ccccc3nc2-1 ; CCC1(OC(=O)OCCOCCOCCOC(=O)CCCCC2CCSS2)C(=O)OCc2c1cc1n(c2=O)Cc2cc3ccccc3nc2-1 ; CCC1(OC(=O)OCCOCCOCCOCCOC(=O)CCCCC2CCSS2)C(=O)OCc2c1cc1n(c2=O)Cc2cc3ccccc3nc2-1 ; CCC1(OC(=O)OCCOCCOCCOCCOCCOCCOC(=O)CCCCC2CCSS2)C(=O)OCc2c1cc1n(c2=O)Cc2cc3ccccc3nc2-1]

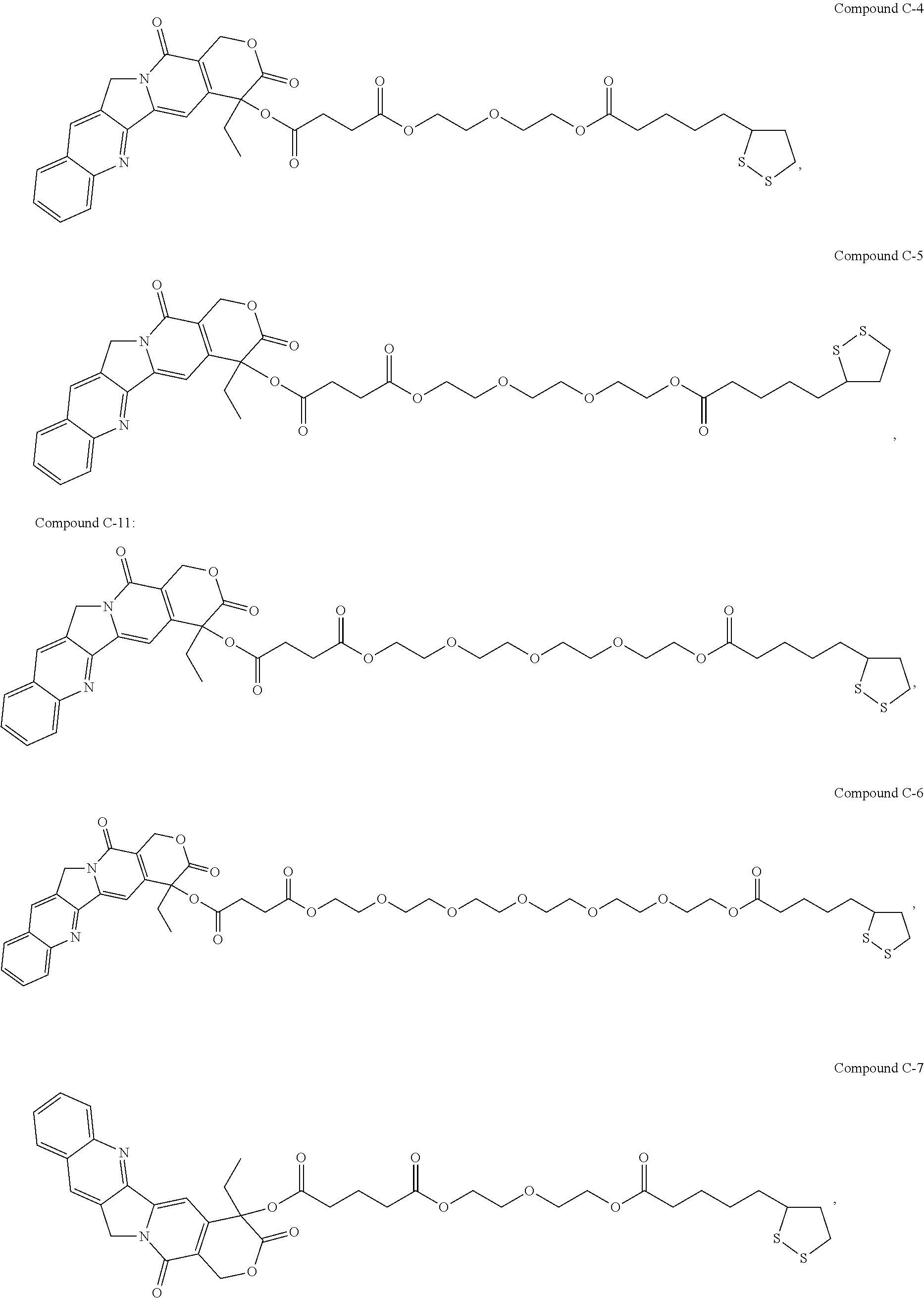 [SMILES: CCC1(OC(=O)CCC(=O)OCCOCCOC(=O)CCCCC2CCSS2)C(=O)OCc2c1cc1n(c2=O)Cc2cc3ccccc3nc2-1 ; CCC1(OC(=O)CCC(=O)OCCOCCOCCOC(=O)CCCCC2CCSS2)C(=O)OCc2c1cc1n(c2=O)Cc2cc3ccccc3nc2-1 ; CCC1(OC(=O)CCC(=O)OCCOCCOCCOCCOC(=O)CCCCC2CCSS2)C(=O)OCc2c1cc1n(c2=O)Cc2cc3ccccc3nc2-1 ; CCC1(OC(=O)CCC(=O)OCCOCCOCCOCCOCCOCCOC(=O)CCCCC2CCSS2)C(=O)OCc2c1cc1n(c2=O)Cc2cc3ccccc3nc2-1 ; CCC1(OC(=O)CCCC(=O)OCCOCCOC(=O)CCCCC2CCSS2)C(=O)OCc2c1cc1n(c2=O)Cc2cc3ccccc3nc2-1]

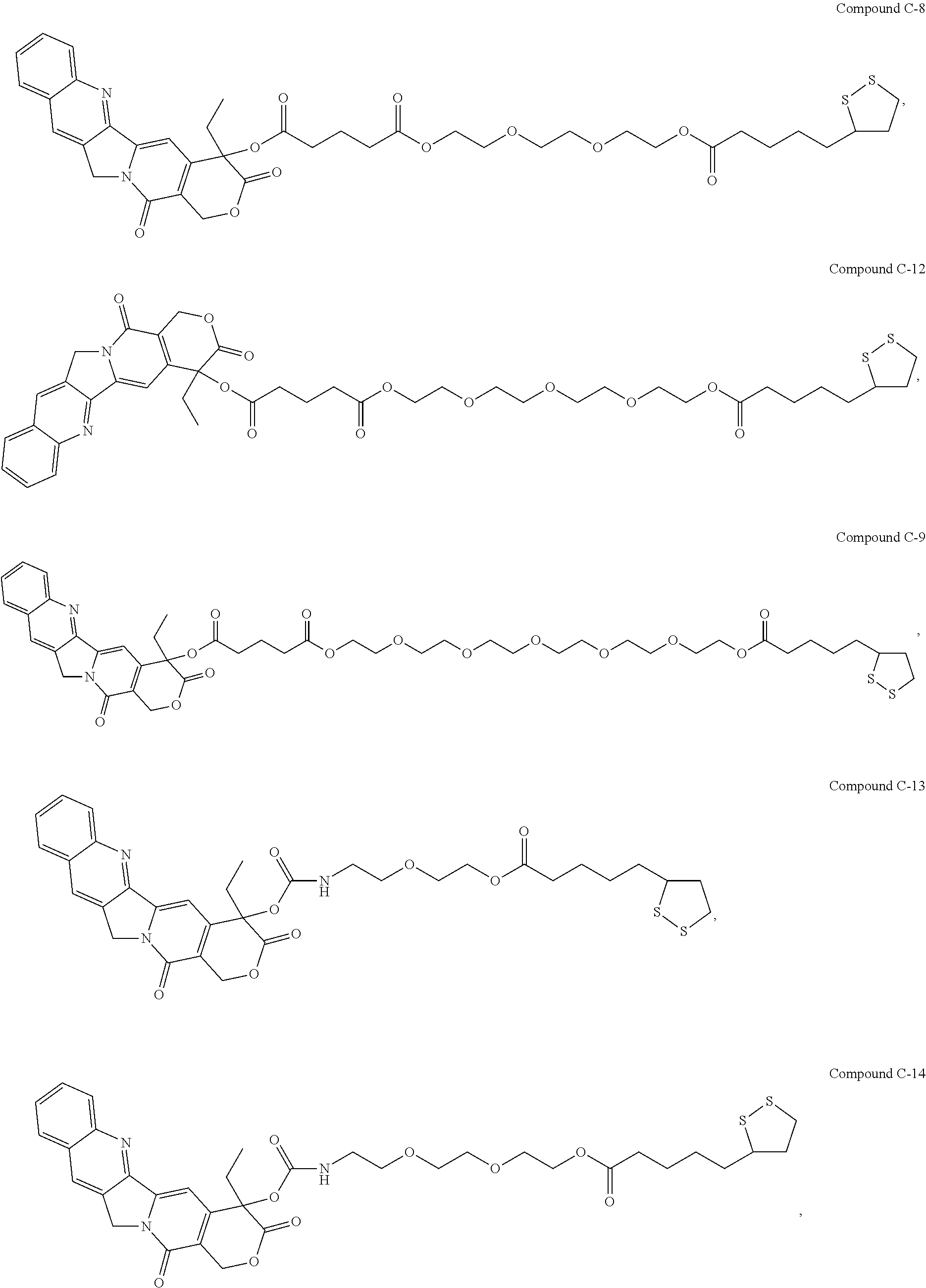 [SMILES: CCC1(OC(=O)CCCC(=O)OCCOCCOCCOC(=O)CCCCC2CCSS2)C(=O)OCc2c1cc1n(c2=O)Cc2cc3ccccc3nc2-1 ; CCC1(OC(=O)CCCC(=O)OCCOCCOCCOCCOC(=O)CCCCC2CCSS2)C(=O)OCc2c1cc1n(c2=O)Cc2cc3ccccc3nc2-1 ; CCC1(OC(=O)CCCC(=O)OCCOCCOCCOCCOCCOCCOC(=O)CCCCC2CCSS2)C(=O)OCc2c1cc1n(c2=O)Cc2cc3ccccc3nc2-1 ; CCC1(OC(=O)NCCOCCOC(=O)CCCCC2CCSS2)C(=O)OCc2c1cc1n(c2=O)Cc2cc3ccccc3nc2-1 ; CCC1(OC(=O)NCCOCCOCCOC(=O)CCCCC2CCSS2)C(=O)OCc2c1cc1n(c2=O)Cc2cc3ccccc3nc2-1]

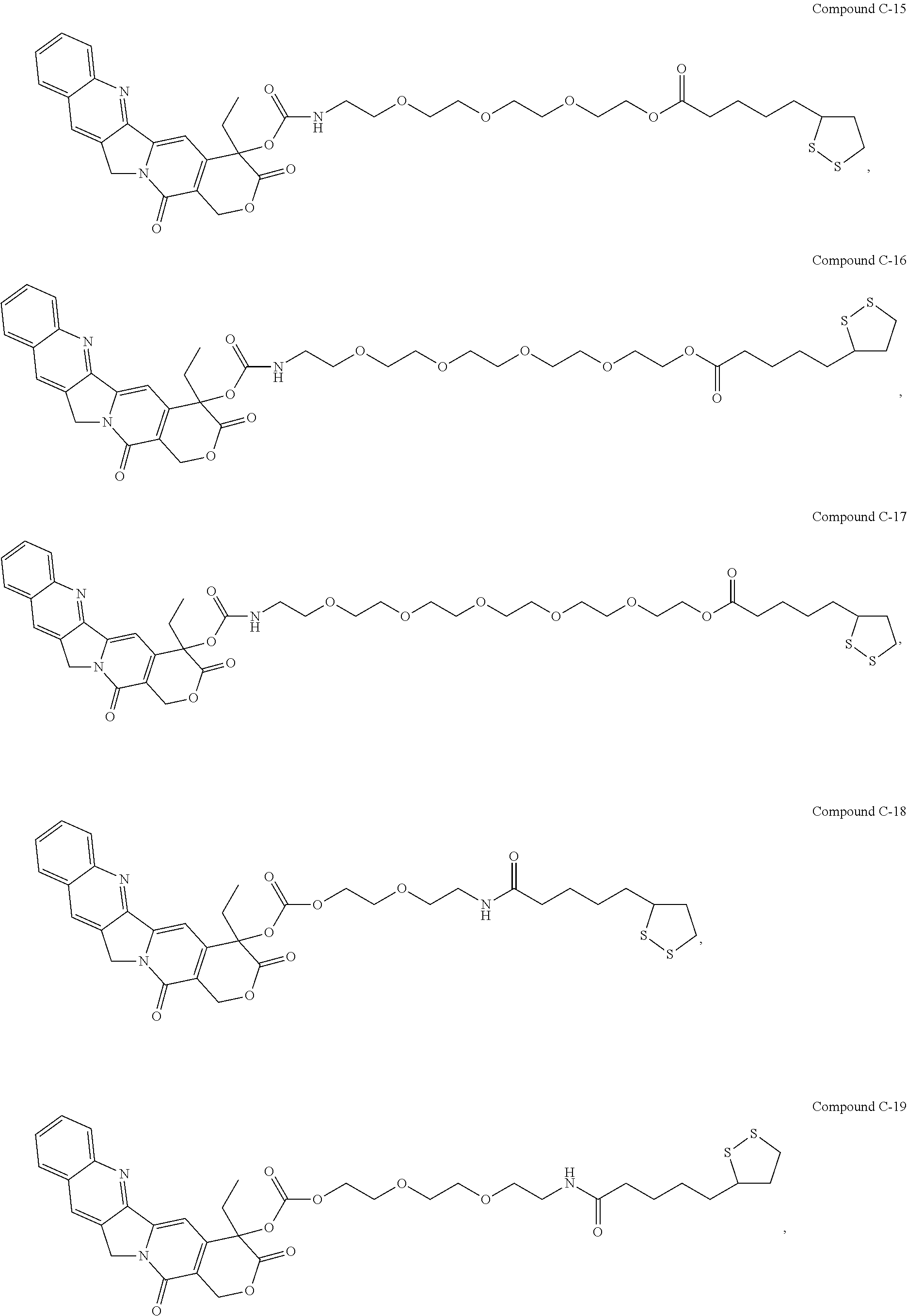 [SMILES: CCC1(OC(=O)NCCOCCOCCOCCOC(=O)CCCCC2CCSS2)C(=O)OCc2c1cc1n(c2=O)Cc2cc3ccccc3nc2-1 ; CCC1(OC(=O)NCCOCCOCCOCCOCCOC(=O)CCCCC2CCSS2)C(=O)OCc2c1cc1n(c2=O)Cc2cc3ccccc3nc2-1 ; CCC1(OC(=O)NCCOCCOCCOCCOCCOCCOC(=O)CCCCC2CCSS2)C(=O)OCc2c1cc1n(c2=O)Cc2cc3ccccc3nc2-1 ; CCC1(OC(=O)OCCOCCNC(=O)CCCCC2CCSS2)C(=O)OCc2c1cc1n(c2=O)Cc2cc3ccccc3nc2-1 ; CCC1(OC(=O)OCCOCCOCCNC(=O)CCCCC2CCSS2)C(=O)OCc2c1cc1n(c2=O)Cc2cc3ccccc3nc2-1]

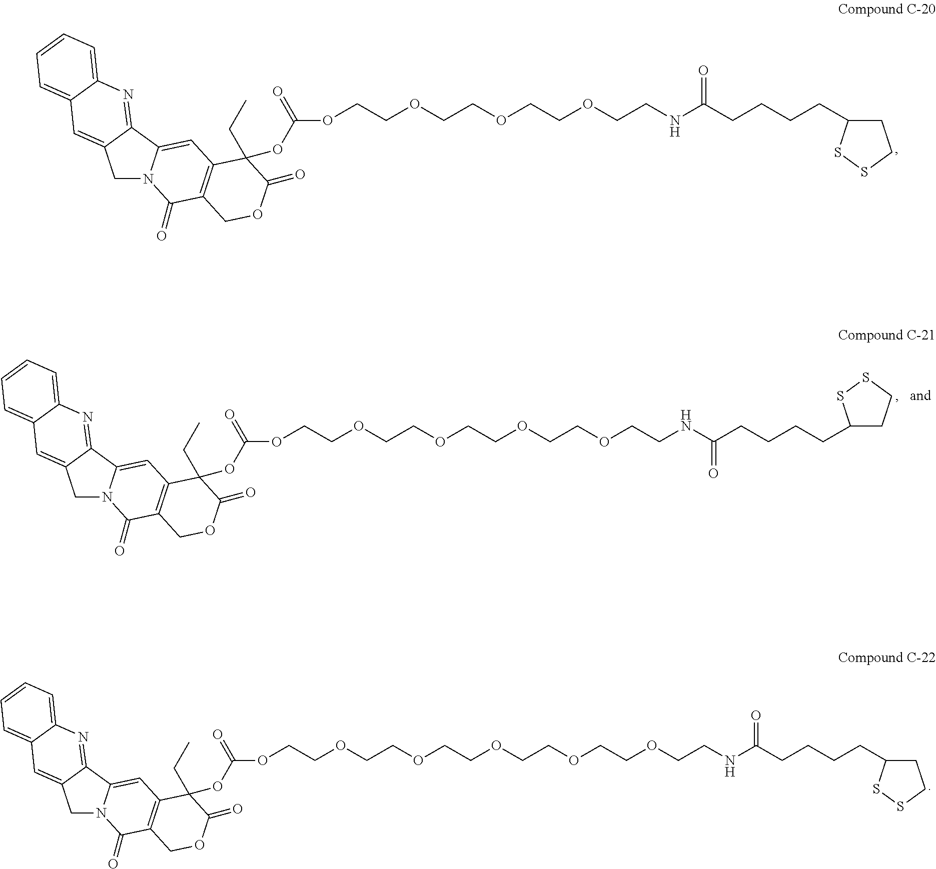 [SMILES: CCC1(OC(=O)OCCOCCOCCOCCNC(=O)CCCCC2CCSS2)C(=O)OCc2c1cc1n(c2=O)Cc2cc3ccccc3nc2-1 ; CCC1(OC(=O)OCCOCCOCCOCCOCCNC(=O)CCCCC2CCSS2)C(=O)OCc2c1cc1n(c2=O)Cc2cc3ccccc3nc2-1 ; CCC1(OC(=O)OCCOCCOCCOCCOCCOCCNC(=O)CCCCC2CCSS2)C(=O)OCc2c1cc1n(c2=O)Cc2cc3ccccc3nc2-1]

31. A method of treating cancer in a subject in need thereof, comprising:
providing the nanosphere of claim 19; and
administering a therapeutically effective amount of the nanosphere to the subject to treat the cancer.

32. The method of claim 31, wherein the cancer is brain cancer.

33. A method of detecting or diagnosing cancer in a subject in need thereof comprising:
providing the nanosphere of claim 19;
administering an effective amount of the nanosphere to the subject; and
imaging the subject to diagnose the cancer.

34. The nanosphere of claim 1, wherein the therapeutic agent is selected from the group consisting of: a chemotherapeutic agent, statin, nonsteroidal anti-inflammatory drug (NSAID), erythropoietin, peptide, antisense nucleic acid, DNA, RNA, protein, and combinations thereof.

35. The nanosphere of claim 1, wherein the therapeutic agent is selected from the group consisting of paclitaxel, doxorubicin, temozolomide, 5-fluorouracil, camptothecin, and combinations thereof.

36. The nanosphere of claim 1, wherein the imaging agent is selected from the group consisting of: fluorescent dye, antibody against a protein overexpressed in cancer, and combinations thereof.
